SEQUENCE LISTING (52 sequences)

SEQ ID NO: 1 - DNA, 36462 bp, chimpanzee adenovirus serotype Pan5
  CDS (13898)..(15490): L2 Penton
catcatcaataatatacctcaaacttttggtgcgcgttaatatgcaaatgaggtatttgaatttggggatgcggggcggtgattggctgcgggagcggcgaccgttaggggcggggcgggtgacgttttgatgacgtggccgtgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaattccgcgtttatggcagggaggagtatttgccgagggccgagtagactttgaccgattacgtgggggtttcgattaccgtatttttcacctaaatttccgcgtacggtgtcaaagtccggtgtttttacgtaggtgtcagctgatcgccagggtatttaaacctgcgctctctagtcaagaggccactcttgagtgccagcgagtagagttttctcctccgcgccgcgagtcagatctacactttgaaagatgaggcacctgagagacctgcccggtaatgttttcctggctactgggaacgagattctggaactggtggtggacgccatgatgggtgacgaccctccggagccccctaccccatttgaagcgccttcgctgtacgatttgtatgatctggaggtggatgtgcccgagaacgaccccaacgaggaggcggtgaatgatttgtttagcgatgccgcgctgctggctgccgagcaggctaatacggactctggctcagacagcgattcctctctccataccccgagacccggcagaggtgagaaaaagatccccgagcttaaaggggaagagctcgacctgcgctgctatgaggaatgcttgcctccgagcgatgatgaggaggacgaggaggcgattcgagctgcagcgaaccagggagtgaaaacagcgagcgagggctttagcctggactgtcctactctgcccggacacggctgtaagtcttgtgaatttcatcgcatgaatactggagataagaatgtgatgtgtgccctgtgctatatgagagcttacaaccattgtgtttacagtaagtgtgattaactttagctggggaggcagagggtgactgggtgctgactggtttatttatgtatatgttttttatgtgtaggtcccgtctctgacgtagatgagacccccactacagagtgcatttcatcacccccagaaattggcgaggaaccgcccgaagatattattcatagaccagttgcagtgagagtcaccgggcgtagagcagctgtggagagtttggatgacttgctacagggtggggatgaacctttggacttgtgtacccggaaacgccccaggcactaagtgccacacatgtgtgtttacttaaggtgatgtcagtatttatagggtgtggagtgcaataaaatccgtgttgactttaagtgcgtggtttatgactcaggggtggggactgtgggtatataagcaggtgcagacctgtgtggtcagttcagagcaggactcatggagatctggacagtcttggaagactttcaccagactagacagctgctagagaactcatcggagggagtctcttacctgtggagattctgcttcggtgggcctctagctaagctagtctatagggccaagcaggattataaggatcaatttgaggatattttgagagagtgtcctggtatttttgactctctcaacttgggccatcagtctcactttaaccagagtattctgagagcccttgacttttctactcctggcagaactaccgccgcggtagccttttttgcctttatccttgacaaatggagtcaagaaacccatttcagcagggattaccgtctggactgcttagcagtagctttgtggagaacatggaggtgccagcgcctgaatgcaatctccggctacttgccagtacagccggtagacacgctgaggatcctgagtctccagtcaccccaggaacaccaacgccgccagcagccgcagcaggagcagcagcaagaggaggaccgagaagagaacctgagagccggtctggaccctccggtggcggaggaggaggagtagctgacttgtttcccgagctgcgccgggtgctgactaggtcttccagtggacgggagagggggattaagcgggagaggcatgaggagactagccacagaactgaactgactgtcagtctgatgagtcgcaggcgcccagaatcggtgtggtggcatgaggtgcagtcgcaggggatagatgaggtctcagtgatgcatgagaaatattccctagaacaagtcaagacttgttggttggagcccgaggatgattgggaggtagccatcaggaattatgccaagctggctctgaggccagacaagaagtacaagattaccaaactgattaatatcagaaattcctgctacatttcagggaatggggccgaggtggagatcagtacccaggagagggtggccttcagatgctgcatgatgaatatgtacccgggggtggtgggcatggagggagtcacctttatgaacgcgaggttcaggggtgatgggtataatggggtggtctttatggccaacaccaagctgacagtgcacggatgctccttctttggcttcaataacatgtgcattgaggcctggggcagtgtttcagtgaggggatgcagtttttcagccaactggatgggggtcgtgggcagaaccaagagcatggtgtcagtgaagaaatgcctgttcgagaggtgccacctgggggtgatgagcgagggcgaagccaaagtcaaacactgcgcctctaccgagacgggctgctttgtactgatcaagggcaatgccaaagtcaagcataatatgatctgtggggcctcggatgagcgcggctaccagatgctgacctgcgccggtgggaacagccatatgctagccaccgtgcatgtggcctcgcacccccgcaagacatggcccgagttcgagcacaacgtcatgacccgctgcaatgtgcacctggggtcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttgggactgggtgggagcctgcatgatgggcagaatgactaaaatctgtgtttttctgcgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcgaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgctgctgctgatggcccagctcgaggccctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgctgcacgatgtccttgaggaggagactgatggccacgggcagccccttggtgtaggtgttgacgaacctgttgagctgggagggatgcatgcggggggagatgagatgcatcttggcctggatcttgagattggcgatgttcccgcccagatcccgccgggggttcatgttgtgcaggaccaccagcacggtgtatccggtgcacttggggaatttgtcatgcaacttggaagggaaggcgtgaaagaatttggagacgcccttgtgaccgcccaggttttccatgcactcatccatgatgatggcgatgggcccgtgggcggcggcttgggcaaagacgtttcgggggtcggacacatcgtagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcagccggtggggccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgcccccaagcgagaggagctcttgcagcgaggcgaagtttttcagcggcttgagcccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccaggggcgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtctggccggtcggggtcaaaaacgaggtttcctccgtgctttttgatgcgtttcttacctctggtctccatgagctcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagagcagcttggcgatggagcgcatggtctggttcttttccttgtcggcgcgctccttggcggcgatgttgagctgcacgtactcgcgcgccacgcacttccattcggggaagacggtggtgagcttgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagatcgtccagcgccgcttgccagtcgcgcacggccagcgcgcgctcgtaggggctgaggggcgtgccccagggcatggggtgcgtgagcgcggaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagcccggtgccgaggttggagcgctgcggcttttcggcgcggtagacgatctggcggaagatggcgtgggagttggaggagatggtgggcctctggaagatgttgaagtgggcgtggggcagtccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgaccttgaggaactggtgcttgaagtcgaggtcgtcgcagccgccctgctcccagagctggaagtccgtgcgcttcttgtaggcggggttgggcaaagcgaaagtaacatcgttgaagaggatcttgcccgcgcggggcatgaagttgcgagtgatgcggaaaggctggggcacctcggcccggttgttgatgacctgggcggcgaggacgatctcgtcgaagccgttgatgttgtgcccgacgatgtagagttccacgaatcgcgggcggcccttgacgtggggcagcttcttgagctcgtcgtaggtgagctcggcggggtcgctgaggccgtgctgctcgagggcccagtcggcgaggtgggggttggcgccgaggaaggaagtccagagatccacggccagggcggtctgcaagcggtcccggtactgacggaactgctggcccacggccattttttcgggggtgacgcagtagaaggtgcgggggtcgccgtgccagcggtcccacttgagctggagggcgaggtcgtgggcgagctcgacgagcggcgggtccccggagagtttcatgaccagcatgaaggggacgagctgcttgccgaaggaccccatccaggtgtaggtttccacgtcgtaggtgaggaagagcctttcggtgcgaggatgcgagccgatggggaagaactggatctcctgccaccagttggaggaatggctgttgatgtgatggaagtagaaatgccgacggcgcgccgagcactcgtgcttgtgtttatacaagcgtccgcagtgctcgcaacgctgcacgggatgcacgtgctgcacgagctgtacctgggttcctttgacgaggaatttcagtgggcagtggagcgctggcggctgcatctggtgctgtactacgtcctggccatcggcgtggccatcgtctgcctcgatggtggtcatgctgacgaggccgcgcgggaggcaggtccagacctcggctcggacgggtcggagagcgaggacgagggcgcgcaggccggagctgtccagggtcctgagacgctgcggagtcaggtcagtgggcagcggcggcgcgcggttgacttgcaggagcttttccagggcgcgcgggaggtccagatggtacttgatctccacggcgccgttggtggcgacgtccacggcttgcagggtcccgtgcccctggggcgccaccaccgtgccccgtttcttcttgggtgctggcggcggcggctccatgcttagaagcggcggcgaggacgcgcgccgggcggcaggggcggctcggggcccggaggcaggggcggcaggggcacgtcggcgccgcgcgcgggcaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcgaccggcgcgctcgacggtggccgcgaggtcgttggagatgcggcccatgagctgcgagaaggcgttcatgccggcctcgttccagacgcggctgtagaccacggctccgtcggggtcgcgcgcgcgcatgaccacctgggcgaggttgagctcgacgtggcgcgtgaagaccgcgtagttgcagaggcgctggtagaggtagttgagcgtggtggcgatgtgctcggtgacgaagaagtacatgatccagcggcggagcggcatctcgctgacgtcgcccagggcttccaagcgctccatggcctcgtagaagtccacggcgaagttgaaaaactgggagttgcgcgccgagacggtcaactcctcctccagaagacggatgagctcggcgatggtggcgcgcacctcgcgctcgaaggccccggggggctcctcttcttccatctcctcctcctcttccatctcctccactaacatctcttctacttcctcctcaggaggcggcggcgggggaggggccctgcgtcgccggcggcgcacgggcagacggtcgatgaagcgctcgatggtctccccgcgccggcgacgcatggtctcggtgacggcgcgcccgtcctcgcggggccgcagcgtgaagacgccgccgcgcatctccaggtggccgccgggggggtctccgttgggcagggagagggcgctgacgatgcatcttatcaattggcccgtagggactccgcgcaaggacctgagcgtctcgagatccacgggatccgaaaaccgctgaacgaaggcttcgagccagtcgcagtcgcaaggtaggctgagcccggtttcttgttcttcgggtatttggtcgggaggcgggcgggcgatgctgctggtgatgaagttgaagtaggcggtcctgagacggcggatggtggcgaggagcaccaggtccttgggcccggcttgctggatgcgcagacggtcggccatgccccaggcgtggtcctgacacctggcgaggtccttgtagtagtcctgcatgagccgctccacgggcacctcctcctcgcccgcgcggccgtgcatgcgcgtgagcccgaacccgcgctgcggctggacgagcgccaggtcggcgacgacgcgctcggcgaggatggcctgctggatctgggtgagggtggtctggaagtcgtcgaagtcgacgaagcggtggtaggctccggtgttgatggtgtaggagcagttggccatgacggaccagttgacggtctggtggccggggcgcacgagctcgtggtacttgaggcgcgagtaggcgcgcgtgtcgaagatgtagtcgttgcaggtgcgcacgaggtactggtatccgacgaggaagtgcggcggcggctggcggtagagcggccatcgctcggtggcgggggcgccgggcgcgaggtcctcgagcatgaggcggtggtagccgtagatgtacctggacatccaggtgatgccggcggcggtggtggaggcgcgcgggaactcgcggacgcggttccagatgttgcgcagcggcaggaagtagttcatggtggccgcggtctggcccgtgaggcgcgcgcagtcgtggatgctctagacatacgggcaaaaacgaaagcggtcagcggctcgactccgtggcctggaggctaagcgaacgggttgggctgcgcgtgtaccccggttcgagtccctgctcgaatcaggctggagccgcagctaacgtggtactggcactcccgtctcgacccaagcctgctaacgaaacctccaggatacggaggcgggtcgttttggccattttcgtcaggccggaaatgaaactagtaagcgcggaaagcggccgtccgcgatggctcgctgccgtagtctggagaaagaatcgccagggttgcgttgcggtgtgccccggttcgagcctcagcgctcggcgccggccggattccgcggctaacgtgggcgtggctgccccgtcgtttccaagaccccttagccagccgacttctccagttacggagcgagcccctctttttcttgtgtttttgccagatgcatcccgtactgcggcagatgcgcccccaccctccaccacaaccgcccctaccgcagcagcagcaacagccggcgcttctgcccccgccccagcagcagcagccagccactaccgcggcggccgccgtgagcggagccggcgttcagtatgacctggccttggaagagggcgaggggctggcgcggctgggggcgtcgtcgccggagcggcacccgcgcgtgcagatgaaaagggacgctcgcgaggcctacgtgcccaagcagaacctgttcagagacaggagcggcgaggagcccgaggagatgcgcgcctcccgcttccacgcggggcgggagctgcggcgcggcctggaccgaaagcgggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcacgctgatcgcgcgcgaggaggtgaccctgggcctgatgcacctgtgggacctgctggaggccatcgtgcagaaccccacgagcaagccgctgacggcgcagctgtttctggtggtgcagcacagtcgggacaacgagacgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagcctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagccgccggcgcgagctgagcgaccaggagctgatgcacagcctgcagcgggccctgaccggggccgggaccgagggggagagctactttgacatgggcgcggacctgcgctggcagcctagccgccgggccttggaagctgccggcggttccccctacgtggaggaggtggacgatgaggaggaggagggcgagtacctggaagactgatggcgcgaccgtatttttgctagatgcagcaacagccaccgccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccgactctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagacgaacctggaccgcatggtgaccgacgtgcgcgaggcggtgtcgcagcgcgagcggttccaccgcgagtcgaacctgggctccatggtggcgctgaacgccttcctgagcacgcagcccgccaacgtgccccggggccaggaggactacaccaacttcatcagcgcgctgcggctgatggtggccgaggtgccccagagcgaggtgtaccagtcggggccggactacttcttccagaccagtcgccagggcttgcagaccgtgaacctgagccaggctttcaagaacttgcagggactgtggggcgtgcaggccccggtcggggaccgcgcgacggtgtcgagcctgctgacgccgaactcgcgcctgctgctgctgctggtggcgcccttcacggacagcggcagcgtgagccgcgactcgtacctgggctacctgcttaacctgtaccgcgaggccatcgggcaggcgcacgtggacgagcagacctaccaggagatcacccacgtgagccgcgcgctgggccaggaggacccgggcaacctggaggccaccctgaacttcctgctgaccaaccggtcgcagaagatcccgccccagtacgcgctgagcaccgaggaggagcgcatcctgcgctacgtgcagcagagcgtggggctgttcctgatgcaggagggggccacgcccagcgccgcgctcgacatgaccgcgcgcaacatggagcccagcatgtacgcccgcaaccgcccgttcatcaataagctgatggactacttgcatcgggcggccgccatgaactcggactactttaccaacgccatcttgaacccgcactggctcccgccgcccgggttctacacgggcgagtacgacatgcccgaccccaacgacgggttcctgtgggacgacgtggacagcagcgtgttctcgccgcgccccaccaccaccaccgtgtggaagaaagagggcggggaccggcggccgtcctcggcgctgtccggtcgcgcgggtgctgccgcggcggtgcccgaggccgccagccccttcccgagcctgcccttttcgctgaacagcgtgcgcagcagcgagctgggtcggctgacgcggccgcgcctgctgggcgaggaggagtacctgaacgactccttgcttcggcccgagcgcgagaagaacttccccaataacgggatagagagcctggtggacaagatgagccgctggaagacgtacgcgcacgagcacagggacgagccccgagctagcagcagcaccggcgccacccgtagacgccagcggcacgacaggcagcggggtctggtgtgggacgatgaggattccgccgacgacagcagcgtgttggacttgggtgggagtggtggtggtaacccgttcgctcacctgcgcccccgtatcgggcgcctgatgtaagaatctgaaaaaataaaagacggtactcaccaaggccatggcgaccagcgtgcgttcttctctgttgtttgtagtagtatgatgaggcgcgtgtacccggagggtcctcctccctcgtacgagagcgtgatgcagcaggcggtggcggcggcgatgcagcccccgctggaggcgccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaacgatttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcgcgcaagacccccaacggggtcacagtaacagatggtagtcaggacgagctgacctacgagtgggtggagtttgagctgcccgagggcaacttctcggtgaccatgaccatcgatctgatgaacaacgccatcatcgacaactacttggcggtggggcggcagaacggggtgctggagagcgacatcggcgtgaagttcgacacgcgcaacttccggctgggctgggaccccgtgaccgagctggtgatgccgggcgtgtacaccaacgaggccttccaccccgacatcgtcctgctgcccggctgcggcgtggacttcaccgagagccgcctcagcaacctgctgggcatccgcaagcggcagcccttccaggagggcttccagatcctgtacgaggacctggaggggggcaacatccccgcgctgctggacgtggacgcctacgagaaaagcaaggaggatagcgccgccgcggcgaccgcagccgtggccaccgcctctaccgaggtgcggggcgataattttgctagcgccgcgacactggcagcggccgaggcggctgaaaccgaaagtaagatagtgatccagccggtggagaaggacagcaaggagaggagctacaacgtgctcgcggacaagaaaaacaccgcctaccgcagctggtacctggcctacaactacggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgttcgcccgcccgcgcccaccattaccaccgtcagtgaaaacgttcctgctctcacagatcacgggaccctgccgctgcgcagcagtatccggggagtccagcgcgtgaccgtcactgacgccagacgccgcacctgcccctacgtctacaaggccctgggcgtagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcgctcgcgcaccaccgtcgacgacgtgatcgaccaggtggtggccgacgcgcgcaactacacgcccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcctccggcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaattcaaggaagagatgctccaggtcatcgcgcctgagatctacggcccggcggcggtgaaggaggaaagaaagccccgcaaactgaagcgggtcaaaaaggacaaaaaggaggaggaagatgtggacggactggtggagtttgtgcgcgagttcgccccccggcggcgcgtgcagtggcgcgggcggaaagtgaaaccggtgctgcgacccggcaccacggtggtcttcacgcccggcgagcgttccggctccgcctccaagcgctcctacgacgaggtgtacggggacgaggacatcctcgagcaggcggccgaacgtctgggcgagtttgcttacggcaagcgcagccgccccgcgcccttgaaagaggaggcggtgtccatcccgctggaccacggcaaccccacgccgagcctgaagccggtgaccctgcagcaggtgctgcctggtgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccggcaccggcttccaccacccgccgaagacgcaagtacggcgcggccagcctgctgatgcccaactacgcgctgcatccttccatcatccccacgccgggctaccgcggcacgcgcttctaccgcggctacaccagcagccgccgccgcaagaccaccacccgccgccgccgtcgtcgcacccgccgcagcagcaccgcgacttccgccgccgccctggtgcggagagtgtaccgcagcgggcgcgagcctctgaccctgccgcgcgcgcgctaccacccgagcatcgccatttaactaccgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatgcccatcatcgccgcggcgatcggggcgatccccggcatagcttccgtggcggtgcaggcctctcagcgccactgagacacagcttggaaaatttgtaataaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagcagccagcccgcgaccctggacttgcctccgcctgcttcccgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtatatgtatgtccgccgaccagaaggaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcttacaacagcctggctcccaagggagcgcccaacacttgccagtggacatataaagctgatggtgatactggtacagaaaaaacctatacatatggaaatgcgcctgtgcaaggcattagtattacaaaagatggtattcaacttggaactgacactgatgatcagcccatttatgcagataaaacttatcaaccagagcctcaagtgggtgatgctgaatggcatgacatcactggtactgatgaaaaatatggaggcagagctctcaagcctgacaccaaaatgaagccctgctatggttcttttgccaagcctaccaataaagaaggaggtcaggcaaatgtgaaaaccgaaacaggcggtaccaaagaatatgacattgacatggcattcttcgataatcgaagtgcagctgcggctggcctggccccagaaattgttttgtatactgagaatgtggatctggaaactccagatactcatattgtatacaaggcgggcacagatgacagcagctcttctatcaatttgggtcagcagtccatgcccaacagacccaactacattggctttagagacaactttatcgggctcatgtactacaacagcactggcaacatgggcgtgctggctggtcaggcctcccagctgaatgctgtggtggacttgcaggacagaaacactgaactgtcctaccagctcttgcttgactctctgggcgacagaaccaggtatttcagtatgtggaatcaggcggtggacagctatgaccccgatgtgcgcattattgaaaatcacggtgtggaggatgaactccctaactattgcttccccctggatgctgtgggtagaactgatacttaccagggaattaaggccaatggtgctgatcaaaccacctggaccaaagatgatactgttaatgatgctaatgaattgggcaagggcaatcctttcgccatggagatcaacatccaggccaacctgtggcggaacttcctctacgcgaacgtggcgctgtacctgcccgactcctacaagtacacgccggccaacatcacgctgccgaccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagttcttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagaggggtacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggacagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacgcccttctcggccggcaacgccaccacctaagccccgctcttgcttcttgcaagatgacggcctgtgcgggctccggcgagcaggagctcagggccatcctccgcgacctgggctgcgggccctgcttcctgggcaccttcgacaagcgcttcccgggattcatggccccgcacaagctggcctgcgccatcgtcaacacggccggccgcgagaccgggggcgagcactggctggccttcgcctggaacccgcgctcccacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgcctactttcgctcccaccgcgcgcgcatcgagaaggccaccgccttcgaccgcatgaatcaagacatgtaaaccgtgtgtgtatgtgaatgctttattcataataaacagcacatgtttatgccaccttttctgaggctctgactttatttagaaatcgaaggggttctgccggctctcggcgtgccccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgccctccacgtccagatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcatcgcagcttgccctcggcctcggtgcacccgtgcagccacagcgcgcagccggtgcactcccagttcttgtgggcgatctgggagtgcgagtgcacgaagccctgcaggaagcggcccatcatcgtggtcagggtcttgttgctggtgaaggtcagcgggatgccgcggtgctcctcgttcacatacaggtggcagatgcggcggtacacctcgccctgctcgggcatcagctggaaggcggacttcaggtcgctctccacgcggtaccggtccatcagcagcgtcatgacttccatgcccttctcccaggccgagacgatcggcaggctcagggggttcttcaccgccgttgtcatcttagtcgccgccgctgaggtcagggggtcgttctcgtccagggtctcaaacactcgcttgccgtccttctcggtgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttcttcttggccgtcgtccgagaccacgcggcggtaggcatgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaacaagcatggagactcagccatcgtcgccaacatcgccatctgcccccgccgccgccgacgagaaccagcagcagaatgaaagcttaaccgccccgccgcccagccccacctccgacgccgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagcagcaggctgggctcgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggtcccggcgcccgcctacctgatatcgcctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtgctcaagcgcacggtcgagctgacccacttcgcctacccggcgctcaacctgccccccaaggtcatgagcgccgtcatggaccaggtgctcatcaagcgcgcctcgcccctctcggatgaggacatgcaggaccccgagagctcggacgagggcaagcccgtggtcagcgacgagcagctggcgcgctggctgggagcgagtagcaccccccagagcttggaagagcggcgcaagctcatgatggccgtggtcctggtgaccgtggagctggagtgtctgcgccgcttcttcgccgacgcagagaccctgcgcaaggtcgaggagaacctgcactacctcttcaggcacgggtttgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctgaaggccctgtggaccgggttcgacgagcgcaccaccgcctcggacctggccgacctcatcttccccgagcgcctgcggctgacgctgcgcaacggactgcccgactttatgagtcaaagcatgttgcaaaactttcgctctttcatcctcgaacgctccgggatcctgcccgccacctgctccgcgctgccctcggacttcgtgccgctgaccttccgcgagtgccccccgccgctctggagccactgctacctgctgcgcctggccaactacctggcctaccactcggacgtgatcgaggacgtcagcggcgagggtctgctcgagtgccactgccgctgcaacctctgcacgccgcaccgctccctggcctgcaacccccagctgctgagcgagacccagatcatcggcaccttcgagttgcaaggccccggcgagggcaaggggggtctgaaactcaccccggggctgtggacctcggcctacttgcgcaagttcgtgcccgaggactaccatcccttcgagatcaggttctacgaggaccaatcccagccgcccaaggccgaactgtcggcctgcgtcatcacccagggggccatcctggcccaattgcaagccatccagaaatcccgccaagaatttctgctgaaaaagggccacggggtctacctggacccccagaccggagaggagctcaaccccagcttcccccaggatgccccgaggaagcagcaagaagctgaaagtggagctgccgccgccggaggatttggaggaagactgggagagcagtcaggcagaggaggaggagatggaagactgggacagcactcaggcagaggaggacagcctgcaagacagtctggaagacgaggtggaggaggaggcagaggaagaagcagccgccgccagaccgtcgtcctcggcggagaaagcaagcagcacggataccatctccgctccgggtcggggtcgcggcgaccgggcccacagtaggtgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccgccgctacctgctcttccaccgcggggtgaacttcccccgcaacatcttgcattactaccgtcacctccacagcccctactactgtttccaagaagaggcagaaacccagcagcagcagaaaaccagcggcagcagcagctagaaaatccacagcggcggcaggtggactgaggatcgcagcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagcgccgggcccgcgatgatctcacgggtgaatgacatccgcgcccgccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccggcccacggagtgcggatcatcgtcgaagggggcctcgactcccacctgcttcggatcttcagccagcgaccgatcctggtcgagcgcgagcaaggacagacccttctgaccctgtactgcatctgcaaccaccccggcctgcatgaaagtctttgttgtctgctgtgtactgagtataataaaagctgagatcagcgactactccggactcgattgtggtgttcctgctatcaaccggtccctgttcttcaccgggaacgagaccgagctccagcttcagtgtaagccccacaagaagtacctcacctggctgttccagggctccccgatcgccgttgtcaaccactgcgacaacgacggagtcctgctgagcggccccgccaaccttactttttccacccgcagaagcaagctccagctcttccaacccttcctccccgggacctatcagtgcgtctcgggaccctgccatcacaccttccacctgatcccgaataccacagcgccgctccccgctactaacaaccaaactacccaccatcgccaccgtcgcgacctttctgaatctaacactaccacccacaccggaggtgagctccgaggtcgaccaacctctgggatttactacggcccctgggaggtggtggggttaatagcgctaggcctagttgtgggtgggcttttggctctctgctacctatacctcccttgctgttcgtacttagtggtgctgtgttgctggtttaagaaatggggaagatcaccctagtgagctgcggtgcgctggtggcggtggtggtgttttcgattgtgggactgggcggcgcggctgtagtgaaggagaaggccgatccctgcttgcatttcaatcccgacaattgccagctgagttttcagcccgatggcaatcggtgcgcggtgctgatcaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaacaatactttcatttttgcgcacatgtgcgacacggtcatgtggatgagcaagcagtacgatatgtggccccccacgaaggagaacatcgtggtcttctccatcgcttacagcgcgtgcacggcgctaatcaccgctatcgtgtgcctgagcattcacatgctcatcgctattcgccccagaaataatgccgaaaaagagaaacagccataacacgttttttcacacacctttttcagaccatggcctctgttaaatttttgcttttatttgccagtctcattactgttataagtaatgagaaactcactatttacattggcactaaccacactttagacggaattccaaaatcctcatggtattgctattttgatcaagatccagacttaactatagaactgtgtggtaacaagggaaaaaatacaagcattcatttaattaactttaattgcggagacaatttgaaattaattaatatcactaaagagtatggaggtatgtattactatgttgcagaaaataacaacatgcagttttatgaagttactgtaactaatcccaccacacctagaacaacaacaaccaccaccacaaaaactacacctgttaccactatgcagctcactaccaataacatttttgccatgcgtcaaatggtcaacaatagcactcaacccaccccacccagtgaggaaattcccaaatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggccttttaattttttctatcattacctctgctctatgcaattctgacaatgaggacgttactgtcgttgtcggaaccaattatacactgaaaggtccagcgaagggtatgctttcgtggtattgctggtttggaactgacgagcaacagacagagctctgcaatgctcaaaaaggcaaaacctcaaattctaaaatctctaattatcaatgcaatggcactgacttagtactgctcaatgtcacgaaagcatatgctggcagctacacctgccctggagatgatactgagaacatgattttttacaaagtggaagtggttgatcccactactccacctccacccaccacaactactcacaccacacacacagaacaaaccacagcagaggaggcagcaaagttagccttgcaggtccaagacagttcatttgttggcattacccctacacctgatcagcggtgtccggggctgctcgtcagcggcattgtcggtgtgctttcgggattagcagtcataatcatctgcatgttcatttttgcttgctgctatagaaggctttaccgacaaaaatcagacccactgctgaacctctatgtttaattttttccagagccatgaaggcagttagcactctagttttttgttctttgattggcactgtttttagtgttagctttttgaaacaaatcaatgttactgagggggaaaatgtgacactggtaggcgtagagggtgctcaaaataccacctggacaaaattccatctagatgggtggaaagaaatttgcacctggaatgtcagtacttatacatgtgaaggagttaatcttaccattgtcaatgtcagccaaattcaaaagggttggattaaagggcaatctgttagtgttagcaatagtgggtactatacccagcatactcttatctatgacattatagttataccactgcctacacctagcccacctagcactaccacacagacaacccacactacacaaacaaccacatacagtacatcaaatcagcctaccaccactacaacagcagaggttgccagctcgtctggggtccgagtggcatttttgatgttggccccatctagcagtcccactgctagtaccaatgagcagactactgaatttttgtccactgtcgagagccacaccacagctacctcgagtgccttctctagcaccgccaatctatcctcgctttcctctacaccaatcagtcccgctactactcctacccccgctattctccccactcccctgaagcaaacagacggcgacatgcaatggcagatcaccctgctcattgtgatcgggttggtcatcctggccgtgttgctctactacatcttctgccgccgcattcccaacgcgcaccgcaagccggcctacaagcccatcgttgtcgggcagccggagccgcttcaggtggaagggggtctaaggaatcttctcttctcttttacagtatggtgattgaattatgattcctagacaaatcttgatcactattcttatctgcctcctccaagtctgtgccaccctcgctctggtggccaacgccagtccagactgtattgggcccttcgcctcctacgtgctctttgccttcatcacctgcatctgctgctgtagcatagtctgcctgcttatcaccttcttccagttcattgactggatctttgtgcgcatcgcctacctgcgccaccacccccagtaccgcgaccagcgagtggcgcggctgctcaggatcctctgataagcatgcgggctctgctacttctcgcgcttctgctgttagtgctcccccgtcccgtcgacccccggacccccacccagtcccccgaggaggtccgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcaacctcaggcacacgcactaccaccaccaccacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggacggcatagccatccaccagtgcaagaaaggcatcttctgcctggtgaaacaggccaagatctcctacgaggtcacccagaccgaccatcgcctctcctacgagctcctgcagcagcgccagaagttcacctgcctggtcggagtcaaccccatcgtcatcacccagcagtcgggcgataccaaggggtgcatccactgctcctgcgactcccccgactgcgtccacactctgatcaagaccctctgcggcctccgcgacctcctccccatgaactaatcacccccttatccagtgaaataaagatcatattgatgatttgagtttaataaaaataaagaatcacttacttgaaatctgataccaggtctctgtccatgttttctgccaacaccacttcactcccctcttcccagctctggtactgcaggccccggcgggctgcaaacttcctccacaccctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgctgtccctgcgtctggccgatcccgtcaccaccaagaacggggaaatcaccctcaagctgggagatggggtggacctcgactcctcgggaaaactcatctccaacacggccaccaaggccgccgcccctctcagtttttccaacaacaccatttcccttaacatggatacccctttttacaacaacaatggaaagttaggcatgaaagtcactgctccactgaagatactagacacagacttgctaaaaacacttgttgtagcttatggacaaggtttaggaacaaacaccactggtgcccttgttgcccaactagcatccccacttgcttttgatagcaatagcaaaattgcccttaatttaggcaatggaccattgaaagtggatgcaaatagactgaacatcaattgcaatagaggactctatgttactaccacaaaagatgcactggaagccaatataagttgggctaatgctatgacatttataggaaatgccatgggtgtcaatattgatacacaaaaaggcttgcaatttggcaccactagtaccgtcgcagatgttaaaaacgcttaccccatacaaatcaaacttggagctggtctcacatttgacagcacaggtgcaattgttgcatggaacaaagatgatgacaagcttacactatggaccacagccgacccctctccaaattgtcacatatattctgaaaaggatgctaagcttacactttgcttgacaaagtgtggcagtcagattctgggcactgtttccctcatagctgttgatactggcagtttaaatcccataacaggaacagtaaccactgctcttgtctcacttaaattcgatgcaaatggagttttgcaaagcagctcaacactagactcagactattggaatttcagacagggagatgttacacctgctgaagcctatactaatgctataggtttcatgcccaatctaaaagcataccctaaaaacacaagtggagctgcaaaaagtcacattgttgggaaagtgtacctacatggggatacaggcaaaccactggacctcattattactttcaatgaaacaagtgatgaatcttgcacttactgtattaactttcaatggcagtggggggctgatcaatataaaaatgaaacacttgccgtcagttcattcaccttttcctatattgctaaagaataaaccccactctgtaccccatctctgtctatggaaaaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgtcgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacaggaccaccaggttgttcaacagtccatagttcaacacgctccagccgaaactcatcgcgggaaggatgctacccacgtggccgtcgtaccagatcctcaggtaaatcaagtggcgccccctccagaacacgctgcccatgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccgacaatggcaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgttatgatgcagttgctttcggacattttcgtacttgctgtagcagaacctggtccgggcgctgcacaccgatcgccggcggcggtcccggcgcttggaacgctcggtgttgaagttgtaaaacagccactctctcagaccgtgcagcagatctagggcctcaggagtgatgaagatcccatcatgcctgatggctctaatcacatcgaccaccgtggaatgggccagacccagccagatgatgcaattttgttgggtttcggtgacggcgggggagggaagaacaggaagaaccatgattaacttttaatccaaacggtctcggagcacttcaaaatgaagatcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctaagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcgcccaggcaatttttaagaaaatcaacaaaagaaaaatcctccaggtgcacgtttagagcctcgggaacaacgatggagtaaatgcaagcggtgcgttccagcatggttagttagctgatctgtagaaaaaaacaaaaatgaacattaaaccatgctagcctggcgaacaggtgggtaaatcgttctctccagcaccaggcaggccacggggtctccggcacgaccctcgtaaaaattgtcgctatgattgaaaaccatcacagagagacgttcccggtggccggcgtgaatgattcgacaagatgaatacacccccggaacattggcgtccgcgagtgaaaaaaagcgcccaaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaactgccgtcatttccgggttcccacgctacgtcatcaaaattcgactttcaaattccgtcgaccgttaaaaacgtcgcccgccccgcccctaacggtcgccgctcccgcagccaatcaccgccccgcatccccaaattcaaatacctcatttgcatattaacgcgcaccaaaagtttgaggtatattattgatgatg

SEQ ID NO: 2 - protein, 530 aa, chimpanzee adenovirus serotype Pan5
MMRRVYPEGPPPSYESVMQQAVAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVTVTDGSQDELTYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQILYEDLEGGNIPALLDVDAYEKSKEDSAAAATAAVATASTEVRGDNFASAATLAAAEAAETESKIVIQPVEKDSKERSYNVLADKKNTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGVVAPRVLSSRTF

SEQ ID NO: 3 - protein, 933 aa, chimpanzee adenovirus serotype Pan5
MATPSMLPQWAYMHIAGQDASEYLSPGLVQFARATDTYFSLGNKFRNPTVAPTHDVTTDRSQRLTLRFVPVDREDNTYSYKVRYTLAVGDNRVLDMASTYFDIRGVLDRGPSFKPYSGTAYNSLAPKGAPNTCQWTYKADGDTGTEKTYTYGNAPVQGISITKDGIQLGTDTDDQPIYADKTYQPEPQVGDAEWHDITGTDEKYGGRALKPDTKMKPCYGSFAKPTNKEGGQANVKTETGGTKEYDIDMAFFDNRSAAAAGLAPEIVLYTENVDLETPDTHIVYKAGTDDSSSSINLGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDAVGRTDTYQGIKANGADQTTWTKDDTVNDANELGKGNPFAMEINIQANLWRNFLYANVALYLPDSYKYTPANITLPTNTNTYDYMNGRVVAPSLVDAYINIGARWSLDPMDNVNPFNHHRNAGLRYRSMLLGNGRYVPFHIQVPQKFFAIKSLLLLPGSYTYEWNFRKDVNMILQSSLGNDLRTDGASIAFTSINLYATFFPMAHNTASTLEAMLRNDTNDQSFNDYLSAANMLYPIPANATNVPISIPSRNWAAFRGWSFTRLKTRETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVASVTQKKFLCDRVMWRIPFSSNFMSMGALTDLGQNMLYANSAHALDMNFEVDPMDESTLLYVVFEVFDVVRVHQPHRGVIEAVYLRTPFSAGNATT

SEQ ID NO: 4 - protein, 445 aa, chimpanzee adenovirus serotype Pan5
MSKKRVRVDDDFDPVYPYDADNAPTVPFINPPFVSSDGFQEKPLGVLSLRLADPVTTKNGEITLKLGDGVDLDSSGKLISNTATKAAAPLSFSNNTISLNMDTPFYNNNGKLGMKVTAPLKILDTDLLKTLVVAYGQGLGTNTTGALVAQLASPLAFDSNSKIALNLGNGPLKVDANRLNINCNRGLYVTTTKDALEANISWANAMTFIGNAMGVNIDTQKGLQFGTTSTVADVKNAYPIQIKLGAGLTFDSTGAIVAWNKDDDKLTLWTTADPSPNCHIYSEKDAKLTLCLTKCGSQILGTVSLIAVDTGSLNPITGTVTTALVSLKFDANGVLQSSSTLDSDYWNFRQGDVTPAEAYTNAIGFMPNLKAYPKNTSGAAKSHIVGKVYLHGDTGKPLDLIITFNETSDESCTYCINFQWQWGADQYKNETLAVSSFTFSYIAKE

SEQ ID NO: 5 - DNA, 36604 bp, chimpanzee adenovirus serotype Pan6
  CDS (13878)..(15467): L2 Penton
catcatcaataatatacctcaaacttttggtgcgcgttaatatgcaaatgagctgtttgaatttggggagggaggaaggtgattggctgcgggagcggcgaccgttaggggcggggcgggtgacgttttgatgacgtggctatgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacgtaggcgtcagctgatcgccagggtatttaaacctgcgctctctagtcaagaggccactcttgagtgccagcgagtagagttttctcctccgcgccgcgagtcagatctacactttgaaagatgaggcacctgagagacctgcccggtaatgttttcctggctactgggaacgagattctggaattggtggtggacgccatgatgggtgacgaccctccagagccccctaccccatttgaggcgccttcgctgtacgatttgtatgatctggaggtggatgtgcccgagagcgaccctaacgaggaggcggtgaatgatttgtttagcgatgccgcgctgctggctgccgagcaggctaatacggactctggctcagacagcgattcctctctccataccccgagacccggcagaggtgagaaaaagatccccgagcttaaaggggaagagctcgacctgcgctgctatgaggaatgcttgcctccgagcgatgatgaggaggacgaggaggcgattcgagctgcggtgaaccagggagtgaaaactgcgggcgagagctttagcctggactgtcctactctgcccggacacggctgtaagtcttgtgaatttcatcgcatgaatactggagataagaatgtgatgtgtgccctgtgctatatgagagcttacaaccattgtgtttacagtaagtgtgattaactttagttgggaaggcagagggtgactgggtgctgactggtttatttatgtatatgtttttttatgtgtaggtcccgtctctgacgtagatgagacccccacttcagagtgcatttcatcacccccagaaattggcgaggaaccgcccgaagatattattcatagaccagttgcagtgagagtcaccgggcggagagcagctgtggagagtttggatgacttgctacagggtggggatgaacctttggacttgtgtacccggaaacgccccaggcactaagtgccacacatgtgtgtttacttaaggtgatgtcagtatttatagggtgtggagtgcaataaaatccgtgttgactttaagtgcgtgttttatgactcaggggtggggactgtgggtatataagcaggtgcagacctgtgtggtcagttcagagcaggactcatggagatctggactgtcttggaagactttcaccagactagacagttgctagagaactcatcggagggagtctcttacctgtggagattctgcttcggtgggcctctagctaagctagtctatagggccaaacaggattataaggaacaatttgaggatattttgagagagtgtcctggtatttttgactctctcaacttgggccatcagtctcactttaaccagagtattctgagagcccttgacttttctactcctggcagaactaccgccgcggtagccttttttgcctttattcttgacaaatggagtcaagaaacccatttcagcagggattaccgtctggactgcttagcagtagctttgtggagaacatggaggtgccagcgcctgaatgcaatctccggctacttgccagtacagccggtagacacgctgaggatcctgagtctccagtcaccccaggaacaccaacgccgccagcagccgcagcaggagcagcagcaagaggaggaccgagaagagaacccgagagccggtctggaccctccggtggcggaggaggaggagtagctgacttgtttcccgagctgcgccgggtgctgactaggtcttccagtggacgggagagggggattaagcgggagaggcatgaggagactagccacagaactgaactgactgtcagtctgatgagccgcaggcgcccagaatcggtgtggtggcatgaggtgcagtcgcaggggatagatgaggtctcggtgatgcatgagaaatattccctagaacaagtcaagacttgttggttggagcccgaggatgattgggaggtagccatcaggaattatgccaagctggctctgaagccagacaagaagtacaagattaccaaactgattaatatcagaaattcctgctacatttcagggaatggggccgaggtggagatcagtacccaggagagggtggccttcagatgttgtatgatgaatatgtacccgggggtggtgggcatggagggagtcacctttatgaacacgaggttcaggggtgatgggtataatggggtggtctttatggccaacaccaagctgacagtgcacggatgctccttctttggcttcaataacatgtgcatcgaggcctggggcagtgtttcagtgaggggatgcagcttttcagccaactggatgggggtcgtgggcagaaccaagagcaaggtgtcagtgaagaaatgcctgttcgagaggtgccacctgggggtgatgagcgagggcgaagccaaagtcaaacactgcgcctctaccgagacgggctgctttgtgctgatcaagggcaatgcccaagtcaagcataacatgatctgtggggcctcggatgagcgcggctaccagatgctgacctgcgccggtgggaacagccatatgctggccaccgtgcatgtggcctcgcacccccgcaagacatggcccgagttcgagcacaacgtcatgacccgctgcaatgtgcacctgggctcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttggggctgggtgtgagcctgcatgaggggcagaatgactaaaatctgtggttttctgtgtgttgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcgaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgctgctgctgatggcccagctcgaggccctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcggggatggtgttgtaaatcacccagtcatagcaggggcgcagggcgtggtgctgcacgatgtccttgaggaggagactgatggccacgggcagccccttggtgtaggtgttgacgaacctgttgagctgggagggatgcatgcggggggagatgagatgcatcttggcctggatcttgagattggcgatgttcccgcccagatcccgccgggggttcatgttgtgcaggaccaccagcacggtgtatccggtgcacttggggaatttgtcatgcaacttggaagggaaggcgtgaaagaatttggagacgcccttgtgaccgcccaggttttccatgcactcatccatgatgatggcgatgggcccgtgggcggcggcctgggcaaagacgtttcgggggtcggacacatcgtagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcaaccggtggggccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagtccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccagggccgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcggggcggttggggtcaaaaacgaggtttcctccgtgctttttgatgcgtttcttacctctggtctccatgagctcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatttggtcagaaaagacgatctttttgttgtcgagcttggtggcgaaggagccgtagagggcgttggagagcagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagattgtccagcgccgcttgccagtcgcgcacggccagcgcgcgctcgtaggggctgaggggcgtgccccagggcatggggtgcgtgagcgcggaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgtgccgaggttggagcgttgcggcttttcggcgcggtagacgatctggcggaagatggcgtgggagttggaggagatggtgggcctttggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcatacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgaccttgaggaactggtgcttgaagtcgaggtcgtcgcagccgccctgctcccagagttggaagtccgtgcgcttcttgtaggcggggttaggcaaagcgaaagtaacatcgttgaagaggatcttgcccgcgcggggcatgaagttgcgagtgatgcggaaaggctggggcacctcggcccggttgttgatgacctgggcggcgaggacgatctcgtcgaagccgttgatgttgtgcccgacgatgtagagttccacgaatcgcgggcggcccttgacgtggggcagcttcttgagctcgtcgtaggtgagctcggcggggtcgctgagcccgtgctgctcgagggcccagtcggcgacgtgggggttggcgctgaggaaggaagtccagagatccacggccagggcggtctgcaagcggtcccggtactgacggaactgttggcccacggccattttttcgggggtgacgcagtagaaggtgcgggggtcgccgtgccagcggtcccacttgagctggagggcgaggtcgtgggcgagctcgacgagcggcgggtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccagttggaggaatggctgttgatgtgatggaagtagaaatgccgacggcgcgccgagcactcgtgcttgtgtttatacaagcgtccgcagtgctcgcaacgctgcacgggatgcacgtgctgcacgagctgtacctgggttcctttggcgaggaatttcagtgggcagtggagcgctggcggctgcatctcgtgctgtactacgtcttggccatcggcgtggccatcgtctgcctcgatggtggtcatgctgacgagcccgcgcgggaggcaggtccagacctcggctcggacgggtcggagagcgaggacgagggcgcgcaggccggagctgtccagggtcctgagacgctgcggagtcaggtcagtgggcagcggcggcgcgcggttgacttgcaggagcttttccagggcgcgcgggaggtccagatggtacttgatctccacggcgccgttggtggctacgtccacggcttgcagggtgccgtgcccctggggcgccaccaccgtgccccgtttcttcttgggcgctgcttccatgtcggtcagaagcggcggcgaggacgcgcgccgggcggcaggggcggctcggggcccggaggcaggggcggcaggggcacgtcggcgccgcgcgcgggcaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctcgacggtggccgcgaggtcgttggagatgcggcccatgagctgcgagaaggcgttcatgccggcctcgttccagacgcggctgtagaccacggctccgtcggggtcgcgcgcgcgcatgaccacctgggcgaggttgagctcgacgtggcgcgtgaagaccgcgtagttgcagaggcgctggtagaggtagttgagcgtggtggcgatgtgctcggtgacgaagaagtacatgatccagcggcggagcggcatctcgctgacgtcgcccagggcttccaagcgttccatggcctcgtagaagtccacggcgaagttgaaaaactgggagttgcgcgccgagacggtcaactcctcctccagaagacggatgagctcggcgatggtggcgcgcacctcgcgctcgaaggccccggggggctcctcttccatctcctcctcttcctcctccactaacatctcttctacttcctcctcaggaggcggtggcgggggaggggccctgcgtcgccggcggcgcacgggcagacggtcgatgaagcgctcgatggtctccccgcgccggcgacgcatggtctcggtgacggcgcgcccgtcctcgcggggccgcagcatgaagacgccgccgcgcatctccaggtggccgccgggggggtctccgttgggcagggagagggcgctgacgatgcatcttatcaattgacccgtagggactccgcgcaaggacctgagcgtctcgagatccacgggatccgaaaaccgctgaacgaaggcttcgagccagtcgcagtcgcaaggtaggctgagcccggtttcttgttcttcgggtatttggtcgggaggcgggcgggcgatgctgctggtgatgaagttgaagtaggcggtcctgagacggcggatggtggcgaggagcaccaggtccttgggcccggcttgctggatgcgcagacggtcggccatgccccaggcgtggtcctgacacctggcgaggtccttgtagtagtcctgcatgagccgctccacgggcacctcctcctcgcccgcgcggccgtgcatgcgcgtgagcccgaacccgcgctgcggctggacgagcgccaggtcggcgacgacgcgctcggtgaggatggcctgctggatctgggtgagggtggtctggaagtcgtcgaagtcgacgaagcggtggtaggctccggtgttgatggtgtaggagcagttggccatgacggaccagttgacggtctggtggccgggtcgcacgagctcgtggtacttgaggcgcgagtaggcgcgcgtgtcgaagatgtagtcgttgcaggcgcgcacgaggtactggtatccgacgaggaagtgcggcggcggctggcggtagagcggccatcgctcggtggcgggggcgccgggcgcgaggtcctcgagcatgaggcggtggtagccgtagatgtacctggacatccaggtgatgccggcggcggtggtggaggcgcgcgggaactcgcggacgcggttccagatgttgcgcagcggcaggaagtagttcatggtggccgcggtctggcccgtgaggcgcgcgcagtcgtggatgctctagacatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgctaacgaaacctccaggatacggaggcgggtcgttttttggccttggtcgctggtcatgaaaaactagtaagcgcggaaagcggccgcccgcgatggctcgctgccgtagtctggagaaagaatcgccagggttgcgttgcggtgtgccccggttcgagcctcagcgctcggcgccggccggattccgcggctaacgtgggcgtggctgccccgtcgtttccaagaccccttagccagccgacttctccagttacggagcgagcccctctttttttttcttgtgtttttgccagatgcatcccgtactgcggcagatgcgcccccaccctccaccacaaccgcccctaccgcagcagcagcaacagccggcgcttctgcccccgccccagcagcagccagccactaccgcggcggccgccgtgagcggagccggcgttcagtatgacctggccttggaagagggcgaggggctggcgcggctgggggcgtcgtcgccggagcggcacccgcgcgtgcagatgaaaagggacgctcgcgaggcctacgtgcccaagcagaacctgttcagagacaggagcggcgaggagcccgaggagatgcgcgcctcccgcttccacgcggggcgggagctgcggcgcggcctggaccgaaagcgggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcacgctgatcgcgcgcgaggaggtgaccctgggcctgatgcacctgtgggacctgctggaggccatcgtgcagaaccccacgagcaagccgctgacggcgcagctgtttctggtggtgcagcacagtcgggacaacgagacgttcagggaggcgctgctgaatatcaccgagcccgagggccgctggctcctggacctggtgaacattttgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagccgccggcgcgagctgagcgaccaggagctgatgcacagcctgcagcgggccctgaccggggccgggaccgagggggagagctactttgacatgggcgcggacctgcgctggcagcccagccgccgggccttggaagctgccggcggttccccctacgtggaggaggtggacgatgaggaggaggagggcgagtacctggaagactgatggcgcgaccgtatttttgctagatgcagcaacagccaccgccgccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggtgacgaggccgggctggtgtacaacgcgctgctggagcgcgtggcccgctacaacagcaccaacgtgcagacgaacctggaccgcatggtgaccgacgtgcgcgaggcggtgtcgcagcgcgagcggttccaccgcgagtcgaacctgggctccatggtggcgctgaacgccttcctgagcacgcagcccgccaacgtgccccggggccaggaggactacaccaacttcatcagcgcgctgcggctgatggtggccgaggtgccccagagcgaggtgtaccagtcggggccggactacttcttccagaccagtcgccagggcttgcagaccgtgaacctgagccaggctttcaagaacttgcagggactgtggggcgtgcaggccccggtcggggaccgcgcgacggtgtcgagcctgctgacgccgaactcgcgcctgctgctgctgctggtggcgcccttcacggacagcggcagcgtgagccgcgactcgtacctgggctacctgcttaacctgtaccgcgaggccatcggacaggcgcacgtggacgagcagacctaccaggagatcacccacgtgagccgcgcgctgggccaggaggacccgggcaacctggaggccaccctgaacttcctgctgaccaaccggtcgcagaagatcccgccccagtacgcgctgagcaccgaggaggagcgcatcctgcgctacgtgcagcagagcgtggggctgttcctgatgcaggagggggccacgcccagcgcggcgctcgacatgaccgcgcgcaacatggagcccagcatgtacgcccgcaaccgcccgttcatcaataagctgatggactacttgcatcgggcggccgccatgaactcggactactttaccaacgccatcttgaacccgcactggctcccgccgcccgggttctacacgggcgagtacgacatgcccgaccccaacgacgggttcctgtgggacgacgtggacagcagcgtgttctcgccgcgtccaggaaccaatgccgtgtggaagaaagagggcggggaccggcggccgtcctcggcgctgtccggtcgcgcgggtgctgccgcggcggtgcccgaggccgccagccccttcccgagcctgcccttttcgctgaacagcgtgcgcagcagcgagctgggtcggctgacgcgaccgcgcctgctgggcgaggaggagtacctgaacgactccttgttgaggcccgagcgcgagaagaacttccccaataacgggatagagagcctggtggacaagatgagccgctggaagacgtacgcgcacgagcacagggacgagccccgagctagcagcgcaggcacccgtagacgccagcggcacgacaggcagcggggactggtgtgggacgatgaggattccgccgacgacagcagcgtgttggacttgggtgggagtggtggtaacccgttcgctcacctgcgcccccgtatcgggcgcctgatgtaagaatctgaaaaaataaaagacggtactcaccaaggccatggcgaccagcgtgcgttcttctctgttgtttgtagtagtatgatgaggcgcgtgtacccggagggtcctcctccctcgtacgagagcgtgatgcagcaggcggtggcggcggcgatgcagcccccgctggaggcgccttacgtgcccccgcggtacctggcgcctacggaggggcggaacagcattcgttactcggagctggcacccttgtacgataccacccggttgtacctggtggacaacaagtcggcagacatcgcctcgctgaactaccagaacgaccacagcaacttcctgaccaccgtggtgcagaacaacgatttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcgcgcaagacccccaacggggtggatgatgattatgatggtagtcaggacgagctgacctacgagtgggtggagtttgagctgcccgagggcaacttctcggtgaccatgaccatcgatctgatgaacaacgccatcatcgacaactacttggcggtggggcggcagaacggggtgctggagagcgacatcggcgtgaagttcgacacgcgcaacttccggctgggctgggaccccgtgaccgagctggtgatgccgggcgtgtacaccaacgaggccttccaccccgacatcgtcctgctgcccggctgcggcgtggacttcaccgagagccgcctcagcaacctgctgggcatccgcaagcggcagcccttccaggagggcttccagatcctgtacgaggacctggaggggggcaacatccccgcgctcttggatgtcgaagcctacgagaaaagcaaggaggatagcaccgccgcggcgaccgcagccgtggccaccgcctctaccgaggtgcggggcgataattttgctagcgctgcggcagcggccgaggcggctgaaaccgaaagtaagatagtcatccagccggtggagaaggacagcaaggacaggagctacaacgtgctcgcggacaagaaaaacaccgcctaccgcagctggtacctggcctacaactacggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgtcactgacgccagacgccgcacctgcccctacgtctacaaggccctgggcgtagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcgctcgcgcaccaccgtcgacgacgtgatcgaccaggtggtggccgacgcgcgcaactacacgcccgccgccgcgcccgtctccaccgtggacgccgtcatcgacagcgtggtggccgacgcgcgccggtacgcccgcaccaagagccggcggcggcgcatcgcccggcggcaccggagcacccccgccatgcgcgcggcgcgagccttgctgcgcagggccaggcgcacgggacgcagggccatgctcagggcggccagacgcgcggcctccggcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggcggcggtgaaggaggaaagaaagccccgcaaactgaagcgggtcaaaaaggacaaaaaggaggaggaagatgacggactggtggagtttgtgcgcgagttcgccccccggcggcgcgtgcagtggcgcgggcggaaagtgaaaccggtgctgcggcccggcaccacggtggtcttcacgcccggcgagcgttccggctccgcctccaagcgctcctacgacgaggtgtacggggacgaggacatcctcgagcaggcggtcgagcgtctgggcgagtttgcgtacggcaagcgcagccgccccgcgcccttgaaagaggaggcggtgtccatcccgctggaccacggcaaccccacgccgagcctgaagccggtgaccctgcagcaggtgctaccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccagcaccagcttccaccagcactcgccgaagacgcaagtacggcgcggccagcctgctgatgcccaactacgcgctgcatccttccatcatccccacgccgggctaccgcggcacgcgcttctaccgcggctacaccagcagccgccgccgcaagaccaccacccgccgccgtcgtcgcagccgccgcagcagcaccgcgacttccgccttggtgcggagagtgtatcgcagcgggcgcgagcctctgaccctgccgcgcgcgcgctaccacccgagcatcgccatttaactaccgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacaaaaaagcatggatttgtaataaaaaaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagaacagaacttccagcagaaggtggttgatggcctggcctcaggcatcaacggggtggttgacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggcctgcccaccacgcggcccgtggcgcctctggccaccggagtgctgaaacccagcagcagccagcccgcgaccctggacttgcctccgcctcgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtatatgtatgtccgccgaccagaaggaggagtgtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggaccggggccctagcttcaaaccctactctggcaccgcctacaacagcctagctcccaagggagctcccaattccagccagtgggagcaagcaaaaacaggcaatgggggaactatggaaacacacacatatggtgtggccccaatgggcggagagaatattacaaaagatggtcttcaaattggaactgacgttacagcgaatcagaataaaccaatttatgccgacaaaacatttcaaccagaaccgcaagtaggagaagaaaattggcaagaaactgaaaacttttatggcggtagagctcttaaaaaagacacaaacatgaaaccttgctatggctcctatgctagacccaccaatgaaaaaggaggtcaagctaaacttaaagttggagatgatggagttccaaccaaagaattcgacatagacctggctttctttgatactcccggtggcaccgtgaacggtcaagacgagtataaagcagacattgtcatgtataccgaaaacacgtatttggaaactccagacacgcatgtggtatacaaaccaggcaaggatgatgcaagttctgaaattaacctggttcagcagtctatgcccaacagacccaactacattgggttcagggacaactttatcggtcttatgtactacaacagcactggcaatatgggtgtgcttgctggtcaggcctcccagctgaatgctgtggttgatttgcaagacagaaacaccgagctgtcctaccagctcttgcttgactctttgggtgacagaacccggtatttcagtatgtggaaccaggcggtggacagttatgaccccgatgtgcgcatcatcgaaaaccatggtgtggaggatgaattgccaaactattgcttccccttggacggctctggcactaacgccgcataccaaggtgtgaaagtaaaagatggtcaagatggtgatgttgagagtgaatgggaaaatgacgatactgttgcagctcgaaatcaattatgtaaaggtaacattttcgccatggagattaatctccaggctaacctgtggagaagtttcctctactcgaacgtggccctgtacctgcccgactcctacaagtacacgccgaccaacgtcacgctgccgaccaacaccaacacctacgattacatgaatggcagagtgacacctccctcgctggtagacgcctacctcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctaggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctgaagacccgcgagacgccctcgctcggctccgggttcgacccctacttcgtctactcgggctccatcccctacctagacggcaccttctacctcaaccacaccttcaagaaggtctccatcaccttcgactcctccgtcagctggcccggcaacgaccgcctcctgacgcccaacgagttcgaaatcaagcgcaccgtcgacggagagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgtcgtccgagtgcaccagccccaccgcggcgtcatcgaagccgtctacctgcgcacgcccttctcggccggcaacgccaccacctaagccgctcttgcttcttgcaagatgacggcgggctccggcgagcaggagctcagggccatcctccgcgacctgggctgcgggccctgcttcctgggcaccttcgacaagcgcttccctggattcatggccccgcacaagctggcctgcgccatcgtgaacacggccggccgcgagaccgggggcgagcactggctggccttcgcctggaacccgcgctcccacacatgctacctcttcgaccccttcgggttctcggacgagcgcctcaagcagatctaccagttcgagtacgagggcctgctgcgtcgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttactgacgggggtgcccaacggcatgctccagtcgccccaggtggaacccaccctgcgccgcaaccaggaagcgctctaccgcttcctcaatgcccactccgcctactttcgctcccaccgcgcgcgcatcgagaaggccaccgccttcgaccgcatgaatcaagacatgtaaaaaaccggtgtgtgtatgtgaatgctttattcataataaacagcacatgtttatgccaccttctctgaggctctgactttatttagaaatcgaaggggttctgccggctctcggcatggcccgcgggcagggatacgttgcggaactggtacttgggcagccacttgaactcggggatcagcagcttgggcacggggaggtcggggaacgagtcgctccacagcttgcgcgtgagttgcagggcgcccagcaggtcgggcgcggagatcttgaaatcgcagttgggacccgcgttctgcgcgcgagagttgcggtacacggggttgcagcactggaacaccatcagggccgggtgcttcacgcttgccagcaccgtcgcgtcggtgatgccctccacgtccagatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagccggcgtcgtgcacgcagcagcgcgcgtcgttgttggccagctgcaccacgctgcgcccccagcggttctgggtgatcttggcccggttggggttctccttcagcgcgcgctgcccgttctcgctcgccacatccatctcgatagtgtgctccttctggatcatcacggtcccgtgcaggcaccgcagcttgccctcggcttcggtgcagccgtgcagccacagcgcgcagccggtgcactcccagttcttgtgggcgatctgggagtgcgagtgcacgaagccctgcaggaagcggcccatcatcgcggtcagggtcttgttgctggtgaaggtcagcgggatgccgcggtgctcctcgttcacatacaggtggcagatgcggcggtacacctcgccctgctcgggcatcagctggaaggcggacttcaggtcgctctccacgcggtaccggtccatcagcagcgtcatcacttccatgcccttctcccaggccgaaacgatcggcaggctcagggggttcttcaccgccattgtcatcttagtcgccgccgccgaggtcagggggtcgttctcgtccagggtctcaaacactcgcttgccgtccttctcgatgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcgatgtgctgggagagcgcgagttctcgttcaccacgactatttcttcttcttggccgtcgtccgagaccacgcggcggtaggcatgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaacaacaagcatggagactcagccatcgtcgccaacatcgccatctgcccccgccgccaccgccgacgagaaccagcagcagaatgaaagcttaaccgccccgccgcccagccccacctccgacgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagaacgagcagaaccaggctgggcacgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgtgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcacctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtcctcaagcgcacggtcgagctgacccacttcgcctacccggcgctcaacctgccccccaaggtcatgagcgccgtcatggaccaggtgctcatcaagcgcgcctcgcccctctcggaggaggagatgcaggaccccgagagttcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttctttgccgacgcggagaccctgcgcaaggtcgaggagaacctgcactacctcttcaggcacgggttcgtgcgccaggcctgcaagatctccaacgtggagctgaccaacctggtctcctacatgggcatcctgcacgagaaccgcctggggcaaaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgtaccaccgcctcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcgctgccctcggacttcgtgccgctgaccttccgcgagtgccccccgccgctctggagccactgctacttgctgcgcctggccaactacctggcctaccactcggacgtgatcgaggacgtcagcggcgagggtctgctggagtgccactgccgctgcaacctctgcacgccgcaccgctccctggcctgcaacccccagctgctgagcgagacccagatcatcggcaccttcgagttgcaaggccccggcgacggcgagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacttggacccccagaccggagaggagctcaaccccagcttcccccaggatgccccgaggaagcagcaagaagctgaaagtggagctgccgccgccggaggatttggaggaagactgggagagcagtcaggcagaggaggaggagatggaagactgggacagcactcaggcagaggaggacagcctgcaagacagtctggaggaggaagacgaggtggaggaggcagaggaagaagcagccgccgccagaccgtcgtcctcggcggagaaagcaagcagcacggataccatctccgctccgggtcggggtcgcggcggccgggcccacagtaggtgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagaaaaccagcggcagcagcagctagaaaatccacagcggcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggcggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccagcccacggagtgcggatcatcgtcgaagggggcctcgactcccacctgcttcggatcttcagccagcgaccgatcctggtcgagcgcgaacaaggacagacccttcttactttgtactgcatctgcaaccaccccggcctgcatgaaagtctttgttgtctgctgtgtactgagtataataaaagctgagatcagcgactactccggactcgattgtggtgttcctgctatcaaccggtccctgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcaggaccctgccatcacaccttccacctgatcccgaataccacagcgccgctccccgctactaacaaccaaactacccaccaacgccaccgtcgcgacctttcctctgaatctaataccactaccggaggtgagctccgaggtcgaccaacctctgggatttactacggcccctgggaggtggtggggttaatagcgctaggcctagttgcgggtgggcttttggttctctgctacctatacctcccttgctgttcgtacttagtggtgctgtgttgctggtttaagaaatggggaagatcaccctagtgagctgcggtgcgctggtggcggtgttgctttcgattgtgggactgggcggcgcggctgtagtgaaggagaaggccgatccctgcttgcatttcaatcccaacaaatgccagctgagttttcagcccgatggcaatcggtgcgcggtactgatcaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaataatactttcatttttgcgcacatgtgcaacacggtcatgtggatgagcaagcagtacgatatgtggccccccacgaaggagaacatcgtggtcttctccatcgcttacagcctgtgcacggcgctaatcaccgctatcgtgtgcctgagcattcacatgctcatcgctattcgccccagaaataatgccgagaaagagaaacagccataacacgttttttcacacaccttgtttttacagacaatgcgtctgttaaattttttaaacattgtgctcagtattgcttatgcctctggttatgcaaacatacagaaaaccctttatgtaggatctgatggtacactagagggtacccaatcacaagccaaggttgcatggtatttttatagaaccaacactgatccagttaaactttgtaagggtgaattgccgcgtacacataaaactccacttacatttagttgcagcaataataatcttacacttttttcaattacaaaacaatatactggtacttattacagtacaaactttcatacaggacaagataaatattatactgttaaggtagaaaatcctaccactcctagaactaccaccaccaccactactgcaaagcccactgtgaaaactacaactaggaccaccacaactacagaaaccaccaccagcacaacacttgctgcaactacacacacacacactaagctaaccttacagaccactaatgatttgatcgccctgctgcaaaagggggataacagcaccacttccaatgaggagatacccaaatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggcctttttagtttttctatcattacctctgctctttgtgaatcagtggatagagatgttactattaccactggttctaattatacactgaaagggccaccctcaggtatgctttcgtggtattgctattttggaactgacactgatcaaactgaattatgcaattttcaaaaaggcaaaacctcaaactctaaaatctctaattatcaatgcaatggcactgatctgatactactcaatgtcacgaaagcatatggtggcagttattattgccctggacaaaacactgaagaaatgattttttacaaagtggaagtggttgatcccactacaccacccaccaccacaactattcataccacacacacagaacaaacaccagaggcaacagaagcagagttggccttccaggttcacggagattcctttgctgtcaatacccctacacccgatcagcggtgtccggggccgctagtcagcggcattgtcggtgtgctttcgggattagcagtcataatcatctgcatgttcatttttgcttgctgctatagaaggctttaccgacaaaaatcagacccactgctgaacctctatgtttaattttttccagagccatgaaggcagttagcgctctagttttttgttctttgattggcattgtttttaatagtaaaattaccagagttagctttattaaacatgttaatgtaactgaaggagataacatcacactagcaggtgtagaaggtgctcaaaacaccacctggacaaaataccatctaggatggagagatatttgcacctggaatgtaacttattattgcataggagttaatcttaccattgttaacgctaaccaatctcagaatgggttaattaaaggacagagtgttagtgtgaccagtgatgggtactatacccagcatagttttaactacaacattactgtcataccactgcctacgcctagcccacctagcactaccacacagacaaccacatacagtacatcaaatcagcctaccaccactacagcagcagaggttgccagctcgtctggggtccgagtggcatttttgatgttggccccatctagcagtcccactgctagtaccaatgagcagactactgaatttttgtccactgtcgagagccacaccacagctacctccagtgccttctctagcaccgccaatctctcctcgctttcctctacaccaatcagccccgctactactcctagccccgctcctcttcccactcccctgaagcaaacagacggcggcatgcaatggcagatcaccctgctcattgtgatcgggttggtcatcctggccgtgttgctctactacatcttctgccgccgcattcccaacgcgcaccgcaagccggcctacaagcccatcgttatcgggcagccggagccgcttcaggtggaagggggtctaaggaatcttctcttctcttttacagtatggtgattgaactatgattcctagacaattcttgatcactattcttatctgcctcctccaagtctgtgccaccctcgctctggtggccaacgccagtccagactgtattgggcccttcgcctcctacgtgctctttgccttcgtcacctgcatctgctgctgtagcatagtctgcctgcttatcaccttcttccagttcattgactggatctttgtgcgcatcgcctacctgcgccaccacccccagtaccgcgaccagcgagtggcgcagctgctcaggctcctctgataagcatgcgggctctgctacttctcgcgcttctgctgttagtgctcccccgtcccgtcgacccccggtcccccactcagtcccccgaggaggttcgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcagcaacctcaggcacacgcactaccaccaccacagcctaggccacaatacatgcccatattagactatgaggccgagccacagcgacccatgctccccgctattagttacttcaatctaaccggcggagatgactgacccactggccaataacaacgtcaacgaccttctcctggacatggacggccgcgcctcggagcagcgactcgcccaacttcgcattcgtcagcagcaggagagagccgtcaaggagctgcaggacggcatagccatccaccagtgcaagagaggcatcttctgcctggtgaaacaggccaagatctcctacgaggtcacccagaccgaccatcgcctctcctacgagctcctgcagcagcgccagaagttcacctgcctggtcggagtcaaccccatcgtcatcacccagcagtcgggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgatttaaataaaaaaaataatcatttgatttgaaataaagatacaatcatattgatgatttgagtttaacaaaaataaagaatcacttacttgaaatctgataccaggtctctgtccatgttttctgccaacaccacctcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggctgaccccgtcaccaccaagaacggggaaatcaccctcaagctgggagagggggtggacctcgactcgtcgggaaaactcatctccaacacggccaccaaggccgccgcccctctcagtatttcaaacaacaccatttcccttaaaactgctgcccctttctacaacaacaatggaactttaagcctcaatgtctccacaccattagcagtatttcccacatttaacactttaggcataagtcttggaaacggtcttcagacttcaaataagttgttgactgtacaactaactcatcctcttacattcagctcaaatagcatcacagtaaaaacagacaaagggctatatattaactccagtggaaacagaggacttgaggctaatataagcctaaaaagaggactagtttttgacggtaatgctattgcaacatatattggaaatggcttagactatggatcttatgatagtgatggaaaaacaagacccgtaattaccaaaattggagcaggattaaattttgatgctaacaaagcaatagctgtcaaactaggcacaggtttaagttttgactccgctggtgccttgacagctggaaacaaacaggatgacaagctaacactttggactacccctgacccaagccctaattgtcaattactttcagacagagatgccaaatttactctctgtcttacaaaatgcggtagtcaaatactaggcactgtggcagtggcggctgttactgtaggatcagcactaaatccaattaatgacacagtcaaaagcgccatagttttccttagatttgattccgatggtgtactcatgtcaaactcatcaatggtaggtgattactggaactttagggagggacagaccactcaaagtgtagcctatacaaatgctgtgggattcatgccaaatataggtgcatatccaaaaacccaaagtaaaacacctaaaaatagcatagtcagtcaggtatatttaactggagaaactactatgccaatgacactaaccataactttcaatggcactgatgaaaaagacacaaccccagttagcacctactctatgacttttacatggcagtggactggagactataaggacaaaaatattacctttgctaccaactcattctctttttcctacatcgcccaggaataatcccacccagcaagccaaccccttttcccaccacctttgtctatatggaaactctgaaacagaaaaataaagttcaagtgttttattgaatcaacagttttacaggactcgagcagttatttttcctccaccctcccaggacatggaatacaccaccctctccccccgcacagccttgaacatctgaatgccattggtgatggacatgcttttggtctccacgttccacacagtttcagagcgagccagtctcggatcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggttcgggtccagggactccctcagcatgatgcccacggccctcagcatcagtcgtctggtgcggcgggcgcagcagcgcatgcgaatctcgctcaggtcactgcagtacgtgcaacacaggaccaccaggttgttcaacagtccatagttcaacacgctccagccgaaactcatcgcgggaaggatgctacccacgtggccgtcgtaccagatcctcaggtaaatcaagtggcgctccctccagaagacgctgcccatgtacatgatctccttgggcatgtggcggttcaccacctcccggtaccacatcaccctctggttgaacatgcagccccggatgatcctgcggaaccacagggccagcaccgccccgcccgccatgcagcgaagagaccccggatcccggcaatgacaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttctcgaccgtgtcatgatgcagttgctttcggacattttcgtacttgctgtagcagaacctggtccgggcgctgcacaccgatcgccggcggcggtctcggcgcttggaacgctcggtgttaaagttgtaaaacagccactctctcagaccgtgcagcagatctagggcctcaggagtgatgaagatcccatcatgcctgatagctctgatcacatcgaccaccgtggaatgggccaggcccagccagatgatgcaattttgttgggtttcggtgacggcgggggagggaagaacaggaagaaccatgattaacttttaatccaaacggtctcggagcacttcaaaatgaaggtcacggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaaccatcatgttacactcctgcaccatccccagataattttcatttttccagccttgaatgattcgaactagttcctgaggtaaatccaagccagccatgataaaaagctcgcgcagagcaccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcgtctccgaaatttttagccataggacccccaggaataagagaagggcaagccacattacagataaaccgaagtcccccccagtgagcattgccaaatgtaagattgaaataagcatgctggctagacccggtgatatcttccagataactggacagaaaatcgggtaagcaatttttaagaaaatcaacaaaagaaaaatcttccaggtgcacgtttagggcctcgggaacaacgatggagtaagtgcaaggggtgcgttccagcatggttagttagctgatctgtaaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgagaagaagcatacacccccggaacattggagtccgtgagtgaaaaaaagcggccgaggaagcaatgaggcactacaacgctcactctcaagtccagcaaagcgatgccatgcggatgaagcacaaaattttcaggtgcgtaaaaaatgtaattactcccctcctgcacaggcagcgaagctcccgatccctccagatacacatacaaagcctcagcgtccatagcttaccgagcggcagcagcagcggcacacaacaggcgcaagagtcagagaaaagactgagctctaacctgtccgcccgctctctgctcaatatatagccccagatctacactgacgtaaaggccaaagtctaaaaatacccgccaaataatcacacacgcccagcacacgcccagaaaccggtgacacactcagaaaaatacgcgcacttcctcaaacggccaaactgccgtcatttccgggttcccacgctacgtcatcaaaacacgactttcaaattccgtcgaccgttaaaaacatcacccgccccgcccctaacggtcgccgctcccgcagccaatcaccttcctccctccccaaattcaaacagctcatttgcatattaacgcgcaccaaaagtttgaggtatattattgatgatg

SEQ ID NO: 6 - protein, 529 aa, chimpanzee adenovirus serotype Pan6
MMRRVYPEGPPPSYESVMQQAVAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVDDDYDGSQDELTYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQILYEDLEGGNIPALLDVEAYEKSKEDSTAAATAAVATASTEVRGDNFASAAAAAEAAETESKIVIQPVEKDSKDRSYNVLADKKNTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGVVAPRVLSSRTF

SEQ ID NO: 7 - protein, 942 aa, chimpanzee adenovirus serotype Pan6
MATPSMLPQWAYMHIAGQDASEYLSPGLVQFARATDTYFSLGNKFRNPTVAPTHDVTTDRSQRLTLRFVPVDREDNTYSYKVRYTLAVGDNRVLDMASTYFDIRGVLDRGPSFKPYSGTAYNSLAPKGAPNSSQWEQAKTGNGGTMETHTYGVAPMGGENITKDGLQIGTDVTANQNKPIYADKTFQPEPQVGEENWQETENFYGGRALKKDTNMKPCYGSYARPTNEKGGQAKLKVGDDGVPTKEFDIDLAFFDTPGGTVNGQDEYKADIVMYTENTYLETPDTHVVYKPGKDDASSEINLVQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDGSGTNAAYQGVKVKDGQDGDVESEWENDDTVAARNQLCKGNIFAMEINLQANLWRSFLYSNVALYLPDSYKYTPTNVTLPTNTNTYDYMNGRVTPPSLVDAYLNIGARWSLDPMDNVNPFNHHRNAGLRYRSMLLGNGRYVPFHIQVPQKFFAIKSLLLLPGSYTYEWNFRKDVNMILQSSLGNDLRTDGASIAFTSINLYATFFPMAHNTASTLEAMLRNDTNDQSFNDYLSAANMLYPIPANATNVPISIPSRNWAAFRGWSFTRLKTRETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVASVTQKKFLCDRVMWRIPFSSNFMSMGALTDLGQNMLYANSAHALDMNFEVDPMDESTLLYVVFEVFDVVRVHQPHRGVIEAVYLRTPFSAGNATT

SEQ ID NO: 8 - protein, 443 aa, chimpanzee adenovirus serotype Pan6
MSKKRVRVDDDFDPVYPYDADNAPTVPFINPPFVSSDGFQEKPLGVLSLRLADPVTTKNGEITLKLGEGVDLDSSGKLISNTATKAAAPLSISNNTISLKTAAPFYNNNGTLSLNVSTPLAVFPTFNTLGISLGNGLQTSNKLLTVQLTHPLTFSSNSITVKTDKGLYINSSGNRGLEANISLKRGLVFDGNAIATYIGNGLDYGSYDSDGKTRPVITKIGAGLNFDANKAIAVKLGTGLSFDSAGALTAGNKQDDKLTLWTTPDPSPNCQLLSDRDAKFTLCLTKCGSQILGTVAVAAVTVGSALNPINDTVKSAIVFLRFDSDGVLMSNSSMVGDYWNFREGQTTQSVAYTNAVGFMPNIGAYPKTQSKTPKNSIVSQVYLTGETTMPMTLTITFNGTDEKDTTPVSTYSMTFTWQWTGDYKDKNITFATNSFSFSYIAQE

SEQ ID NO: 9 - DNA, 36535 bp, chimpanzee adenovirus serotype Pan7
  CDS (13874)..(15469): L2 Penton
catcatcaataatatacctcaaacttttggtgcgcgttaatatgcaaatgagctgtttgaatttggggagggaggaaggtgattggccgagagacgggcgaccgttaggggcggggcgggtgacgtttttaatacgtggccgtgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacgtaggcgtcagctgatcgccagggtatttaaacctgcgctctctagtcaagaggccactcttgagtgccagcgagtagagttttctcctccgcgccgcgagtcagatctacactttgaaagatgaggcacctgagagacctgcccggtaatgttttcctggctactgggaacgagattctggaattggtggtggacgccatgatgggtggcgaccctcctgagccccctaccccatttgaggcgccttcgctgtacgatttgtatgatctggaggtggatgtgcccgagaacgaccccaacgaggaggcggtgaatgatttgtttagcgatgccgcgctgctggctgccgagcaggctaatacggactctggctcagacagcgattcctctctccataccccgagacccggcagaggtgagaaaaagatccccgagcttaaaggggaagagctcgacctgcgctgctatgaggaatgcttgcctccgagcgatgatgaggaggacgaggaggcgattcgagctgcatcgaaccagggagtgaaagctgcgggcgaaagctttagcctggactgtcctactctgcccggacacggctgtaagtcttgtgaatttcatcgcatgaatactggagataagaatgtgatgtgtgccctgtgctatatgagagcttacaaccattgtgtttacagtaagtgtgattaactttagttgggaaggcagagggtgactgggtgctgactggtttatttatgtatatgtttttttatgtgtaggtcccgtctctgacgtagatgagacccccacttcagagtgcatttcatcacccccagaaattggcgaggaaccgcccgaagatattattcatagaccagttgcagtgagagtcaccgggcggagagcagctgtggagagtttggatgacttgctacagggtggggatgaacctttggacttgtgtacccggaaacgccccaggcactaagtgccacacatgtgtgtttacttaaggtgatgtcagtatttatagggtgtggagtgcaataaaatccgtgttgactttaagtgcgtggtttatgactcaggggtggggactgtgggtatataagcaggtgcagacctgtgtggtcagttcagagcaggactcatggagatctggacggtcttggaagactttcaccagactagacagctgctagagaactcatcggagggggtctcttacctgtggagattctgcttcggtgggcctctagctaagctagtctatagggccaaacaggattataaggatcaatttgaggatattttgagagagtgtcctggtatttttgactctctcaacttgggccatcagtctcactttaaccagagtattctgagagcccttgacttttctactcctggcagaactaccgccgcggtagccttttttgcctttatccttgacaaatggagtcaagaaacccatttcagcagggattaccgtctggactgcttagcagtagctttgtggagaacatggaggtgccagcgcctgaatgcaatctccggctacttgccagtacagccggtagacacgctgaggatcctgagtctccagtcaccccaggaacaccaacgccgccagcagccgcagcaggagcagcagcaagaggaggaggaggatcgagaagagaacccgagagccggtctggaccctccggtggcggaggaggaggagtagctgacttgtttcccgagctgcgccgggtgctgactaggtcttccagtggacgggagagggggattaagcgggagaggcatgaggagactagccacagaactgaactgactgtcagtctgatgagccgcaggcgcccagaatcggtgtggtggcatgaggttcagtcgcaggggatagatgaggtctcggtgatgcatgagaaatattccctggaacaagtcaagacttgttggttggagcctgaggatgattgggaggtagccatcaggaattatgccaagctggctctgaagccagacaagaagtacaagattaccaaactgattaatatcagaaattcctgctacatttcagggaatggggccgaggtggagatcagtacccaggagagggtggccttcagatgttgtatgatgaatatgtacccgggggtggtgggcatggagggagtcacctttatgaacgcgaggttcaggggtgatgggtataatggggtggtctttatggccaacaccaagctgacagtgcacggatgctccttctttgggttcaataacatgtgcatcgaggcctggggcagtgtttcagtgaggggatgcagcttttcagccaactggatgggggtcgtgggcagaaccaagagcaaggtgtcagtgaagaaatgcctgttcgagaggtgccacctgggggtgatgagcgagggcgaagccaaagtcaaacactgcgcctctactgagacgggctgctttgtgctgatcaagggcaatgcccaagtcaagcataacatgatctgtggggcctcggatgagcgcggctaccagatgctgacctgcgccggtgggaacagccatatgctggccaccgtgcatgtgacctcgcacccccgcaagacatggcccgagttcgagcacaacgtcatgacccgatgcaatgtgcacctggggtcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttgggggaggtggagggcttgtatgaggggcagaatgactaaaatctgtgtttttctgtgtgttgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcgaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgacttccaccaataatcccgccagcctgaacgaggagaagctgctgctgctgatggcccagctcgaggccctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggattttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgctgcacgatgtccttgaggaggagactgatggccacgggcagccccttggtgtaggtgttgacgaacctgttgagctgggagggatgcatgcggggggagatgagatgcatcttggcctggatcttgagattggcgatgttcccgcccagatcccgccgggggttcatgttgtgcaggaccaccagcacggtgtatccggtgcacttggggaatttgtcatgcaacttggaagggaaggcgtgaaagaatttggagacgcccttgtgaccgcccaggttttccatgcactcatccatgatgatggcgatgggcccgtgggcggcggcctgggcaaagacgtttcgggggtcggacacatcgtagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcagccggtggggccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagyccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccagggtcgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcgggccggttggggtcaaaaacgaggtttcctccgtgctttttgatgcgtttcttacctctggtctccatgagctcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggctggcataacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagaggagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcagaagctcggggtcgaagtagctgatgcaggtgtccagatcgtccagcgccgcttgccagtcgcgcacggccagcgcgcgctcgtaggggctgaggggcgtgccccagggcatggggtgcgtgagcgcggaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgtgccgaggttggagcgttgcggcttttcggcgcggtagacgatctggcggaagatggcgtgggagttggaggagatggtgggcctctggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgtgcggccttgcgcagggaggtgtgggtgagggcgaaggtgtcgcgcaccatgaccttgaggaactggtgcttgaagtcgaggtcgtcgcagccgccctgctcccagagctggaagtccgtgcgcttcttgtaggcggggttgggcaaagcgaaagtaacatcgttgaagaggatcttgcccgcgcggggcatgaagttgcgagtgatgcggaaaggctggggcacctcggcccggttgttgatgacctgggcggcgaggacgatctcgtcgaagccgttgatgttgtgcccgacgatgtagagttccacgaatcgcgggcggcccttaacgtggggcagcttcttgagctcgtcgtaggtgagctcggcggggtcgctgagcccgtgctgctcgagggcccagtcggcgacgtgggggttggcgctgaggaaggaagtccagagatccacggccagggcggtctgcaagcggtcccggtactgacggaactgctggcccacggccattttttcgggggtgacgcagtagaaggtgcgggggtcgccgtgccagcggtcccacttgagctggagggcgaggtcgtgggcgagctcgacgagcggcgggtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccagttggaggaatggctgttgatgtgatggaagtagaaatgccgacggcgcgccgagcactcgtgcttgtgtttatacaagcgtccgcagtgctcgcaacgctgcacgggatgcacgtgctgcacgagctgtacctgggttcctttgacgaggaatttcagtgggcagtggagcgctggcggctgcatctggtgctgtactacgtcctggccatcggcgtggccatcgtctgcctcgatggtggtcatgctgacgagcccgcgcgggaggcaggtccagacttcggctcggacgggtcggagagcgaggacgagggcgcgcaggccggagctgtccagggtcctgagacgctgcggagtcaggtcagtgggcagcggcggcgcgcggttgacttgcaggagcttttccagggcgcgcgggaggtccagatggtacttgatctccacggcgccgttggtggcgacgtccacggcttgcagggtcccgtgcccctggggcgccaccaccgtgccccgtttcttcttgggcgctgcttccatgccggtcagaagcggcggcgaggacgcgcgccgggcggcaggggcggctcgggacccggaggcaggggcggcaggggcacgtcggcgccgcgcgcgggcaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctcgacggtggccgcgaggtcgttggagatgcggcccatgagctgcgagaaggcgttcatgccggcctcgttccagacgcggctgtagaccacggctccgtcggggtcgcgcgcgcgcatgaccacctgggcgaggttgagctcgacgtggcgcgtgaagaccgcgtagttgcagaggcgctggtagaggtagttgagcgtggtggcgatgtgctcggtgacgaagaagtacatgatccagcggcggagcggcatctcgctgacgtcgcccagggcttccaagcgctccatggcctcgtagaagtccacggcgaagttgaaaaactgggagttgcgcgccgagacggtcaactcctcctccagaagacggatgagctcagcgatggtggcgcgcacctcgcgctcgaaggccccggggggctcctcttcttccatctcttcctcctccactaacatctcttctacttcctcctcaggaggcggcggcgggggaggggccctgcgtcgccggcggcgcacgggcagacggtcgatgaagcgctcgatggtctccccgcgccggcgacgcatggtctcggtgacggcgcgcccgtcctcgcggggccgcagcgtgaagacgccgccgcgcatctccaggtggccgccgggggggtctccgttgggcagggagagggcgctgacgatgcatcttatcaattggcccgtagggactccgcgcaaggacctgagcgtctcgagatccacgggatccgaaaaccgctgaacgaaggcttcgagccagtcgcagtcgcaaggtaggctgagcccggtttcttgttcttcggggatttcgggaggcgggcgggcgatgctgctggtgatgaagttgaagtaggcggtcctgagacggcggatggtggcgaggagcaccaggtccttgggcccggcttgctggatgcgcagacggtcggccatgccccaggcgtggtcctgacacctggcgaggtccttgtagtagtcctgcatgagccgctccacgggcacctcctcctcgcccgcgcggccgtgcatgcgcgtgagcccgaacccgcgctggggctggacgagcgccaggtcggcgacgacgcgctcggcgaggatggcctgctgtatctgggtgagggtggtctggaagtcgtcgaagtcgacgaagcggtggtaggctccggtgttgatggtataggagcagttggccatgacggaccagttgacggtctggtggccgggtcgcacgagctcgtggtacttgaggcgcgagtaggcgcgcgtgtcgaagatgtagtcgttgcaggtgcgcacgaggtactggtatccgacgaggaagtgcggcggcggctggcggtagagcggccatcgctcggtggcgggggcgccgggcgcgaggtcctcgagcatgaggcggtggtagccgtagatgtacctggacatccaggtgatgccggcggcggtggtggaggcgcgcgggaactcgcggacgcggttccagatgttgcgcagcggcaggaagtagttcatggtggccgcggtctggcccgtgaggcgcgcgcagtcgtggatgctctagacatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgctaacgaaacctccaggatacggaggcgggtcgttttttggccttggtcgctggtcatgaaaaactagtaagcgcggaaagcgaccgcccgcgatggctcgctgccgtagtctggagaaagaatcgccagggttgcgttgcggtgtgccccggttcgagcctcagcgctcggcgccggccggattccgcggctaacgtgggcgtggctgccccgtcgtttccaagaccccttagccagccgacttctccagttacggagcgagcccctctttttcttgtgtttttgccagatgcatcccgtactgcggcagatgcgcccccaccctccacctcaaccgcccctaccgccgcagcagcagcaacagccggcgcttctgcccccgccccagcagcagccagccactaccgcggcggccgccgtgagcggagccggcgttcagtatgacctggccttggaagagggcgaggggctggcgcggctgggggcgtcgtcgccggagcggcacccgcgcgtgcagatgaaaagggacgctcgcgaggcctacgtgcccaagcagaacctgttcagagacaggagcggcgaggagcccgaggagatgcgcgcctcccgcttccacgcggggcgggagctgcggcgcggcctggaccgaaagcgggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcacgctgatcgcgcgcgaggaggtgaccctgggcctgatgcacctgtgggacctgctggaggccatcgtgcagaaccccacgagcaagccgctgacggcgcagctgtttctggtggtgcagcacagtcgggacaacgagacgttcagggaggcgctgctgaatatcaccgagcccgagggccgctggctcctggacctggtgaacattctgcagagcatcgtggtgcaggagcgcgggctgccgctgtccgagaagctggcggctatcaacttctcggtgctgagcctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagccgccggcgcgagctgagcgaccaggagctgatgcacagcctgcagcgggccctgaccggggccgggaccgagggggagagctactttgacatgggcgcggacctgcgctggcagcccagccgccgggccttggaagctgccggcggttccccctacgtggaggaggtggacgatgaggaggaggagggcgagtacctggaagactgatggcgcgaccgtatttttgctagatgcagcaacagccaccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagacgaacctggaccgcatggtgaccgacgtgcgcgaggcggtgtcgcagcgcgagcggttccaccgcgagtcgaacctgggctccatggtggcgctgaacgccttcctgagcacgcagcccgccaacgtgccccggggccaggaggactacaccaacttcatcagcgcgctgcggctgatggtggccgaggtgccccagagcgaggtgtaccagtcggggccggactacttcttccagaccagtcgccagggcttgcagaccgtgaacctgagccaggctttcaagaacttgcagggactgtggggcgtgcaggccccggtcggggaccgcgcgacggtgtcgagcctgctgacgccgaactcgcgcctgctgctgctgctggtggcgcccttcacggacagcggcagcgtgagccgcgactcgtacctgggctacctgcttaacctgtaccgcgaggccatcgggcaggcgcacgtggacgagcagacctaccaggagatcacccacgtgagccgcgcgctgggccaggaggacccgggcaacctggaggccaccctgaacttcctgctgaccaaccggtcgcagaagatcccgccccagtacgcgctgagcaccgaggaggagcgcatcctgcgctacgtgcagcagagcgtggggctgttcctgatgcaggagggggccacgcccagcgccgcgctcgacatgaccgcgcgcaacatggagcccagcatgtacgctcgcaaccgcccgttcatcaataagctgatggactacttgcatcgggcggccgccatgaactcggactactttaccaacgccatcttgaacccgcactggctcccgccgcccgggttctacacgggcgagtacgacatgcccgaccccaacgacgggttcctgtgggacgacgtggacagcagcgtgttctcgccgcgccccgccaccaccgtgtggaagaaagagggcggggaccggcggccgtcctcggcgctgtccggtcgcgcgggtgctgccgcggcggtgcctgaggccgccagccccttcccgagcctgcccttttcgctgaacagcgtgcgcagcagcgagctgggtcggctgacgcggccgcgcctgctgggcgaggaggagtacctgaacgactccttgttgaggcccgagcgcgagaagaacttccccaataacgggatagagagcctggtggacaagatgagccgctggaagacgtacgcgcacgagcacagggacgagccccgagctagcagcagcgcaggcacccgtagacgccagcgacacgacaggcagcggggtctggtgtgggacgatgaggattccgccgacgacagcagcgtgttggacttgggtgggagtggtggtggtaacccgttcgctcacttgcgcccccgtatcgggcgcctgatgtaagaatctgaaaaaataaaaaacggtactcaccaaggccatggcgaccagcgtgcgttcttctctgttgtttgtagtagtatgatgaggcgcgtgtacccggagggtcctcctccctcgtacgagagcgtgatgcagcaggcggtggcggcggcgatgcagcccccgctggaggcgccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaacgatttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcgcgcaagacccccaatggggtcgcggtggatgagaattatgatggtagtcaggacgagctgacttacgagtgggtggagtttgagctgcccgagggcaacttctcggtgaccatgaccatcgatctgatgaacaacgccatcatcgacaactacttggcggtggggcgtcagaacggggtgctggagagcgacatcggcgtgaagttcgacacgcgcaacttccggctgggctgggaccccgtgaccgagctggtgatgccgggcgtgtacaccaacgaggccttccaccccgacatcgtcctgctgcccggctgcggcgtggacttcaccgagagccgcctcagcaacctgctgggcatccgcaagcggcagcccttccaggagggcttccagatcctgtacgaggacctggaggggggcaacatccccgcgctcttggatgtcgaagcctatgagaaaagcaaggaggaggccgccgcagcggcgaccgcagccgtggccaccgcctctaccgaggtgcggggcgataattttgctagcgccgcggcagtggccgaggcggctgaaaccgaaagtaagatagtcatccagccggtggagaaggacagcaaggacaggagctacaacgtgctcgcggacaagaaaaacaccgcctaccgcagctggtacctggcctacaactacggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgtcactgacgccagacgccgcacctgcccctacgtctacaaggccctgggcgtagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcgctcgcgcaccaccgtcgacgacgtgatcgaccaggtggtggccgacgcgcgcaactacacgcccgccgccgcgcccgcctccaccgtggacgccgtcatcgacagcgtggtggccgatgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcctccggcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggtgaaggaggaaagaaagccccgcaaactgaagcgggtcaaaaaggacaaaaaggaggaggaagatgtggacggactggtggagtttgtgcgcgagttcgccccccggcggcgcgtgcagtggcgcgggcggaaagtgaaaccggtgctgcggcccggcaccacggtggtcttcacgcccggcgagcgttccggctccgcctccaagcgctcctacgacgaggtgtacggggacgaggacatcctcgagcaggcggtcgagcgtctgggcgagtttgcttacggcaagcgcagccgccccgcgcccttgaaagaggaggcggtgtccatcccgctggaccacggcaaccccacgccgagcctgaagccggtgaccctgcagcaggtgctgccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccggcgccggcttccaccactcgccgaagacgcaagtacggcgcggccagcctgctgatgcccaactacgcgctgcatccttccatcatccccacgccgggctaccgcggcacgcgcttctaccgcggctacaccagcagccgccgcaagaccaccacccgccgccgccgtcgtcgcacccgccgcagcagcaccgcgacttccgccgccgccctggtgcggagagtgtaccgcagcgggcgcgagcctctgaccctgccgcgcgcgcgctaccacccgagcatcgccatttaactctgccgtcgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacagcttggaaaatttgtaataaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagccagcccgcgaccctggacttgcctccgcctgcttcccgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtatatgtatgtccgccgaccagaaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcgggggcccagcttcaaaccctactccggcaccgcctacaacagcctggctcccaagggagcgcccaacacttgccagtggacatataaagctggtgatactgatacagaaaaaacctatacatatggaaatgcacctgtgcaaggcattagcattacaaaggatggtattcaacttggaactgacagcgatggtcaggcaatctatgcagacgaaacttatcaaccagagcctcaagtgggtgatgctgaatggcatgacatcactggtactgatgaaaaatatggaggcagagctcttaagcctgacaccaaaatgaagccttgctatggttcttttgccaagcctaccaataaagaaggaggccaggcaaatgtgaaaaccgaaacaggcggtaccaaagaatatgacattgacatggcattcttcgataatcgaagtgcagctgccgccggcctagccccagaaattgttttgtatactgagaatgtggatctggaaactccagatacccatattgtatacaaggcaggtacagatgacagtagctcttctatcaatttgggtcagcagtccatgcccaacagacccaactacattggcttcagagacaactttatcggtctgatgtactacaacagcactggcaatatgggtgtactggctggacaggcctcccagctgaatgctgtggtggacttgcaggacagaaacaccgaactgtcctaccagctcttgcttgactctctgggtgacagaaccaggtatttcagtatgtggaatcaggcggtggacagttatgaccccgatgtgcgcattattgaaaatcacggtgtggaggatgaacttcctaactattgcttccccctggatgctgtgggtagaactgatacttaccagggaattaaggccaatggtgataatcaaaccacctggaccaaagatgatactgttaatgatgctaatgaattgggcaagggcaatcctttcgccatggagatcaacatccaggccaacctgtggcggaacttcctctacgcgaacgtggcgctgtacctgcccgactcctacaagtacacgccggccaacatcacgctgcccaccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgataccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagaggggtacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacgcccttctcggccggcaacgccaccacctaagcctcttgcttcttgcaagatgacggcctgcgcgggctccggcgagcaggagctcagggccatcctccgcgacctgggctgcgggccctgcttcctgggcaccttcgacaagcgcttcccgggattcatggccccgcacaagctggcctgcgccatcgtcaacacggccggccgcgagaccgggggcgagcactggctggccttcgcctggaacccgcgctcccacacctgctacctcttcgaccccttcgggttctcggacgagcgcctcaagcagatctaccagttcgagtacgagggcctgctgcgtcgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgcctactttcgctcccaccgcgcgcgcatcgagaaggccaccgccttcgaccgcatgaatcaagacatgtaatccggtgtgtgtatgtgaatgctttattcatcataataaacagcacatgtttatgccaccttctctgaggctctgactttatttagaaatcgaaggggttctgccggctctcggcatggcccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggagatcttgaaatcgcagttgggacccgcgttctgcgcgcgagagttacggtacacggggttgcagcactggaacaccatcagggccgggtgcttcacgctcgccagcaccgtcgcgtcggtgatgccctccacgtccagatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagccagcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcaccgcagcttgccctcggcctcggtgcacccgtgcagccacagcgcgcagccggtgctctcccagttcttgtgggcgatctgggagtgcgagtgcacgaagccctgcaggaagcggcccatcatcgtggtcagggtcttgttgctggtgaaggtcagcggaatgccgcggtgctcctcgttcacatacaggtggcagatacggcggtacacctcgccctgctcgggcatcagctggaaggcggacttcaggtcgctctccacgcggtaccggtccatcagcagcgtcatcacttccatgcccttctcccaggccgaaacgatcggcaggctcagggggttcttcaccgttgtcatcttagtcgccgccgccgaagtcagggggtcgttctcgtccagggtctcaaacactcgcttgccgtccttctcggtgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttctccttggccgtcgtccgagaccacgcggcggtaggcatgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaagcatggagactcagccatcgtcgccaacatcgccatctgcccccgccgccgccgacgagaaccagcagcagcagaatgaaagcttaaccgccccgccgcccagccccacctccgacgccgcagccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagaaccaggctgggctcgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcgcctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtcctcaagcgcacggtcgagctgacccacttcgcctacccggcgctcaacctgccccccaaggtcatgagcgccgtcatggaccaggtgctcatcaagcgcgcctcgcccctctcggaggaggagatgcaggaccccgagagctcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttcttcgccgacgcggagaccctgcgcaaggtcgaggagaacctgcactacctcttcagacacgggttcgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgcaccaccgccgcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcgctgccctcggacttcgtgccgctgaccttccgcgagtgccccccgccgctctggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaaggccccggcgagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacttggacccccagaccggagaggagctcaaccccagcttcccccaggatgccccgaggaagcagcaagaagctgaaagtggagctgccgccgccgccggaggatttggaggaagactgggagagcagtcaggcagaggaggaggagatggaagactgggacagcactcaggcagaggaggacagcctgcaagacagtctggaggaggaagacgaggtggaggaggcagaggaagaagcagccgccgccagaccgtcgtcctcggcggaggaggagaaagcaagcagcacggataccatctccgctccgggtcggggtcgcggcggccgggcccacagtagatgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagcagcagcagaaaaccagcggcagcagctagaaaatccacagcggcggcaggtggactgaggatcgcggcgaacgagccggcgcagacccgggagctgaggaaccggatctttcccaccctctatgccatcttccagcagagtcgggggcaagagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcggctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggtctcgactcccacctgcttcggatcttcagccagcgtccgatcctggccgagcgcgagcaaggacagacccttctgaccctgtactgcatctgcaaccaccccggcctgcatgaaagtctttgttgtctgctgtgtactgagtataataaaagctgagatcagcgactactccggacttccgtgtgttcctgctatcaaccagtccctgttcttcaccgggaacgagaccgagctccagctccagtgtaagccccacaagaagtacctcacctggctgttccagggctctccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgtcgctccccgctactaacaaccaaactacccaccaacgccaccgtcgcgacctttcctctgggtctaataccactaccggaggtgagctccgaggtcgaccaacctctgggatttactacggcccctgggaggtggtagggttaatagcgctaggcctagttgcgggtgggcttttggctctctgctacctatacctcccttgctgttcgtacttagtggtgctgtgttgctggtttaagaaatggggaagatcaccctagtgagctgcggtgtgctggtggcggtggtgctttcgattgtgggactgggcggcgcggctgtagtgaaggagaaggccgatccctgcttgcatttcaatcccgacaaatgccagctgagttttcagcccgatggcaatcggtgcgcggtgctgatcaagtgcggatgggaatgcgagaacgtgagaatcgagtacaataacaagactcggaacaatactctcgcgtccgtgtggcagcccggggaccccgagtggtacaccgtctctgtccccggtgctgacggctccccgcgcaccgtgaataatactttcatttttgcgcacatgtgcgacacggtcatgtggatgagcaagcagtacgatatgtggccccccacgaaggagaacatcgtggtcttctccatcgcttacagcgtgtgcacggcgctaatcaccgctatcgtgtgcctgagcattcacatgctcatcgctattcgccccagaaataatgccgaaaaagaaaaacagccataacacgttttttcacacacctttttcagaccatggcctctgttaaatttttgcttttatttgccagtctcattgccgtcattcatggaatgagtaatgagaaaattactatttacactggcactaatcacacattgaaaggtccagaaaaagccacagaagtttcatggtattgttattttaatgaatcagatgtatctactgaactctgtggaaacaataacaaaaaaaatgagagcattactctcatcaagtttcaatgtggatctgacttaaccctaattaacatcactagagactatgtaggtatgtattatggaactacagcaggcatttcggacatggaattttatcaagtttctgtgtctgaacccaccacgcctagaatgaccacaaccacaaaaactacacctgttaccactatacagctcactaccaatggctttcttgccatgcttcaagtggctgaaaatagcaccagcattcaacccaccccacccagtgaggaaattcccagatccatgattggcattattgttgctgtagtggtgtgcatgttgatcatcgccttgtgcatggtgtactatgccttctgctacagaaagcacagactgaacgacaagctggaacacttactaagtgttgaattttaattttttagaaccatgaagatcctaggccttttagttttttctatcattacctctgctctatgcaattctgacaatgaggacgttactgtcgttgtcggatcaaattatacactaaaaggtccagcaaaaggtatgctttcgtggtattgttggttcggaactgacgagcaacagacagaactttgcaatgctcaaaaaggcaaaacctcaaattctaaaatctctaattatcaatgcaatggcactgacttagtattgctcaatgtcacgaaagcatatgctggcagttacacctgccctggagatgatgccgacaatatgattttttacaaagtggaagtggttgatcccactactccaccgcccaccaccacaactactcataccacacacacagaacaaacaccagaggcagcagaagcagagttggccttccaggttcacggagattcctttgctgtcaatacccctacacccgatcagcggtgtccggggctgctcgtcagcggcattgtcggtgtgctttcgggattagcagtcataatcatctgcatgttcatttttgcttgctgctatagaaggctttaccgacaaaaatcagacccactgctgaacctctatgtttaattttttccagagccatgaaggcagttagcgctctagttttttgttctttgattggcattgtttttagtgctgggtttttgaaaaatcttaccatttatgaaggtgagaatgccactctagtgggcatcagtggtcaaaatgtcagctggctaaaataccatctagatgggtggaaagacatttgcgattggaatgtcactgtgtatacatgtaatggagttaacctcaccattactaatgccacccaagatcagaatggtaggtttaagggccagagtttcactagaaataatgggtatgaatcccataacatgtttatctatgacgtcactgtcatcagaaatgagactgccaccaccacacagatgcccactacacacagttctaccactactaccatgcaaaccacacagacaaccactacatcaactcagcatatgaccaccactacagcagcaaagccaagtagtgcagcgcctcagccccaggctttggctttgaaagctgcacaacctagtacaactactaggaccaatgagcagactactgaatttttgtccactgtcgagagccacaccacagctacctccagtgccttctctagcaccgccaatctctcctcgctttcctctacaccaatcagtcccgctactactcccaccccagctcttctccccactcccctgaagcaaactgaggacagcggcatgcaatggcagatcaccctgctcattgtgatcgggttggtcatcctggccgtgttgctctactacatcttctgccgccgcattcccaacgcgcaccgcaaaccggcctacaagcccatcgttatcgggcagccggagccgcttcaggtggaagggggtctaaggaatcttctcttctcttttacagtatggtgattgaactatgattcctagacaattcttgatcactattcttatctgcctcctccaagtctgtgccaccctcgctctggtggccaacgccagtccagactgtattgggcccttcgcctcctacgtgctctttgccttcatcacctgcatctgctgctgtagcatagtctgcctgcttatcaccttcttccagttcattgactggatctttgtgcgcatcgcctacctgcgccaccacccccagtaccgcgaccagcgagtggcgcggctgctcaggctcctctgataagcatgcgggctctgctacttctcgcgcttctgctgttagtgctcccccgccccgtcgacccccggtcccccactcagtcccccgaagaggtccgcaaatgcaaattccaagaaccctggaaattcctcaaatgctaccgccaaaaatcagacatgcttcccagctggatcatgatcattgggatcgtgaacattctggcctgcaccctcatctcctttgtgatttacccctgctttgactttggttggaactcgccagaggcgctctatctcccgcctgaacctgacacaccaccacagcaacctcaggcacacgcactaccaccaccacagcctaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggacggcatagccatccaccagtgcaagaaaggcatcttctgcctggtgaaacaggccaagatctcctacgaggtcaccccgaccgaccatcgcctctcctacgagctcctgcagcagcgccagaagttcacctgcctggtcggagtcaaccccatcgtcatcacccagcagtcgggcgataccaaggggtgcatccactgctcctgcgactcccccgactgcgtccacactctgatcaagaccctctgcggcctccgcgacctcctccccatgaactaatcacccccttatccagtgaaataaatatcatattgatgatgatttaaataaaaaataatcatttgatttgaaataaagatacaatcatattgatgatttgagttttaaaaaataaagaatcacttacttgaaatctgataccaggtctctgtccatgttttctgccaacaccacctcactcccctcttcccagctctggtactgcagaccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgctgtccctgcgactggctgaccccgtcaccaccaagaacggggaaatcaccctcaagctgggagagggggtggacctcgactcctcgggaaaactcatctccaacacggccaccaaggccgccgcccctctcagtttttccaacaacaccatttcccttaacatggatacccctctttataccaaagatggaaaattatccttacaagtttctccaccgttaaacatattaaaatcaaccattctgaacacattagctgtagcttatggatcaggtttaggactgagtggtggcactgctcttgcagtacagttggcctctccactcacttttgatgaaaaaggaaatattaaaattaacctagccagtggtccattaacagttgatgcaagtcgacttagtatcaactgcaaaagaggggtcactgtcactacctcaggagatgcaattgaaagcaacataagctggcctaaaggtataagatttgaaggtaatggcatagctgcaaacattggcagaggattggaatttggaaccactagtacagagactgatgtcacagatgcatacccaattcaagttaaattgggtactggccttacctttgacagtacaggcgccattgttgcttggaacaaagaggatgataaacttacattatggaccacagccgacccctcgccaaattgcaaaatatactctgaaaaagatgccaaactcacactttgcttgacaaagtgtggaagtcaaattctgggtactgtgactgtattggcagtgaataatggaagtctcaacccaatcacaaacacagtaagcactgcactcgtctccctcaagtttgatgcaagtggagttttgctaagcagctccacattagacaaagaatattggaacttcagaaagggagatgttacacctgctgagccctatactaatgctataggttttatgcctaacataaaggcctatcctaaaaacacatctgcagcttcaaaaagccatattgtcagtcaagtttatctcaatggggatgaggccaaaccactgatgctgattattacttttaatgaaactgaggatgcaacttgcacctacagtatcacttttcaatggaaatgggatagtactaagtacacaggtgaaacacttgctaccagctccttcaccttctcctacatcgcccaagaatgaacactgtatcccaccctgcatgccaacccttcccaccccactctgtctatggaaaaaactctgaagcacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacaggaccaccaggttgttcaacagtccatagttcaacacgctccagccgaaactcatcgcgggaaggatgctacccacgtggccgtcgtaccagatcctcaggtaaatcaagtggcgctccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtcccggcgcttggaacgctcggtgttgaaattgtaaaacagccactctctcagaccgtgcagcagatctagggcctcaggagtgatgaagatcccatcatgcctgatagctctgatcacatcgaccaccgtggaatgggccagacccagccagatgatgcaattttgttgggtttcggtgacggcgggggagggaagaacaggaagaaccatgattaacttttaatccaaacggtctcggagcacttcaaaatgaaggtcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctaagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcacccaggcaatttttaagaaaatcaacaaaagaaaaatcctccaggtgcacgtttagagcctcgggaacaacgatgaagtaaatgcaagcggtgcgttccagcatggttagttagctgatctgtaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaatcctcaggtgcgtacaaaatgtaattactcccctcctgcacaggcagcgaagcccccgatccctccagatacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaactgccgtcatttccgggttcccacgctacgtcatcggaattcgactttcaaattccgtcgaccgttaaaaacgtcacccgccccgcccctaacggtcgcccgtctctcggccaatcaccttcctccctccccaaattcaaacagctcatttgcatattaacgcgcaccaaaagtttgaggtatattattgatgatg

SEQ ID NO: 10 - protein, 531 aa, chimpanzee adenovirus serotype Pan7
MMRRVYPEGPPPSYESVMQQAVAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVAVDENYDGSQDELTYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQILYEDLEGGNIPALLDVEAYEKSKEEAAAAATAAVATASTEVRGDNFASAAAVAEAAETESKIVIQPVEKDSKDRSYNVLADKKNTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGVVAPRVLSSRTF

SEQ ID NO: 11 - protein, 932 aa, chimpanzee adenovirus serotype Pan7
MATPSMLPQWAYMHIAGQDASEYLSPGLVQFARATDTYFSLGNKFRNPTVAPTHDVTTDRSQRLTLRFVPVDREDNTYSYKVRYTLAVGDNRVLDMASTYFDIRGVLDRGPSFKPYSGTAYNSLAPKGAPNTCQWTYKAGDTDTEKTYTYGNAPVQGISITKDGIQLGTDSDGQAIYADETYQPEPQVGDAEWHDITGTDEKYGGRALKPDTKMKPCYGSFAKPTNKEGGQANVKTETGGTKEYDIDMAFFDNRSAAAAGLAPEIVLYTENVDLETPDTHIVYKAGTDDSSSSINLGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDAVGRTDTYQGIKANGDNQTTWTKDDTVNDANELGKGNPFAMEINIQANLWRNFLYANVALYLPDSYKYTPANITLPTNTNTYDYMNGRVVAPSLVDAYINIGARWSLDPMDNVNPFNHHRNAGLRYRSMLLGNGRYVPFHIQVPQKFFAIKSLLLLPGSYTYEWNFRKDVNMILQSSLGNDLRTDGASIAFTSINLYATFFPMAHNTASTLEAMLRNDTNDQSFNDYLSAANMLYPIPANATNVPISIPSRNWAAFRGWSFTRLKTRETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVASVTQKKFLCDRVMWRIPFSSNFMSMGALTDLGQNMLYANSAHALDMNFEVDPMDESTLLYVVFEVFDVVRVHQPHRGVIEAVYLRTPFSAGNATT

SEQ ID NO: 12 - protein, 443 aa, chimpanzee adenovirus serotype Pan7
MSKKRVRVDDDFDPVYPYDADNAPTVPFINPPFVSSDGFQEKPLGVLSLRLADPVTTKNGEITLKLGEGVDLDSSGKLISNTATKAAAPLSFSNNTISLNMDTPLYTKDGKLSLQVSPPLNILKSTILNTLAVAYGSGLGLSGGTALAVQLASPLTFDEKGNIKINLASGPLTVDASRLSINCKRGVTVTTSGDAIESNISWPKGIRFEGNGIAANIGRGLEFGTTSTETDVTDAYPIQVKLGTGLTFDSTGAIVAWNKEDDKLTLWTTADPSPNCKIYSEKDAKLTLCLTKCGSQILGTVTVLAVNNGSLNPITNTVSTALVSLKFDASGVLLSSSTLDKEYWNFRKGDVTPAEPYTNAIGFMPNIKAYPKNTSAASKSHIVSQVYLNGDEAKPLMLIITFNETEDATCTYSITFQWKWDSTKYTGETLATSSFTFSYIAQE

SEQ ID NO: 13 - protein, 338 aa, simian serotype C1
APKGAPNTSQWLDKGVTTTDNNTENGDEEDEVAEEGEEEKQATYTFGNAPVKAEAEITKEGLPIGLEVPSEGDPKPIYADKLYQPEPQVGEESWTDTDGTDEKYGGRALKPETKMKPCYGSFAKPTNVKGGQAKVKKVEEGKVEYDIDMNFFDLRSQKTGLKPKIVMYAENVDLETPDTHVVYKPGASDASSHANLGQQSMPNRPNYIGFRDNFIGLMYYNSTGNMGVLAGQASQLNAVVDLQDRNTELSYQLLLDSLGDRTRYFSMWNQAVDSYDPDVRVIENHGVEDELPNYCFPLDGVGPRTDSYKGIETNGDENTTWKDLDPNGISELAKGNPF

SEQ ID NO: 14 - protein, 315 aa, chimpanzee adenovirus Pan-9
APKGAPNTCQWTYKADGETATEKTYTYGNAPVQGINITKDGIQLGTDTDDQPIYADKTYQPEPQVGDAEWHDITGTDEKYGGRALKPDTKMKPCYGSFAKPTNKEGGQANVKTGTGTTKEYDIDMAFFDNRSAAAAGLAPEIVLYTENVDLETPDTHIVYKAGTDDSSSSINLGQQAMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDAVGRTDTYQGIKANGTDQTTWTKDDSVNDANEIGKGNPF

SEQ ID NO: 15 - protein, 315 aa, chimpanzee adenovirus Pan-5
APKGAPNTCQWTYKADGDTGTEKTYTYGNAPVQGISITKDGIQLGTDTDDQPIYADKTYQPEPQVGDAEWHDITGTDEKYGGRALKPDTKMKPCYGSFAKPTNKEGGQANVKTETGGTKEYDIDMAFFDNRSAAAAGLAPEIVLYTENVDLETPDTHIVYKAGTDDSSSSINLGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDAVGRTDTYQGIKANGADQTTWTKDDTVNDANELGKGNPF

SEQ ID NO: 16 - protein, 324 aa, chimpanzee adenovirus Pan-6
APKGAPNSSQWEQAKTGNGGTMETHTYGVAPMGGENITKDGLQIGTDVTANQNKPIYADKTFQPEPQVGEENWQETENFYGGRALKKDTKMKPCYGSYARPTNEKGGQAKLKVGDDGVPTKEFDIDLAFFDTPGGTVNGQDEYKADIVMYTENTYLETPDTHVVYKPGKDDASSEINLVQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDGSGTNAAYQGVKVKDGQDGDVESEWENDDTVAARNQLCKGNIF

SEQ ID NO: 17 - protein, 314 aa, chimpanzee adenovirus Pan-7
APKGAPNTCQWTYKAGDTDTEKTYTYGNAPVQGISITKDGIQLGTDSDGQAIYADETYQPEPQVGDAEWHDITGTDEKYGGRALKPDTKMKPCYGSFAKPTNKEGGQANVKTETGGTKEYDIDMAFFDNRSAAAAGLAPEIVLYTENVDLETPDTHIVYKAGTDDSSSSINLGQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDAVGRTDTYQGIKANGDNQTTWTKDDTVNDANELGKGNPF

SEQ ID NO: 18 - protein, 179 aa, chimpanzee adenovirus Pan9
TLWTTPDPSPNCQILAENDAKLTLCLTKCGSQILATVSVLVVGSGNLNPITGTVSSAQVFLRFDANGVLLTEHSTLKKYWGYRQGDSIDGTPYTNAVGFMPNLKAYPKSQSSTTKNNIVGQVYMNGDVSKPMLLTITLNGTDDSNSTYSMSFSYTWTNGSYVGATFGANSYTFSYIAQE

SEQ ID NO: 19 - protein, 185 aa, chimpanzee adenovirus Pan6
TLWTTPDPSPNCQLLSDRDAKFTLCLTKCGSQILGTVAVAAVTVGSALNPINDTVKSAIVFLRFDSDGVLMSNSSMVGDYWNFREGQTTQSVAYTNAVGFMPNIGAYPKTQSKTPKNSIVSQVYLTGETTMPMTLTITFNGTDEKDTTPVSTYSMTFTWQWTGDYKDKNITFATNSFSFSYIAQE

SEQ ID NO: 20 - protein, 179 aa, chimpanzee adenovirus Pan7
TLWTTADPSPNCKIYSEKDAKLTLCLTKCGSQILGTVTVLAVNNGSLNPITNTVSTALVSLKFDASGVLLSSSTLDKEYWNFRKGDVTPAEPYTNAIGFMPNIKAYPKNTSAASKSHIVSQVYLNGDEAKPLMLIITFNETEDATCTYSITFQWKWDSTKYTGETLATSSFTFSYIAQE

SEQ ID NO: 21 - protein, 179 aa, chimpanzee adenovirus Pan5
TLWTTADPSPNCHIYSEKDAKLTLCLTKCGSQILGTVSLIAVDTGSLNPITGTVTTALVSLKFDANGVLQSSSTLDSDYWNFRQGDVTPAEAYTNAIGFMPNLKAYPKNTSGAAKSHIVGKVYLHGDTGKPLDLIITFNETSDESCTYCINFQWQWGADQYKNETLAVSSFTFSYIAKE

SEQ ID NO: 22 - protein, 183 aa, human adenovirus Ad 2
TLWTTPDPSPNCRIHSDNDCKFTLVLTKCGSQVLATVAALAVSGDLSSMTGTVASVSIFLRFDQNGVLMENSSLKKHYWNFRNGNSTNANPYTNAVGFMPNLLAYPKTQSQTAKNNIVSQVYLHGDKTKPMILTITLNGTSESTETSEVSTYSMSFTWSWESGKYTTETFATNSYTFSYIAQE

SEQ ID NO: 23 - protein, 182 aa, human adenovirus Ad 5
TLWTTPAPSPNCRLNAEKDAKLTLVLTKCGSQILATVSVLAVKGSLAPISGTVQSAHLIIRFDENGVLINNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTESYIAQE

SEQ ID NO: 24 - DNA, 34264 bp, simian adenovirus SV-1
  CDS (12454)..(13965): L2 Penton
tccttattctggaaacgtgccaatatgataatgagcggggaggagcgaggcggggccggggtgacgtgcggtgacgtggggtgacgcggggtggcgcgagggcggggcgggagtggggaggcgcttagtttttacgtatgcggaaggaggttttataccggaagttgggtaatttgggcgtatacttgtaagttttgtgtaatttggcgcgaaaaccgggtaatgaggaagttgaggttaatatgtactttttatgactgggcggaatttctgctgatcagcagtgaactttgggcgctgacggggaggtttcgctacgtggcagtaccacgagaaggctcaaaggtcccatttattgtactcctcagcgttttcgctgggtatttaaacgctgtcagatcatcaagaggccactcttgagtgccggcgagtagagttttctcctccgcgctgccgcgatgaggctggttcccgagatgtacggtgttttctgcagcgagacggcccggaactcagatgagctgcttaatacagatctgctggatgttcccaactcgcctgtggcttcgcctccgtcgcttcatgatcttttcgatgtggaagtggatccaccgcaagatcccaacgaggacgcggtaaacagtatgttccctgaatgtctgtttgaggcggctgaggagggttctcacagcagtgaagagagcagacggggagaggaactggacttgaaatgctacgaggaatgtctgccttctagcgattctgaaacggaacagacagggggagacggctgtgagtcggcaatgaaaaatgaacttgtattagactgtccagaacatcctggtcatggctgccgtgcctgtgcttttcatagaaatgccagcggaaatcctgagactctatgtgctctgtgttatctgcgccttaccagcgattttgtatacagtaagtaaagtgttttcattggcgtacggtaggggattcgttgaagtgctttgtgacttattatgtgtcattatttctaggtgacgtgtccgacgtggaaggggaaggagatagatcaggggctgctaattctccttgcactttgggggctgtggttccagttggcatttttaaaccgagtggtggaggagaacgagccggaggagaccgagaatctgagagccggcctggaccctccagtggaagactaggtgctgaggatgatcctgaagaggggactagtgggggtgctaggaaaaagcaaaaaactgagcctgaacctagaaactttttgaatgagttgactgtaagcctaatgaatcggcagcgtcctgagacggtgttttggactgagttggaggatgagttcaagaagggggaattaaacctcttgtacaagtatgggtttgagcagttgaaaactcactggttggagccgtgggaggatatggaaatggctctagacacctttgctaaagtggctctgcggccggataaagtttacactattcgccgcactgttaatataaaaaagagtgtttatgttatcggccatggagctctggtgcaggtgcagaccccagaccgggtggctttcaattgcggcatgcagagtttgggccccggggtgataggtttgaatggagttacatttcaaaatgtcaggtttactggtgatgattttaatggctctgtgtttgtgactagcacccagctaaccctccacggtgtttacttttttaactttaacaatacatgtgtggagtcatggggtagggtgtctctgaggggctgcagttttcatggttgctggaaggcggtggtgggaagaattaaaagtgtcatgtctgtgaagaaatgcatatttgaacgctgtgtgatagctctagcagtagaggggtacggacggatcaggaataacgccgcatctgagaatggatgttttcttttgctgaaaggtacggccagcgttaagcataatatgatttgcggcagcggcctgtgcccctcgcagctcttaacttgcgcagatggaaactgtcacaccttgcgcaccgtgcacatagtgtcccactcgcgccgcacctggccaacatttgagcacaatatgctcatgcgttgcgccgttcacctaggtgctagacgcggcgtgtttatgccttatcaatgtaactttagtcatactaagattttgctggaaactgattccttccctcgagtatgtttcaatggggtgtttgacatgtcaatggaactttttaaagtgataagatatgatgaaaccaagtctcgttgtcgctcatgtgaatgcggagctaatcatttgaggttgtatcctgtaaccctgaacgttaccgaggagctgaggacggaccaccacatgctgtcttgcctgcgtaccgactatgaatccagcgatgaggagtgaggtgaggggcggagccacaaagggtataaaggggcatgaggggtgggcgcggtgtttcaaaatgagcgggacgacggacggcaatgcgtttgaggggggagtgttcagcccatatctgacatctcgtcttccttcctgggcaggagttcgtcagaatgtagtgggctccaccgtggacggacggccggtcgcccctgcaaattccgccaccctcacctatgccaccgtgggatcatcgttggacactgccgcggcagctgccgcttctgctgccgcttctactgctcgcggcatggcggctgattttggactatataaccaactggccactgcagctgtggcgtctcggtctctggttcaagaagatgccctgaatgtgatcttgactcgcctggagatcatgtcacgtcgcctggacgaactggctgcgcagatatcccaagctaaccccgataccgcttcagaatcttaaaataaagacaaacaaatttgttgaaaagtaaaatggctttatttgttttttttggctcggtaggctcgggtccacctgtctcggtcgttaaggactttgtgtatgttttccaaaacacggtacagatgggcttggatgttcaagtacatgggcatgaggccatctttggggtggagataggaccactgaagagcgtcatgttccggggtggtattgtaaatcacccagtcgtagcagggtttttgagcgtggaactggaatatgtccttcaggagcaggctaatggccaagggtagacccttagtgtaggtgtttacaaagcggttgagctgggagggatgcatgcggggggagatgatatgcatcttggcttggattttgaggttagctatgttaccacccaggtctctgcgggggttcatgttatgaaggaccaccagcacggtatagccagtgcatttggggaacttgtcatgcagtttggaggggaaggcgtggaagaatttagatacccccttgtgcccccctaggttttccatgcactcatccataataatggcaatgggacccctggcggccgctttagcaaacacgttttgggggttggaaacatcatagttttgctctagagtgagctcatcataggccatctttacaaagcggggtaggagggtgcccgactgggggatgatagttccatctgggcctggagcgtagttgccctcacagatctgcatctcccaggccttaatttccgagggggggatcatgtccacctggggggcgataaaaaacacggtttctggcggggggttaatgagctgggtggaaagcaagttacgcaacagctgggatttgccgcaaccggtgggaccgtagatgaccccgatgacgggttgcagctggtagttcagagaggaacagctgccgtcggggcgcaggaggggagctacctcattcatcatgcttctgacatgtttattttcactcactaagttttgcaagagcctctccccacccagggataagagttcttccaggctgttgaagtgtttcagcggtttcaggccgtcggccatgggcatcttttcaagcgactgacgaagcaagtacagtcggtcccagagctcggtgacgtgctctatggaatctcgatccagcagacttcttggtttcgggggttgggccgactttcgctgtagggcaccagccggtgggcgtccagggccgcgagggttctgtccttccagggtctcagcgttcgggtgagggtggtctcggtgacggtgaagggatgagccccgggctgggcgcttgcgagggtgcgcttcaggctcatcctgctggtgctgaagcgggcgtcgtctccctgtgagtcggccagatagcaacgaagcatgaggtcgtagctgagggactcggccgcgtgtcccttggcgcgcagctttcccttggaaacgtgctgacatttggtgcagtgcagacacttgagggcgtagagttttggggccaggaagaccgactcgggcgagtaggcgtcggctccgcactgagcgcagacggtctcgcactccaccagccacgtgagctcgggtttagcgggatcaaaaaccaagttgcctccattttttttgatgcgtttcttaccttgcgtctccatgagtctgtgtcccgcttccgtgacaaaaaggctgtcggtatccccgtagaccgacttgagggggcgatcttccaaaggtgttccgaggtcttccgcgtacaggaactgggaccactccgagacaaaggctcgggtccaggctaacacgaaggaggcgatctgcgaggggtatctgtcgttttcaatgagggggtccaccttttccagggtgtgcagacacaggtcgtcctcctccgcgtccacgaaggtgattggcttgtaagtgtaggtcacgtgacccgcacccccccaaggggtataaaagggggcgtgcccactctccccgtcactttcttccgcatcgctgtggaccagagccagctgttcgggtgagtaggccctctcaaaagccggcatgatttcggcgctcaagttgtcagtttctacaaacgaggtggatttgatattcacgtgccccgcggcgatgcttttgatggtggaggggtccatctgatcagaaaacacgatctttttattgtcaagtttggtggcgaaagacccgtagagggcgttggaaagcaacttggcgatggagcgcagggtctgatttttctcccgatcggccctctccttggcggcgatgttgagttgcacgtactcgcgggccacgcaccgccactcggggaacacggcggtgcgctcgtcgggcaggatgcgcacgcgccagccgcggttgtgcagggtgatgaggtccacgctggtggccacctccccgcggaggggctcgttggtccaacacaatcgccccccttttctggagcagaacggaggcaggggatctagcaagttggcgggcggggggtcggcgtcgatggtaaatatgccgggtagcagaattttattaaaataatcgatttcggtgtccgtgtcttgcaacgcgtcttcccacttcttcaccgccagggccctttcgtagggattcaggggcggtccccagggcatggggtgggtcagggccgaggcgtacatgccgcagatgtcgtacacgtacaggggctccctcaacaccccgatgtaagtggggtaacagcgccccccgcggatgctggctcgcacgtagtcgtacatctcgtgagagggagccatgagcccgtctcccaagtgggtcttgtggggtttttcggcccggtagaggatctgcctgaagatggcgtgggagttggaagagatagtggggcgttggaagacgttaaagttggctccgggcagtcccacggagtcttggatgaactgggcgtaggattcccggagcttgtccaccagggctgcggttaccagcacgtcgagagcgcagtagtccaacgtctcgcggaccaggttgtaggccgtctcttgttttttctcccacagttcgcgattgaggaggtattcctcgcggtctttccagtactcttcggcgggaaatcctttttcgtccgctcggtaagaacctaacatgtaaaattcgttcacggctttgtatggacaacagcctttttctaccggcagggcgtacgcttgagcggcctttctgagagaggtgtgggtgagggcgaaggtgtcccgcaccatcactttcaggtactgatgtttgaagtccgtgtcgtcgcaggcgccctgttcccacagcgtgaagtcggtgcgctttttctgcctgggattggggagggcgaatgtgacgtcgttaaagaggattttcccggcgcggggcatgaagttgcgagagatcctgaagggtccgggcacgtccgagcggttgttgatgacttgcgccgccaggacgatctcgtcgaagccgttgatgttgtggcccacgatgtaaagttcgataaagcgcggctgtcccttgagggccggcgcttttttcaactcctcgtaggtgagacagtccggcgaggagagacccagctccgcccgggcccagtcggagagctgagggttagccgcgaggaaagagctccacaggtcaagggctagcagagtttgcaagcggtcgcggaactcgcgaaactttttccccacggccattttctccggcgtcaccacgtagaaagtgcaggggcggtcgttccagacgtcccatcggagctctagggccagctcgcaggcttgacgaacgagggtctcctcgcccgagacgtgcatgaccagcatgaagggtaccaactgtttcccgaacgagcccatccatgtgtaggtttctacgtcgtaggtgacaaagagccgctgggtgcgcgcgtgggagccgatcgggaagaagctgatctcctgccaccagttggaggaatgggtgttgatgtggtgaaagtagaagtcccgccggcgcacagagcattcgtgctgatgtttgtaaaagcgaccgcagtagtcgcagcgctgcacgctctgtatctcctgaatgagatgcgcttttcgcccgcgcaccagaaaccggagggggaagttgagacgggggcttggtggggcggcatccccttcgccttggcggtgggagtctgcgtctgcgccctccttctctgggtggacgacggtggggacgacgacgccccgggtgccgcaagtccagatctccgccacggaggggcgcaggcgttgcaggaggggacgcagctgcccgctgtccagggagtcgagggcggccgcgctgaggtcggcgggaagcgtttgcaagttcactttcagaagaccggtaagagcgtgagccaggtgcacatggtacttgatttccaggggggtgttggaagaggcgtccacggcgtagaggaggccgtgtccgcgcggggccaccaccgtgccccgaggaggttttatctcactcgtcgagggcgagcgccggggggtagaggcggctctgcgccggggggcagcggaggcagtggcacgttttcgtgaggattcggcagcggttgatgacgagcccggagactgctggcgtgggcgacgacgcggcggttgaggtcctggatgtgccgtctctgcgtgaagaccaccggcccccgggtcctgaacctgaaagagagttccacagaatcaatgtctgcatcgttaacggcggcctgcctgaggatctcctgtacgtcgcccgagttgtcttgataggcgatctcggccatgaactgctccacttcttcctcgcggaggtcgccgtggcccgctcgctccacggtggcggccaggtcgttggagatgcgacgcatgagttgagagaaggcgttgaggccgttctcgttccacacgcggctgtacaccacgtttccgaaggagtcgcgcgctcgcatgaccacctgggccacgttgagttccacgtggcgggcgaagacggcgtagtttctgaggcgctggaagaggtagttgagcgtggtggcgatgtgctcgcagacgaagaagtacatgatccagcgccgcagggtcatctcgttgatgtctccgatggcttcgagacgctccatggcctcgtagaagtcgacggcgaagttgaaaaattgggagttgcgggcggccaccgtgagttcttcttgcaggaggcggatgagatcggcgaccgtgtcgcgcacctcctgctcgaaagcgccccgaggcgcctctgcttcttcctccggctcctcctcttccaggggcacgggttcctccggcagctctgcgacggggacggggcggcgacgtcgtcgtctgaccggcaggcggtccacgaagcgctcgatcatttcgccgcgccggcgacgcatggtctcggtgacggcgcgtccgttttcgcgaggtcgcagttcgaagacgccgccgcgcagagcgcccccgtgcagggagggtaagtggttagggccgtcgggcagggacacggcgctgacgatgcattttatcaattgctgcgtaggcactccgtgcagggatctgagaacgtcgaggtcgacgggatccgagaacttctctaggaaagcgtctatccaatcgcagtcgcaaggtaagctgaggacggtgggccgctggggggcgtccgcgggcagttgggaggtgatgctgctgatgatgtaattaaagtaggcggtcttcaggcggcggatggtggcgaggaggaccacgtctttgggcccggcctgttgaatgcgcaggcgctcggccatgccccaggcctcgctctgacagcgacgcaggtctttgtagtagtcttgcatcagtctctccaccggaacctctgcttctcccctgtctgccatgcgagtcgagccgaacccccgcaggggctgcagcaacgctaggtcggccacgaccctctcggccagcacggcctgttggatctgcgtgagggtggtctggaagtcgtccaggtccacgaagcggtgataggcccccgtgttgatggtgtaggtgcagttggccatgacggaccagttgacgacttgcatgccgggttgggtgatctccgtgtacttgaggcgcgagtaggcgcgggactcgaacacgtagtcgttgcatgtgcgtaccagatactggtagccaaccaggaagtggggaggcggttctcggtacaggggccagccgactgtggcgggggcgccgggggacaggtcgtccagcatgaggcgatggtagtggtagatgtagcgggagagccaggtgatgccggccgaggtggtcgcggccctggtgaattcgcggacgcggttccagatgttgcgcagggggcgaaagcgctccatggtgggcacgctctgccccgtgaggcgggcgcaatcttgtacgctctagatggaaaaaagacagggcggtcatcgactcccttccgtagctcggggggtaaagtcgcaagggtgcggcggcggggaaccccggttcgagaccggccggatccgccgctcccgatgcgcctggccccgcatccacgacgtccgcgtcgagacccagccgcgacgctccgccccaatacggaggggagtcttttggtgttttttcgtagatgcatccggtgctgcggcagatgcgacctcagacgcccaccaccaccgccgcggcggcagtaaacctgagcggaggcggtgacagggaggaggaggagctggctttagacctggaagagggagaggggctggcccggctgggagcgccgtccccagagagacaccctagggttcagctcgtgagggacgccaggcaggcttttgtgccgaagcagaacctgtttagggaccgcagcggtcaggaggcggaggagatgcgcgattgcaggtttcgggcgggtagagagctgagggcgggcttcgatcgggagcggctcctgagggcggaggatttcgagcccgacgagcgttctggggtgagcccggcccgcgctcacgtctcggcggccaacctggtgagcgcgtacgagcagacggtgaacgaggagcgcaacttccaaaagagctttaacaatcacgtgaggaccctgatcgcgagggaggaggtgaccatcgggctgatgcatctgtgggacttcgtggaggcctacgtgcagaacccggccagcaaacctctgacggcccagctgttcctgatcgtgcagcacagccgcgacaacgagacgttccgcgacgccatgttgaacatcgcggagcccgagggtcgctggctcttggatctgattaacatcctgcagagcatcgtggtgcaggagaggggcctcagcttagcggacaaggtggcggccattaactattcgatgcagagcctggggaagttctacgctcgcaagatctacaagagcccttacgtgcccatagacaaggaggtgaagatagacagcttttacatgcgcatggcgctgaaggtgctgacgctgagcgacgacctcggcgtgtaccgtaacgacaagatccacaaggcggtgagcgccagccgccggcgggagctgagcgacagggagctgatgcacagcctgcagagggcgctggcgggcgccggggacgaggagcgcgaggcttacttcgacatgggagccgatctgcagtggcgtcccagcgcgcgcgccttggaggcggcgggctaccccgacgaggaggatcgggacgatttggaggaggcaggcgagtacgaggacgaagcctgaccgggcaggtgttgttttagatgcagcggccggcggacggggccaccgcggatcccgcacttttggcatccatgcagagtcaaccttcgggcgtgaccgcctccgatgactgggcggcggccatggaccgcattatggcgctgactacccgcaaccccgaggcttttagacagcaaccccaggccaaccgtttttcggccatcttggaagcggtggtgccctcccgcaccaaccccacacacgagaaagtcctgactatcgtgaacgccctggtagacagcaaggccatccgccgcgacgaggcgggcttgatttacaacgctctgctggaacgggtggcgcgctacaacagcactaacgttcagaccaatctggatcgcctcaccaccgacgtgaaggaggcgctggctcagaaggagcggtttctgagggacagcaatctgggctctctggtggcactcaacgccttcctgagcacgcagccggccaacgtgccccgcgggcaggaggactacgtgagcttcatcagcgctctgaggctgctggtgtccgaggtgccccagagcgaggtgtatcagtctgggccggattacttcttccagacgtcccgacagggcttgcaaacggtgaacctgactcaggcctttaaaaacttgcaaggcatgtggggcgttaaggccccggtgggcgatcgagccaccatctccagtctgctgacccccaacactcgcctgctgctgctcttgatcgcgccgttcaccaacagtagcactatcagccgtgactcgtacctgggtcatctcatcactttgtaccgcgaggccatcggtcaggctcagatcgacgagcacacatatcaggagatcactaacgtgagccgggccctgggtcaggaagataccggcagcctggaagccacgttgaactttttgctaaccaaccggaggcaaaaaataccctcccagtttacgttaagcgccgaggaggagaggattctgcgatacgtgcagcagtccgtgagtctgtacttgatgcgggagggcgccaccgcttccacggctttagacatgacggctcggaacatggaaccgtccttttactccgcccaccggccgttcattaaccgtctgatggactacttccatcgcgcggccgccatgaacggggagtacttcaccaatgccatcctgaatccgcattggatgcccccgtccggcttctacaccggcgagtttgacctgcccgaagccgacgacggctttctttgggacgacgtgtccgacagcattttcacgccgggcaatcgccgattccagaagaaggagggcggagacgagctccccctctccagcgtggaggcggcctctaggggagagagtccctttcccagtctgtcttccgccagcagtggtcgggtaacgcgcccgcggttgccgggggagagcgactacctgaacgaccccttgctgcggccggctaggaagaaaaatttccccaacaacggggtggaaagcttggtggataaaatgaatcgttggaagacctacgcccaggagcagcgggagtgggaggacagtcagccgcgaccgctggttccgccgcactggcgtcgtcagagagaagacccggacgactccgcagacgatagtagcgtgttggacctgggagggagcggagccaacccctttgctcacttgcaacccaaggggcgttccagtcgcctctactaataaaaaagacgcggaaacttaccagagccatggccacagcgtgtgtcctttcttcctctctttcttcctcggcgcggcagaatgagaagagcggtgagagtcacgccggcggcgtatgagggtccgcccccttcttacgaaagcgtgatgggatcagcgaacgtgccggccacgctggaggcgccttacgttcctcccagatacctgggacctacggagggcagaaacagcatccgttactccgagctggcacccctgtacgataccaccaaggtgtacctggtggacaacaagtcggcggacatcgcctccctgaattatcaaaacgatcacagcaattttctgactaccgtggtgcagaacaatgacttcaccccgacggaggcgggcacgcagaccattaactttgacgagcgttcccgctggggcggtcagctgaaaaccatcctgcacaccaacatgcccaacatcaacgagttcatgtccaccaacaagttcagggccaggctgatggttaaaaaggctgaaaaccagcctcccgagtacgaatggtttgagttcaccattcccgagggcaactattccgagaccatgactatcgatctgatgaacaatgcgatcgtggacaattacctgcaagtggggaggcagaacggggtattggaaagcgatatcggcgtaaaatttgataccagaaacttccgactggggtgggatcccgtgaccaagctggtgatgccaggcgtgtacaccaacgaggcttttcaccccgacatcgtgctgctgccggggtgcggtgtggacttcactcagagccgtttgagtaacctgttagggatcagaaagcgccgccccttccaagagggctttcagatcatgtatgaggacctggaaggaggtaacattccaggtttgctagacgtgccggcgtatgaagagagtgttaaacaggcggaggcgcagggacgagagattcgaggcgacacctttgccacggaacctcacgaactggtaataaaacctctggaacaagacagtaaaaaacggagttacaacattatatccggcactatgaataccttgtaccggagctggtttctggcttacaactacggggatcccgaaaagggagtgagatcatggaccatactcaccaccacggacgtgacctgcggctcgcagcaagtgtactggtccctgccggatatgatgcaagacccggtcaccttccgcccctccacccaagtcagcaacttcccggtggtgggcaccgagctgctgcccgtccatgccaagagcttctacaacgaacaggccgtctactcgcaactcattcgccagtccaccgcgcttacccacgtgttcaatcgctttcccgagaaccagattctggtgcgccctcccgctcctaccattaccaccgtcagtgaaaacgttcccgccctcacagatcacggaaccctgccgctgcgcagcagtatcagtggagttcagcgcgtgaccatcaccgacgccagacgtcgaacctgtccctacgtttacaaagctcttggcgtagtggctcctaaagtgctctctagtcgcaccttctaaacatgtccatcctcatctctcccgataacaacaccggctggggactgggctccggcaagatgtacggcggagccaaaaggcgctccagtcagcacccagttcgagttcggggccacttccgtgctccctggggagcttacaagcgaggactctcgggccgaacggcggtagacgataccatagatgccgtgattgccgacgcccgccggtacaaccccggaccggtcgctagcgccgcctccaccgtggattccgtgatcgacagcgtggtagctggcgctcgggcctatgctcgccgcaagaggcggctgcatcggagacgtcgccccaccgccgccatgctggcagccagggccgtgctgaggcgggcccggagggtaggcagaagggctatgcgccgcgctgccgccaacgccgccgccgggagggcccgccgacaggctgcccgccaggctgctgccgccatcgctagcatggccagacccaggagagggaacgtgtactgggtgcgcgattctgtgacgggagtccgagtgccggtgcgcagccgacctccccgaagttagaagatccaagctgcgaagacggcggtactgagtctccctgttgttatcagcccaacatgagcaagcgcaagtttaaagaagaactgctgcagacgctggtgcctgagatctatggccctccggacgtgaagcctgacattaagccccgcgatatcaagcgtgttaaaaagcgggaaaagaaagaggaactcgcggtggtagacgatggcggagtggaatttattaggagtttcgccccgcgacgcagggttcaatggaaagggcggcgggtacaacgcgttttgaggccgggcaccgcggtagtttttaccccgggagagcggtcggccgttaggggtttcaaaaggcagtacgacgaggtgtacggcgacgaggacatattggaacaggcggctcaacagatcggagaatttgcctacggaaagcgttcgcgtcgcgaagacctggccatcgctttagacagcggcaaccccacgcccagcctcaaacctgtgacgctgcagcaggtgctccccgtgagcgccagcacggacagcaagaggggaataaaaagagaaatggaagatctgcagcccaccatccagctcatggtccctaaacggcagaggctggaagaggtcctggagaaaatgaaagtggacccaagcatagagccggacgtcaaagtcaggccgatcaaagaagtggcccctggtctcggggtgcagacggtggatatccagatccccgtcacgtcagcttcgaccgccgtggaagccatggaaacgcaaacggaaacccctgccgcgatcggtaccagggaagtggcgttgcaaaccgacccctggtacgaatacgccgcccctcggcgtcagaggcgacccgctcgttacggccccgccaacgccatcatgccagaatatgcgctgcatccgtctatcctgcccacccccggctaccggggagtgacgtatcgcccgtcaggaacccgccgccgaacccgtcgccgccgccgctcccgtcgtgctctggcccccgtgtcggtgcgccgcgtaacacgccggggaaagacagttaccattcccaacccgcgctaccaccctagcatcctttaatgactctgccgttttgcagatggctctgacttgccgcgtgcgccttcccgttccgcactatcgaggaagatctcgtcgtaggagaggcatggcgggtagtggtcgccggcgggctttgcgcaggcgcatgaaaggcggaattttacccgctctgatacccataatcgccgccgccatcggtgccatacccggcgtcgcttcagtggccttgcaagcagctcgtaataaataaacgaaggcttttgcacttatgtcctggtcctgactattttatgcagaaagagcatggaagacatcaattttacgtcgctggctccgcggcacggctcgcggccgctcatgggcacctggaacgacatcggcaccagtcagctcaacgggggcgctttcaattgggggagcctttggagcggcattaaaaactttggctccacgattaaatcctacggcagcaaagcctggaacagtagtgctggtcagatgctccgagataaactgaaggacaccaacttccaagaaaaagtggtcaatggggtggtgaccggcatccacggcgcggtagatctcgccaaccaagcggtgcagaaagagattgacaggcgtttggaaagctcgcgggtgccgccgcagagaggggatgaggtggaggtcgaggaagtagaagtagaggaaaagctgcccccgctggagaaagttcccggtgcgcctccgagaccgcagaagcgacccaggccagaactagaagaaactctggtgacggagagcaaggagcctccctcgtacgagcaagccttgaaagagggcgcctctccaccctacccaatgacaaaaccgatcgcgcctatggctcggccggtgtacgggaaggactacaagcctgtcacgctagagctccccccgccgccaccgccgccccccacgcgcccgaccgttcccccccccctgccggctccgtcggcgggacccgtgtccgcacccgtcgccgtgcctctgccagccgcccgcccagtggccgtggccactgccagaaaccccagaggccagagaggagccaactggcaaagcacgctgaacagcatcgtgggcctgggagtgaaaagcctgaaacgccgccgttgctattattaaaagtgtagctaaaaaatttcccgttgtatacgcctcctatgttaccgccagagacgcgtgactgtcgccgcgagcgccgctttcaagatggccaccccatcgatgatgccgcagtggtcttacatgcacatcgccgggcaggacgcctcggagtacctgagccccggtctcgtgcagttcgcccgcgccaccgacacctacttcagcttgggaaacaagtttagaaaccccaccgtggcccccacccacgatgtaaccacggaccgctcgcaaaggctgaccctgcgttttgtgcccgtagaccgggaggacaccgcgtactcttacaaagtgcgctacacgctggccgtaggggacaaccgagtgctggacatggccagcacctactttgacatccggggagtgctggatcgcggtcccagttttaagccctactcgggtaccgcgtacaattccctggctcccaagggcgctcccaaccctgcagaatggacgaattcagacagcaaagttaaagtgagggcacaggcgccttttgttagctcgtatggtgctacagcgattacaaaagagggtattcaggtgggagtaaccttaacagactccggatcaacaccacagtatgcagataaaacgtatcagcctgagccgcaaattggagaactacagtggaacagcgatgttggaaccgatgacaaaatagcaggaagagtgctaaagaaaacaacgcccatgttcccttgttacggctcatatgccaggcccactaatgaaaaaggaggacaggcaacaccgtccgctagtcaagacgtgcaaaatcccgaattacaattttttgcctctactaatgtcgccaatacaccaaaagcagttctatatgcggaggacgtgtcaattgaagcgccagacactcacttggtgttcaaaccaacagtcactgaaggcattacaagttcagaggctctactgacccaacaagctgctcccaaccgtccaaactacatagcctttagagataattttattggtctcatgtactacaatagcacaggtaacatgggagtactggcaggccaggcttctcagctaaatgcagttgttgacctgcaagacagaaatactgagctgtcctaccaactcatgttggacgccctcggagaccgcagtcggtacttttctatgtggaaccaagctgtggatagttacgatcctgatgtaagaatcatagaaaaccatggcgtagaagatgaattgcctaattattgctttcctttgggaggcatggcagtaaccgacacctactcgcctataaaggttaatggaggaggcaatggatgggaagccaataacggcgttttcaccgaaagaggagtggaaataggttcagggaacatgtttgccatggagattaacctgcaagccaacctatggcgtagctttctgtactccaatattgggctgtacctgccagactctctcaaaatcactcctgacaacatcacactcccagagaacaaaaacacctatcagtatatgaacggtcgcgtgacgccacccgggctggttgacacctacgttaacgtgggcgcgcgctggtcccccgatgtcatggacagtattaacccttttaatcaccaccgcaacgccggactccgctaccgttccatgctcctgggaaacggacgctacgtgcccttccacatccaggtgccccagaaattctttgcaattaaaaacctgctgctgctccccggttcctacacctacgagtggaacttccgcaaggacgtgaacatgatcttgcagagctcgctgggcaatgacctgcgagtggacggggccagcatccgcttcgacagcatcaacctgtacgccaactttttccccatggcccacaacacggcctccaccctggaagccatgctgcgcaacgacaccaacgaccaatctttcaacgactacctgtgcgcggccaacatgctgtaccccatccccgccaacgccaccagcgtgcccatctccattccctctcgcaactgggcagccttcaggggctggagtttcacccgcctcaaaaccaaggagaccccctcgctgggctccgggttcgacccctacttcgtctactccggctccatcccctacctggacggcaccttctacctcaaccatactttcaaaaaggtgtcaatcatgttcgactcctccgtcagctggcccggcaacgaccgtctgctgacgcccaacgagttcgaaatcaagcgttcggtggacggtgaagggtacaacgtggctcagagcaacatgaccaaggactggttcctgattcagatgctcagccactacaacatcggctaccagggcttctacgtgcccgaaaattacaaggaccgcatgtactctttcttcagaaacttccaacccatgagccgccaaattgtagattcaacggcttacactaattatcaggatgtgaaactgccataccagcataacaactcagggttcgtgggctacatgggacccaccatgcgagaggggcaggcctacccggccaactatccctatcccctgattggggccaccgccgtgcccagcctcacgcagaaaaagttcctctgcgaccgggtgatgtggaggatccccttctctagcaacttcatgtctatgggctccctcaccgacctggggcagaacatgctgtacgccaactccgctcacgccttggatatgacctttgaggtggatcccatggatgagcccacgcttctctatgttctgtttgaagtcttcgacgtggtgcgcatccaccagccgcaccgcggcgtcatcgaggccgtctacctgcgcacacctttctctgccggtaacgccaccacctaaagaagccgatgggctccagcgaacaggagctgcaggccattgttcgcgacctgggctgcgggccctactttttgggcaccttcgacaagcgttttcccggcttcatgtccccccacaagccggcctgtgccatcgttaacacggccggacgggagaccgggggggtccactggctcgccttcgcctggaacccgcgtaaccgcacctgctacctgttcgacccttttggtttctccgacgaaaggctgaagcagatctaccagttcgagtacgaggggctcctcaagcgcagcgctctggcctccacgcccgaccactgcgtcaccctggaaaagtccacccaaacggtccaggggcccctctcggccgcctgcgggctcttctgttgcatgtttttgcacgccttcgtgcactggcctcacacccccatggatcacaaccccaccatggatctgctcaccggagtgcccaacagcatgcttcacagcccccaggtcgcccccaccctgcgccgtaaccaggaacacctgtatcgctttctggggaaacactctgcctattttcgccgccaccggcagcgcatcgaacgggccacggccttcgaaagcatgagccaaagagtgtaatcaataaaaaacatttttatttgacatgatacgcgcttctggcgttttattaaaaatcgaagggttcgagggaggggtcctcgtgcccgctggggagggacacgttgcgatactggaaacgggcgctccaacgaaactcggggatcaccagccgcggcaggggcacgtcttctaggttctgcttccaaaactgccgcaccagctgcagggctcccatgacgtcgggcgccgatatcttgaagtcgcagttagggccggagctcccgcggctgttgcggaacacggggttggcacactggaacaccagcacgccggggttgtggatactggccagggccgtcgggtcggtcacctccgacgcatccagatcctcggcgttgctcagggcaaacggggtcagcttgcacatctgccgcccaatctggggtactaggtcgcgcttgttgaggcagtcgcagcgcagagggatcaggatgcgtcgctgcccgcgttgcatgatagggtaactcgccgccaggaactcctccatttgacggaaggccatctgggctttgccgccctcggtgtagaatagcccgcaggacttgctagagaatacgttatgaccgcagttgacgtcctccgcgcagcagcgggcgtcttcgttcttcagctgaaccacgttgcggccccaacggttctggaccaccttggctctagtggggtgctccttcagcgcccgctgtccgttctcgctggttacatccatttccaacacgtgctccttgcagaccatctccactccgtggaagcaaaacaggacgccctcctgctgggtactgcgatgctcccatacggcgcatccggtgggctcccagctcttgtgttttacccccgcgtaggcttccatgtaagccataaggaatctgcccatcagctcggtgaaggtcttctggttggtgaaggttagcggcaggccgcggtgctcctcgttcaaccaagtttgacagatcttgcggtacaccgctccctggtcgggcagaaacttaaaagccgctctgctgtcgttgtctacgtggaacttctccattaacatcatcatggtttccatacccttctcccacgctgtcaccagtggtttgctgtcggggttcttcaccaacacggcggtagaggggccctcgccggccccgacgtccttcatggtcattctttgaaactccacggagccgtccgcgcgacgtactctgcgcaccggagggtagctgaagcccacctccaccacggtgccttcgccctcgctgtcggagacaatctccggggatggcggcggcgcgggtgtcgccttgcgagccttcttcttgggagggagctgaggcgcctcctgctcgcgctcggggctcatctcccgcaagtagggggtaatggagctgcctgcttggttctgacggttggccattgtatcctaggcagaaagacatggagcttatgcgcgaggaaactttaaccgccccgtcccccgtcagcgacgaagatgtcatcgtcgaacaggacccgggctacgttacgccgcccgaggatctggaggggcctgaccggcgcgacgctagtgagcggcaggaaaatgagaaagaggaggcctgctacctcctggaaggcgacgttttgctaaagcatttcgccaggcagagcaccatagttaaggaggccttgcaagaccgctccgaggtgcccttggacgtcgccgcgctctcccaggcctacgaggcgaaccttttctcgcctcgagtgcctccgaagagacagcccaacggcacctgcgagcccaacccgcgactcaacttctaccccgtgttcgccgtaccagaggcgctggccacctatcacatttttttcaaaaaccaacgcatccccctatcgtgccgggccaaccgcaccgcggccgataggaatctcaggcttaaaaacggagccaacatacctgatatcacgtcgctggaggaagtgcccaagattttcgagggtctgggtcgagatgagaagcgggcggcgaacgctctgcagaaagaacagaaagagagtcagaacgtgctggtggagctggagggggacaacgcgcgtctggccgtcctcaaacgctgcatagaagtctcccacttcgcctaccccgccctcaacttgccacccaaagttatgaaatcggtcatggatcagctgctcatcaagagagctgagcccctggatcccgaccaccccgaggcggaaaactcagaggacggaaagcccgtcgtcagcgacgaggagctcgagcggtggctggaaaccagggacccccaacagttgcaagagaggcgcaagatgatgatggcggccgtgctggtcaccgtggagctggaatgcctgcaacggtttttcagcgacgtggagacgctacgcaaaatcggggaatccctgcactacaccttccgccagggctacgtccgccaggcctgcaagatctccaacgtggagctcagcaacctggtctcctacatgggcatcctccacgagaaccggctggggcagagcgtgctgcactgcaccttgcaaggcgaggcgcggcgggactacgtgcgagactgcatctacctcttcctcaccctcacctggcagaccgccatgggcgtctggcagcagtgcttggaagagagaaacctcaaagagctagacaaactcctctgccgccagcggcgcgccctgtggtccggtttcagcgagcgcacggtcgccagcgctctggcggacatcatcttcccggagcgcctgatgaaaaccttgcaaaacggcctgccggatttcatcagtcaaagcattttgcaaaacttccgctcttttgtcctggaacgctccgggatcttgcccgccatgagctgcgcgctaccttctgactttgtccccctctcctaccgcgagtgccctcccccactgtggagccactgctacctcttccaactggccaactttctggcctaccactccgacctcatggaagacgtaagcggagagggtttactggagtgccactgccgctgcaacctgtgcaccccccacagatcgctggcctgcaacaccgagctactcagcgaaacccaggtcataggtaccttcgagatccaggggccccagcagcaagagggtgcttccggcttgaagctcactccggcgctgtggacctcggcttacttacgcaaatttgtagccgaggactaccacgcccacaaaattcagttttacgaagaccaatctcgaccaccgaaagcccccctcacggcctgcgtcatcacccagagcaagatcctggcccaattgcaatccatcaaccaagcgcgccgcgatttccttttgaaaaagggtcggggggtgtacctggacccccagaccggcgaggaactcaacccgtccacactctccgtcgaagcagcccccccgagacatgccgcccaagggaaccgccaagcagctgatcgctcggcagagagcgaagaagcaagagctgctccagcagcaggtggaggacgaggaagagatgtgggacagccaggcagaggaggtgtcagaggacgaggaggagatggaaagctgggacagcctagacgaggaggaggacgagctttcagaggaagaggcgaccgaagaaaaaccacctgcatccagcgcgccttctctgagccgacagccgaagccccggcccccgacgcccccggccggctcactcaaagccagccgtaggtgggacgccaccgaatctccagcggcagcggcaacggcagcgggtaaggccaaacgcgagcggcgggggtattgctcctggcgggcccacaaaagcagtattgtgaactgcttgcaacactgcgggggaaacatctcctttgcccgacgctacctcctcttccatcacggtgtggccttccctcgcaacgttctctattattaccgtcatctctacagcccctacgaaacgctcggagaaaaaagctaaggcctcctccgccgcgaggaaaaactccgccgccgctgccgccgccaaggatccaccggccaccgaagagctgagaaagcgcatctttcccactctgtatgctatctttcagcaaagccgcgggcagcaccctcagcgcgaactgaaaataaaaaaccgctccttccgctcgctcacccgcagctgtctgtaccacaagagagaagaccagctgcagcgcaccctggacgacgccgaagcactgttcagcaaatactgctcagcgtctcttaaagactaaaagacccgcgctttttccccctcggccgccaaaacccacgtcatcgccagcatgagcaaggagattcccaccccctacatgtggagctatcagccccagatgggcctggccgcgggggccgcccaggactactccagcaagatgaactggctcagcgccggcccccacatgatctcacgagttaacggcatccgagcccaccgaaaccagattctcttagaacaggcggcaatcaccgccacaccccggcgccaactcaacccgcctagttggcccgccgcccaggtgtatcaggaaaatccccgcccgaccacagtcctcctgccacgcgacgcggaggccgaagtcctcatgactaactctggggtacaattagcgggcgggtccaggtacgccaggtacagaggtcgggccgctccttactctcccgggagtataaagagggtgatcattcgaggccgaggtatccagctcaacgacgagacggtgagctcctcaaccggtctcagacctgacggagtcttccagctcggaggagcgggccgctcttccttcaccactcgccaggcctacctgaccctgcagagctcttcctcgcagccgcgctccgggggaatcggcactctccagttcgtggaagagttcgttccctccgtctacttcaaccccttctccggctcgcctggacgctacccggacgccttcattcccaactttgacgcagtgagtgaatccgtggacggctacgactgatgacagatggtgcggccgtgagagctcggctgcgacatctgcatcactgccgtcagcctcgctgctacgctcgggaggcgatcgtcttcagctactttgagctgccggacgagcaccctcagggtccggctcacgggttgaaactcgagatcgagaacgcgctcgagtctcgcctcatcgacaccttcaccgcccgacctctcctggtagaaatccaacgggggatcactaccatcaccctgttctgcatctgccccacgcccggattacatgaagatctgtgttgtcatctttgcgctcagtttaataaaaactgaactttttgccgcaccttcaacgccatctgtgatttctacaacaaaaagttcttctggcaaaggtacacaaactgtattttattctaattctacctcatctatcgtgctgaactgcgcctgcactaacgaacttatccagtggattgcaaacggtagtgtgtgcaagtacttttgggggaacgatatagttagtagaaataacagcctttgcgagcactgcaactcctccacactaatcctttatcccccatttgttactggatggtatatgtgcgttggctccggtttaaatcctagttgctttcataagtggtttctacaaaaagagacccttcccaacaattctgtttcttttttcgccctatcctactgctgttctccctctggttactctttcaaacctctaattggtattttagctttgatactcataatctttattaactttataataattaacaacttacagtaaacatgcttgttctactgctcgccacatctttcgctctctctcacgccagaacaagtattgttggcgcaggttacaatgcaactcttcaatctgcttacatgccagattccgaccagataccccatattacgtggtacttacaaacctccaaacctaattcttcattttatgaaggaaacaaactctgcgatgactccgacaacagaacgcacacatttccccacccttcactacaattcgaatgcgtaaacaaaagcttgaagctttacaacttaaagccttcagattctggcttgtaccatgctgtagttgaaaaaagtaatttagaagtccacagtgattacattgaattgacggttgtggacctgccacctccaaaatgtgaggtttcctcctcttaccttgaagttcaaggcgtggatgcctactgcctcatacacattaactgcagcaactctaaatatccagctagaatttactataatggacaggaaagtaatcttttttattatttaacaacaagcgctggtaacggtaaacagttacctgactattttactgctgttgttgaattttccacctacagagaaacgtatgccaagcggccttacaatttctcatacccgtttaacgacctttgcaatgaaatacaagcgctcgaaactggaactgattttactccaattttcattgctgccattgttgtaagcttaattaccattattgtcagcctagcattttactgcttttacaagcccaaaaaccctaagtttgaaaaacttaaactaaaacctgtcattcaacaagtgtgattttgttttccagcatggtagctgcatttctacttctcctctgtctacccatcattttcgtctcttcaactttcgccgcagtttcccacctggaaccagagtgcctaccgccttttgacgtgtatctgattctcacctttgtttgttgtatatccatttgcagtatagcctgcttttttataacaatctttcaagccgccgactatttttacgtgcgaattgcttactttagacaccatcctgaatacagaaatcaaaacgttgcctccttactttgtttggcatgattaagttattgctgatacttaattatttacccctaatcaactgtaattgtccattcaccaaaccctggtcattctacacctgttatgataaaatccccgacactcctgttgcttggctttacgcagccaccgccgctttggtatttatatctacttgccttggagtaaaattgtattttattttacacactgggtggctacatcccagagaagatttacctagatatcctcttgtaaacgcttttcaattacagcctctgcctcctcctgatcttcttcctcgagctccctctattgtgagctactttcaactcaccggtggagatgactgactctcaggacattaatattagtgtggaaagaatagctgctcagcgtcagcgagaaacgcgagtgttggaatacctggaactacagcaacttaaagagtcccactggtgtgagaaaggagtgctgtgccatgttaagcaggcagccctttcctacgatgtcagcgttcagggacatgaactgtcttacactttgcctttgcagaaacaaaccttctgcaccatgatgggctctacctccatcacaatcacccaacaagccgggcctgtagagggggctatcctctgtcactgtcacgcacctgattgcatgtccaaactaatcaaaactctctgtgctttaggtgatatttttaaggtgtaaatcaataataaacttaccttaaatttgacaacaaatttctggtgacatcattcagcagcaccactttaccctcttcccagctctcgtatgggatgcgatagtgggtggcaaacttcctccaaaccctaaaagaaatattggtatccacttccttgtcctcacccacaattttcatcttttcatagatgaaaagaaccagagttgatgaagacttcaaccccgtctacccctatgacaccacaaccactcctgcagttccctttatatcacccccctttgtaaacagcgatggtcttcaggaaaaccccccaggtgttttaagtctgcgaatagctaaacccctatatttcgacatggagagaaaactagccctttcacttggaagagggttgacaattaccgccgccggacaattagaaagtacgcagagcgtacaaaccaacccaccgttgataattaccaacaacaacacactgaccctacgtcattctccccccttaaacctaactgacaatagcttagtgctaggctactcgagtcctctccgcgtcacagacaacaaacttacatttaacttcacatcaccactccgttatgaaaatgaaaaccttacttttaactatacagagcctcttaaacttataaataacagccttgccattgacatcaattcctcaaaaggccttagtagcgtcggaggctcactagctgtaaacctgagttcagacttaaagtttgacagcaacggatccatagcttttggcatacaaaccctgtggaccgctccgacctcgactggcaactgcaccgtctacagcgagggcgattccctacttagtctctgtttaaccaaatgcggagctcacgtcttaggaagtgtaagtttaaccggtttaacaggaaccataacccaaatgactgatatttctgtcaccattcaatttacatttgacaacaatggtaagctactaagctctccacttataaacaacgcctttagtattcgacagaatgacagtacggcctcaaaccctacctacaacgccctggcgtttatgcctaacagtaccatatatgcaagagggggaggtggtgaaccacgaaacaactactacgtccaaacgtatcttaggggaaatgttcaaaaaccaatcattcttactgtaacctacaactcagtcgccacaggatattccttatcttttaagtggactgctcttgcacgtgaaaagtttgcaaccccaacaacctcgttttgctacattacagaacaataaaaccgtgtaccccaccgtttcgtttttttcagatgaaacgggcgagagttgatgaagacttcaacccagtgtacccttatgaccccccacatgctcctgttatgcccttcattactccaccttttacctcctcggatgggttgcaggaaaaaccacttggagtgttaagtttaaactacagagatcccattactacgcaaaatgagtctcttacaattaaactaggaaacggcctcactctagacaaccagggacaactaacatcaaccgctggcgaagtagaacctccactcactaacgctaacaacaaacttgcactggtctatagcgatcctttagcagtaaagcgcaacagcctaaccttatcgcacaccgctccccttgttattgctgataactctttagcattgcaagtttcagagcctatttttataaatgacaaggacaaactagccctgcaaacagccgcgccccttgtaactaacgctggcacccttcgcttacaaagcgccgcccctttaggcattgcagaccaaaccctaaaactcctgtttaccaaccctttgtacttgcagaataactttctcacgttagccattgaacgaccccttgccattaccaatactggaaagctggctctacagctctccccaccgctacaaacagcagacacaggcttgactttgcaaaccaacgtgccattaactgtaagcaacgggaccctaggcttagccataaagcgcccacttattattcaggacaacaacttgtttttggacttcagagctcccctgcgtcttttcaacagcgacccagtactagggcttaacttttacacccctcttgcggtacgcgatgaggcgctcactgttaacacaggccgcggcctcacagtgagttacgatggtttaattttaaatcttggtaaggatcttcgctttgacaacaacaccgtttctgtcgctcttagtgctgctttgcctttacaatacactgatcagcttcgccttaacgtgggcgctgggctgcgttacaatccagtgagtaagaaattggacgtgaaccccaatcaaaacaagggtttaacctgggaaaatgactacctcattgtaaagctaggaaatggattaggttttgatggcgatggaaacatagctgtttctcctcaagttacatcgcctgacaccttatggaccactgccgacccatcccccaattgttccatctacactgatttagatgccaaaatgtggctctcgttggtaaaacaagggggtgtggttcacggttctgttgctttaaaagcattgaaaggaaccctattgagtcctacggaaagcgccattgttattatactacattttgacaattatggagtgcgaattctcaattatcccactttgggcactcaaggcacgttgggaaataatgcaacttggggttataggcagggagaatctgcagacactaatgtactcaatgcactagcatttatgcccagttcaaaaaggtacccaagagggcgtggaagcgaagttcagaatcaaactgtgggctacacttgtatacagggtgacttttctatgcccgtaccgtaccaaatacagtacaactatggaccaactggctactcctttaaatttatttggagaactgtttcaagacaaccatttgacatcccatgctgttttttctcttacattacggaagaataaaacaactttttctttttattttctttttattttacacgcacagtaaggcttcctccacccttccatctcacagcatacaccagcctctcccccttcatggcagtaaactgttgtgagtcagtccggtatttgggagttaagatccaaacagtctctttggtgatgaaacatggatccgtgatggacacaaatccctgggacaggttctccaacgtttcggtaaaaaactgcatgccgccctacaaaacaaacaggttcaggctctccacgggttatctccccgatcaaactcagacagagtaaaggtgcgatgatgttccactaaaccacgcaggtggcgctgtctgaacctctcggtgcgactcctgtgaggctggtaagaagttagattgtccagcagcctcacagcatggatcatcagtctacgagtgcgtctggcgcagcagcgcatctgaatctcactgagattccggcaagaatcgcacaccatcacaatcaggttgttcatgatcccatagctgaacacgctccagccaaagctcattcgctccaacagcgccaccgcgtgtccgtccaaccttactttaacataaatcaggtgtctgccgcgtacaaacatgctacccgcatacagaacctcccggggcaaacccctgttcaccacctgcctgtaccagggaaacctcacatttatcagggagccatagatagccattttaaaccaattagctaacaccgccccaccagctctacactgaagagaaccgggagagttacaatgacagtgaataatccatctctcataacccctaatggtctgatggaaatccagatctaacgtggcacagcagatacacactttcatatacattttcatcacatgtttttcccaggccgttaaaatacaatcccaatacacgggccactcctgcagtacaataaagctaatacaagatggtatactcctcacctcactaacattgtgcatgttcatattttcacattctaagtaccgagagttctcctctacaacagcactgccgcggtcctcacaaggtggtagctggtgacgattgtaaggagccagtctgcagcgataccgtctgtcgcgttgcatcgtagaccagggaccgacgcacttcctcgtacttgtagtagcagaaccacgtccgctgccagcacgtctccaagtaacgccggtccctgcgtcgctcacgctccctcctcaacgcaaagtgcaaccactcttgtaatccacacagatccctctcggcctccggggcgatgcacacctcaaacctacagatgtctcggtacagttccaaacacgtagtgagggcgagttccaaccaagacagacagcctgatctatcccgacacactggaggtggaggaagacacggaagaggcatgttattccaagcgattcaccaacgggtcgaaatgaagatcccgaagatgacaacggtcgcctccggagccctgatggaatttaacagccagatcaaacattatgcgattttccaggctatcaatcgcggcctccaaaagagcctggacccgcacttccacaaacaccagcaaagcaaaagcgttattatcaaactcttcgatcatcaagctgcaggactgtacaatgcccaagtaattttcatttctccactcgcgaatgatgtcgcggcaaatagtctgaaggttcatgccgtgcatattaaaaagctccgaaagggcgccctctatagccatgcgtagacacaccatcatgactgcaagatatcgggctcctgagacacctgcagcagatttaacagacccaggtcaggttgctctccgcgatcgcgaatctccatccgcaaagtcatttgcaaataattaaatagatctgcgccgactaaatctgttaactccgcgctaggaactaaatcaggtgtggctacgcagcacaaaagttccagggatggcgccaaactcactagaaccgctcccgagtagcaaaactgatgaatgggagtaacacagtgtaaaatgttcagccaaaaatcactaagctgctcctttaaaaagtccagtacttctatattcagttcgtgcaagtactgaagcaactgtgcgggaatatgcacagcaaaaaaaatagggcggctcagatacatgttgacctaaaataaaaagaatcattaaactaaagaagcctggcgaacggtgggatatatgacacgctccagcagcaggcaagcaaccggctgtccccgggaaccgcggtaaaattcatccgaatgattaaaaagaacaacagagacttcccaccatgtactcggttggatctcctgagcacagagcaatacccccctcacattcatatccgctacagaaaaaaaacgtcccagatacccagcgggaatatccaacgacagctgcaaagacagcaaaacaatccctctgggagcaatcacaaaatcctccggtgaaaaaagcacatacatattagaataaccctgttgctggggcaaaaaggcccgtcgtcccagcaaatgcacataaatatgttcatcagccattgccccgtcttaccgcgtaaacagccacgaaaaaatcgagctaaaatccacccaacagcctatagctatatatacactccacccaatgacgctaataccgcaccacccacgaccaaagttcacccacacccacaaaacccgcgaaaatccagcgccgtcagcacttccgcaatttcagtctcacaacgtcacttccgcgcgccttttcactttcccacacacgcccttcgcccgcccgccctcgcgccaccccgcgtcaccccacgtcaccgcacgtcaccccggccccgcctcgctcctccccgctcattatcatattggcacgtttccagaataaggtatattattgatgcagcaaaacaatccctctgggagcaatcacaaaatcctccggtgaaaaaagcacatacatattagaataaccctgttgctggggcaaaaaggcccgtcgtcccagcaaatgcacataaatatgttcatcagccattgccccgtcttaccgcgtaaacagccacgaaaaaatcgagctaaaatccacccaacagcctatagctatatatacactccacccaatgacgctaataccgcaccacccacgaccaaagttcacccacacccacaaaacccgcgaaaatccagcgccgtcagcacttccgcaatttcagtctcacaacgtcacttccgcgcgccttttcactttcccacacacgcccttcgcccgcccgccctcgcgccaccccgcgtcaccccacgtcaccgcacgtcaccccggccccgcctcgctcctccccgctcattatcatattggcacgtttccagaataaggtatattattgatgca

SEQ ID NO: 25 - protein, 503 aa, simian adenovirus SV-1
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVKKAENQPPEYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPGLLDVPAYEESVKQAEAQGREIRGDTFATEPHELVIKPLEQDSKKRSYNIISGTMNTLYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 26 - protein, 931 aa, simian adenovirus SV-1
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPAEWTNSDSKVKVRAQAPFVSSYGATAITKEGIQVGVTLTDSGSTPQYADKTYQPEPQIGELQWNSDVGTDDKIAGRVLKKTTPMFPCYGSYARPTNEKGGQATPSASQDVQNPELQFFASTNVANTPKAVLYAEDVSIEAPDTHLVFKPTVTEGITSSEALLTQQAAPNRPNYIAFRDNFIGLMYYNSTGNMGVLAGQASQLNAVVDLQDRNTELSYQLMLDALGDRSRYFSMWNQAVDSYDPDVRIIENHGVEDELPNYCFPLGGMAVTDTYSPIKVNGGGNGWEANNGVFTERGVEIGSGNMFAMEINLQANLWRSFLYSNIGLYLPDSLKITPDNITLPENKNTYQYMNGRVTPPGLVDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQIVDSTAYTNYQDVKLPYQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 27 - protein, 363 aa, simian adenovirus SV-1
MKRTRVDEDFNPVYPYDTTTTPAVPFISPPFVNSDGLQENPPGVLSLRIAKPLYFDMERKLALSLGRGLTITAAGQLESTQSVQTNPPLIITNNNTLTLRHSPPLNLTDNSLVLGYSSPLRVTDNKLTFNFTSPLRYENENLTFNYTEPLKLINNSLAIDINSSKGLSSVGGSLAVNLSSDLKFDSNGSIAFGIQTLWTAPTSTGNCTVYSEGDSLLSLCLTKCGAHVLGSVSLTGLTGTITQMTDISVTIQFTFDNNGKLLSSPLINNAFSIRQNDSTASNPTYNALAFMPNSTIYARGGGGEPRNNYYVQTYLRGNVQKPIILTVTYNSVATGYSLSFKWTALAREKFATPTTSFCYITEQ

SEQ ID NO: 28 - protein, 560 aa, simian adenovirus SV-1
MKRARVDEDFNPVYPYDPPHAPVMPFITPPFTSSDGLQEKPLGVLSLNYRDPITTQNESLTIKLGNGLTLDNQGQLTSTAGEVEPPLTNANNKLALVYSDPLAVKRNSLTLSHTAPLVIADNSLALQVSEPIFINDKDKLALQTAAPLVTNAGTLRLQSAAPLGIADQTLKLLFTNPLYLQNNFLTLAIERPLAITNTGKLALQLSPPLQTADTGLTLQTNVPLTVSNGTLGLAIKRPLIIQDNNLFLDFRAPLRLFNSDPVLGLNFYTPLAVRDEALTVNTGRGLTVSYDGLILNLGKDLRFDNNTVSVALSAALPLQYTDQLRLNVGAGLRYNPVSKKLDVNPNQNKGLTWENDYLIVKLGNGLGFDGDGNIAVSPQVTSPDTLWTTADPSPNCSIYTDLDAKMWLSLVKQGGVVHGSVALKALKGTLLSPTESAIVIILHFDNYGVRILNYPTLGTQGTLGNNATWGYRQGESADTNVLNALAFMPSSKRYPRGRGSEVQNQTVGYTCIQGDFSMPVPYQIQYNYGPTGYSFKFIWRTVSRQPFDIPCCFFSYITEE

SEQ ID NO: 29 - DNA, 31044 bp, simian adenovirus SV-25
  CDS (12284)..(13801): Penton
catcatcaataatataccttattctggaaacgtgccaatatgataatgagcggggaggagcgaggcggggccggggtgacgtgcggtgacgcggggtggcgcgagggcggggcgaagggcgcgggtgtgtgtgtgggaggcgcttagtttttacgtatgcggaaggaggttttataccggaagatgggtaatttgggcgtatacttgtaagttttgtgtaatttggcgcgaaaactgggtaatgaggaagttgaggttaatatgtactttttatgactgggcggaatttctgctgatcagcagtgaactttgggcgctgacggggaggtttcgctacgtgacagtaccacgagaaggctcaaaggtcccatttattgtactcttcagcgttttcgctgggtatttaaacgctgtcagatcatcaagaggccactcttgagtgctggcgagaagagttttctcctccgtgctgccacgatgaggctggtccccgagatgtacggtgtttttagcgacgagacggtgcgtaactcagatgacctgctgaattcagacgcgctggaaatttccaattcgcctgtgctttcgccgccgtcacttcacgacctgtttgtgttttggctcaacgcttagcaacgtgttatatagggtcaagaaggagcaggagacgcagtttgctaggctgttggccgatactcctggagtttttgtggctctggatctaggccatcactctcttttccaagagaaaattatcaaaaacttaacttttacgtctcctggtcgcacggttgcttccgctgcctttattacctatattttggatcaatggagcaacagcgacagccacctgtcgtgggagtacatgctggattacatgtcgatggcgctgtggagggccatgctgcggaggagggtttgcatttacttgcgggcgcagcctccgcggctggaccgagtggaggaggaggacgagccgggggagaccgagaacctgagggccgggctggaccctccaacggaggactaggtgctgaggatgatcccgaagaggggactagtggggctaggaagaagcaaaagactgagtctgaacctcgaaactttttgaatgagttgactgtgagtttgatgaatcgtcagcgtccggagacaattttctggtctgaattggaggaggaattcaggaggggggaactgaacctgctatacaagtatgggtttgaacagttaaaaactcactggttggagccgtgggaggattttgaaaccgccttggacacttttgctaaagtggctctgcggccggataaggtttacactatccgccgcactgttaacataaagaagagtgtttatgttataggccatggagctctggtgcaggtgcaaaccgtcgaccgggtggcctttagttgcggtatgcaaaatctgggccccggggtgataggcttaaatggtgtaacatttcacaatgtaaggtttactggtgaaagttttaacggctctgtgtttgcaaataacacacagctgacgctccacggcgtttacttttttaactttaataacacatgtgtggagtcgtggggcagggtgtctttgaggggctgctgttttcacggctgctggaaggcggtggtgggaagacttaaaagtgtaacatctgtaaaaaaatgcgtgtttgagcggtgtgtgttggctttaactgtggagggctgtggacgcattaggaataatgcggcgtctgagaatggatgttttcttttgctaaaaggcacggctagtattaagcataacatgatatgcggcagcggtctgtacccttcacagctgttaacttgcgcggatggaaactgtcagaccttgcgcaccgtgcacatagcgtcccaccagcgccgcgcctggccaacattcgagcacaatatgcttatgcgttgtgccgtccacttgggccctaggcgaggcgtgtttgtgccttaccagtgtaactttagccataccaagattttactagaacctgataccttctctcgagtgtgtttcaatggggtgtttgacatgtcaatggaactgtttaaagtgataagatatgatgaatccaagtctcgttgtcgcccatgtgaatgcggagctaatcatctgaggttgtatcctgtaaccctaaacgttaccgaggagctgaggacggatcaccacatgttgtcctgcctgcgcaccgactatgaatccagcgacgaggagtgaggtgaggggcggagccacaaagggtataaaggggcgtgaggggtgggtgtgatgattcaaaatgagcgggacgacggacggcaacgcgtttgagggtggagtgttcagcccttatctgacatctcgtcttccttcctgggcaggagtgcgtcagaatgtagtgggctccaccgtggacggacgaccggtcgcccctgcaaattccgccaccctcacctatgccaccgtgggatcatcgttggacactgccgcggcagctgccgcttctgctgccgcttctactgctcgcggcatggcggctgattttggactgtataaccaactggccactgcagctgtggcgtctcggtctctggttcaagaagatgccctgaatgtgatcctgactcgcctggagatcatgtcacgtcgcttggacgaactggctgcgcagatatcccaagctaaccccgataccacttcagaatcctaaaataaagacaaacaaatatgttgaaaagtaaaatggctttatttgttttttttggctcggtaggctcgggtccacctgtctcggtcgttaagaactttgtgtatgttttccaaaacacggtacagatgggcttggatgttcaagtacatgggcatgaggccatctttggggtgaagataggaccattgaagagcgtcatgctccggggtggtgttgtaaattacccagtcgtagcagggtttctgggcgtggaactggaagatgtcctttaggagtaggctgatggccaagggcaggcccttagtgtaggtgtttacaaagcggttaagctgggagggatgcatgcggggggagatgatatgcatcttggcttggatcttgaggttagctatgttaccacccaggtctctgcgggggttcatgttatgaaggaccaccagcacggtgtagccggtgcatttggggaacttgtcatgcagtttggaggggaaggcgtggaagaatttagagacccccttgtggccccctaggttttccatgcactcatccataatgatggcaatgggacccctggcggccgctttggcaaacacgttttgggggttggaaacatcatagttttgctctagagtgagctcatcataggccatcttaacaaagcggggtaggagggtgcccgactgggggatgatagttccatctgggcctggggcgtagttaccctcacagatctgcatctcccaggccttaatttccgaggggggtatcatgtccacctggggggcaataaagaacacggtttctggcgggggattgatgagctgggtggaaagcaagttacgcagcagttgagatttgccacagccggtggggccgtagatgaccccgatgacgggttgcagctggtagttgagagaggaacagctgccgtcggggcgcaggaggggggctacctcattcatcatgcttctaacatgtttattttcactcactaagttttgcaagagcctctccccacccagggataagagttcttccaggctgttgaagtgtttcagcggttttaggccgtcggccatgggcatcttttcgagcgactgacgaagcaagtacagtcggtcccagagctcggtgacgtgctctatggaatctcgatccagcagacttcttggttgcgggggttgggtcgactttcgctgtagggcaccagccggtgggcgtccagggccgcgagggttctgtccttccagggtctcagcgtccgggtgagggtggtctcggtgacggtgaagggatgagccccgggctgggcgcttgcgagggtgcgcttcaggctcatcctgctggtgctgaagcggacgtcgtctccctgtgagtcggccagatagcaacgaagcatgaggtcgtagctgagggactcggccgcgtgtcccttggcgcgcagctttcccttggaaacgtgctgacatttggtgcagtgcagacattggagggcgtagagtttgggggccaggaagaccgactcgggcgagtaggcgtcggctccgcactgagcgcagacggtctcgcactccactagccacgtgagctcgggtttagcgggatcaaaaaccaagttgcctccattttttttgatgcgtttcttaccttgcgtttccatgagtttgtggcccgcttccgtgacaaaaaggctgtcggtgtctccgtagacagacttgagggggcgatcttccaaaggtgttccgaggtcttccgcgtacaggaactgggaccactccgagacgaaggctctggtccaggctaacacgaaggaggcaatctgcgaggggtatctgtcgttttcaatgagggggtccaccttttccagggtgtgcagacacaggtcgtcctcctccgcgtccacgaaggtgattggcttgtaagtgtaggtcacgtgatctgcaccccccaaaggggtataaaagggggcgtgcccaccctctccgtcactttcttccgcatcgctgtggaccagagccagctgttcgggtgagtaggccctctcaaaagccggcatgatctcggcgctcaagttgtcagtttctacaaacgaggtggatttgatattcacgtgccccgcggcgatgcttttgatggtggaggggtccatctgatcagaaaacacgatctttttgttgtcaagtttggtggcgaaagacccgtagagggcgttggaaagcaacttggcgatggagcgcagggtctgatttttctcccgatcggccctctccttggcggcgatgttgagttgcacgtactcccgggccgcgcaccgccactcggggaacacggcggtgcgctcgtcgggcaggatgcgcacgcgccagccgcgattgtgcagggtgatgaggtccacgctggtagccacctccccgcggaggggctcgttggtccaacacaatcgccccccttttctggagcagaacggaggcaggggatctagcaagttggcgggcggggggtcggcgtcgatggtgaagataccgggtagcaggatcttattaaaataatcgatttcggtgtccgtgtcttgcaacgcgtcttcccacttcttcaccgccagggccctttcgtagggattcaggggcggtccccagggcatggggtgggtcagggccgaggcgtacatgccgcagatgtcatacacgtacaggggttccctcaacaccccgatgtaagtggggtaacagcgccccccgcggatgctggctcgcacgtagtcgtacatctcgcgcgagggagccatgaggccgtctcccaagtgggtcttgtggggtttttcggcccggtagaggatctgtctgaagatggcgtgggagttggaagagatggtggggcgttggaagacgttaaagttggccccgggtagtcccacggagtcttggatgaactgggcgtaggattcccggagtttgtccaccagggcggcggtcaccagcacgtcgagagcgcagtagtccaacgtctcgcggaccaggttgtaggccgtctcttgttttttctcccacagttcgcggttgaggaggtattcctcgcggtctttccagtactcttcggcgggaaatcctttttcgtccgctcggtaagaacctaacatgtaaaattcgttcaccgctttgtatggacaacagcctttttctaccggcagggcgtacgcttgagcggcctttctgagagaggtgtgggtgagggcgaaggtgtcccgcaccatcactttcaggtactgatgtttgaagtccgtgtcgtcgcaggcgccctgttcccacagcgtgaagtcggtgcgctttttctgcctgggattggggagggcgaaggtgacatcgttaaagagtattttcccggcgcggggcatgaagttgcgagagatcctgaagggcccgggcacgtccgagcggttgttgatgacctgcgccgccaggacgatctcgtcgaagccgttgatgttgtgacccacgatgtaaagttcgatgaagcgcggctgtcccttgagggccggcgcttttttcaactcctcgtaggtgagacagtccggcgaggagagacccagctcagcccgggcccagtcggagagttgaggattagccgcaaggaaggagctccatagatccaaggccaggagagtttgcaagcggtcgcggaactcgcggaactttttccccacggccattttctccggtgtcactacgtaaaaggtgttggggcggttgttccacacgtcccatcggagctctagggccagctcgcaggcttggcgaacgagggtctcctcgccagagacgtgcatgaccagcataaagggtaccaactgtttcccgaacgagcccatccatgtgtaggtttctacgtcgtaggtgacaaagagccgctgggtgcgcgcgtgggagccgatcggaaagaagctgatctcctgccaccagctggaggaatgggtgttaatgtggtggaagtagaagtcccgccggcgcacagagcattcgtgctgatgtttgtaaaagcgaccgcagtagtcgcagcgctgcacgctctgtatctcctgaacgagatgcgcttttcgcccgcgcaccagaaaccggagggggaagttgagacggggggctggtggggcgacatccccttcgccttggcggtgggagtctgcgtctgcgtcctccttctctgggtggacgacggtggggacgacgacgccccgggtgccgcaagtccagatctccgccacggaggggtgcaggcgctgcaggaggggacgcagctgcccgctgtccagggagtcgagggaagtcgcgctgaggtcggcgggaagcgtttgcaagttcactttcagaagaccggtaagagcgtgagccaggtgcagatggtacttgatttccaggggggtgttggatgaagcgtccacggcgtagaggagtccgtgtccgcgcggggccaccaccgtgccccgaggaggttttatctcactcgtcgagggcgagcgccggggggtagaggcggctctgcgccggggggcagcggaggcagaggcacgttttcgtgaggattcggcagcggttgatgacgagcccggagactgctggcgtgggcgacgacgcggcggttgaggtcctggatgtgccgtctctgcgtgaagaccaccggcccccgggtcctgaacctaaagagagttccacagaatcaatgtctgcatcgttaacggcggcctgcctgaggatctcctgcacgtcgcccgagttgtcctgataggcgatctcggccatgaactgttccacttcttcctcgcggaggtcaccgtggcccgctcgctccacggtggcggccaggtcgttggagatgcggcgcatgagttgagagaaggcgttgaggccgttctcgttccacacgcggctgtacaccacgtttccgaaggagtcgcgcgctcgcatgaccacctgggccacgttgagttccacgtggcgggcgaagacggcgtagtttctgaggcgctggaagaggtagttgagcgtggtggcgatgtgctcgcagacgaagaagtacataatccagcgccgcagggtcatctcgttgatgtctccgatggcttcgagacgctccatggcctcgtagaagtcgacggcgaagttgaaaaattgggagttgcgggcggccaccgtgagttcttcttgcaggaggcggatgagatcggcgaccgtgtcgcgcacctcctgttcgaaagcgccccgaggcgcctctgcttcttcctccggctcctcctcttccaggggctcgggttcctccggcagctctgcgacggggacggggcggcgacgtcgtcgtctgaccggcaggcggtccacgaagcgctcgatcatttcgccgcgccggcgacgcatggtctcggtgacggcgcgtccgttttcgcgaggtcgcagttcgaagacgccgccgcgcagagcgcccccgtgcagggagggtaagtggttagggccgtcgggcagggacacggcgctgacgatgcattttatcaattgctgcgtaggcactccgtgcagggatctgagaacgtcgaggtcgacgggatccgagaacttctctaggaaagcgtctatccaatcgcaatcgcaaggtaagctgagaacggtgggtcgctggggggcgttcgcgggcagttgggaggtgatgctgctgatgatgtaattaaagtaggcggtcttcaggcggcggatggtggcgaggaggaccacgtctttgggcccggcctgttgaatgcgcaggcgctcggccatgccccaggcctcgctctgacagcgacgcaggtctttgtagaagtcttgcatcagtctctccaccggaacctctgcttctcccctgtctgccatgcgagtcgagccgaacccccgcaggggctgcagcaacgctaggtcggccacgaccctttcggccagcacggcctgttgaatctgcgtgagggtggcctggaagtcgtccaggtccacgaagcggtgataggcccccgtgttgatggtgtaggtgcagttggccatgacggaccagttgacgacttgcatgccgggttgggtgatctccgtgtacttgaggcgcgagtaggccctggactcgaacacgtagtcgttgcatgtgcgcaccagatactggtagccgaccaggaagtgaggaggcggctctcggtacaggggccagccaacggtggcgggggcgccgggggacaggtcgtccagcatgaggcggtggtagtggtagatgtagcgggagagccaggtgatgccggccgaggtggttgcggccctggtgaattcgcggacgcggttccagatgttgcgcaggggaccaaagcgctccatggtgggcacgctctgccccgtgaggcgggcgcaatcttgtacgctctagatggaaaaaagacagggcggtcatcgactcctttccgtagcttggggggtaaagtcgcaagggtgcggcggcggggaaccccggttcgagaccggccggatccgccgctcccgatgcgcctggccccgcatccacgacgtccgcgccgagacccagccgcgacgctccgccccaatacggaggggagtcttttggtgttttttcgtagatgcatccggtgctgcggcagatgcgaccccagacgcccactaccaccgccgtggcggcagtaaacctgagcggaggcggtgacagggaggaggaagagctggctttagacctggaagagggagaggggctggcccggctgggagcgccatccccagagagacaccctagggttcagctcgtgagggacgccaggcaggcttttgtgccgaagcagaacctgtttagggaccgcagcggtcaggaggcggaggagatgcgcgattgcaggtttcgggcgggcagagagctcagggcgggcttcgatcgggagcggctcctgagggcggaggatttcgagcccgacgagcgttctggggtgagcccggcccgcgctcacgtatcggcggccaacctggtgagcgcgtacgagcagacggtgaacgaggagcgcaacttccaaaagagctttaacaatcacgtgaggaccctgatcgcgagggaggaggtgaccatcgggctgatgcatctgtgggacttcgtggaggcctacgtgcagaacccggctagcaaacccctgacggcccagctgttcctgatcgtgcagcacagccgcgacaacgagacgttccgcgacgccatgttgaacatcgcggagcccgagggtcgctggctcttggatctgattaacatcctgcagagcatcgtggtgcaggagaggggcctgagtttagcggacaaggtggcggccattaactattcgatgcagagcctggggaagttctacgctcgcaagatctacaagagcccttacgtgcccatagacaaggaggtgaagatagacagcttttacatgcgcatggcgctgaaggtgctgacgctgagcgacgacctcggcgtgtaccgtaacgacaagatccacaaggcggtgagcgccagccgccggcgggagctgagcgacagggagctgatgcacagcctgcagagggcgctggcgggcgccggggacgaggagcgcgaggcttacttcgacatgggagccgatctgcagtggcgtcccagcgcgcgcgccttggaggcggcgggttatcccgacgaggaggatcgggacgatttggaggaggcaggcgagtacgaggacgaagcctgaccgggcaggtgttgttttagatgcagcggccggcggacgggaccaccgcggatcccgcacttttggcatccatgcagagtcaaccttcgggcgtgaccgcctccgatgactgggcggcggccatggaccgcatcatggcgctgaccacccgcaaccccgaggcttttaggcagcaaccccaggccaaccgtttttcggccatcttggaagcggtggtgccgtcgcgcaccaacccgacgcacgagaaagtcctgactatcgtgaacgccctggtagacagcaaggccatccgccgtgacgaggcgggcttgatttacaacgctcttttggaacgcgtggcgcgctacaacagcactaacgtgcagaccaatctggaccgcctcaccaccgacgtgaaggaggcgctggcgcagaaggagcggtttctgagggacagtaatctgggctctctggtggcactgaacgccttcctgagctcacagccggccaacgtgccccgcgggcaggaggattacgtgagcttcatcagcgctctgagactgctggtgtccgaggtgccccagagcgaggtgtaccagtctgggccggattactttttccagacgtcccgacagggcttgcaaacggtgaacctgactcaggcctttaaaaacttgcaaggcatgtggggggtcaaggccccggtgggcgatcgcgccactatctccagtctgctgacccccaacactcgcctgctgctgctcttgatcgcaccgtttaccaacagtagcactatcagccgtgactcgtacctgggtcatctcatcactctgtaccgcgaggccatcggccaggctcagatcgacgagcatacgtatcaggagattactaacgtgagccgtgccctgggtcaggaagataccggcagcctggaagccacgttgaactttttgctaaccaaccggaggcaaaaaataccctcccagttcacgttaagcgccgaggaggagaggattctgcgatacgtgcagcagtccgtgagcctgtacttgatgcgcgagggcgccaccgcttccacggctttagacatgacggctcggaacatggaaccgtccttttactccgcccaccggccgttcattaaccgtctgatggactacttccatcgcgcggccgccatgaacggggagtacttcaccaatgccatcctgaatccgcattggatgcccccgtccggcttctacaccggggagtttgacctgcccgaagccgacgacggctttctgtgggacgacgtgtccgatagcattttcacgccggctaatcgccgattccagaagaaggagggcggagacgagctccccctctccagcgtggaagcggcctcaaggggagagagtccctttccaagtctgtcttccgccagtagcggtcgggtaacgcgtccacggttgccgggggagagcgactacctgaacgaccccttgctgcgaccggctagaaagaaaaattttcccaataacggggtggaaagcttggtggataaaatgaatcgttggaagacgtacgcccaggagcagcgggagtgggaggacagtcagccgcggccgctggtaccgccgcattggcgtcgccagagagaagacccggacgactccgcagacgatagtagcgtgttggacctgggagggagcggagccaacccctttgctcacttgcaacccaaggggcgctcgagtcgcctgtattaataaaaaagacgcggaaacttaccagagccatggccacagcgtgtgtgctttcttcctctctttcttcctcggcgcggcagaatgagaagagcggtgagagtcacgccggcggcgtatgagggcccgcccccttcttacgaaagcgtgatgggatcagcgaacgtgccggccacgctggaggcgccttacgttcctcccagatacctgggacctacggagggcagaaacagcatccgttactccgagctggcgcccctgtacgataccaccaaggtgtacctggtggacaacaagtcggcggacatcgcctccctgaattaccaaaacgatcacagtaactttctgactaccgtggtgcagaacaatgacttcaccccgacggaggcgggcacgcagaccattaactttgacgagcgttcccgctggggcggtcagctgaaaaccatcctgcacaccaacatgcccaacatcaacgagttcatgtccaccaacaagttcagggctaagctgatggtagaaaaaagtaatgcggaaactcggcagccccgatacgagtggttcgagtttaccattccagagggcaactattccgaaactatgactatcgatctcatgaataacgcgatcgtggacaattacctgcaagtggggagacagaacggggtgctggaaagcgatatcggcgtgaaattcgataccagaaacttccgactggggtgggatcccgtgaccaagctggtgatgccaggcgtgtacaccaacgaggcttttcacccggacatcgtgctgctgccggggtgcggtgtggacttcactcagagccgtttgagtaacctgttaggaattagaaagcgccgccccttccaagagggctttcaaatcatgtatgaggacctggagggaggtaatatacccgccttactggacgtgtcgaagtacgaagctagcatacaacgcgccaaagcggagggtagagagattcggggagacacctttgcggtagctccccaggacctggaaatagtgcctttaactaaagacagcaaagacagaagctacaatattataaacaacacgacggacaccctgtatcggagctggtttctggcttacaactacggagaccccgagaaaggagtgagatcatggaccatactcaccaccacggacgtgacctgtggctcgcagcaagtgtactggtccctgccggatatgatgcaagacccggtcaccttccgcccctccacccaagtcagcaacttcccggtggtgggcaccgagctgctgcccgtccatgccaagagcttctacaacgagcaggccgtctactcgcaacttattcgccagtccaccgcgcttacccacgtgttcaatcgctttcccgagaaccagattctggtgcgccctcccgctcctaccattaccaccgtcagtgaaaacgttcccgccctcacagatcacggaaccctgccgctgcgcagcagtatcagtggagttcagcgcgtgaccatcaccgacgccagacgtcgaacctgcccctacgtttacaaagcgcttggcgtggtggctcctaaagttctttctagtcgcaccttctaaaaacatgtccatcctcatctctcccgataacaacaccggctggggactgggctccggcaagatgtacggcggagccaaaaggcgctccagtcagcacccagttcgagttcggggccacttccgcgctccttggggagcttacaagcgaggactctcgggtcgaacggctgtagacgataccatagatgccgtgattgccgacgcccgccggtacaaccccggaccggtcgctagcgccgcctccaccgtggattccgtgatcgacagcgtggtagccggcgctcgggcctatgctcgccgcaagaggcggctgcatcggagacgtcgccccaccgccgccatgctggcagccagggccgtgctgaggcgggcccggagggcaggcagaagggctatgcgccgcgctgccgccaacgccgccgccgggagggcccgccgacaggctgcccgccaggctgccgctgccatcgctagcatggccagacccaggagagggaacgtgtactgggtgcgtgattctgtgacgggagtccgagtgccggtgcgcagccgacctccccgaagttagaagatccaagctgcgaagacggcggtactgagtctccctgttgttatcagcccaacatgagcaagcgcaagtttaaagaagaactgctgcagacgctggtgcctgagatctatggccctccggacgtgaagccagacattaagccccgcgatatcaagcgtgttaaaaagcgggaaaagaaagaggaactcgcggtggtagacgatggcggagtggaatttattaggagtttcgccccgcgacgcagggttcaatggaaagggcggcgggtacaacgcgttttgaggccgggcaccgcggtagtttttaccccgggagagcggtcggccgttaggggtttcaaaaggcagtacgacgaggtgtacggcgacgaggacatattggaacaggcggctcaacagatcggagaatttgcctacggaaagcgttcgcgtcgcgaagacctggccatcgccttagacagcggcaaccccacgcccagcctcaaacccgtgacgctgcagcaggtgcttcccgtgagcgccagcacggacagcaagagggggattaagagagaaatggaagatctgcatcccaccatccaactcatggtccctaaacggcagaggctggaagaggtcctggagaagatgaaagtggaccccagcatagagccggatgtaaaagtcagacctattaaggaagtggcccccggtcttggggtgcaaacggtggacattcaaatccccgtcaccaccgcttcaaccgccgtggaagctatggaaacgcaaacggagacccctgccgcgatcggtaccagggaagtggcgttgcaaacggagccttggtacgaatacgcagcccctcggcgtcagaggcgttccgctcgttacggccccgccaacgccatcatgccagaatatgcgctgcatccgtctattctgcccactcccggataccggggtgtgacgtatcgcccgtctggaacccgccgccgaacccgtcgccgccgccgctcccgtcgcgctctggcccccgtgtcggtgcggcgtgtgacccgccggggaaagacagtcgtcattcccaacccgcgttaccaccctagcatcctttaataactctgccgttttgcagatggctctgacttgccgcgtgcgccttcccgttccgcactatcgaggaagatctcgtcgtaggagaggcatgacgggcagtggtcgccggcgggctttgcgcaggcgcatgaaaggcggaattttacccgccctgatacccataattgccgccgccatcggtgccatacccggcgttgcttcagtggcgttgcaagcagctcgtaataaataaacaaaggcttttgcacttatgacctggtcctgactattttatgcagaaagagcatggaagacatcaattttacgtcgctggctccgcggcacggctcgcggccgctcatgggcacctggaacgacatcggcaccagtcagctcaacgggggcgctttcaattgggggagcctttggagcggcattaaaaactttggctccacgattaaatcctacggcagcaaagcctggaacagtagtgctggtcagatgctccgagataaactgaaggacaccaacttccaagaaaaagtggtcaatggggtggtgaccggcatccacggcgcggtagatctcgccaaccaagcggtgcagaaagagattgacaggcgtttggaaagctcgcgggtgccgccgcagagaggggatgaggtggaggtcgaggaagtagaagtagaggaaaagctgcccccgctggagaaagttcccggtgcgcctccgagaccgcagaagcggcccaggccagaactagaagagactctggtgacggagagcaaggagcctccctcgtacgagcaagccttgaaagagggcgcctctccaccctcctacccgatgactaagccgatcgcacccatggctcgaccggtgtacggcaaggattacaagcccgtcacgctagagctgcccccaccgccccccacgcgcccgaccgtcccccccctgccgactccgtcggcggccgcggcgggacccgtgtccgcaccatccgctgtgcctctgccagccgcccgtccagtggccgtggccactgccagaaaccccagaggccagagaggagccaactggcaaagcacgctgaacagcatcgtgggcctgggagtgaaaagcctgaaacgccgccgttgctattattaaaaaagtgtagctaaaaagtctcccgttgtatacgcctcctatgttaccgccagagacgagtgactgtcgccgcgagcgccgctttcaagatggccaccccatcgatgatgccgcagtggtcttacatgcacatcgccggccaggacgcctcggagtacctgagtcccggcctcgtgcagtttgcccgcgccaccgacacctacttcagcttgggaaacaagtttagaaaccccaccgtggcccccacccacgatgtgaccacggaccgctcgcagaggctgaccctgcgctttgtgcccgtagaccgggaggacaccgcgtactcttacaaagtgcgctacacgttggccgtaggggacaaccgagtgctggacatggccagcacctactttgacatccggggggtgctggatcggggtcccagcttcaagccctattccggcaccgcttacaactccctggcccccaagggagctcccaacccctcggaatggacggacacttccgacaacaaacttaaagcatatgctcaggctccctaccagagtcaaggacttacaaaggatggtattcaggttgggctagttgtgacagagtcaggacaaacaccccaatatgcaaacaaagtgtaccaacccgagccacaaattggggaaaaccaatggaatttagaacaagaagataaagcggcgggaagagtcctaaagaaagatacccctatgtttccctgctatgggtcatatgccaggcccacaaacgaacaaggagggcaggcaaaaaaccaagaagtagatttacagttttttgccactccgggcgacacccagaacacggctaaagtggtactttatgctgaaaatgtcaacctggaaactccagatactcacttagtgtttaaacccgatgacgacagcaccagttcaaaacttcttcttgggcagcaggctgcacctaacagacccaactacataggttttagagataattttattggtttaatgtactacaatagcactggaaacatgggcgtgctggccggacaggcttctcaattgaatgccgtagtcgacttgcaggacagaaacaccgagttgtcctaccagctgatgctggacgcactgggggatcgcagccgatatttttcaatgtggaatcaggcagtagacagctatgacccagacgttagaattatagaaaaccacggagtggaagacgaactgccaaactattgttttcctctgggaggaatggtggtgactgacaattacaactctgtgacgcctcaaaatggaggcagtggaaatacatggcaggcagacaatactacatttagtcaaagaggagcgcagattggctccggaaacatgtttgccctggaaattaacctacaggccaacctctggcgcggcttcttgtattccaatattgggttgtatcttccagactctctgaaaatcacccccgacaacatcacgctgccagaaaacaaaaacacttatcagtacatgaacggtcgcgtaacgccacccgggctcatagacacctatgtaaacgtgggcgcgcgctggtcccccgatgtcatggacagcattaaccccttcaaccaccaccgtaacgcgggcttgcgctaccgctccatgctcttgggcaacggccgttatgtgccttttcacattcaggtgccccaaaaattctttgccattaaaaacctgctgcttctccccggttcctatacctatgagtggaacttccgcaaggatgtcaacatgatcctgcagagctcgctgggtaatgacctgcgagtggacggggccagcatacgctttgacagcattaacctgtatgccaacttttttcccatggcccacaacacggcctctaccctggaagccatgctgcgcaacgacaccaatgaccagtccttcaacgactacctgtgcgcggctaacatgctgtaccccatccccgccaacgccaccagcgtgcccatttctattccttctcggaactgggctgccttcaggggctggagttttactcgcctcaaaaccaaggagactccctcgctgggctccggttttgacccctactttgtttactccggctccattccctacctagatggcaccttttacctcaaccacactttcaaaaaggtgtctattatgtttgactcctcggttagctggcccggcaacgaccgcctgctaacgcccaacgagttcgaaattaagcgttccgtggacggtgaagggtacaacgtggcccagagcaacatgaccaaggactggtttctaattcaaatgctcagtcactataatataggttaccagggcttctatgtgcccgagaactacaaggaccgcatgtactccttcttccgcaacttccaaccaatgagccggcaggtggtagataccgtgacttatacagactacaaagatgtcaagctcccctaccaacacaacaactcagggttcgtgggctacatgggacccaccatgcgagagggacaggcctacccggccaactatccctaccccctgatcggagagactgccgtacccagcctcacgcagaaaaagttcctctgcgaccgggtgatgtggaggatacccttctctagcaactttatgtcgatgggctccctcaccgacctggggcagaacatgctgtacgccaactccgctcacgccttggacatgacttttgaggtggatcccatggatgagcccacgcttctctatgttctgtttgaagtcttcgacgtggtgcgcatccaccagccgcaccgcggcgtcatcgaggccgtctacctgcgcacacctttctctgccggtaacgccaccacctaaagaagctgatgggttccagcgaacaggagttgcaggccattgttcgcgacctgggctgcgggccctgctttttgggcaccttcgacaagcgttttcccggattcatgtccccccacaagccggcctgcgccatcgttaacacggccggacgggagacagggggggtgcactggctcgccttcgcctggaacccgcgcaaccgcacctgctacctgttcgacccttttggtttctccgacgaaaggctgaagcagatctaccaattcgagtacgaggggctcctcaagcgcagcgctctggcctccacgcccgaccactgcgtcaccctggaaaagtccacccagacggtccaggggcccctctcggccgcctgcgggcttttctgttgcatgtttttgcacgccttcgtgcactggcctcacacccccatggagcgcaaccccaccatggatctgctcaccggagtgcccaacagcatgcttcacagtccccaggtcgcccccaccctgcgtcgcaatcaggaccacctgtatcgctttctggggaaacactctgcctatttccgccgccaccggcagcgcatcgaacaggccacggccttcgaaagcatgagccaaagagtgtaatcaataaaaaccgtttttatttgacatgatacgcgcttctggcgtttttattaaaaatcgaagggttcgagggaggggtcctcgtgcccgctggggagggacacgttgcggtactggaatcgggcgctccaacgaaactcggggatcaccagccgcggcagggccacgtcttccatgttctgcttccaaaactgtcgcaccagctgcagggctcccatcacgtcgggcgctgagatcttgaagtcgcagttagggccggagcccccgcggctgttgcggaacacggggttggcacactggaacaccaacacgctggggttgtggatactagccagggccgtcgggtcggtcacctccgatgcatccagatcctcggcattgctcagggcgaacggggtcagcttgcacatctgccgcccgatctggggtaccaggtcgcgcttgttgaggcagtcgcagcgcagagggatgaggatgcgacgctgcccgcgttgcatgatggggtaactcgccgccaggaactcctctatctgacggaaggccatctgggccttgacgccctcggtgaaaaatagcccacaggacttgctggaaaacacgttattgccacagttgatgtcttccgcgcagcagcgcgcatcttcgttcttcagctgaaccacgttgcgaccccagcggttctgaaccaccttggctttcgtgggatgctccttcagcgcccgctgtccgttctcgctggtcacatccatttccaccacgtgctccttgcagaccatctccactccgtggaaacagaacagaatgccctcctgttgggtattgcgatgctcccacacggcgcacccggtggactcccagctcttgtgtttcacccccgcgtaggcttccatgtaagccattagaaatctgcccatcagctcagtgaaggtcttctggttggtgaaggttagcggcaggccgcggtgttcctcgttcaaccaagtttgacagatcttgcggtacacggctccctggtcgggcagaaacttaaaagtcgttctgctctcgttgtccacgtggaacttctccatcaacatcgtcatgacttccatgcccttctcccaggcagtcaccagcggcgcgctctcggggttcttcaccaacacggcggtggaggggccctcgccggccccgacgtccttcatggacattttttgaaactccacggtgccgtccgcgcggcgtactctgcgcatcggagggtagctgaagcccacctccatgacggtgctttcgccctcgctgtcggagacgatctccggggagggcggcggaacgggggcagacttgcgagccttcttcttgggagggagcggaggcacctcctgctcgcgctcgggactcatctcccgcaagtagggggtgatggagcttcctggttggttctgacggttggccattgtatcctaggcagaaagacatggagcttatgcgcgaggaaactttaaccgccccgtcccccgtcagcgacgaagaggtcatcgtcgaacaggacccgggctacgttacgccgcccgaggatctggaggggcccttagacgaccggcgcgacgctagtgagcggcaggaaaatgagaaagaggaggaggagggctgctacctcctggaaggcgacgttttgctaaagcatttcgccaggcagagcaccatactcaaggaggccttgcaagaccgctccgaggtgcccttggacgtcgccgcgctctcccaggcctacgaggcgaaccttttctcgccccgagtgcctccgaagagacagcccaacggcacctgcgagcccaacccgcgactcaacttctaccccgtgttcgccgtgcccgaggcgctggccacctaccacatctttttcaaaaaccagcgcattcccctttcctgccgggccaaccgcaccgcggccgataggaagctaacactcagaaacggagtcagcatacctgatatcacgtcactggaggaagtgcctaagatcttcgagggtctgggtcgagatgagaagcgggcggcgaacgctctgcagaaagaacagaaagagagtcagaacgtgctggtggagctggagggggacaacgcgcgtctgaccgtcctcaaacgttgcatagaagtttcccacttcgcctacccggccctcaacctgccgcccaaagttatgaaatcggtcatggaccagctactcatcaagagagctgagcccctgaatcccgaccaccctgaggcggaaaactcagaggacggaaagcccgtcgtcagcgacgaggagctcgagcggtggctggaaaccagggacccccagcagttgcaagagaggcgcaagatgatgatggcggccgtgctggtcacggtggagctagaatgcctgcaacggtttttcagcgacgtggagacgctacgcaaaatcggggagtccctgcactacaccttccgccagggctacgttcgccaggcctgcaaaatctccaacgtagagctcagcaacctggtttcctacatgggcatcctccacgagaaccggctggggcagagcgtgctgcactgcaccttgcaaggcgaggcgcgaagggactacgtccgagactgcgtctacctcttcctcaccctcacctggcagaccgccatgggcgtgtggcagcagtgcttggaagagagaaacctcaaagagctggacaaactcctctgccgccagcggcgggccctctggaccggcttcagcgagcgcacggtcgcctgcgccctggcagacatcattttcccagaacgcctgatgaaaaccttgcagaacggcctgccggatttcatcagtcagagcatcttgcaaaacttccgctccttcgtcctggagcgctccgggatcttgcccgccatgagctgcgcgctgccttctgactttgtccccctttcctaccgcgagtgccctcccccactgtggagccactgctacctcttccaactggccaactttctggcctaccactccgacctcatggaagacgtgagcggagaggggctgctcgagtgccactgccgctgcaacctctgcaccccccacagatcgctggcctgcaacaccgagctgctcagcgaaacccaggtcataggtaccttcgagatccaggggccccagcagcaagagggtgcttccggcttgaagctcactccggcgctgtggacctcggcttacttacgcaaatttgtagccgaggactaccacgcccacaaaattcagttttacgaagaccaatctcgaccaccgaaagcccccctcacggcctgcgtcatcacccagagcaaaatcctggcccaattgcaatccatcaaccaagcgcgccgagatttccttttgaaaaagggtcggggggtgtacctggacccccagaccggcgaggaactcaacccgtccacactttccgtcgaagcagcccccccgagacatgccacccaagggaaccgccaagcagctgatcgctcggcagagagcgaagaagcaagagctgctccagcagcaggtggaggacgaggaagagctgtgggacagccaggcagaggaggtgtcagaggacgaggaggagatggaaagctgggacagcctagacgaggaggacgagctttcagaggaagaggcgaccgaagaaaaaccacctgcatccagcgcgccttctctgagccgacagccgaagccccggcccccgacgcccccggccggctcactcaaagccagccgtaggtgggacgccaccggatctccagcggcagcggcaacggcagcgggtaaggccaaacgcgagcggcgggggtattgctcctggcggacccacaaaagcagtatcgtgaactgcttgcaacactgcgggggaaacatctcctttgcccgacgctacctcctcttccatcacggtgtggccttccctcgcaacgttctctattattaccgtcatctctacagcccctacgaaacgctcggagaaaaaagctaaggcctcctctgccgcgaggaaaaactccgccgccgctgccgccaaggatccgccggccaccgaggagctgagaaagcgcatctttcccactctgtatgctatctttcagcaaagccgcgggcagcaccctcagcgcgaactgaaaataaaaaaccgctccttccgctcactcacccgcagctgtctgtaccacaagagagaagaccagctgcagcgcaccctggacgacgccgaagcactgttcagcaaatactgctcagcgtctcttaaagactaaaagacccgcgctttttccccctcgggcgccaaaacccacgtcatcgccagcatgagcaaggagattcccaccccttacatgtggagctatcagccccagatgggcctggccgcgggggccgcccaggactactccagcaaaatgaactggctcagcgccggcccccacatgatctcacgagttaacggcatccgagcccaccgaaaccagatcctcttagaacaggcggcaatcaccgccacaccccggcgccaactcaacccgcccagttggcccgccgcccaggtgtatcaggaaactccccgcccgaccacagtcctcctgccacgcgacgcggaggccgaagtcctcatgactaactctggggtacaattagcgggcgggtccaggtacgccaggtacagaggtcgggccgctccttactctcccgggagtataaagagggtgatcattcgaggccgaggtatccagctcaacgacgaggcggtgagctcctcaaccggtctcagacctgacggagtcttccagctcggaggagcgggccgctcttccttcaccactcgccaggcctacctgaccctgcagagctcttcctcgcagccgcgctccgggggaatcggcactctccagttcgtggaagagttcgtcccctccgtctacttcaacccgttttccggctcacctggacgctacccggacgccttcattcccaactttgacgcagtgagtgaatccgtggacggctacgactgatgacagatggtgcggccgtgagagctcggctgcgacatctgcatcactgccgccagcctcgctgctacgctcgggaggcgatcgtgttcagctactttgagctgccggacgagcaccctcagggaccggctcacgggttgaaactcgagattgagaacgcgcttgagtctcacctcatcgacgccttcaccgcccggcctctcctggtagaaaccgaacgcgggatcactaccatcaccctgttctgcatctgccccacgcccggattacatgaagatctgtgttgtcatctttgcgctcagtttaataaaaactgaactttttgccgtaccttcaacgccacgcgttgtttctccttgtgaaaaaaccccaggagtccttaacttacacatagcaaaacccttgtattttaccatagaaaaacaactagccctttcaattggaaaagggttaacaatttctgctacaggacagttggaaagcacagcaagcgtacaggacagcgctacaccacccctacgtggtatttcccctttaaagctgacagacaacggtttaacattaagctattcagatcccctgcgtgtggtaggtgaccaacttacgtttaattttacttctccactacgttacgaaaatggcagtcttacattcaactacacttctcccatgacactaataaacaacagtcttgctattaacgtcaatacctccaaaggcctcagtagtgacaacggcacactcgctgtaaatgttactccagattttagatttaacagctctggtgccttaacttttggcatacaaagtctatggacttttccaaccaaaactcctaactgtaccgtgtttaccgaaagtgactccctgctgagtctttgcttgactaaatgcggagctcacgtacttggaagcgtgagtttaagcggagtggcaggaaccatgctaaaaatgacccacacttctgttaccgttcagttttcgtttgatgacagtggtaaactaatattctctccacttgcgaacaacacttggggtgttcgacaaagcgagagtccgttgcccaacccatccttcaacgctctcacgtttatgccaaacagtaccatttattctagaggagcaagtaacgaacctcaaaacaattattatgtccagacgtatcttagaggcaacgtgcgaaagccaattctactaactgttacctacaactcagttaattcaggatattccttaacttttaaatgggatgctgtcgccaatgaaaaatttgccactcctacatcttcgttttgctatgttgcagagcaataaaaccctgttaccccaccgtctcgtttttttcagatgaaacgagcgagagttgatgaagacttcaacccagtgtacccttatgaccccccatacgctcccgtcatgcccttcattactccgccttttacctcctcggatgggttgcaggaaaaaccacttggagtgttaagtttaaactacagggatcccattactacacaaaatgggtctctcacgttaaaactaggaaacggcctcactctaaacaaccagggacagttaacatcaactgctggcgaagtggagcctccgctcactaatgctaacaacaaacttgcactagcctatagcgaaccattagcagtaaaaagcaaccgcctaactctatcacacaccgctccccttgtcatcgctaataattctttagcgttgcaagtttcagagcctatttttgtaaatgacgatgacaagctagccctgcagacagccgccccccttgtaaccaacgctggcacccttcgcttacagagcgctgcccctttaggattggttgaaaatactcttaaactgctgttttctaaacccttgtatttgcaaaatgattttcttgcattagccattgaacgccccctggctgtagcagccgcaggtactctgaccctacaacttactcctccattaaagactaacgatgacgggctaacactatccacagtcgagccattaactgtaaaaaacggaaacctaggcttgcaaatatcgcgccctttagttgttcaaaacaacggcctttcgcttgctattacccccccgctgcgtttgtttaacagcgaccccgttcttggtttgggcttcacttttcccctagctgtcacaaacaacctcctctccttaaacatgggagacggagttaaacttacctataataaactaacagccaatttgggtagggatttacaatttgaaaacggtgcgattgccgtaacgcttactgccgaattacctttgcaatacactaacaaacttcaactgaatattggagctggccttcgttacaatggagccagcagaaaactagatgtaaacattaaccaaaataaaggcttaacttgggacaacgatgcagttattcccaaactaggatcgggcttacaatttgaccctaatggcaacatcgctgttatccctgaaaccgtgaagccgcaaacgttatggacgactgcagatccctcgcctaactgctcagtgtaccaggacttggatgccaggctgtggctcgctcttgttaaaagtggcgacatggtgcatggaagcattgccctaaaagccctaaaagggacgttgctaaatcctacagccagctacatttccattgtgatatatttttacagcaacggagtcaggcgtaccaactatccaacgtttgacaacgaaggcaccttagctaacagcgccacttggggataccgacaggggcaatctgctaacactaatgtgaccaatgccactgaatttatgcccagctcaagcaggtaccccgtgaataaaggagacaacattcaaaatcaatctttttcatacacctgtattaaaggagattttgctatgcctgtcccgttccgtgtaacatataatcacgccctggaagggtattcccttaagttcacctggcgcgttgtagccaatcaggcctttgatattccttgctgttcattttcatacatcacagaataaaaaaccactttttcattttaatttctttttattttacacgaacagtgagacttcctccacccttccatttgacagcatacaccagcctctcccccttcatagcagtaaactgttgtgaatcagtccggtatttgggagttaaaatccaaacagtctctttggtgatgaaacgtcgatcagtaatggacacaaatccctgggacaggttttccaacgtttcggtgaaaaactgcacaccgccctacaaaacaaacaggttcaggctctccacgggttatctccccgatcaaactcagacagggtaaaggtgcggtggtgttccactaaaccacgcaggtggcgctgtctgaacctctcggtgcgactcctgtgaggctggtaagaagttagattgtccagtagcctcacagcatgtatcatcagtctacgagtgcgtctggcgcagcagcgcatctgaatctcactgagattccggcaagaatcgcacaccatcacaatcaggttgttcatgatcccatagctgaacacgctccagccaaagctcattcgctccaacagcgccaccgcgtgtccgtccaaccttactttaacataaatcaggtgtctgccgcgtacaaacatgctacccacatacagaacttcccggggcaggcccctgttcaccacctgtctgtaccagggaaacctcacatttatcagggagccatagatggccattttaaaccaattagctaataccgccccaccagctctacactgaagagaaccgggagagttacaatgacagtgaataatccatctctcataacccctgatggtctgatgaaaatctagatctaacgtggcacaacaaatacacactttcatatacattttcataacatgtttttcccaggccgttaaaatacaatcccaatacacgggccactcctgcagtacaataaagctaatacaagatggtatactcctcacctcactgacactgtgcatgttcatattttcacattctaagtaccgagagttctcctctacagcagcactgctgcggtcctcacaaggtggtagctggtgatgattgtagggggccagtctgcagcgataccgtctgtcgcgttgcatcgtagaccaggaaccgacgcacctcctcgtacttgtggtagcagaaccacgtccgctgccagcacgtctccacgtaacgccggtccctgcgtcgctcacgctccctcctcaatgcaaagtgcaaccactcttgtaatccacacagatccctctcggcctccggggtgatgcacacctcaaacctacagatgtctcggtacagttccaaacacgtagtgagggcgagttccaaccaagacagacagcctgatctatcccgacacactggaggtggaggaagacacggaagaggcatgttattccaagcgattcaccaacgggtcgaaatgaagatcccgaagatgacaacggtcgcctccggagccctgatggaatttaacagccagatcaaacgttatgcgattctccaagctatcgatcgccgcttccaaaagagcctggacccgcacttccacaaacaccagcaaagcaaaagcactattatcaaactcttcaatcatcaagctgcaggactgtacaatgcctaagtaattttcgtttctccactcgcgaatgatgtcgcggcagatagtctgaaggttcatcccgtgcagggtaaaaagctccgaaagggcgccctctacagccatgcgtagacacaccatcatgactgcaagatatcgggctcctgagacacctgcagcagatttaacagatcaaggtcaggttgctctccgcgatcacgaatctccatccgcaaggtcatttgcaaaaaattaaataaatctatgccgactagatctgtcaactccgcattaggaaccaaatcaggtgtggctacgcagcacaaaagttccagggatggtgccaaactcactagaaccgctcccgagtaacaaaactgatgaatgggagtaacacagtgtaaaatgtgcaaccaaaaatcactaaggtgctcctttaaaaagtccagtacttctatattcagtccgtgcaagtactgaagcaactgtgcgggaatatgcacaacaaaaaaaatagggcggctcagatacatgttgacctaaaataaaaagaatcattaaactaaagaagcttggcgaacggtgggataaatgacacgctccagcagcagacaggcaaccggctgtccccgggaaccgcggtaaaattcatccgaatgattaaaaagaacaacagaaacttcccaccatgtactcggttggatctcctgagcacacagcaatacccccctcacattcatgtccgccacagaaaaaaaacgtcccagatacccagcggggatatccaacgacagctgcaaagacagcaaaacaatccctctgggagcgatcacaaaatcctccggtgaaaaaagcacatacatattagaataaccctgttgctggggcaaaaaggcccggcgtcccagcaaatgcacataaatatgttcatcagccattgccccgtcttaccgcgtaatcagccacgaaaaaatcgagctaaaattcacccaacagcctatagctatatatacactccgcccaatgacgctaataccgcaccacccacgaccaaagttcacccacacccacaaaacccgcgaaaatccagcgccgtcagcacttccgcaatttcagtctcacaacgtcacttccgcgcgccttttcacattcccacacacacccgcgcccttcgccccgccctcgcgccaccccgcgtcaccgcacgtcaccccggccccgcctcgctcctccccgctcattatcatattggcacgtttccagaataaggtatattattgatgatg

SEQ ID NO: 30 - protein, 505 aa, simian adenovirus SV-25
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRAKLMVEKSNAETRQPRYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPALLDVSKYEASIQRAKAEGREIRGDTFAVAPQDLEIVPLTKDSKDRSYNIINNTTDTLYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 31 - protein, 921 aa, simian adenovirus SV-25
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPSEWTDTSDNKLKAYAQAPYQSQGLTKDGIQVGLVVTESGQTPQYANKVYQPEPQIGENQWNLEQEDKAAGRVLKKDTPMFPCYGSYARPTNEQGGQAKNQEVDLQFFATPGDTQNTAKVVLYAENVNLETPDTHLVFKPDDDSTSSKLLLGQQAAPNRPNYIGFRDNFIGLMYYNSTGNMGVLAGQASQLNAVVDLQDRNTELSYQLMLDALGDRSRYFSMWNQAVDSYDPDVRIIENHGVEDELPNYCFPLGGMVVTDNYNSVTPQNGGSGNTWQADNTTFSQRGAQIGSGNMFALEINLQANLWRGFLYSNIGLYLPDSLKITPDNITLPENKNTYQYMNGRVTPPGLIDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQVVDTVTYTDYKDVKLPYQHNNSGFVGYMGPTMREGQAYPANYPYPLIGETAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 32 - protein, 347 aa, simian adenovirus SV-25
MKICVVIFALSLIKTELFAVPSTPRVVSPCEKTPGVLNLHIAKPLYFTIEKQLALSIGKGLTISATGQLESTASVQDSATPPLRGISPLKLTDNGLTLSYSDPLRVVGDQLTFNFTSPLRYENGSLTFNYTSPMTLINNSLAINVNTSKGLSSDNGTLAVNVTPDFRFNSSGALTFGIQSLWTFPTKTPNCTVFTESDSLLSLCLTKCGAHVLGSVSLSGVAGTMLKMTHTSVTVQFSFDDSGKLIFSPLANNTWGVRQSESPLPNPSFNALTFMPNSTIYSRGASNEPQNNYYVQTYLRGNVRKPILLTVTYNSVNSGYSLTFKWDAVANEKFATPTSSFCYVAEQ

SEQ ID NO: 33 - protein, 559 aa, simian adenovirus SV-25
MKRARVDEDFNPVYPYDPPYAPVMPFITPPFTSSDGLQEKPLGVLSLNYRDPITTQNGSLTLKLGNGLTLNNQGQLTSTAGEVEPPLTNANNKLALAYSEPLAVKSNRLTLSHTAPLVIANNSLALQVSEPIFVNDDDKLALQTAAPLVTNAGTLRLQSAAPLGLVENTLKLLFSKPLYLQNDFLALAIERPLAVAAAGTLTLQLTPPLKTNDDGLTLSTVEPLTVKNGNLGLQISRPLVVQNNGLSLAITPPLRLFNSDPVLGLGFTFPLAVTNNLLSLNMGDGVKLTYNKLTANLGRDLQFENGAIAVTLTAELPLQYTNKLQLNIGAGLRYNGASRKLDVNINQNKGLTWDNDAVIPKLGSGLQFDPNGNIAVIPETVKPQTLWTTADPSPNCSVYQDLDARLWLALVKSGDMVHGSIALKALKGTLLNPTASYISIVIYFYSNGVRRTNYPTFDNEGTLANSATWGYRQGQSANTNVTNATEFMPSSSRYPVNKGDNIQNQSFSYTCIKGDFAMPVPFRVTYNHALEGYSLKFTWRVVANQAFDIPCCSFSYITE

SEQ ID NO: 34 - DNA, 34115 bp, simian adenovirus SV-39
  CDS (13448)..(14959): L2 Penton
catcatcaatataacaccgcaagatggcgaccgagttaacatgcaaatgaggtgggcggagttacgcgacctttgtcttgggaacgcggaagtgggcgcggcgggtttcggggaggagcgcggggcggggcgggcgtgtcgcgcggcggtgacgcgccggggacccggaaattgagtagtttttattcattttgcaagtttttctgtacattttggcgcgaaaactgaaacgaggaagtgaaaagtgaaaaatgccgaggtagtcaccgggtggagatctgacctttgccgtgtggagtttacccgctgacgtgtgggtttcggtctctattttttcactgtggttttccgggtacggtcaaaggtccccattttatgactccacgtcagctgatcgctagggtatttaatgcgcctcagaccgtcaagaggccactcttgagtgccggcgagaagagttttctcctccgcgttccgccaactgtgaaaaaatgaggaacttcttgctatctccggggctgccagcgaccgtagccgccgagctgttggaggacattgttaccggagctctgggagacgatcctcaggtgatttctcacttttgtgaagattttagtcttcatgatctctatgatattgatccgggtgttgaggggcaagaggatgaatggctggagtctgtggatgggttttttccggacgctatgctgctagaggctgatttgccaccacctcacaactctcacactgagcccgagtcagctgctattcctgaattgtcatcaggtgaacttgacttggcttgttacgagactatgcctccggagtcggatgaggaggacagcgggatcagcgatcccacggcttttatggtctctaaggcgattgctatactaaaagaagatgatgatggcgatgatggatttcgactggacgctccggcggtgccggggagagactgtaagtcctgtgaataccaccgggatcgtaccggagacccgtctatgttgtgttctctgtgttatctccgtcttaacgctgcttttgtctacagtaagtgttttgtgcttttttaccctgtggctttgttgagtttatttttttctgtgtctcatagggtgttgtttattataggtcctgtttcagatgtggaggaacctgatagtactactggaaatgaggaggaaaagccctccccgccgaaactaactcagcgctgcagacctaatattttgagaccctcggcccagcgtgtgtcatcccggaaacgtgctgctgttaattgcatagaagatttattggaagagcccactgaacctttggacttgtccttaaagcgaccccgcccgcagtagggcgcggtgccagttttttctctctagcttccgggtgactcagtgcaataaaaattttcttggcaacaggtgtatgtgtttactttacgggcgggaagggattaggggagtataaagctggaggggaaaaatctgaggctgtcagatcgagtgagaagttccatggacttgtacgagagcctagagaatctaagttctttgcgacgtttgctggaggaggcctccgacagaacctcttacatttggaggtttctgttcggttcccctctgagtcgctttttgcaccgggtgaagcgagagcacctgacggaatttgatgggcttttagagcagctgcctggactgtttgattctttgaatctcggccaccggacgctgctagaggagaggctttttccacaattggacttttcctctccaggccgtctgtgttcagcgcttgcttttgctgtacatctgttggacagatggaacgagcagacgcagctcagcccgggttacactctggacttcctgacgctatgcctatggaagttcggaatcaggagggggaggaagctgtacgggcgcttggtggagaggcatccgtctctgcgccagcagcgtctgcaagctcaagtgctgctgaggcgggaggatctggaagccatttcggaggaggagagcggcatggaagagaagaatccgagagcggggctggaccctccggcggaggagtaggggggataccggacccttttcctgagttggctttgggggcggtggggggcgcttctgtggtacgtgaggatgaagaggggcgccaacgcggtcagaagagggagcattttgagtcctcgactttcttggctgatgtaaccgtggccctgatggcgaaaaacaggctggaggtggtgtggtacccggaagtatgggaggactttgagaagggggacttgcacctgctggaaaaatataactttgagcaggtgaaaacatactggatgaacccggatgaggactgggaggtggttttgaaccgatacggcaaggtagctctgcgtcccgactgtcgctaccaggttcgcgacaaggtggtcctgcgacgcaacgtgtacctgttgggcaacggcgccaccgtggagatggtggaccccagaaggggtggttttgtggccaatatgcaagaaatgtgccctggggtggtgggcttgtctggggtgacttttcatagtgtgaggtttagcggtagcaattttgggggtgtggttattaccgcgaacactcctgtggtcctgcataattgctacttttttggcttcagcaacacctgtgtggaaatgagggtgggaggcaaagtgcgcgggtgttccttttacgcttgctggaagggggtggtgagccagggtaaggctaaagtgtctgttcacaagtgtatgttggagagatgcaccttgggcatttccagtgagggcttcctccacgccagcgacaacgtggcttctgacaacggctgcgcctttcttatcaagggagggggtcgcatctgtcacaacatgatatgcggccctggggatgtccccccaaagccttaccagatggttacctgcacagatggcaaggtgcgcatgctcaagcctgtgcacattgtgggccaccggcgccaccgctggccagagtttgaacacaatgtgatgacccgctgtagcttgtacctgggaggcaggcgaggagttttcttgcccagacagtgtaacctggcccactgcaacgtgatcatggaacaatccgccgctacccaggtttgctttggaggaatatttgatataagcatggtggtgtataagatcctgcgctacgacgactgtcgggctcgtactcgaacctgcgactgcggagcctctcacctgtgtaacctgactgtgatggggatggtgactgaggaggtgcgactggaccactgtcagcactcttgcctgcgggaggagttttcttcctcggacgaggaggactaggtaggtggttggggcgtggccagcgagagggtgggctataaaggggaggtgtcggctgacgctgtcttctgtttttcaggtaccatgagcggatcaagcagccagaccgcgctgagcttcgacggggccgtgtacagcccctttctgacggggcgcttgcctgcctgggccggagtgcgtcagaatgttaccggttcgaccgtggacggacgtcccgtggatccatctaacgctgcttctatgcgctacgctactatcagcacatctactctggacagcgccgctgccgccgcagccgccacctcagccgctctctccgccgccaagatcatggctattaacccaagcctttacagccctgtatccgtggacacctcagccctggagctttaccggcgagatctagctcaagtggtggaccaactcgcagccgtgagccaacagttgcagctggtgtcgacccgagtggagcaactttcccgccctccccagtaaccgcaaaaattcaataaacagaatttaataaacagcacttgagaaaagtttaaacttgtggttgactttattcctggatagctggggggagggaacggcgggaacggtaagacctggtccatcgttcccggtcgttgagaacacggtggattttttccaagacccgatagaggtgggtctgaacgttgagatacatgggcatgagcccgtctcgggggtggaggtaggcccactgcagggcctcgttttcaggggtggtgttgtaaatgatccagtcgtaggccccccgctgggcgtggtgctggaagatgtccttcagcagcaagctgatggcaacgggaagacccttggtgtaggtgttgacaaagcggttgagttgggaggggtgcatgcggggactgatgaggtgcattttggcctggatcttgaggttggctatgttgccgcccagatcgcgcctgggattcatgttatgcaagaccaccagcaccgagtaaccggtgcagcgggggaatttgtcgtgcagcttggaagggaaagcgtggaagaatttggagacccctcggtgcccgcctaggttttccatgcactcatccatgatgatggcgatgggcccccgggaggcagcctgggcaaaaacgttgcgggggtccgtgacatcgtagttgtggtcctgggtgagttcatcataggacattttgacaaagcgcgggcagagggtcccagactggggaatgatggttccatccggtccgggggcgtagttgccctcgcagatttgcatttcccaggctttgatttcagagggagggatcatgtcaacctggggggcgatgaaaaaaatggtctctggggcgggggtgatgagctgggtggaaagcaggttgcgcaagagctgtgacttgccgcagccggtgggcccgtagatgacagctatgacgggttgcagggtgtagtttagagagctacaactgccatcatccttcaaaagcggggccacactgtttaaaagttctctaacatgtaagttttcccgcactaagtcctgcaggagacgtgaccctcctagggagagaagttcaggaagcgaagcaaagtttttaagtggcttgaggccatcggccaagggcaagttcctgagagtttgactgagcagttccagccggtcccagagctcggttacgtgctctacggcatctcgatccagcagacctcctcgtttcgggggttggggcggctctggctgtagggaatgaggcggtgggcgtccagctgggccatggtgcggtccctccatgggcgcagggttctcttcagggtggtctcggtcacggtgaatgggtgggccccgggctgggcgctggccagggtgcgcttgaggctgaggcggctggtggcgaaccgttgcttttcgtctccctgcaagtcagccaaatagcaacggaccatgagctcatagtccaggctctctgcggcatgtcctttggcgcgaagcttgcctttggaaacgtgcccgcagtttgagcagagcaagcattttagcgcgtagagttttggcgccaagaacacggattccggggaataagcatccccaccgcagttggagcaaacggtttcgcattccaccagccaggtcagctgaggatcttttgggtcaaaaaccaagcgcccgccgttttttttgatgcgcttcctacctcgggtctccatgaggcggtgcccgcgttcggtgacgaagaggctgtcggtgtctccgtagacggaggtcagggcgcgctcctccaggggggtcccgcggtcctcggcgtagagaaactcgcaccactctgacataaacgcccgggtccaggctaggacgaatgaggcgatgtgggaagggtaccggtcgttatcgatgagggggtcggttttttccaaggtgtgcaggcacatgtccccctcgtccgcttccaaaaatgtgattggcttgtaggtgtaagtcacgtgatcctgtccttccgcgggggtataaaagggggcgtttcccccctcctcgtcactctcttccggttcgctgtcgccaaaggccagctgttggggtacgtaaacgcgggtgaaggcgggcatgacctgtgcgctgaggttgtcagtttctatatacgaggaagatttgatggcgagcgcccccgtggagatgcccttgaggtgctcggggcccatttggtcagaaaacacaatctgtcggttatcaagcttggtggcaaaagacccgtagagggcgttggagagcaacttggcgatggagcgctgggtttggtttttttcccggtcggctttttccttggccgcgatgttgagctggacgtactccctggccacgcacttccagccgggaaaaacggccgtgcgctcgtccggcaccagcctcacgctccatccgcggttgtgcagggtgatgacgtcgatgctggtggccacctctccgcgcaggggctcgttggtccagcagaggcgaccgcccttgcgagagcagaaggggggcagggggtcaagcaggcgctcgtccggggggtcggcgtcgatggtaaagatggcgggcagcaggtgtttgtcaaagtaatcgatctgatgcccggggcaacgcagggcggtttcccagtcccgcaccgccaaggcgcgctcgtatggactgaggggggcgccccagggcatgggatgcgtcagggccgaggcgtacatgccgcagatgtcatagacgtaaaggggctcctccaggacgccgaggtaggtggggtagcagcgccccccgcggatgctggcccgtacgtagtcgtagagctcgtgcgagggggccagaaggtggcggctgaggtgagcgcgctggggcttttcatctcggaagaggatctgcctgaagatggcgtgggagttggaggagatggtgggccgctgaaaaatgttgaagcgggcgtcgggcagacccacggcctcgccgataaagtgggcgtaggactcttgcagcttttccaccagggaggcggtgaccagcacgtccagagcgcagtagtccagggtttcccgcacgatgtcataatgctcttcctttttttccttccagaggtctcggttgaagagatactcttcgcggtctttccagtactcttggagaggaaacccgttttcgtctccacggtaagagcccaacatgtaaaactggttgacggcctgatagggacagcatcccttctccacgggcagcgagtaggccagggcggccttgcgcagggaggtgtgagtcagggcaaaggtgtcgcggaccataacttttacaaactggtacttaaagtcccggtcgtcgcacatgcctcgctcccagtctgagtagtctgtgcgctttttgtgcttggggttaggcagggagtaggtgacgtcgttaaagaggattttgccacatctgggcataaagttgcgagagattctgaaggggccgggcacctccgagcggttgttgatgacttgggcagccaggagaatttcgtcgaagccgttgatgttgtgccccacgacgtagaactctatgaaacgcggagcgccgcgcagcagggggcacttttcaagttgctggaaagtaagttcccgcggctcgacgccgtgttccgtgcggctccagtcctccaccgggtttcgctccacaaaatcctgccagatgtggtcgactagcaagagctgcagtcggtcgcgaaattcgcggaattttctgccgatggcttgcttctgggggttcaagcaaaaaaaggtgtctgcgtggtcgcgccaggcgtcccagccgagctcgcgagccagattcagggccagcagcaccagagccggctcaccggtgattttcatgacgaggagaaagggcaccagctgttttccgaacgcgcccatccaggtgtaggtctccacgtcgtaggtgagaaacagacgttcggtccgcgggtgcgatcccagggggaaaaacttgatgggctgccaccattgggagctctgggcgtggatgtgatggaagtaaaagtcccggcggcgcgtggaacattcgtgctggtttttgtaaaagcggccgcagtggtcgcagcgcgagacggagtgaaggctgtgaatcaggtgaatcttgcgtcgctgagggggccccagagccaaaaagcggagcgggaacgaccgcgcggccacttcggcgtccgcaggcaagatggatgagggttccaccgttccccgcccgcggaccgaccagacttccgccagctgcggcttcagttcttgcaccagctctcgcagcgtttcgtcgctgggcgaatcgtgaatacggaagttgtcgggtagaggcgggaggcggtggacttccaggaggtgtgtgagggccggcaggagatgcaggtggtacttgatttcccacggatgacggtcgcgggcgtccaaggcgaagagatgaccgtggggccgcggcgccaccagcgttccgcggggggtctttatcggcggcggggacgggctcccggcggcagcggcggctcgggacccgcgggcaagtcgggcagcggcacgtcggcgtggagctcgggcaggggctggtgctgcgcgcggagctgactggcaaaggctatcacccggcgattgacgtcctggatccggcggcgctgcgtgaagaccaccggacccgtggtcttgaacctgaaagagagttcgacagaatcaatctcggcatcgttaaccgcggcctggcgcaggatttcggccacgtccccggagttgtcttgatacgcgatttctgccatgaactggtcgatttcctcttcctgcaagtctccgtgaccggcgcgttcgacggtggccgcgagatcgttggagatgcggcccatgagctgggaaaaggcattgatgccgacctcgttccacactcggctgtacaccacctctccgtgaacgtcgcgggcgcgcatcaccacctgggcgagattgagttccacgtggcgggcgaaaaccggatagtttcggaggcgctgatacagatagttgagggtggtggcggcgtgctcggccacaaaaaaatacatgatccagcggcggagggtcagctcgttgatgtcgcccagcgcctccaggcgttccatggcctcgtaaaagtccacggcaaagttgaaaaattggctgttcctggccgagaccgtgagctcttcttccaagagccgaatgagatccgccacggtggccctgacttcgcgttcgaaagccccgggtgcctcctccacctcttcctcctcgacttcttcgaccgcttcgggcacctcctcttcctcgaccaccacctcaggcggggctcggcggcgccggcggcggacgggcaggcggtcgacgaaacgctcgatcatttcccccctccgtcgacgcatggtctcggtgacggcgcgaccctgttcgcgaggacgcagggtgaaggcgccgccgccgagcggaggtaacagggagatcggggggcggtcgtgggggagactgacggcgctaactatgcatctgatcaatgtttgcgtagtgacctcgggtcggagcgagctcagcgcttgaaaatccacgggatcggaaaaccgttccaggaacgcgtctagccaatcacagtcgcaaggtaagctgaggaccgtctcgggggcttgtctgttctgtcttcccgcggtggtgctgctgatgaggtagttgaagtaggcgctcttgaggcggcggatggtggacaggagaaccacgtctttgcgcccagcttgctgtatccgcaggcggtcggccatgccccacacttctccttgacagcggcggaggtccttgtagtattcttgcatcagcctttccacgggcacctcgtcttcttcttccgctcggccggacgagagccgcgtcaggccgtacccgcgctgcccctgtggttggagcagggccaggtcggccacgacgcgctcggccagcacggcctgctggatgcgggtgagggtgtcctgaaagtcgtcgagatccacaaagcggtggtacgcgccagtgttgatggtgtaggtgcagttgctcatgacggaccagtttacggtctgggtgccatggcccacggtttccaggtagcggagacgcgagtaggcccgcgtctcgaagatgtagtcgttgcaggtccgcagcaggtactggtagcccaccagcagatgcggcggcggctggcggtagaggggccaccgctgggtggcgggggcgttgggggcgagatcttccaacatgaggcggtgatagccgtagatgtagcgcgacatccaagtgatgccgctggccgtggtgctggcgcgggcgtagtcgcgaacgcggttccagatgtttcgcagcggctggaagtactcgatggtggggcgactctgccccgtgaggcgggcgcagtcggcgatgctctacggggaaaaagaagggccagtgaacaaccgccttccgtagccggaggagaacgcaagggggtcaaagaccaccgaggctcgggttcgaaacccgggtggcggcccgaatacggagggcggttttttgcttttttctcagatgcatcccgtgctgcggcagatgcgtccgaacgcggggtcccagtccccggcggtgcctgcggccgtgacggcggcttctacggccacgtcgcgctccaccccgcctaccacggcccaggcggcggtggctctgcgcggcgcaggggaacccgaagcagaggcggtgttggacgtggaggagggccaggggttggctcggctgggggccctgagtcccgagcggcacccgcgcgtggctctgaagcgcgacgcggcggaggcgtacgtgccgcggagcaatctgtttcgcgaccgcagcggcgaggaggccgaggagatgcgagacttgcgttttcgggcggggagggagttgcgtcacgggctggaccggcagagggttctgagagaggaggactttgaggcggacgagcgcacgggggtgagtcccgcgcgggctcacgtggcggccgccaacctggtgagcgcgtacgagcagacggtcaaggaggagatgaacttccagaagagcttcaatcatcacgtgcgcacgctgattgcgcgcgaagaggtggccatcggcctcatgcatctgtgggattttgtggaggcgtacgttcagaaccccagcagcaagccgctgacggctcagctgttcctcatcgtgcaacatagtcgagacaacgaaacgttcagggaggccatgctgaacattgcagagcctgaggggcgctggctcttggatctcattaacatcttgcagagtatcgtagtgcaggagcgctcgctgagcctggccgacaaggtggctgccatcaactacagcatgctgtcgctgggcaaattttacgcccgcaagatctacaagtctccgttcgtccccatagacaaggaggtgaagatagacagcttttacatgcgcatggcgctcaaggtgctgactctaagcgacgacctgggggtgtaccgcaacgaccgcatacacaaggcggtgagcgccagccgccggcgcgagctgagcgaccgcgagcttttgcacagcctgcatcgggcgttgactggtgccggcagcgccgaggcggccgagtactttgacgccggagcggacttgcgctggcagccatcccgacgcgcgctggaggcggctggcgtcggggagtacggggtcgaggacgacgatgaagcggacgacgagttgggcattgacttgtagccgtttttcgttagatatgtcggcgaacgagccgtctgcggccgccatggtgacggcggcgggcgcgccccaggacccggccacgcgcgcggcgctgcagagtcagccttccggagtgacgcccgcggacgactggtccgaggccatgcgtcgcatcctggcgctgacggcgcgcaaccccgaggcttttcggcagcagccgcaggcaaaccggtttgcggccattttggaagcggtggtgccctccagacccaaccccacccacgaaaaggtgctggccatcgtcaacgccctggcggagaccaaggccatccgcccagacgaggccgggcaggtttacaacgcgctgctagaaagggtgggacgctacaacagctccaacgtgcagaccaatctggaccgcttggtgacggacgtgaaggaggccgtagcccagcgagagcggtttttcaaggaagccaatctgggctcgctggtggccctcaacgccttcctgagcacgctgccggcgaacgtgccccgcggtcaggaggactacgtgaactttctgagcgccctccgcctgatggtggccgaggtgccgcagagcgaggtgtaccagtctggccccaactactacttccagacctcccggcagggcctgcagacggtaaacctgacgcaggcctttcagaacctgcagggcctttggggggtgcgcgctccgctgggcgaccgcagcacggtgtccagcctgctgacccccaatgcccggctgctcttgcttctcattgctccgttcaccgacagcggttccatcagccgcgactcttacctgggacacctgctcaccctgtaccgggaggccatcgggcaggcgcgggtggacgagcagacgtaccaggaaatcaccagcgtgagccgcgcgctggggcaggaggacacgggcagcttggaggcgactctgaacttcctgctgaccaaccggcggcagcgcctacctccccagtacgcgctgaacgcggaggaggagcgcatcctgcgtttcgtgcagcagagcaccgcgctgtacttgatgcgggaaggcgcctctcccagcgcttcgctggacatgacggcggccaacatggagccatcgttctacgccgccaaccgtcccttcgtcaaccggctaatggactatttgcatcgggcggcggccctgaacccggaatactttactaacgtcatcctgaacgaccgttggctgccacctcccggcttctacacgggggagttcgacctcccggaggccaacgacggtttcatgtgggacgacgtggacagcgtgttcctgcccggcaagaaggaggcgggtgactctcagagccaccgcgcgagcctcgcagacctgggggcgaccgggcccgcgtctccgctgcctcgcctgccgagcgccagcagcgccagcgtggggcgggtgagccgtccgcgcctcagcggtgaggaggactggtggaacgatccgctgctccgtccggcccgcaacaaaaacttccccaacaacgggatagaggatttggtagacaaaatgaaccgttggaagacgtatgcccaggagcatcgggagtggcaggcgaggcaacccatgggccctgttctgccgccctctcggcgcccgcgcagggacgaagacgccgacgattcagccgatgacagcagcgtgttggatctgggcgggagcgggaacccctttgcccacctgcaacctcgcggcgtgggtcggcggtggcgctaggaaaaaaaattattaaaagcacttaccagagccatggtaagaagagcaacaaaggtgtgtcctgctttcttcccggtagcaaaatgcgtcgggcggtggcagttccctccgcggcaatggcgttaggcccgcccccttcttacgaaagcgtgatggcagcggccaccctgcaagcgccgttggagaatccttacgtgccgccgcgatacctggagcctacgggcgggagaaacagcattcgttactcggagctgacgcccctgtacgacaccacccgcctgtacctggtggacaacaagtcagcagatatcgccaccttgaactaccagaacgaccacagcaactttctcacgtccgtggtgcagaacagcgactacacgcccgccgaagcgagcacgcagaccattaacttggacgaccgctcgcgctggggcggggacttgaaaaccattctgcacactaacatgcccaacgtgaacgagttcatgtttaccaactcgttcagggctaaacttatggtggcgcacgaggccgacaaggacccggtttatgagtgggtgcagctgacgctgccggaggggaacttttcagagattatgaccatagacctgatgaacaacgccattatcgaccactacctggcggtagccagacagcagggggtgaaagaaagcgagatcggcgtcaagtttgacacgcgcaactttcgtctgggctgggacccggagacggggcttgtgatgccgggggtgtacacgaacgaagctttccatcccgacgtggtcctcttgccgggctgcggggtggactttacctacagccggttaaacaacctgctaggcatacgcaagagaatgccctttcaggaagggtttcagatcctgtacgaggacctggagggcggtaacatcccggccctgctggacgtgccggcgtacgaggagagcatcgccaacgcaagggaggcggcgatcaggggcgataatttcgcggcgcagccccaggcggctccaaccataaaacccgttttggaagactccaaagggcggagctacaacgtaatagccaacaccaacaacacggcttacaggagctggtatctggcttataactacggcgacccggagaagggggttagggcctggaccctgctcaccactccggacgtgacgtgcggttcagagcaggtctactggtcgctgcctgacatgtacgtggaccctgtgacgtttcgctccacgcagcaagttagcaactacccagtggtgggagcggagcttatgccgattcacagcaagagcttttacaacgagcaggccgtctactcacagctcattcgtcagaccaccgccctaacgcacgttttcaaccgcttccccgagaaccaaatcctagtgcgacctccagcgcccaccatcaccaccgtcagcgagaacgtgcccgctctaaccgatcacgggacgctgcctttgcagaacagcatccgcggagttcagcgagttaccatcacggacgcccgtcgtcggacctgtccctacgtctacaaagccttgggaatcgtggccccgcgcgtcctgtcgagtcgcactttctagatgtccatcctcatctctcccagcaacaataccggttggggtctgggcgtgaccaaaatgtacggaggcgccaaacgacggtccccacaacatcccgtgcgagtgcgcgggcactttagagccccatgggggtcgcacacgcgcgggcgcaccggccgaaccaccgtcgacgacgtgatcgatagcgtggtggccgacgcccgcaactaccagcccgctcgatccacggtggacgaagtcatcgacggcgtggtggccgacgccagggcctacgcccgcagaaagtctcgtctgcgccgccgccgttcgctaaagcgccccacggccgccatgaaagccgctcgctctctgctgcgtcgcgcacgtatcgtgggtcgccgcgccgccagacgcgcagccgccaacgccgccgccggccgagtgcgccgccgggccgcccagcaggccgccgccgccatctccagtctatccgccccccgacgcgggaatgtgtactgggtcagggactcggccaccggcgtgcgagttcccgtgagaacccgtcctcctcgtccctgaataaaaagttctaagcccaatcggtgttccgttgtgtgttcagctcgtcatgaccaaacgcaagtttaaagaggagctgctgcaagcgctggtccccgaaatctatgcgccggcgccggacgtgaaaccgcgtcgcgtgaaacgcgtgaagaagcaggaaaagctagagacaaaagaggaggcggtggcgttgggagacggggaggtggagtttgtgcgctcgttcgcgccgcgtcggcgagtgaattggaaggggcgcaaggtgcaacgggtgctgcgtcccggcacggtggtgtctttcaccccgggtgaaaaatccgcctggaagggcataaagcgcgtgtacgatgaggtgtacggggacgaagacattctggagcaggcgctggatagaagcggggagtttgcttacggcaagagggcgaggacgggcgagatcgccatcccgctggacacttccaaccccacccccagtctgaaacccgtgacgctgcaacaggtgttgccggtgagcgccccctcgcgacgcggcataaaacgcgagggcggcgagctgcagcccaccatgcagctcctggttcccaagaggcagaaactagaggacgtactggacatgataaaaatggagcccgacgtgcagcccgatattaaaatccgtcccatcaaagaagtggcgccgggaatgggcgtgcagaccgtggacatccagattcccatgaccagcgccgcacaggcggtagaggccatgcagaccgacgtggggatgatgacggacctgcccgcagctgctgccgccgtggccagcgccgcgacgcaaacggaagccggcatgcagaccgacccgtggacggaggcgcccgtgcagccggccagaagacgcgtcagacggacgtacggccccgtttctggcataatgccggagtacgcgctgcatccttccatcatccccacccccggctaccgggggcgcacctaccgtccgcgacgcagcaccactcgccgccgtcgccgcacggcacgagtcgccaccgccagagtgagacgcgtaacgacacgtcgcggccgccgcttgaccctgcccgtggtgcgctaccatcccagcattctttaaaaaaccgctcctacgttgcagatgggcaagcttacttgtcgactccgtatggccgtgcccggctaccgaggaagatcccgccgacgacggactttgggaggcagcggtttgcgccgccgtcgggcggttcaccggcgcctcaagggaggcattctgccggccctgatccccataatcgccgcagccatcggggccattcccggaatcgccagcgtagcggtgcaggctagccagcgccactgattttactaaccctgtcggtcgcgccgtctctttcggcagactcaacgcccagcatggaagacatcaatttctcctctctggccccgcggcacggcacgcggccgtatatggggacgtggagcgagatcggcacgaaccagatgaacgggggcgctttcaattggagcggtgtgtggagcggcttgaaaaatttcggttccactctgaaaacttacggcaaccgggtgtggaactccagcacggggcagatgctgagggacaagctaaaggacacgcagtttcagcaaaaggtggtggacggcatcgcttcgggcctcaacggcgccgtcgacctggccaaccaggccattcaaaaggaaattaacagccgcctggagccgcggccgcaggtggaggagaacctgccccctctggaggcgctgccccccaagggagagaagcgcccgcggcccgacatggaggagacgctagttactaagagcgaggagccgccatcatacgaggaggcggtgggtagctcgcagctgccgtccctcacgctgaagcccaccacctatcccatgaccaagcccatcgcctccatggcgcgccccgtgggagtcgacccgcccatcgacgcggtggccactttggacctgccgcgccccgaacccggcaaccgcgtgcctcccgtccccatcgctccgccggtttctcgccccgccatccgccccgtcgccgtggccactccccgctatccgagccgcaacgccaactggcagaccaccctcaacagtattgtcggactgggggtgaagtctctgaagcgccgtcgctgtttttaaagcacaatttattaaacgagtagccctgtcttaatccatcgttgtatgtgtgcctatatcacgcgttcagagcctgaccgtccgtcaagatggccactccgtcgatgatgccgcagtggtcgtacatgcacatcgccgggcaggacgcctcggagtacctgagcccgggtctggtgcagtttgcccgtgcgacggaaacctacttctcactgggcaacaagttcaggaaccccaccgtggcgcccacccacgacgtcaccaccgatcggtcccagcgactgacaatccgcttcgtccccgtggacaaggaagacaccgcttactcctacaaaacccgcttcacgctggccgtgggcgacaaccgggtgctagacatggccagtacctactttgacatccgcggcgtgatcgaccgcggacctagcttcaagccttactccggcacggcttacaactcactggctcccaaaggggcgcccaacaacagccaatggaacgccacagataacgggaacaagccagtgtgttttgctcaggcagcttttataggtcaaagcattacaaaagacggagtgcaaatacagaactcagaaaatcaacaggctgctgccgacaaaacttaccaaccagagcctcaaattggagtttccacctgggataccaacgttaccagtaacgctgccggacgagtgttaaaagccaccactcccatgctgccatgttacggttcatatgccaatcccactaatccaaacgggggtcaggcaaaaacagaaggagacatttcgctaaactttttcacaacaactgcggcagcagacaataatcccaaagtggttctttacagcgaagatgtaaaccttcaagcccccgatactcacttagtatataagccaacggtgggagaaaacgttatcgccgcagaagccctgctaacgcagcaggcgtgtcccaacagagcaaactacataggtttccgagataactttatcggtttaatgtattataacagcacagggaacatgggagttctggcaggtcaggcctcgcagttaaacgcagttgtagacctgcaagatcgaaacacggaactgtcctatcagctaatgctagatgctctgggtgacagaactcgatatttctcaatgtggaatcaggccgtggacagctacgatccagacgttaggattatcgagaaccatggggtggaagacgagctgcccaattactgttttccactcccaggcatgggtatttttaactcctacaagggggtaaaaccacaaaatggcggtaatggtaactgggaagcaaacggggacctatcaaatgccaatgagatcgctttaggaaacatttttgccatggaaattaacctccacgcaaacctgtggcgcagcttcttgtacagcaatgtggcgctgtacctgccagacagctataaattcactcccgctaacatcactctgcccgccaaccaaaacacctacgagtatatcaacgggcgcgtcacttctccaaccctggtggacacctttgttaacattggagcccgatggtcgccggatcccatggacaacgtcaacccctttaaccatcaccggaacgcgggcctccgttaccgctccatgctgctgggaaatggacgcgtggtgcctttccacatacaagtgccgcaaaaatttttcgcgattaagaacctcctgcttttgcccggctcctacacttacgagtggagcttcagaaaagacgtgaacatgattctgcagagcaccctgggcaatgatcttcgagtggacggggccagcgtccgcattgacagcgtcaacttgtacgccaactttttccccatggcgcacaacaccgcttctaccttggaagccatgctgcgaaacgacaccaacgaccagtcgtttaacgactacctcagcgcggccaacatgctttatcccattccggccaacgccaccaacgttcccatttccattccctcccgcaactgggcggccttccggggatggagcttcacccgccttaaagccaaggaaacgccttccttgggctccggctttgacccctactttgtgtactcaggcaccattccttacctggacggcagcttttacctcaaccacactttcaaacgtctgtccatcatgttcgattcttccgtaagttggccgggcaacgaccgcctcctgacgccgaacgagttcgaaattaagcgcattgtggacggggaaggctacaacgtggctcaaagtaacatgaccaaagactggtttttaattcaaatgctcagccactacaacatcggctaccaaggcttctatgttcccgagggctacaaggatcggatgtattctttcttccgaaactttcagcccatgagccgccaggtgccggatcccaccgctgccggctatcaagccgttcccctgcccagacaacacaacaactcgggctttgtggggtacatgggcccgaccatgcgcgaaggacagccatacccggccaactacccctatcccctgatcggcgctaccgccgtccccgccattacccagaaaaagtttttgtgcgaccgcgtcatgtggcgcatacctttttccagcaactttatgtcaatgggggccctgaccgacctcggacagaacatgctttacgctaactccgcccatgccctggatatgacttttgaggtggaccccatgaacgagcccacgttgctgtacatgctttttgaggtgttcgacgtggtcagagtgcaccagccgcaccgcggtattatcgaggccgtgtacctgcgcacccccttctctgcgggcaatgccaccacataagccgctgaactagctggtttttaccccagatcccatgggctccacggaagacgaactgcgggccattgtgcgagacctgggctgcggaccctacttcctgggcacctttgacaagcggtttcccgggttcgtgtctcctcgcaaactcgcgtgcgcgatcgtgaataccgccggccgagagaccggaggagagcattggctagctctgggctggaacccccgctcgtccacgtttttcctgttcgacccctttggcttttcagaccaacgcttgaagcagatctatgcatttgaatatgagggtctactcaagcgaagcgcgctggcctcctccgccgatcactgtctaaccctggtaaagagcactcagacggttcagggccctcacagcgccgcctgtggccttttttgttgcatgtttttgcacgcctttgtgaactggccggacacccccatggaaaacaaccccaccatggacctcctgactggcgttcccaactccatgctccaaagccccagcgtgcagaccaccctcctccaaaaccagaaaaatctgtacgcctttctgcacaagcactctccctactttcgccgccatcgggaacaaatagaaaatgcaaccgcgtttaacaaaactctgtaacgtttaataaatgaactttttattgaactggaaaacgggtttgtgatttttaaaaatcaaaggggttgagctggacatccatgtgggaggccggaagggtggtgttcttgtactggtacttgggcagccacttaaactctggaatcacaaacttgggcagcggtatttctgggaagttgtcgtgccacagctggcgggtcagctgaagtgcctgcagaacatcgggggcggagatcttgaagtcgcagtttatctggttcacggcacgcgcgttgcggtacatgggattggcacactgaaacaccagcaggctgggattcttgatgctagccagggccacggcgtcggtcacgtcaccggtgtcttctatgttggacagcgaaaaaggcgtgactttgcaaagctggcgtcccgcgcgaggcacgcaatctcccaggtagttgcactcacagcggatgggcagaagaagatgcttgtggccgcgggtcatgtagggataggccgctgccataaaagcttcgatctgcctgaaagcctgcttggccttgtgcccttcggtataaaaaacaccgcaggacttgttggaaaaggtattactggcgcaagcggcatcgtgaaagcaagcgcgtgcgtcttcgtttcgtaactgcaccacgctgcggccccaccggttctgaatcaccttggccctgccggggttttccttgagagcgcgctggccggcttcgctgcccacatccatttccacgacatgctccttgttaatcatggccagaccgtggaggcagcgcagctcctcgtcatcgtcggtgcagtgatgctcccacacgacgcagccagtgggctcccacttgggcttggaggcctcggcaatgccagaatacaggagaacgtagtggtgcagaaaacgtcccatcatggtgccaaaggttttctggctgctgaaggtcatcgggcagtacctccagtcctcgttaagccaagtgttgcagatcttcctgaagaccgtgtactgatcgggcataaagtggaactcattgcgctcggtcttgtcgatcttatacttttccatcagactatgcataatctccatgcccttttcccaggcgcaaacaatcttggtgctacacgggttaggtatggccaaagtggttggcctctgaggcggcgcttgttcttcctcttgagccctctcccgactgacgggggttgaaagagggtgccccttggggaacggcttgaacacggtctggcccgaggcgtcccgaagaatctgcatcgggggattgctggccgtcatggcgatgatctgaccccggggctcctccacttcgtcctcctcgggactttcctcgtgcttttcgggggacggtacgggagtagggggaagagcgcggcgcgccttcttcttgggcggcagttccggagcctgctcttgacgactggccattgtcttctcctaggcaagaaaaacaagatggaagactctttctcctcctcctcgtcaacgtcagaaagcgagtcttccaccttaagcgccgagaactcccagcgcatagaatccgatgtgggctacgagactccccccgcgaacttttcgccgccccccataaacactaacgggtggacggactacctggccctaggagacgtactgctgaagcacatcaggcggcagagcgttatcgtgcaagatgctctcaccgagcgactcgcggttccgctggaagtggcggaacttagcgccgcctacgagcgaaccctcttctccccaaagactccccccaagaggcaggctaacggcacctgcgagcctaaccctcgactcaacttctaccctgcctttgccgtgccagaggtactggctacgtaccacatttttttccaaaaccacaaaatccctctctcgtgccgcgccaaccgcaccaaagccgatcgcgtgctgcgactggaggaaggggctcgcatacctgagattgcgtgtctggaggaagtcccaaaaatctttgaaggtctgggccgcgacgaaaagcgagcagcaaacgctctggaagagaacgcagagagtcacaacagcgccttggtagaactcgagggcgacaacgccagactggccgtcctcaaacggtccatagaagtcacgcacttcgcctaccccgccgttaacctccctccaaaagttatgacagcggtcatggactcgctgctcataaagcgcgctcagcccttagacccagagcacgaaaacaacagtgacgaaggaaaaccggtggtttctgatgaggagttgagcaagtggctgtcctccaacgaccccgccacgttggaggaacgaagaaaaaccatgatggccgtggtgctagttaccgtgcaattagaatgtctgcagaggttcttttcccacccagagaccctgagaaaagtggaggaaacgctgcactacacatttaggcacggctacgtgaagcaagcctgcaagatttccaacgtagaacttagcaacctcatctcctacctggggatcttgcacgaaaaccgcctcggacaaaacgtgctgcacagcacactgaaaggagaagcccgccgagactatgtgcgagactgcgtgttcctagcgctagtgtacacctggcagagcggaatgggagtctggcagcagtgcctggaggacgaaaacctcaaagagcttgaaaagctgctggtgcgctccagaagggcactgtggaccagttttgacgagcgcaccgccgcgcgagacctagctgatattatttttcctcccaagctggtgcagactctccgggaaggactgccagattttatgagtcaaagcatcttgcaaaacttccgctctttcatcttggaacgctcgggaatcttgcccgccactagctgcgccctacccacagattttgtgcctctccactaccgcgaatgcccaccgccgctgtggccgtacacttacttgcttaaactggccaactttctaatgttccactctgacctggcagaagacgttagcggcgaggggctgctagaatgccactgccgctgcaacctgtgcaccccccaccgctctctagtatgcaacactcccctgctcaatgagacccagatcatcggtacctttgaaatccagggaccctccgacgcggaaaacggcaagcaggggtctgggctaaaactcacagccggactgtggacctccgcctacttgcgcaaatttgtaccagaagactatcacgcccaccaaattaaattttacgaaaaccaatcaaaaccacccaaaagcgagttaacggcttgcgtcattacgcagagcagcatagttgggcagttgcaagccattaacaaagcgcggcaagagtttctcctaaaaaaaggaaaaggggtctacttggacccccagaccggcgaggaactcaacggaccctcctcagtcgcaggttgtgtgccccatgccgcccaaaaagaacacctcgcagtggaacatgccagagacggaggaagaggagtggagcagtgtgagcaacagcgaaacggaggaagagccgtggcccgaggggtgcaacggggaagaggacacggagggacggcgaagtcttcgccgaagaactctcgccgctgcccccgaagtcccagccggccgcctcggcccaagatcccgcacacacccgtagatgggatagcaagaccaaaaagccgggtaagagaaacgctcgcccccgccagggctaccgctcgtggagaaagcacaaaaactgcatcttatcgtgcttgctccagtgcggcggagacgtttcgttcacccgtagatacttgctttttaacaaaggggtggccgtcccccgtaacgtcctccactactaccgtcactcttacagctccgaagcggacggctaagaaaacgcagcagttgccggcgggaggactgcgtctcagcgcccgagaacccccagccaccagggagctccgaaaccgcatatttcccaccctctacgctatctttcagcaaagccgggggcagcagcaagaactgaaaataaaaaaccgcacgctgaggtcgcttacccgaagctgcctctatcacaagagcgaagagcagctgcagcgaaccctggaggacgcagaagcgctgttccagaagtactgcgcgaccaccctaaataactaaaaaagcccgcgcgcgggacttcaaaccgtctgacgtcaccagccgcgcgccaaaatgagcaaagagattcccacgccttacatgtggagttaccagccgcagatgggattagccgccggcgccgcccaggattactccacgaaaatgaactggctcagcgccgggccccacatgatttcccgcgtaaacgacattcgcgcccaccgcaatcagctattgttagaacaggctgctctgaccgccacgccccgtaataacctgaaccctcccagctggccagctgccctggtgtaccaggaaacgcctccacccaccagcgtacttttgccccgtgacgcccaggcggaagtccagatgactaacgcgggcgcgcaattagcgggcggatcccggtttcggtacagagttcacggcgccgcaccctatagcccaggtataaagaggctgatcattcgaggcagaggtgtccagctcaacgacgagacagtgagctcttcgcttggtctacgaccagacggagtgttccagctcgcgggctcgggccgctcttcgttcacgcctcgccaggcatacctgactctgcagagctctgcctctcagcctcgctcgggaggaatcggaccccttcagtttgtggaggagtttgtgccctcggtctactttcagcctttctccggatcgcccggccagtacccggacgagttcatccccaacttcgacgcggtgagtgactctgtggacggttatgactgatgtcgagcccgcttcagtgctagtggaacaagcgcggctcaatcacctggttcgttgccgccgccgctgctgcgtggctcgcgacttgagcttagctctcaagtttgtaaaaaacccgtccgaaaccgggagcgctgtgcacgggttggagctagtgggtcctgagaaggccaccatccacgttctcagaaactttgtggaaaaacccattttggttaaacgagatcaggggccttttgtaatcagcttactctgcacctgtaaccatgttgaccttcacgactattttatggatcatttgtgcgctgaattcaataagtaaagcgaattcttaccaagattatgatgtccatgactgttcctcgccactatacgatgttgtgccagtaaactctcttgtcgacatctatctgaactgttccttttggtccgcacagcttacttggtactacggtgacaccgtcctttctggctcactgggcagctcacacggaataacacttcacctcttttcgccgtttcgatacggaaactacagctgtcgtgccggtacctgcctccacgttttcaatcttcagccctgtccaccgaccaaacttgtatttgtcgactctaagcacttacagctcaactgcagcattctaggccccagtatcttgtggacatacaataaaatcaggttggtggaatttgtctactacccacccagcgcccgcggttttggggaaattcctttccagatctactacaactatcttgccacacattatgcaagtcaacagcaactaaacttgcaagcacccttcacgccaggagagtactcctgtcacgtaggctcctgcacagaaacttttattctcttcaacagatcttctgccattgaacgcttcactactaactactttagaaaccaagttgtgcttttcactgacgaaacccctaacgtcaccctggactgtgcatgtttttctcatgacaccgtaacttggactcttaacaatactctctggctcgcgttcgataaccaaagcttgattgttaaaaattttgatttaacctttactaaaccctctcctcgcgaaatagttatctttgctccttttaatccaaaaactaccttagcctgtcaggttttgtttaagccttgccaaacaaactttaagtttgtttatttgcctccgcaatctgtcaaactcatagaaaaatacaacaaagcgcccgtcttggctcctaaaaccttctaccactggctaacctacacggggctgtttgcactaattgtttttttcctaattaacatttttatatgtttcttgccttcctccttcttttcgcgaacaccgttgccgcagaaagacctctccttattactgtagcgcttgctatacaaaaccaagagtggtcaaccgtgctctcaatctattttcaatttttcattttgtccttaatactttctcttattgtcgttaacaatgatctggagcattggtctcgcctttttttggctgcttagtgcaaaagccactatttttcacaggtatgtggaagaaggaactagcaccctctttacgatacctgaaacaattaaggcggctgatgaagtttcttggtacaaaggctcgctctcagacggcaaccactcattctcaggacagaccctttgcatccaagaaacttattttaaatcagaactacaatacagctgcataaaaaactttttccatctctacaacatctcaaaaccctatgagggtatttacaatgccaaggtttcagacaactccagcacacggaacttttactttaatctgacagttattaaagcaatttccattcctatctgtgagtttagctcccagtttctttctgaaacctactgtttaattactataaactgcactaaaaatcgccttcacaccaccataatctacaatcacacacaatcaccttgggttttaaacctaaaattttctccacacatgccttcgcaatttctcacgcaagttaccgtctctaacataagcaagcagtttggcttttactatcctttccacgaactgtgcgaaataattgaagccgaatatgaaccagactactttacttacattgccattggtgtaatcgttgtttgcctttgctttgttattggggggtgtgtttatttgtacattcagagaaaaatattgctctcgctgtgctcctgcggttacaaagcagaagaaagaattaaaatctctacactttattaatgttttccagaaatggcaaaactaacgctcctacttttgcttctcacgccggtgacgctttttaccatcactttttctgccgccgccacactcgaacctcaatgtttgccaccggttgaagtctactttgtctacgtgttgctgtgctgcgttagcgtttgcagtataacatgttttacctttgtttttcttcagtgcattgactacttctgggtcagactctactaccgcagacacgcgcctcagtatcaaaatcaacaaattgccagactactcggtctgccatgattgtcttgtattttaccctgattttttttcaccttacttgcgcttgtgattttcacttcactcaattttggaaaacgcaatgcttcgacccgcgcctctccaacgactggatgatggctcttgcaattgccacgcttggggcgtttggactttttagtggttttgctttgcattacaaatttaagactccatggacacatggctttctttcagattttccagttacacctactccgccgcctcccccggccatcgacgtgcctcaggttccctcaccttctccatctgtctgcagctactttcatctgtaatggccgacctagaatttgacggagtgcaatctgagcaaagggctatacacttccaacgccagtcggaccgcgaacgcaaaaacagagagctgcaaaccatacaaaacacccaccaatgtaaacgcgggatattttgtattgtaaaacaagctaagctccactacgagcttctatctggcaacgaccacgagctccaatacgtggtcgatcagcagcgtcaaacctgtgtattcttaattggagtttcccccattaaagttactcaaaccaagggtgaaaccaagggaaccataaggtgctcatgtcacctgtcagaatgcctttacactctagttaaaaccctatgtggcttacatgattctatcccctttaattaaataaacttactttaaatctgcaatcacttcttcgtccttgtttttgtcgccatccagcagcaccaccttcccctcttcccaactttcatagcatattttccgaaaagaggcgtactttcgccacaccttaaagggaacgtttacttcgctttcaagctctcccacgattttcattgcagatatgaaacgcgccaaagtggaagaaggatttaaccccgtttatccctatggatattctactccgactgacgtggctcctccctttgtagcctctgacggtcttcaagaaaacccacctggggtcttgtccctaaaaatatccaaacctttaacttttaatgcctccaaggctctaagcctggctattggtccaggattaaaaattcaagatggtaaactagtgggggagggacaagcaattcttgcaaacctgccgcttcaaatcaccaacaacacaatttcactacgttttgggaacacacttgccttgaatgacaataatgaactccaaaccacactaaaatcttcatcgccccttaaaatcacagaccagactctgtcccttaacataggggacagccttgcaattaaagatgacaaactagaaagcgctcttcaagcgaccctcccactctccattagcaacaacaccatcagcctcaacgtgggcaccggactcaccataaatggaaacgttttacaagctgttcccttaaatgctctaagtcccctaactatttccaacaataacatcagcctgcgctatggcagttccctgacggtgcttaacaatgaactgcaaagcaacctcacagttcactcccctttaaaactcaactccaacaactcaatttctctcaacactctatctccgtttagaatcgagaatggtttcctcacgctctatttgggaacaaaatctggcttgctagttcaaaacagtggcttaaaagttcaagcgggctacggcctgcaagtaacagacaccaatgctctcacattaagatatctcgctccactgaccattccagactcgggctcagaacaaggcattcttaaagtaaacactggacagggcctaagtgtgaaccaagctggagcgcttgaaacatccctaggaggtggattaaaatatgctgataacaaaataacctttgatacaggaaacggactgacattatctgaaaataaacttgcagtagctgcaggtagtggtctaacttttagagatggtgccttggtagccacgggaaccgcatttacgcaaacactgtggactacggctgatccgtctcccaactgcacaattatacaggaccgcgacacaaaatttactttggcgcttaccattagtgggagccaagtgctggggacggtttccattattggagtaaaaggccccctttcaagtagcataccgtcagctaccgttacagtacaacttaactttgattccaacggagccctattgagctcctcttcacttaaaggttactgggggtatcgccaaggtccctcaattgacccttaccccataattaatgccttaaactttatgccaaactcactggcttatcccccgggacaagaaatccaagcaaaatgtaacatgtacgtttctacttttttacgaggaaatccacaaagaccaatagttttaaacatcacttttaataatcaaaccagcgggttttccattagatttacatggacaaatttaaccacaggagaagcatttgcaatgcccccatgcactttttcctacattgctgaacaacaataaactatgtaaccctcaccgttaacccgcctccgcccttccattttattttataaaccacccgatccaccttttcagcagtaaacaattgcatgtcagtaggggcagtaaaacttttgggagttaaaatccacacaggttcttcacaagctaagcgaaaatcagttacacttataaaaccatcgctaacatcggacaaagacaagcatgagtccaaagcttccggttctggatcagatttttgttcattaacagcgggagaaacagcttctggaggattttccatctccatctccttcatcagttccaccatgtccaccgtggtcatctgggacgagaacgacagttgtcatacacctcataagtcaccggtcgatgacgaacgtacagatctcgaagaatgtcctgtcgccgcctttcggcagcactgggccgaaggcgaaagcgcccatgtttaacaatggccagcaccgcccgcttcatcaggcgcctagttcttttagcgcaacagcgcatgcgcagctcgctaagactggcgcaagaaacacagcacagaaccaccagattgttcatgatcccataagcgtgctgacaccagcccatactaacaaattgtttcactattctagcatgaatgtcatatctgatgttcaagtaaattaaatggcgcccccttatgtaaacacttcccacgtacaacacctcctttggcatctgataattaaccacctcccgataccaaatacatctctgattaatagtcgccccgtacactacccgattaaaccaagttgccaacataatcccccctgccatacactgcaaagaacctggacggctacaatgacagtgcaaagtccacacctcgttgccatggataactgaggaacgccttaagtcaatagtggcacaactaatacaaacatgtaaatagtgtttcaacaagtgccactcgtatgaggtgagtatcatgtcccagggaacgggccactccataaacactgcaaaaccaacacatcctaccatcccccgcacggcactcacatcgtgcatggtgttcatatcacagtccggaagctgaggacaaggaaaagtctcgggagcattttcatagggcggtagtgggtactccttgtaggggttcagtcggcaccggtatctcctcaccttctgggccataacacacaagttgagatctgatttcaaggtactttctgaatgaaaaccaagtgctttcccaacaatgtatccgatgtcttcggtccccgcgtcggtagcgctccttgcagtacacacggaacaaccactcacgcaggcccagaagacagttttccgcggacggtgacaagttaatccccctcagtctcagagccaatatagtttcttccacagtagcataggccaaacccaaccaggaaacacaagctggcacgtcccgttcaacgggaggacaaggaagcagaggcagaggcataggcaaagcaacagaatttttattccaactggtcacgtagcacttcaaacaccaggtcacgtaaatggcagcgatcttgggtttcctgatggaacataacagcaagatcaaacatgagacgattctcaaggtgattaaccacagctggaattaaatcctccacgcgcacatttagaaacaccagcaatacaaaagcccggttttctccgggatctatcatagcagcacagtcatcaattagtcccaagtaattttcccgtttccaatctgttataatttgcagaataatgccctgtaaatccaagccggccatggcgaaaagctcagataatgcactttccacgtgcattcgtaaacacaccctcatcttgtcaatccaaaaagtcttcttcttgagaaacctgtagtaaattaagaatcgccaggttaggctcgatgcctacatcccggagcttcattctcagcatgcactgcaaatgatccagcagatcagaacagcaattagcagccagctcatccccggtttccagttccggagttcccacggcaattatcactcgaaacgtgggacaaatcgaaataacatgagctcccacgtgagcaaaagccgtagggccagtgcaataatcacagaaccagcggaaaaaagattgcagctcatgtttcaaaaagctctgcagatcaaaattcagctcatgcaaataacacagtaaagtttgcggtatagtaaccgaaaaccacacgggtcgacgttcaaacatctcggcttacctaaaaaagaagcacatttttaaaccacagtcgcttcctgaacaggaggaaatatggtgcggcgtaaaaccagacgcgccaccggatctccggcagagccctgataatacagccagctgtggttaaacagcaaaacctttaattcggcaacggttgaggtctccacataatcagcgcccacaaaaatcccatctcgaacttgctcgcgtagggagctaaaatggccagtatagccccatggcacccgaacgctaatctgcaagtatatgagagccaccccattcggcgggatcacaaaatcagtcggagaaaacaacgtatacaccccggactgcaaaagctgttcaggcaaacgcccctgcggtccctctcggtacaccagcaaagcctcgggtaaagcagccatgccaagcgcttaccgtgccaagagcgactcagacgaaaaagtgtactgaggcgctcagagcagcggctatatactctacctgtgacgtcaagaaccgaaagtcaaaagttcacccggcgcgcccgaaaaaacccgcgaaaatccacccaaaaagcccgcgaaaaacacttccgtataaaatttccgggttaccggcgcgtcaccgccgcgcgacacgcccgccccgccccgcgctcctccccgaaacccgccgcgcccacttccgcgttcccaagacaaaggtcgcgtaactccgcccacctcatttgcatgttaactcggtcgccatcttgcggtgttatattgatgatg

SEQ ID NO: 35 - protein, 503 aa, simian adenovirus SV-39
MRRAVAVPSAAMALGPPPSYESVMAAATLQAPLENPYVPPRYLEPTGGRNSIRYSELTPLYDTTRLYLVDNKSADIATLNYQNDHSNFLTSVVQNSDYTPAEASTQTINLDDRSRWGGDLKTILHTNMPNVNEFMFTNSFRAKLMVAHEADKDPVYEWVQLTLPEGNFSEIMTIDLMNNAIIDHYLAVARQQGVKESEIGVKFDTRNFRLGWDPETGLVMPGVYTNEAFHPDVVLLPGCGVDFTYSRLNNLLGIRKRMPFQEGFQILYEDLEGGNIPALLDVPAYEESIANAREAAIRGDNFAAQPQAAPTIKPVLEDSKGRSYNVIANTNNTAYRSWYLAYNYGDPEKGVRAWTLLTTPDVTCGSEQVYWSLPDMYVDPVTFRSTQQVSNYPVVGAELMPIHSKSFYNEQAVYSQLIRQTTALTHVFNRFPENQILVRPPAPTITTVSENVPALTDHGTLPLQNSIRGVQRVTITDARRRTCPYVYKALGIVAPRVLSSRTF

SEQ ID NO: 36 - protein, 917 aa, simian adenovirus SV-39
MATPSMMPQWSYMHIAGQDASEYLSPGLVQFARATETYFSLGNKFRNPTVAPTHDVTTDRSQRLTIRFVPVDKEDTAYSYKTRFTLAVGDNRVLDMASTYFDIRGVIDRGPSFKPYSGTAYNSLAPKGAPNNSQWNATDNGNKPVCFAQAAFIGQSITKDGVQIQNSENQQAAADKTYQPEPQIGVSTWDTNVTSNAAGRVLKATTPMLPCYGSYANPTNPNGGQAKTEGDISLNFFTTTAAADNNPKVVLYSEDVNLQAPDTHLVYKPTVGENVIAAEALLTQQACPNRANYIGFRDNFIGLMYYNSTGNMGVLAGQASQLNAVVDLQDRNTELSYQLMLDALGDRTRYFSMWNQAVDSYDPDVRIIENHGVEDELPNYCFPLPGMGIFNSYKGVKPQNGGNGNWEANGDLSNANEIALGNIFAMEINLHANLWRSFLYSNVALYLPDSYKFTPANITLPANQNTYEYINGRVTSPTLVDTFVNIGARWSPDPMDNVNPFNHHRNAGLRYRSMLLGNGRVVPFHIQVPQKFFAIKNLLLLPGSYTYEWSFRKDVNMILQSTLGNDLRVDGASVRIDSVNLYANFFPMAHNTASTLEAMLRNDTNDQSFNDYLSAANMLYPIPANATNVPISIPSRNWAAFRGWSFTRLKAKETPSLGSGFDPYFVYSGTIPYLDGSFYLNHTFKRLSIMFDSSVSWPGNDRLLTPNEFEIKRIVDGEGYNVAQSNMTKDWFLIQMLSHYNIGYQGFYVPEGYKDRMYSFFRNFQPMSRQVPDPTAAGYQAVPLPRQHNNSGFVGYMGPTMREGQPYPANYPYPLIGATAVPAITQKKFLCDRVMWRIPFSSNFMSMGALTDLGQNMLYANSAHALDMTFEVDPMNEPTLLYMLFEVFDVVRVHQPHRGIIEAVYLRTPFSAGNATT

SEQ ID NO: 37 - protein, 533 aa, simian adenovirus SV-39
MKRAKVEEGFNPVYPYGYSTPTDVAPPFVASDGLQENPPGVLSLKISKPLTFNASKALSLAIGPGLKIQDGKLVGEGQAILANLPLQITNNTISLRFGNTLALNDNNELQTTLKSSSPLKITDQTLSLNIGDSLAIKDDKLESALQATLPLSISNNTISLNVGTGLTINGNVLQAVPLNALSPLTISNNNISLRYGSSLTVLNNELQSNLTVHSPLKLNSNNSISLNTLSPFRIENGFLTLYLGTKSGLLVQNSGLKVQAGYGLQVTDTNALTLRYLAPLTIPDSGSEQGILKVNTGQGLSVNQAGALETSLGGGLKYADNKITFDTGNGLTLSENKLAVAAGSGLTFRDGALVATGTAFTQTLWTTADPSPNCTIIQDRDTKFTLALTISGSQVLGTVSIIGVKGPLSSSIPSATVTVQLNFDSNGALLSSSSLKGYWGYRQGPSIDPYPIINALNFMPNSLAYPPGQEIQAKCNMYVSTFLRGNPQRPIVLNITFNNQTSGFSIRFTWTNLTTGEAFAMPPCTFSYIAEQQ

SEQ ID NO: 38 - DNA, 50 bp, Artificial sequence
  oligomer SV25T
aatttaaatacgtagcgcactagtcgcgctaagcgcggatatcatttaaa

SEQ ID NO: 39 - DNA, 49 bp, Artificial sequence
  oligomer SV25B
tatttaaatgatatccgcgcttaagcgcgactagtgcgctacgtattta

SEQ ID NO: 40 - protein, 9 aa, Artificial sequence
  Synthetic peptide which carries the immunodominant CD8+ T cell epitope for the H-2d haplotype
AMQMLKETI

SEQ ID NO: 41 - DNA, 19 bp, Artificial sequence
  5′ primer for the rabies virus glycoprotein
aagcatttccgcccaacac

SEQ ID NO: 42 - DNA, 22 bp, Artificial sequence
  3′ primer for rabies virus glycoprotein
ggttagtggagcagtaggtaga

SEQ ID NO: 43 - DNA, 25 bp, Artificial sequence
  5′ primer for glutaraldehyde-3-phosphate dehydrogenase (GAPDH)
ggtgaaggtcggtgtgaacggattt

SEQ ID NO: 44 - DNA, 25 bp, Artificial Sequence
  3′ primer for GAPDH
aatgccaaagttgtcatggatgacc

SEQ ID NO: 45 - DNA, 7228 bp, Artificial Sequence
  Modified HIV-1 gag sequence
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcgtcgacagagagatgggtgcgagagcgtcagtattaagcgggggagaattagatcgatgggaaaaaattcggttaaggccagggggaaagaagaagtacaagctaaagcacatcgtatgggcaagcagggagctagaacgattcgcagttaatcctggcctgttagaaacatcagaaggctgtagacaaatactgggacagctacaaccatcccttcagacaggatcagaggagcttcgatcactatacaacacagtagcaaccctctattgtgtgcaccagcggatcgagatcaaggacaccaaggaagctttagacaagatagaggaagagcaaaacaagtccaagaagaaggcccagcaggcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaggacctgaacacgatgttgaacaccgtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagagatctacaagaggtggataatcctgggattgaacaagatcgtgaggatgtatagccctaccagcattctggacataagacaaggaccaaaggaaccctttagagactatgtagaccggttctataaaactctaagagctgagcaagcttcacaggaggtaaaaaattggatgacagaaaccttgttggtccaaaatgcgaacccagattgtaagaccatcctgaaggctctcggcccagcggctacactagaagaaatgatgacagcatgtcagggagtaggaggacccggccataaggcaagagttttgtagggatccactagttctagactcgagggggggcccggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaccccccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcccctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctgctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 46 - protein, 363 aa, Artificial Sequence
  Synthetic Construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 47 - protein, 311 aa, Chimpanzee type adenovirus
NTCQWTYKADGETATEKTYTYGNAPVQGINITKDGIQLGTDTDDQPIYADKTYQPEPQVGDAEWHDITGTDEKYGGRALKPDTKMKPCYGSFAKPTNKEGGQANVKTGTGTTKEYDIDMAFFDNRSAAAAGLAPEIVLYTENVDLETPDTHIVYKAGTDDSSSSINLGQQAMPNRPVYIGFRDNFIGLMYYNSTGNMGVLAGQASQLNAVVDLQDRNTELSYQLLLDSLGDRTRYFSMWNQAVDSYDPDVRIIENHGVEDELPNYCFPLDAVGRTDTYQGIKANGTDQTTWTKDDSVNDANEIGKGNPFAM

SEQ ID NO: 48 - protein, 314 aa, Human adenovirus type 4
NTCQWKDSDSKMHTFGAAAMPGVTGKKIEADGLPIRIDSTSGTDTVIYADKTFQPEPQVGNDSWVDTNGAEEKYGGRALKDTTKMNPCYGSFAKPTNKEGGQANLKDSEPAATTPNYDIDLAFFDSKTIVANYDPDIVMYTENVDLQTPDTHIVYKPGTEDTSSESNLGQQAMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLNGVGLTDTYQGVKVKTDAGSEKWDKDDTTVSNANEIHVGNPFAM

SEQ ID NO: 49 - protein, 318 aa, Human adenovirus type 16
NTCQWKDSDSKMHTFGVAAMPGVTGKKIEADGLPIGIDSTSGTDTVIYADKTFQPEPQVGNASWVDANGTEEKYGGRALKDTTKMKPCYGSFAKPTNKEGGQANLKDSETAATTPNYDIDLAFFDNKNIAANYDPDIVMYTENVDLQTPDTHIVYKPGTEDTSSESNLGQQAMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLNGVGFTDTYQGVKVKTDAVAGTSGTQWDKDDTTVSTANEIHGGNPFAM

SEQ ID NO: 50 - protein, 323 aa, Human adenovirus type 3
NTSQWIVTTNGDNAVTTTTNTFGIASMKGGNITKEGLQIGKDITTTEGEEKPIYADKTYQPEPQVGEESWTDTDGTNEKFGGRALKPATNMKPCYGSFARPTNIKGGQAKNRKVKPTTEGGVETEEPDIDMEFFDGRDAVAGALAPEIVLYTENVNLETPDSHVVYKPETSNNSHANLGQQAMPNRPNYIGFRDNFVGLMYYNSTGNMGVLAGQASQLNAVVDLQDRNTELSYQLLLDSLGDRTRYFSMWNQAVDSYDPDVRIIENHGIEDELPNYCFPLNGIGPGHTYQGIKKVKTDDTNGWEKDANVAPANEITIGNNLAM

SEQ ID NO: 51 - protein, 315 aa, Human adenovirus type 7
NTSQWIVTAGEERAVTTTTNTFGIASMKGDNITKEGLEIGKDITADNKPIYADKTYQPEPQVGEESWTDTDGTNEKFGGRALKPATKMKPCYGSFARPTNIKGGQAKNRKVKPTEGDVETEEPDIDMEFFDGREAADAFSPEIVLYTENVNLETPDSHVVYKPGTSDDNSHANLGQQAMPNRPNYIGFRDNFVGLMYYNSTGNMGVLAGQASQLNAVVDLQDRNTELSYQLLLDSLGDRTRYFSMWNQAVDSYDPDVRIIENHGIEDELPNYCFPLDGIGPAKTYQGIKSKDNGWEKDDNVSKSNEIAIGNNQAM

SEQ ID NO: 52 - protein, 345 aa, Human adenovirus type 2
NSCEWEQTEDSGRAVAEDEEEEDEDEEEEEEEQNARDQATKKTHVYAQAPLSGETLTKSGLQIGSKNAETQAKPVYADPSYQPEPQIGESQWNEADANAAGGRVLKKTTPMKPYGSYARPTNPFGGQSVLVPDEKGVPLPKVDLQFFSNTTSLNDRQGNATKPKVVLYSEDVNMETPDTHLSYKPGKGDENSKAMLGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGIGVTDTYQAIKANGNGSGDNGDTTWTKDETFATRNEIGVGNNFAM

(19) United States
(12) Patent Application Publication
(10) Pub. No.: US 2012/0093853 A1
(43) Pub. Date: Apr. 19, 2012

(54) Simian Adenovirus Vectors and Methods of Use

(75) Inventors: James M. Wilson, Gladwyne, PA (US); Guangping Gao, Westborough, MA (US); Soumitra Roy, Wayne, PA (US)

(73) Assignee: The Trustees of the University of Pennsylvania, Philadelphia, PA (US)

(21) Appl. No.: 13/084,596

(22) Filed: Apr. 12, 2011

Related U.S. Application Data
(63) Continuation of application No. 11/978,477, filed on Oct. 29, 2007, now abandoned.
Application No. 11/978,477 is a continuation of application No. 10/739,096, filed on Dec. 19, 2003, now abandoned.
Application No. 10/739,096 is a continuation-in-part of application No. PCT/US02/15239, filed on May 13, 2002.
Application No. 10/739,096 is a continuation-in-part of application No. PCT/US02/33645, filed on Nov. 20, 2002.
(60) Provisional application No. 60/304,843, filed on Jul. 12, 2001.
(60) Provisional application No. 60/300,131, filed on Jun. 22, 2001.
(60) Provisional application No. 60/366,798, filed on Mar. 22, 2002.
(60) Provisional application No. 60/331,951, filed on Nov. 21, 2001.

(51) Int. Cl. A61K 31/7088; A61K 39/00; A61P 37/04; A61P 31/18; A61P 31/14; A61P 31/16; A61P 31/22; A61P 31/20; A61P 31/04; A61P 31/10; A61P 33/02; A61P 33/10; A61P 33/12; A61P 35/00; C12N 15/861

(52) U.S. Cl. 424/199.1; 435/320.1; 514/44 R

(57) ABSTRACT

A recombinant vector comprises a simian adenovirus capsid and a heterologous gene under the control of regulatory sequences. A cell line which expresses simian adenovirus gene(s) is also disclosed. Methods of using the vectors and cell lines are provided.

### CROSS-REFERENCE TO RELATED APPLICATIONS

This is a continuation of U.S. patent application Ser. No. 11/978,477, filed Oct. 29, 2007 which is a continuation of U.S. patent application Ser. No. 10/739,096, filed Dec. 19, 2003, now abandoned. U.S. patent application Ser. No. 10/739,096 is a continuation-in-part of International Patent Application No. PCT/US02/15239, filed May 13, 2002, which claims the benefit under 35 USC 119(e) of U.S. Patent Application No. 60/304,843, filed Jul. 12, 2001 and now expired, and U.S. Patent Application No. 60/300,131, filed Jun. 22, 2001 and now expired, all of which are incorporated by reference. U.S. patent application Ser. No. 10/739,096 is also a continuation-in-part of International Patent Application No. PCT/US02/33645, filed Nov. 20, 2002, which claims the benefit under 35 USC 119(e) of U.S. Patent Application No. 60/366,798, filed Mar. 22, 2002 and now expired, and U.S. Patent Application No. 60/331,951, filed Nov. 21, 2001 and now expired, all of which are incorporated by reference.

### STATEMENT REGARDING FEDERALLY SPONSORED RESEARCH OR DEVELOPMENT

This invention was made with government support under grant numbers P30 DK 47757-08 and P01 HL59407-02 awarded by the National Institute of Health and grant number AI 49766-01 awarded by the NIAID. The government has certain rights in the invention.

### BACKGROUND OF THE INVENTION

Adenovirus is a double-stranded DNA virus with a genome size of about 36 kilobases (kb), which has been widely used for gene transfer applications due to its ability to achieve highly efficient gene transfer in a variety of target tissues and large transgene capacity. Conventionally, E1 genes of adenovirus are deleted and replaced with a transgene cassette consisting of the promoter of choice, cDNA sequence of the gene of interest and a poly A signal, resulting in a replication defective recombinant virus.

Adenoviruses have a characteristic morphology with an icosahedral capsid consisting of three major proteins, hexon (II), penton base (III) and a knobbed fibre (IV), along with a number of other minor proteins, VI, VIII, IX, Ma and IVa2 [W. C. Russell, J. Gen Virol., 81:2573-2604 (November 2000)]. The virus genome is a linear, double-stranded DNA with a terminal protein attached covalently to the 5′ termini, which have inverted terminal repeats (ITRs). The virus DNA is intimately associated with the highly basic protein VII and a small peptide termed mu. Another protein, V, is packaged with this DNA-protein complex and provides a structural link to the capsid via protein VI. The virus also contains a virus-encoded protease, which is necessary for processing of some of the structural proteins to produce mature infectious virus.

Recombinant adenoviruses have been described for delivery of molecules to host cells. See, U.S. Pat. No. 6,083,716, which describes the genome of two chimpanzee adenoviruses.

What is needed in the art are more effective vectors which avoid the effect of pre-existing immunity to selected adenovirus serotypes in the population and/or which are useful for repeat administration and for titer boosting by second vaccination, if required.

### SUMMARY OF THE INVENTION

The present invention provides simian adenovirus vectors and methods of using same for delivery of heterologous molecules to desired cells, in compositions, and for viral production. Also provided are the isolated nucleic acid sequences and amino acid sequences of six simian adenoviruses, vectors containing these sequences, and cell lines expressing simian adenovirus genes.

The methods of the invention involve delivering one or more selected heterologous gene(s) to a mammalian patient by administering a vector of the invention. Because the various vector constructs are derived from simian rather than from human adenoviruses, the immune system of the non-simian human or veterinary patient is not primed to respond immediately to the vector as a foreign antigen. Use of the compositions of this invention thus permits a more stable expression of the selected transgene when administered to a non-simian patient. Use of the compositions of this invention for vaccination permits presentation of a selected antigen for the elicitation of protective immune responses. Without wishing to be bound by theory, the ability of the adenoviruses of the invention to transduce human dendritic cells is at least partially responsible for the ability of the recombinant constructs of the invention to induce an immune response. The recombinant simian adenoviruses of this invention may also be used for producing heterologous gene products in vitro. Such gene products are themselves useful in a variety for a variety of purposes such as are described herein.

These and other embodiments and advantages of the invention are described in more detail below.

### BRIEF DESCRIPTION OF THE DRAWINGS

FIG. 1 summarizes the genetic organization of the chimpanzee adenovirus C68 genome. In FIG. 1A the genome of the C68 chimpanzee adenovirus is schematically represented by the box at the top. The inverted terminal repeats are shaded black and the early regions are shaded gray. The arrowheads above the box indicate the direction of expression of the early genes. The line below the box represents the division of the genome into 100 map units. The arrows below the line represent the five late gene regions and the proteins encoded in each region. The numbers below the box or arrows indicate the start (promoter or initiation codon) and end (canonical PolyA signal) for each region. * represents the E2A late promoter. FIG. 1B illustrates the PstI clones; FIG. 1C illustrates the BamHI clones. FIG. 1D illustrates the HindIII clones. For parts 1B-1D, the unshaded regions indicate that a fragment was cloned into a plasmid vector, as listed in Table 1, while the shaded regions indicate that the restriction fragment was not cloned. For each section the fragment name, alphabetical with A being the largest fragment, and the fragment size are listed above the box and the fragment end points are listed below the box.

FIG. 2 provides a multiple sequence alignment of hexon proteins. The deduced amino acid sequences of highly similar human adenovirus hexons were compared with the chimpanzee adenovirus using CLUSTAL X. Serotypes and subgroups are indicated on the left margin, followed by the residue number. The numbering refers to the amino acid position with respect to the start of translation. Amino acids are shaded with respect to C68 [SEQ ID NO:47] to highlight sequence similarities (gray) and identities (black). The seven hypervariable regions within loop domains DE1 and FG1 are labeled along the bottom and correspond to the following Ad2 sequences in the alignment: HVR1, 137-188; HVR2, 194-204; HVR3, 222-229; HVR4, 258-271; HVR5, 278-294; HVR6, 316-327; and HVR7, 433-465. The GenBank accession numbers for the sequences shown are as follow: AAD03657 (Ad4, SEQ ID NO:48), S37216 (Ad16, SEQ ID NO:49), S39298 (Ad3, SEQ ID NO:50), AAD03663 (Ad7, SEQ ID NO:51), and NP040525 (Ad2, SEQ ID NO:52).

FIG. 3 provides an alignment of the amino acid sequences of the L1 and a portion of the L2 loops of the capsid protein hexon of the chimpanzee adenovirus C1 [SEQ ID NO:13], chimpanzee adenovirus C68 (Pan-9) [SEQ ID NO:14], and the novel Pan5 [SEQ ID NO:15], Pan6 [SEQ ID NO: 16] and Pan7 [SEQ ID NO: 17] chimpanzee adenovirus sequences of the invention. The intervening conserved region is part of the pedestal domain conserved between adenovirus serotypes.

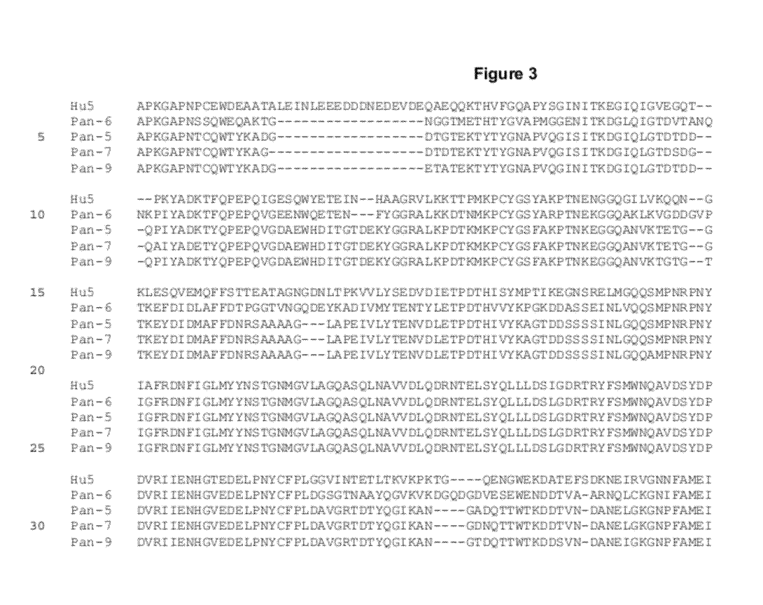

FIG. 4 provides an alignment of the amino acid sequences of the fiber knob domains of chimpanzee C68 (Pan-9) [SEQ ID NO:18], Pan-6 [SEQ ID NO:19], Pan-7 [SEQ ID NO:20], and Pan-5 [SEQ ID NO:21] and the human adenoviruses serotypes 2 [SEQ ID NO:22] and 5 [SEQ ID NO:23].

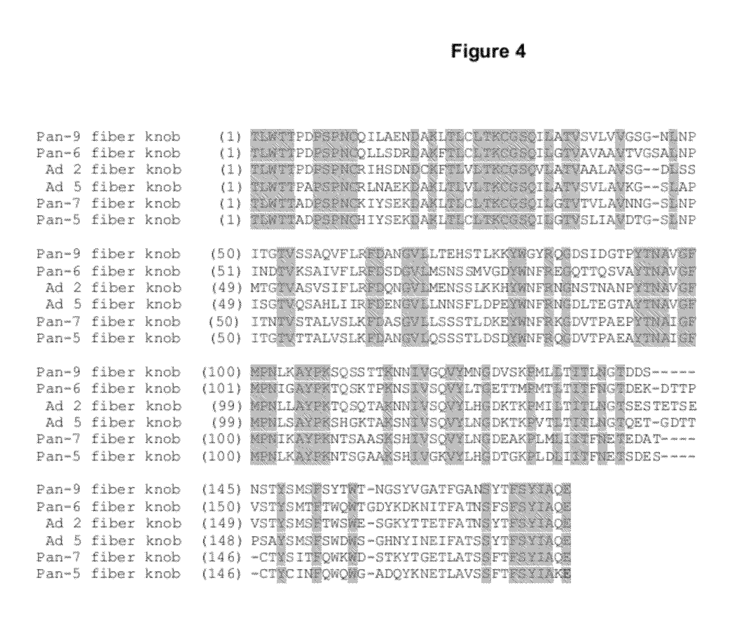

### DETAILED DESCRIPTION OF THE INVENTION

The present invention provides novel adenovirus-based compositions for use in delivering a heterologous molecule for therapeutic or vaccine purposes. Such therapeutic or vaccine compositions contain the adenoviral vectors carrying an inserted heterologous molecule.

The invention further provides novel nucleic acid and amino acid sequences from Ad Pan5 [SEQ ID NO:1-4, 15 and 21], Ad Pan6 [SEQ ID NO: 5-8, 16, 19], and Ad serotype Pan7 [SEQ ID NO: 9-12, 17, 20], which were originally isolated from chimpanzee lymph nodes. In several instances throughout the specification, these adenoviruses are alternatively termed herein C5, C6 and C7, respectively. Also provided are sequences from adenovirus SV1 [SEQ ID NO: 24-28], which was originally isolated from the kidney cells of cynomolgus monkey. The invention also provides sequences of adenoviruses SV-25 [SEQ ID NO:29-33] and SV-39 [SEQ ID NO: 34-37], which were originally isolated from rhesus monkey kidney cells. Also, the invention provides packaging cell lines to produce vectors based upon these sequences for use in the in vitro production of recombinant proteins or fragments or other reagents. In addition, novel sequences of the invention are useful in providing the essential helper functions required for production of recombinant adeno-associated viral (AAV) vectors. Thus, the invention provides helper constructs, methods and cell lines which use these sequences in such production methods.

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 95 to 99% of the aligned sequences.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, or a fragment thereof which is at least 8 amino acids, or more desirably, at least 15 amino acids in length. Examples of suitable fragments are described herein.

The term “percent sequence identity” or “identical” in the context of nucleic acid sequences refers to the residues in the two sequences that are the same when aligned for maximum correspondence. The length of sequence identity comparison may be over the full-length of the genome (e.g., about 36 kbp), the full-length of an open reading frame of a gene, protein, subunit, or enzyme [see, e.g., the tables providing the adenoviral coding regions], or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length, and may be up to about 700 amino acids. Examples of suitable fragments are described herein.

Identity is readily determined using such algorithms and computer programs as are defined herein at default settings. Preferably, such identity is over the full length of the protein, enzyme, subunit, or over a fragment of at least about 8 amino acids in length. However, identity may be based upon shorter regions, where suited to the use to which the identical gene product is being put.

As described herein, alignments are performed using any of a variety of publicly or commercially available Multiple Sequence Alignment Programs, such as “Clustal W”, accessible through Web Servers on the internet. Alternatively, Vector NTI utilities are also used. There are also a number of algorithms known in the art that can be used to measure nucleotide sequence identity, including those contained in the programs described above. As another example, polynucleotide sequences can be compared using Fasta, a program in GCG Version 6.1. Fasta provides alignments and percent sequence identity of the regions of the best overlap between the query and search sequences. For instance, percent sequence identity between nucleic acid sequences can be determined using Fasta with its default parameters (a word size of 6 and the NOPAM factor for the scoring matrix) as provided in GCG Version 6.1, herein incorporated by reference. Similarly programs are available for performing amino acid alignments. Generally, these programs are used at default settings, although one of skill in the art can alter these settings as needed. Alternatively, one of skill in the art can utilize another algorithm or computer program that provides at least the level of identity or alignment as that provided by the referenced algorithms and programs.

As used throughout this specification and the claims, the term “comprise” and its variants including, “comprises”, “comprising”, among other variants, is inclusive of other components, elements, integers, steps and the like. The term “consists of” or “consisting of” are exclusive of other components, elements, integers, steps and the like.

### I. The Simian Adenovirus Sequences

A variety of sources of chimpanzee adenovirus sequences are available from the American Type Culture Collection, 10801 University Boulevard, Manassas, Va. 20110-2209, and other sources. Desirable chimpanzee strains Pan 5 [ATCC VR-591], Pan 6 [ATCC VR-592], and Pan 7 [ATCC VR-593]. Particularly desirable chimpanzee adenovirus strains, are chimpanzee adenovirus strain Bertha or C1 [ATCC Accession No. VR-20] and chimpanzee adenovirus, strain Pan 9 or CV68 [ATCC VR-594]. For convenience, the virus CV68 is referred to throughout this specification as “C68”. The viruses were originally isolated from feces [C1, Rowe et al, Proc. Soc. Exp. Med., 91:260 (1956)] or mesenteric lymph node [C68, Basnight et al, Am. J. Epidemiol., 94:166 (1971)] of infected chimpanzees. The sequences of these strains, and the location of the adenovirus genes E1a, E1b, E2a, E2b, E3, E4, L1, L2, L3, L4 and L5 are provided in U.S. Pat. No. 6,083,716, which is incorporated by reference herein. Optionally, non-chimpanzee simian adenoviral sequences may be used in preparing the recombinant vectors of the invention. Such non-chimpanzee adenovirus include those obtained from baboon adenovirus strains [e.g., ATCC VR-275], adenovirus strains isolated from rhesus monkeys [e.g., ATCC VR-209, ATCC VR-275, ATCC VR-353, ATCC VR-355], and adenovirus strains isolated from African green monkeys [e.g., ATCC VR-541; ATCC VR-941; ATCC VR-942; ATCC VR-943].

In one embodiment, the recombinant chimpanzee (or other simian) adenoviruses described herein may contain adenoviral sequences derived from one, more than one simian adenoviral strain. These sequences may be obtained from natural sources, produced recombinantly, synthetically, or by other genetic engineering or chemical methods.

The recombinant simian adenoviruses useful in this invention are viral particles that are composed of recombinant simian adenoviruses sequences carrying a heterologous molecule and/or simian adenovirus capsid proteins. These simian adenoviruses, and particularly the chimpanzee C68 and C1 sequences, are also useful in forming hybrid vectors with other simian and non-simian adenoviruses, and in forming pseudotyped recombinant viruses, i.e., recombinant viruses with an adenoviral vector carrying a heterologous molecule which is packaged in a heterologous capsid protein of simian origin.

In certain embodiments, the invention provides nucleic acid sequences and amino acid sequences of Pan8, Pan6, Pan7, SV1, SV25 and SV39, which are isolated from the other viral material with which they are associated in nature.

A. Nucleic Acid Sequences

The Pan8 nucleic acid sequences of the invention include nucleotides 1 to 36462 of SEQ ID NO:1. The Pan6 nucleic acid sequences of the invention include nucleotides 1 to 36604 of SEQ ID NO: 5. The Pan7 nucleic acid sequences of the invention include nucleotides 1 to 36535 of SEQ ID NO: 9. The SV1 nucleic acid sequences of the invention include nucleotides 1 to 34264 of SEQ ID NO: 24. The SV25 nucleic acid sequences of the invention include nucleotides 1 to 31044 of SEQ ID NO: 29. The SV39 nucleic acid sequences of the invention include nucleotides 1 to 34115 of SEQ ID NO: 34. See, Sequence Listing, which is incorporated by reference herein.

The nucleic acid sequences of the invention further encompass the strand which is complementary to the sequences of SEQ ID NO: 5, 9, 24, 29 and 34, as well as the RNA and cDNA sequences corresponding to the sequences of these sequences figures and their complementary strands. Further included in this invention are nucleic acid sequences which are greater than 95 to 98%, and more preferably about 99 to 99.9% homologous or identical to the Sequence Listing. Also included in the nucleic acid sequences of the invention are natural variants and engineered modifications of the sequences provided in SEQ ID NO: 5, 9, 24, 29 and 34 and their complementary strands. Such modifications include, for example, labels that are known in the art, methylation, and substitution of one or more of the naturally occurring nucleotides with a degenerate nucleotide.

The invention further encompasses fragments of the sequences of Pan8, Pan6, Pan7, SV1, SV25 and SV39, their complementary strand, cDNA and RNA complementary thereto. Suitable fragments are at least 15 nucleotides in length, and encompass functional fragments, i.e., fragments which are of biological interest. For example, a functional fragment can express a desired adenoviral product or may be useful in production of recombinant viral vectors. Such fragments include the gene sequences and fragments listed in the tables below.

The following tables provide the transcript regions and open reading frames in the simian adenovirus sequences of the invention. For certain genes, the transcripts and open reading frames (ORFs) are located on the strand complementary to that presented in SEQ ID NO: 5, 9, 24, 29 and 34. See, e.g., E2b, E4 and E2a. The calculated molecular weights of the encoded proteins are also shown. Note that the E1a open reading frame Pan8 [nt 576-1436 of SEQ ID NO:1], Pan6 [nt 576 to 1437 of SEQ ID NO: 5] and Pan7 [nt 576 to 1437 of SEQ ID NO: 9] contain internal splice sites. These splice sites are noted in the following tables.

Ad Pan-5 [SEQ ID NO: 1]

| | Start | End | M.W. |
|---|---|---|---|
| Regions | (nt) | (nt) | (Daltons) |

| ITR | | 1 | 120 | — |
|---|---|---|---|---|
| E1a | Transcript | 478 | | — |
| | 13S | 576-664, 1233-1436 | | 28120 |
| | 12S | 576-1046, 1233-1436 | | 24389 |
| | 9S | 576-644, 1233-1436 | | 9962 |
| | Transcript | | 1516 | — |
| E1b | Transcript | 1552 | | — |
| | Small T | 1599 | 2171 | 22317 |
| | Large T | 1904 | 3412 | 55595 |
| | IX | 3492 | 3920 | 14427 |
| | Transcript | | 3959 | — |
| E2b | Transcript | 10349 | | — |
| | PTP | 10349 | 8451 | 72930 |
| | Polymerase | 8448 | 5083 | 127237 |
| | IVa2 | 5604 | 3980 | 50466 |
| | Transcript | | 3960 | |
| 28.1 kD | | 5155 | 5979 | 28141 |
| Agnoprotein | | 7864 | 8580 | 25755 |
| L1 | Transcript | 10849 | | — |
| | 52/55D | 10851 | 12025 | |
| | IIIa | 12050 | 13819 | 65669 |
| | Transcript | | 13832 | — |
| | Transcript | 13894 | | — |
| L2 | Penton | 13898 | 15490 | 59292 |
| | VII | 15494 | 16078 | 21478 |
| | V | 16123 | 17166 | 39568 |
| | Mu | 17189 | 17422 | 8524 |
| | Transcript | | 17442 | — |
| | Transcript | 17488 | | — |
| L3 | VI | 17491 | 18222 | 26192 |
| | Hexon | 18315 | 21116 | 104874 |
| | Endoprotease | 20989 | 21783 | 28304 |
| | Transcript | | 21811 | — |
| E2a | Transcript | 26782 | | — |
| | DBP | 23386 | 21845 | 57358 |
| | Transcript | | 21788 | — |
| L4 | Transcript | 23406 | | — |
| | 100 kD | 23412 | 25805 | 88223 |
| | 33 kD homolog | 25525 | 26356 | 24538 |
| | VIII | 26428 | 27111 | 24768 |
| | Transcript | | 27421 | — |
| E3 | Transcript | 26788 | | — |
| | Orf #1 | 27112 | 27432 | 12098 |
| | Orf #2 | 27386 | 28012 | 23040 |
| | Orf #3 | 27994 | 28527 | 19525 |
| | Orf #4 | 28557 | 29156 | 22567 |
| | Orf #5 | 29169 | 29783 | 22267 |
| | Orf #6 | 29798 | 30673 | 31458 |
| | Orf #7 | 30681 | 30956 | 10477 |
| | Orf #8 | 30962 | 31396 | 16523 |
| | Orf #9 | 31389 | 31796 | 15236 |
| | Transcript | | 31837 | — |
| L5 | Transcript | 32032 | | — |
| | Fiber | 32035 | 33372 | 47670 |
| | Transcript | | 33443 | — |
| E4 | Transcript | 36135 | | — |
| | Orf 7 | 33710 | 33462 | 9191 |
| | Orf 6 | 34615 | 33710 | 35005 |
| | Orf 4 | 34886 | 34521 | 13878 |
| | Orf 3 | 35249 | 34896 | 13641 |
| | Orf 2 | 35635 | 35246 | 14584 |
| | Orf 1 | 36050 | 35676 | 13772 |
| | Transcript | | 33437 | — |
| ITR | | 36343 | 36462 | — |

Ad Pan-6 [SEQ ID NO: 5]

| | | Start | End | M.W. |
|---|---|---|---|---|
| Regions | | (nt) | (nt) | (Daltons) |
| ITR | | 1 | 123 | — |
| E1a | Transcript | 478 | | — |

| | | |
|---|---|---|
| 13S | 576-1143, 1229-1437 | 28291 |
| 12S | 576-1050, 1229-1437 | 24634 |
| 9S | 576-645, 1229-1437 | 10102 |

| | Transcript | | 1516 | — |
|---|---|---|---|---|
| E1b | Transcript | 1553 | | — |
| | Small T | 1600 | 2172 | 22315 |
| | LargeT | 1905 | 3413 | 55594 |
| | IX | 3498 | 3926 | 14427 |
| | Transcript | | 3965 | — |
| E2b | Transcript | 10341 | | — |
| | PTP | 10340 | 8451 | 72570 |
| | Polymerase | 8445 | 5089 | 126907 |
| | IVa2 | 5610 | 3986 | 50452 |
| | Transcript | | 3966 | — |
| L1 | Transcript | 10838 | | — |
| | 52/55 kD | 10840 | 12012 | 44205 |
| | IIIa | 12036 | 13799 | 65460 |
| | Transcript | | 13812 | — |
| 28.1 kd | | 5161 | 5985 | 28012 |
| Agnoprotein | | 7870 | 8580 | 25382 |
| L2 | Transcript | 13874 | | — |
| | Penton | 13878 | 15467 | 59314 |
| | VII | 15471 | 16055 | 21508 |
| | V | 16100 | 17137 | 39388 |
| | Mu | 17160 | 17393 | 8506 |
| | Transcript | | 17415 | — |
| L3 | Transcript | 17466 | | — |
| | VI | 17469 | 18188 | 25860 |
| | Hexon | 18284 | 21112 | 106132 |
| | Endoprotease | 21134 | 21754 | 23445 |
| | Transcript | | 21803 | — |
| E2a | Transcript | 26780 | | — |
| | DBP | 23375 | 21837 | 57299 |
| | Transcript | | 21780 | — |
| L4 | Transcript | 23398 | | — |
| | 100 kD | 23404 | 25806 | 88577 |
| | 33 kD homolog | 25523 | 26357 | 24609 |
| | VIII | 26426 | 27109 | 24749 |
| | Transcript | | 27419 | — |
| E3 | Transcript | 26786 | | — |
| | Orf #1 | 27110 | 27430 | 12098 |
| | Orf #2 | 27384 | 28007 | 22880 |
| | Orf #3 | 27989 | 28519 | 19460 |
| | Orf #4 | 28553 | 29236 | 25403 |
| | Orf #5 | 29249 | 29860 | 22350 |
| | Orf #6 | 29875 | 30741 | 31028 |
| | Orf #7 | 30749 | 31024 | 10469 |
| | Orf #8 | 31030 | 31464 | 16540 |
| | Orf #9 | 31457 | 31864 | 15264 |
| | Transcript | | 31907 | — |
| L5 | Transcript | 32159 | | |
| | Fiber | 32162 | 33493 | 47364 |
| | Transcript | | 33574 | — |
| E4 | Transcript | 36276 | | — |
| | Orf 7 | 33841 | 33593 | 9177 |
| | Orf 6 | 34746 | 33841 | 35094 |
| | Orf 4 | 35017 | 34652 | 13937 |
| | Orf 3 | 35380 | 35027 | 13627 |
| | Orf 2 | 35766 | 35377 | 14727 |
| | Orf 1 | 36181 | 35807 | 13739 |
| | Transcript | | 33558 | — |
| ITR | | 36482 | 36604 | — |

Ad Pan-7 [SEQ ID NO: 9]

| Regions | | Start (nt) | End (nt) | M.W. (Daltons) |
|---|---|---|---|---|
| ITR | | 1 | 132 | — |
| E1a | Transcript | 478 | | — |
| | 13S | 576-1143, 1229-1437 | | 28218 |
| | 12S | 576-1050, 1229-1437 | | 24561 |
| | 9S | 576-645, | | 10102 |
| | | 1229-1437 | | |
| | Transcript | | 1516 | — |
| E1b | Transcript | 1553 | | — |
| | Small T | 1600 | 2178 | 22559 |
| | LargeT | 1905 | 3419 | 55698 |
| | IVa2 | 3992 | 5616 | 50210 |
| | Transcript | | 3971 | — |
| E2b | Transcript | 10341 | | — |
| | PTP | 10340 | 8457 | 72297 |
| | Polymerase | 8451 | 5095 | 126994 |
| | IX | 3504 | 3932 | 14441 |
| | Transcript | | 3972 | — |
| 28.1 kD | | 5167 | 5991 | 28028 |
| Agnoprotein | | 7876 | 8586 | 25424 |
| L1 | Transcript | 10834 | | |
| | 52/55 kD | 10836 | 12011 | 44302 |
| | IIIa | 12035 | 13795 | 65339 |
| | Transcript | | 13808 | — |
| L2 | Transcript | 13870 | | — |
| | Penton | 13874 | 15469 | 59494 |
| | VII | 15473 | 16057 | 21339 |
| | V | 16102 | 17139 | 39414 |
| | Mu | 17167 | 17400 | 8506 |
| | Transcript | | 17420 | — |
| L3 | Transcript | 17467 | | — |
| | VI | 17470 | 18198 | 26105 |
| | Hexon | 18288 | 21086 | 104763 |
| | Endoprotease | 21106 | 21732 | 23620 |
| | Transcript | | 21781 | — |
| E2a | Transcript | 26764 | | — |
| | DBP | 23353 | 21815 | 57199 |
| | Transcript | | 21755 | — |
| L4 | Transcript | 23370 | | — |
| | 100 kD | 23376 | 25781 | 88520 |
| | 33 kD | 25489 | 26338 | 25155 |
| | homolog | | | |
| | VIII | 26410 | 27093 | 24749 |
| | Transcript | | 27403 | — |
| E3 | Transcript | 26770 | | — |
| | Orf #1 | 27094 | 27414 | 12056 |
| | Orf #2 | 27368 | 27988 | 22667 |
| | Orf #3 | 27970 | 28500 | 19462 |
| | Orf #4 | 28530 | 29150 | 22999 |
| | Orf #5 | 29163 | 29777 | 22224 |
| | Orf #6 | 29792 | 30679 | 32153 |
| | Orf #7 | 30687 | 30962 | 10511 |
| | Orf #8 | 30968 | 31399 | 16388 |
| | Orf #9 | 31392 | 31799 | 15205 |
| | Transcript | | 31842 | — |
| L5 | Transcript | 32091 | | — |
| | Fiber | 32094 | 33425 | 47344 |
| | Transcript | | 33517 | — |
| E4 | Transcript | 36208 | | — |
| | Orf 7 | 33784 | 33536 | 9191 |
| | Orf 6 | 34689 | 33784 | 35063 |
| | Orf 4 | 34960 | 34595 | 13879 |
| | Orf 3 | 35323 | 34970 | 13641 |
| | Orf 2 | 35709 | 35320 | 14644 |
| | Orf 1 | 36123 | 35749 | 13746 |
| | Transcript | | 33501 | — |
| ITR | | 36404 | 36535 | — |

| | Ad SV-1 | | Ad SV-25 | | Ad SV-39 | |
|---|---|---|---|---|---|---|
| | [SEQ ID NO: 24] | | [SEQ ID NO: 29] | | [SEQ ID NO: 34] | |
| Region | Start | End | Start | End | Start | End |
| ITR | 1 | 106 | 1 | 133 | 1 | 150 |
| E1a | 352 | 1120 | — | — | 404 | 1409 |
| E1b | 1301 | 2891 | 359 | 2273 | 1518 | 3877 |
| E2b | 9257 | 2882 | 9087 | 2754 | 10143 | 3868 |
| E2a | 24415 | 20281 | 24034 | 20086 | 25381 | 21228 |
| E3 | 24974 | 27886 | 24791 | 25792 | 25790 | 29335 |
| E4 | 33498 | 30881 | 30696 | 28163 | 33896 | 31157 |
| ITR | 34145 | 34264 | 30912 | 31044 | 33966 | 34115 |
| ITR | 1 | 106 | 1 | 133 | 1 | 150 |
| L1 | 9513 | 12376 | 9343 | 12206 | 10416 | 13383 |
| L2 | 12453 | 15858 | 12283 | 15696 | 13444 | 16877 |
| L3 | 15910 | 20270 | 15748 | 20080 | 17783 | 21192 |
| L4 | 21715 | 25603 | 21526 | 25420 | 22659 | 26427 |
| L5 | 28059 | 30899 | 25320 | 28172 | 29513 | 31170 |
| ITR | 34145 | 34264 | 30912 | 31044 | 33966 | 34115 |

| | | Ad SV-1, SEQ ID NO: 24 | | |
|---|---|---|---|---|
| | Protein | Start | End | M.W. |
| ITR | | 1 | 106 | — |
| E1a | 13S | 459 | 953 | 18039 |
| | 12S | | | |
| E1b | Small T | | | |
| | LargeT | 1301 | 2413 | 42293 |
| | IX | 2391 | 2885 | 16882 |
| E2b | IVa2 | 4354 | 2924 | 54087 |
| | Polymerase | 6750 | 4027 | 102883 |
| | PTP | 9257 | 7371 | 72413 |
| | Agno-protein | 6850 | 7455 | 20984 |
| L1 | 52/55 kD | 9515 | 10642 | 42675 |
| | IIIa | 10663 | 12372 | 636568 |
| L2 | Penton | 12454 | 13965 | 56725 |
| | VII | 13968 | 14531 | 20397 |
| | V | 14588 | 15625 | 39374 |
| | Mu | 15645 | 15857 | 7568 |
| L3 | VI | 15911 | 16753 | 30418 |
| | Hexon | 16841 | 19636 | 104494 |
| | Endoprotease | 19645 | 20262 | 23407 |
| 2a | DBP | 21700 | 20312 | 52107 |
| L4 | 100 kD | 21721 | 24009 | 85508 |
| | VIII | 24591 | 25292 | 25390 |
| E3 | Orf #1 | 25292 | 25609 | 11950 |
| | Orf #2 | 25563 | 26081 | 18940 |
| | Orf #3 | 26084 | 26893 | 30452 |
| | Orf #4 | 26908 | 27180 | 10232 |
| | Orf #5 | 27177 | 17512 | 12640 |
| | Orf #6 | 27505 | 27873 | 13639 |
| L5 | Fiber #2 | 28059 | 29150 | 39472 |
| | Fiber #1 | 29183 | 30867 | 61128 |
| E4 | Orf 7 | 31098 | 30892 | 7837 |
| | Orf 6 | 31982 | 31122 | 33921 |
| | Orf 4 | 32277 | 31915 | 14338 |
| | Orf 3 | 32629 | 32279 | 13386 |
| | Orf 2 | 33018 | 32626 | 14753 |
| | Orf 1 | 33423 | 33043 | 14301 |
| ITR | | 34145 | 34264 | |

| | | Ad SV-25, SEQ ID NO: 29 | | | Ad SV-39, SEQ ID NO: 34 | | |
|---|---|---|---|---|---|---|---|
| | protein | Start | End | M.W. | Start | End | M.W. |
| ITR | | 1 | 133 | — | 1 | 150 | — |
| E1a | 13S | | | | 492 | 1355 | 28585 |
| | 12S | | | | 492 | 1355 | 25003 |
| E1b | Small T | 478 | 1030 | 20274 | 1518 | 2075 | 21652 |
| | Large T | 829 | 2244 | 52310 | 1823 | 3349 | 55534 |
| | IX | 2306 | 2716 | 13854 | 3434 | 3844 | 14075 |
| E2b | IVa2 | 4208 | 2755 | 54675 | 3912 | 5141 | 46164 |
| | Polymerase | 6581 | 3858 | 102839 | 7753 | 5033 | 103988 |
| | PTP | 9087 | 7207 | 71326 | 10143 | 8335 | 69274 |
| | Agno-protein | 6681 | 7139 | 16025 | — | — | — |
| L1 | 52/55 kD | 9345 | 10472 | 42703 | 10418 | 11608 | 44232 |
| | IIIa | 10493 | 12202 | 63598 | 11574 | 13364 | 66078 |
| L2 | Penton | 12284 | 13801 | 56949 | 13448 | 14959 | 56292 |
| | VII | 13806 | 14369 | 20369 | 14960 | 15517 | 20374 |
| | V | 14426 | 15463 | 39289 | 15567 | 16628 | 39676 |
| | Mu | 15483 | 15695 | 7598 | 16650 | 16871 | 7497 |
| L3 | VI | 15749 | 16591 | 30347 | 16925 | 17695 | 28043 |
| | Hexon | 16681 | 19446 | 104035 | 17785 | 20538 | 102579 |
| | Endoprotease | 19455 | 20072 | 23338 | 20573 | 21181 | 22716 |
| 2a | DBP | 21511 | 20123 | 52189 | 22631 | 21231 | 53160 |
| L4 | 100 kD | 21532 | 23829 | 85970 | 22659 | 25355 | 100362 |
| | VIII | 24408 | 25109 | 25347 | 25410 | 26108 | 25229 |
| E3 | Orf #1 | 25109 | 25426 | 11890 | 26375 | 27484 | 42257 |
| | Orf #2 | | | | 27580 | 28357 | 29785 |
| | Orf #3 | | | | 28370 | 28645 | 10514 |
| | Orf #4 | | | | 28863 | 29333 | 18835 |
| | Orf #5 | | | | | | |
| | Orf #6 | | | | | | |
| L5 | Fiber #2 | 25380 | 26423 | 37529 | | | |
| | Fiber #1 | 26457 | 28136 | 60707 | 29515 | 31116 | 56382 |
| E4 | Orf 7 | | | | 31441 | 31118 | 11856 |
| | Orf 6 | 29255 | 28395 | 33905 | 32292 | 31438 | 33437 |
| | Orf 4 | 29550 | 29188 | 14399 | 32587 | 32222 | 13997 |
| | Orf 3 | 29902 | 29552 | 13284 | 32954 | 32607 | 13353 |
| | Orf 2 | 30291 | 29899 | 14853 | 33348 | 32959 | 14821 |
| | Orf 1 | 30316 | 30696 | 14301 | 33764 | 33378 | 14235 |
| ITR | | 30912 | 31044 | | 33966 | 34115 | |

The simian adenoviruses described herein are useful as therapeutic agents and in construction of a variety of vector systems and host cells. As used herein, a vector includes any suitable nucleic acid molecule including, naked DNA, a plasmid, a virus, a cosmid, or an episome. These sequences and products may be used alone or in combination with other adenoviral sequences or fragments, or in combination with elements from other adenoviral or non-adenoviral sequences. The adenoviral sequences of the invention are also useful as antisense delivery vectors, gene therapy vectors, or vaccine vectors. Thus, the invention further provides nucleic acid molecules, gene delivery vectors, and host cells that contain the Ad sequences of the invention.

For example, the invention encompasses a nucleic acid molecule containing simian Ad ITR sequences of the invention. In another example, the invention provides a nucleic acid molecule containing simian Ad sequences of the invention encoding a desired Ad gene product. Still other nucleic acid molecule constructed using the sequences of the invention will be readily apparent to one of skill in the art, in view of the information provided herein.

In one embodiment, the simian Ad gene regions identified herein may be used in a variety of vectors for delivery of a heterologous molecule to a cell. For example, vectors are generated for expression of an adenoviral capsid protein (or fragment thereof) for purposes of generating a viral vector in a packaging host cell. Such vectors may be designed for expression in trans. Alternatively, such vectors are designed to provide cells which stably contain sequences which express desired adenoviral functions, e.g., one or more of E1a, E1b, the terminal repeat sequences, E2a, E2b, E4, E4ORF6 region.

In addition, the adenoviral gene sequences and fragments thereof are useful for providing the helper functions necessary for production of helper-dependent viruses (e.g., adenoviral vectors deleted of essential functions or adeno-associated viruses (AAV)). For such production methods, the simian adenoviral sequences of the invention are utilized in such a method in a manner similar to those described for the human Ad. However, due to the differences in sequences between the simian adenoviral sequences of the invention and those of human Ad, the use of the sequences of the invention essentially eliminate the possibility of homologous recombination with helper functions in a host cell carrying human Ad E1 functions, e.g., 293 cells, which may produce infectious adenoviral contaminants during rAAV production.

Methods of producing rAAV using adenoviral helper functions have been described at length in the literature with human adenoviral serotypes. See, e.g., U.S. Pat. No. 6,258,595, U.S. Pat. No. 6,083,716, and the references cited therein. See, also, U.S. Pat. No. 5,871,982; WO 99/14354; WO 99/15685; WO 99/47691. These methods may also be used in production of non-human serotype AAV, including non-human primate AAV serotypes. The simian adenoviral gene sequences of the invention which provide the necessary helper functions (e.g., E1a, E1b, E2a and/or E4ORF6) can be particularly useful in providing the necessary adenoviral function while minimizing or eliminating the possibility of recombination with any other adenoviruses present in the rAAV-packaging cell which are typically of human origin. Thus, selected genes or open reading frames of the adenoviral sequences of the invention may be utilized in these rAAV production methods.

Alternatively, recombinant adenoviral simian vectors of the invention may be utilized in these methods. Such recombinant adenoviral simian vectors may include, e.g., a hybrid chimp Ad/AAV in which chimp Ad sequences flank a rAAV expression cassette composed of, e.g., AAV 3′ and/or 5′ ITRs and a transgene under the control of regulatory sequences which control its expression. One of skill in the art will recognize that still other simian adenoviral vectors and/or gene sequences of the invention will be useful for production of rAAV and other viruses dependent upon adenoviral helper.

In still another embodiment, nucleic acid molecules are designed for delivery and expression of selected adenoviral gene products in a host cell to achieve a desired physiologic effect. For example, a nucleic acid molecule containing sequences encoding an adenovirus E1a protein of the invention may be delivered to a subject for use as a cancer therapeutic. Optionally, such a molecule is formulated in a lipid-based carrier and preferentially targets cancer cells. Such a formulation may be combined with other cancer therapeutics (e.g., cisplatin, taxol, or the like). Still other uses for the adenoviral sequences provided herein will be readily apparent to one of skill in the art.

In addition, one of skill in the art will readily understand that the Ad sequences of the invention can be readily adapted for use for a variety of viral and non-viral vector systems for in vitro, ex vivo or in vivo delivery of therapeutic and immunogenic molecules. For example, the Pan8, Pan6, Pan7, SV1, SV25 and/or SV39 simian Ad genomes of the invention can be utilized in a variety of rAd and non-rAd vector systems. Such vectors systems may include, e.g., plasmids, lentiviruses, retroviruses, poxviruses, vaccinia viruses, and adeno-associated viral systems, among others. Selection of these vector systems is not a limitation of the present invention.

The invention further provides molecules useful for production of the simian and simian-derived proteins of the invention. Such molecules which carry polynucleotides including the simian Ad DNA sequences of the invention can be in the form of naked DNA, a plasmid, a virus or any other genetic element.

B. Simian Adenoviral Proteins of the Invention

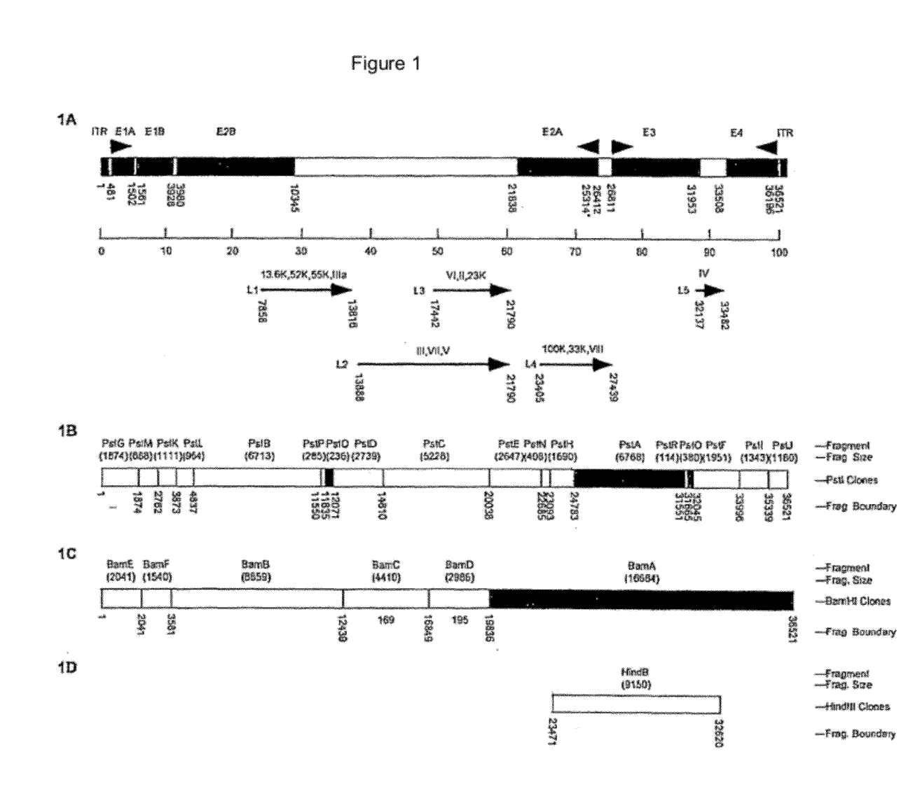

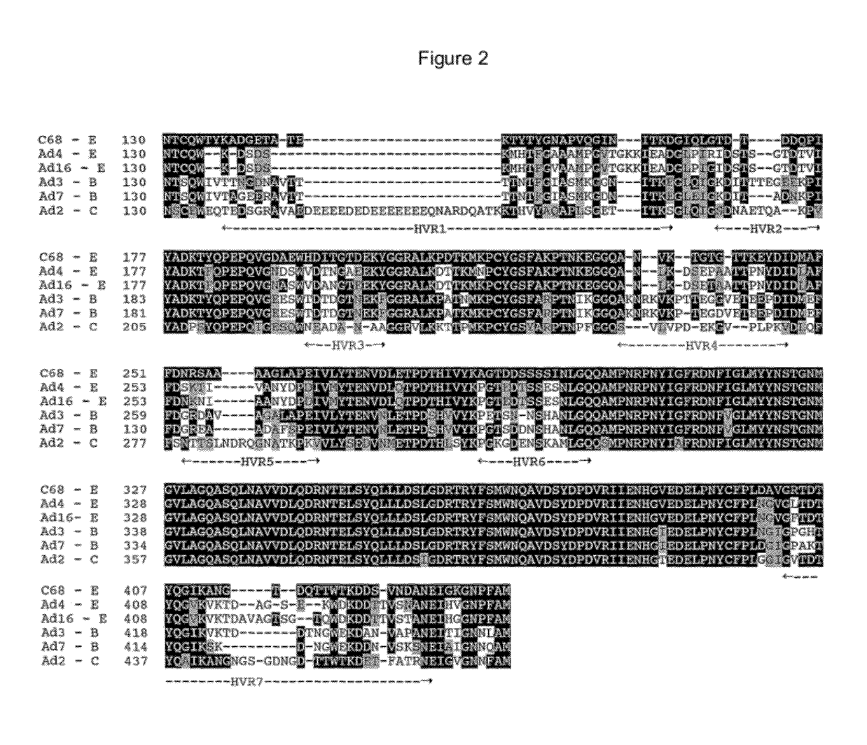

The invention further provides gene products of the above adenoviruses, such as proteins, enzymes, and fragments thereof, which are encoded by the adenoviral nucleic acids of the invention. The invention further encompasses Pan8, Pan6 and Pan7, SV1, SV25 and SV39 proteins, enzymes, and fragments thereof, having the amino acid sequences encoded by these nucleic acid sequences which are generated by other methods. Such proteins include those encoded by the open reading frames identified in the tables above, in FIGS. 1 and 2, and fragments thereof.

Thus, in one aspect, the invention provides unique simian adenoviral proteins which are substantially pure, i.e., are free of other viral and proteinaceous proteins. Preferably, these proteins are at least 10% homogeneous, more preferably 60% homogeneous, and most preferably 95% homogeneous.

In one embodiment, the invention provides unique simian-derived capsid proteins. As used herein, a simian-derived capsid protein includes any adenoviral capsid protein that contains a Pan8, Pan6, Pan7, SV1, SV25 or SV39 capsid protein or a fragment thereof, as defined above, including, without limitation, chimeric capsid proteins, fusion proteins, artificial capsid proteins, synthetic capsid proteins, and recombinantly capsid proteins, without limitation to means of generating these proteins.

Suitably, these simian-derived capsid proteins contain one or more Pan8, Pan6, Pan7, SV1, SV25 or SV39 regions or fragments thereof (e.g., a hexon, penton, fiber or fragment thereof) in combination with capsid regions or fragments thereof of different adenoviral serotypes, or modified simian capsid proteins or fragments, as described herein. A “modification of a capsid protein associated with altered tropism” as used herein includes an altered capsid protein, i.e., a penton, hexon or fiber protein region, or fragment thereof, such as the knob domain of the fiber region, or a polynucleotide encoding same, such that specificity is altered. The simian-derived capsid may be constructed with one or more of the simian Ad of the invention or another Ad serotypes which may be of human or non-human origin.

Such Ad may be obtained from a variety of sources including the ATCC, commercial and academic sources, or the sequences of the Ad may be obtained from GenBank or other suitable sources.

The amino acid sequences of the simian adenoviruses penton proteins of the invention are provided herein. The AdPan5 penton protein is provided in SEQ ID NO:2. The AdPan7 penton is provided in SEQ ID NO:6. The AdPan6 penton is provided in SEQ ID NO:10. The SV1 penton is provided in SEQ ID NO:25. The SV25 penton protein is provided in SEQ ID NO:30. The SV39 penton is provided in SEQ ID NO:35. Suitably, any of these penton proteins, or unique fragments thereof, may be utilized for a variety of purposes. Examples of suitable fragments include the penton having N-terminal and/or C-terminal truncations of about 50, 100, 150, or 200 amino acids, based upon the amino acid numbering provided above and in SEQ ID NO:2; SEQ ID NO:6; SEQ ID NO:25; SEQ ID NO:30, or SEQ ID NO:35. Other suitable fragments include shorter internal, C-terminal, or N-terminal fragments. Further, the penton protein may be modified for a variety of purposes known to those of skill in the art.

The invention further provides the amino acid sequences of the hexon protein of Pan5 [SEQ ID NO:3], Pan6 [SEQ ID NO:7], Pan 7 [SEQ ID NO:11], SV1 [SEQ ID NO:26], SV25 [SEQ ID NO:31], and/or SV39 [SEQ ID NO:36]. Suitably, this hexon protein, or unique fragments thereof, may be utilized for a variety of purposes. Examples of suitable fragments include the hexon having N-terminal and/or C-terminal truncations of about 50, 100, 150, 200, 300, 400, or 500 amino acids, based upon the amino acid numbering provided above and in SEQ ID NO: 3, 7, 11, 26, 31 and 36. Other suitable fragments include shorter internal, C-terminal, or N-terminal fragments. For example, one suitable fragment the loop region (domain) of the hexon protein, designated DE1 and FG1, or a hypervariable region thereof. Such fragments include the regions spanning amino acid residues about 125 to 443; about 138 to 441, or smaller fragments, such as those spanning about residue 138 to residue 163; about 170 to about 176; about 195 to about 203; about 233 to about 246; about 253 to about 264; about 287 to about 297; and about 404 to about 430 of the simian hexon proteins, with reference to SEQ ID NO: 3, 7, 11, 26, 31 or 36. Other suitable fragments may be readily identified by one of skill in the art. Further, the hexon protein may be modified for a variety of purposes known to those of skill in the art. Because the hexon protein is the determinant for serotype of an adenovirus, such artificial hexon proteins would result in adenoviruses having artificial serotypes. Other artificial capsid proteins can also be constructed using the chimp Ad penton sequences and/or fiber sequences of the invention and/or fragments thereof.

In one example, it may be desirable to generate an adenovirus having an altered hexon protein utilizing the sequences of a hexon protein of the invention. One suitable method for altering hexon proteins is described in U.S. Pat. No. 5,922,315, which is incorporated by reference. In this method, at least one loop region of the adenovirus hexon is changed with at least one loop region of another adenovirus serotype. Thus, at least one loop region of such an altered adenovirus hexon protein is a simian Ad hexon loop region of the invention (e.g. Pan7). In one embodiment, a loop region of the Pan7 hexon protein is replaced by a loop region from another adenovirus serotype. In another embodiment, the loop region of the Pan7 hexon is used to replace a loop region from another adenovirus serotype. Suitable adenovirus serotypes may be readily selected from among human and non-human serotypes, as described herein. Pan7 is selected for purposes of illustration only; the other simian Ad hexon proteins of the invention may be similarly altered, or used to alter another Ad hexon. The selection of a suitable serotype is not a limitation of the present invention. Still other uses for the hexon protein sequences of the invention will be readily apparent to those of skill in the art.

The invention further encompasses the fiber proteins of the simian adenoviruses of the invention. The fiber protein of AdPan 5 has the amino acid sequence of SEQ ID NO:4. The fiber protein AdPan6 has the amino acid sequence of SEQ ID NO: 8. The fiber protein of AdPan7 has the amino acid sequence of SEQ ID NO: 12. SV-1 has two fiber proteins; fiber 2 has the amino acid sequence of SEQ ID NO:27 and fiber 1 has the amino acid sequence of SEQ ID NO:28. SV-25 also has two fiber proteins; fiber 2 has the amino acid sequence of SEQ ID NO:32 and fiber 1 has the amino acid sequence of SEQ ID NO:33. The fiber protein of SV-39 has the amino acid sequence of SEQ ID NO:37.

Suitably, this fiber protein, or unique fragments thereof, may be utilized for a variety of purposes. One suitable fragment is the fiber knob, which spans about amino acids 247 to 425 of SEQ ID NO: 4, 8, 12, 28, 32, 33 and 37. See FIG. 2. Examples of other suitable fragments include the fiber having N-terminal and/or C-terminal truncations of about 50, 100, 150, or 200 amino acids, based upon the amino acid numbering provided above and in SEQ ID NO: 4, 8, 12, 28, 32, 33 and 37. Still other suitable fragments include internal fragments. Further, the fiber protein may be modified using a variety of techniques known to those of skill in the art.

The invention further encompasses unique fragments of the proteins of the invention which are at least 8 amino acids in length. However, fragments of other desired lengths can be readily utilized. In addition, the invention encompasses such modifications as may be introduced to enhance yield and/or expression of a Pan8, Pan6, Pan7, SV1, SV25 or SV39 gene product, e.g., construction of a fusion molecule in which all or a fragment of the Pan8, Pan6, Pan7, SV1, SV25 or SV39 gene product is fused (either directly or via a linker) with a fusion partner to enhance. Other suitable modifications include, without limitation, truncation of a coding region (e.g., a protein or enzyme) to eliminate a pre- or pro-protein ordinarily cleaved and to provide the mature protein or enzyme and/or mutation of a coding region to provide a secretable gene product. Still other modifications will be readily apparent to one of skill in the art. The invention further encompasses proteins having at least about 95% to 99% identity to the Pan8, Pan6, Pan7, SV1, SV25 or SV39 proteins provided herein.

As described herein, vectors of the invention containing the adenoviral capsid proteins of the invention are particularly well suited for use in applications in which the neutralizing antibodies diminish the effectiveness of other Ad serotype based vectors, as well as other viral vectors. The rAd vectors of the invention are particularly advantageous in readministration for repeat gene therapy or for boosting immune response (vaccine titers).

Under certain circumstances, it may be desirable to use one or more of the Pan8, Pan6, Pan7, SV1, SV25 and/or SV39 gene products (e.g., a capsid protein or a fragment thereof) to generate an antibody. The term “an antibody,” as used herein, refers to an immunoglobulin molecule which is able to specifically bind to an epitope. Thus, the antibodies of the invention bind, preferably specifically and without cross-reactivity, to a Pan8, Pan6, Pan7, SV1, SV25 or SV39 epitope. The antibodies in the present invention exist in a variety of forms including, for example, high affinity polyclonal antibodies, monoclonal antibodies, synthetic antibodies, chimeric antibodies, recombinant antibodies and humanized antibodies. Such antibodies originate from immunoglobulin classes IgG, IgM, IgA, IgD and IgE.

Such antibodies may be generated using any of a number of methods know in the art. Suitable antibodies may be generated by well-known conventional techniques, e.g. Kohler and Milstein and the many known modifications thereof. Similarly desirable high titer antibodies are generated by applying known recombinant techniques to the monoclonal or polyclonal antibodies developed to these antigens [see, e.g., PCT Patent Application No. PCT/GB85/00392; British Patent Application Publication No. GB2188638A; Amit et al., 1986 Science, 233:747-753; Queen et al., 1989 Proc. Nat'l. Acad. Sci. USA, 86:10029-10033; PCT Patent Application No. PCT/WO9007861; and Riechmann et al., Nature, 332:323-327 (1988); Huse et al, 1988a Science, 246:1275-1281]. Alternatively, antibodies can be produced by manipulating the complementarity determining regions of animal or human antibodies to the antigen of this invention. See, e.g., E. Mark and Padlin, “Humanization of Monoclonal Antibodies”, Chapter 4, The Handbook of Experimental Pharmacology, Vol. 113, The Pharmacology of Monoclonal Antibodies, Springer-Verlag (June, 1994); Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; and Bird et al., 1988, Science 242:423-426. Further provided by the present invention are anti-idiotype antibodies (Ab2) and anti-anti-idiotype antibodies (Ab3). See, e.g., M. Wettendorff et al., “Modulation of anti-tumor immunity by anti-idiotypic antibodies.” In Idiotypic Network and Diseases, ed. by J. Cerny and J. Hiernaux, 1990 J. Am. Soc. Microbiol., Washington D.C.: pp. 203-229]. These anti-idiotype and anti-anti-idiotype antibodies are produced using techniques well known to those of skill in the art. These antibodies may be used for a variety of purposes, including diagnostic and clinical methods and kits.

Under certain circumstances, it may be desirable to introduce a detectable label or a tag onto a Pan5, Pan6, Pan7, SV1, SV25 or SV39 gene product, antibody or other construct of the invention. As used herein, a detectable label is a molecule which is capable, alone or upon interaction with another molecule, of providing a detectable signal. Most desirably, the label is detectable visually, e.g. by fluorescence, for ready use in immunohistochemical analyses or immunofluorescent microscopy. For example, suitable labels include fluorescein isothiocyanate (FITC), phycoerythrin (PE), allophycocyanin (APC), coriphosphine-O (CPO) or tandem dyes, PE-cyanin-5 (PC5), and PE-Texas Red (ECD). All of these fluorescent dyes are commercially available, and their uses known to the art. Other useful labels include a colloidal gold label. Still other useful labels include radioactive compounds or elements. Additionally, labels include a variety of enzyme systems that operate to reveal a colorimetric signal in an assay, e.g., glucose oxidase (which uses glucose as a substrate) releases peroxide as a product which in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase which reacts with ATP, glucose, and NAD+ to yield, among other products, NADH that is detected as increased absorbance at 340 nm wavelength. Other label systems that are utilized in the methods of this invention are detectable by other means, e.g., colored latex microparticles [Bangs Laboratories, Indiana] in which a dye is embedded are used in place of enzymes to form conjugates with the target sequences provide a visual signal indicative of the presence of the resulting complex in applicable assays.

Methods for coupling or associating the label with a desired molecule are similarly conventional and known to those of skill in the art. Known methods of label attachment are described [see, for example, Handbook of Fluorescent probes and Research Chemicals, 6th Ed., R. P. M. Haugland, Molecular Probes, Inc., Eugene, Oreg., 1996; Pierce Catalog and Handbook, Life Science and Analytical Research Products, Pierce Chemical Company, Rockford, Ill., 1994/1995]. Thus, selection of the label and coupling methods do not limit this invention.

The sequences, proteins, and fragments of the invention may be produced by any suitable means, including recombinant production, chemical synthesis, or other synthetic means. Suitable production techniques are well known to those of skill in the art. See, e.g., Sambrook et al, Molecular Cloning: A Laboratory Manual, Cold Spring Harbor Press (Cold Spring Harbor, N.Y.). Alternatively, peptides can also be synthesized by the well known solid phase peptide synthesis methods (Merrifield, J. Am. Chem. Soc., 85:2149 (1962); Stewart and Young, Solid Phase Peptide Synthesis (Freeman, San Francisco, 1969) pp. 27-62). These and other suitable production methods are within the knowledge of those of skill in the art and are not a limitation of the present invention.

In addition, one of skill in the art will readily understand that the Ad sequences of the invention can be readily adapted for use for a variety of viral and non-viral vector systems for in vitro, ex vivo or in vivo delivery of therapeutic and immunogenic molecules. For example, in one embodiment, the simian Ad capsid proteins and other simian adenovirus proteins described herein are used for non-viral, protein-based delivery of genes, proteins, and other desirable diagnostic, therapeutic and immunogenic molecules. In one such embodiment, a protein of the invention is linked, directly or indirectly, to a molecule for targeting to cells with a receptor for adenoviruses. Preferably, a capsid protein such as a hexon, penton, fiber or a fragment thereof having a ligand for a cell surface receptor is selected for such targeting. Suitable molecules for delivery are selected from among the therapeutic molecules described herein and their gene products. A variety of linkers including, lipids, polyLys, and the like may be utilized as linkers. For example, the simian penton protein may be readily utilized for such a purpose by production of a fusion protein using the simian penton sequences in a manner analogous to that described in Medina-Kauwe L K, et al, Gene Ther. 2001 May; 8(10):795-803 and Medina-Kauwe L K, et al, Gene Ther. 2001 December; 8(23): 1753-1761. Alternatively, the amino acid sequences of simian Ad protein IX may be utilized for targeting vectors to a cell surface receptor, as described in US Patent Application 20010047081. Suitable ligands include a CD40 antigen, an RGD-containing or polylysine-containing sequence, and the like. Still other simian Ad proteins, including, e.g., the hexon protein and/or the fiber protein, may be used for used for these and similar purposes. Still other adenoviral proteins of the invention may be used as alone, or in combination with other adenoviral protein, for a variety of purposes which will be readily apparent to one of skill in the art. In addition, still other uses for the adenoviral proteins of the invention will be readily apparent to one of skill in the art.

### II. Recombinant Adenoviral Vectors

The compositions of this invention include vectors that deliver a heterologous molecule to cells, either for therapeutic or vaccine purposes. As used herein, a vector may include any genetic element including, without limitation, naked DNA, a phage, transposon, cosmid, episome, plasmid, or a virus. Such vectors contain simian adenovirus DNA of any of the serotypes described herein, (e.g., Pan8, Pan6, Pan7, baboon adenovirus ATCC-VR 275, Rhesus monkey strains, ATCC-VR 209, ATCC-VR 275, ATCC VR 353, ATCC VR 355, and African Green Monkey strains ATCC VR-541, ATCC VR 941, ATCC VR 942, and ATCC 943, SV1, SV25 and/or SV39) and a minigene. By “minigene” is meant the combination of a selected heterologous gene and the other regulatory elements necessary to drive translation, transcription and/or expression of the gene product in a host cell.

Typically, an adenoviral vector of the invention is designed such that the minigene is located in a nucleic acid molecule that contains other adenoviral sequences in the region native to a selected adenoviral gene. The minigene may be inserted into an existing gene region to disrupt the function of that region, if desired. Alternatively, the minigene may be inserted into the site of a partially or fully deleted adenoviral gene. For example, the minigene may be located in the site of such as the site of a functional E1 deletion or functional E3 deletion, among others that may be selected. The term “functionally deleted” or “functional deletion” means that a sufficient amount of the gene region is removed or otherwise damaged, e.g., by mutation or modification, so that the gene region is no longer capable of producing functional products of gene expression. If desired, the entire gene region may be removed. Other suitable sites for gene disruption or deletion are discussed elsewhere in the application.

For example, for a production vector useful for generation of a recombinant virus, the vector may contain the minigene and either the 5′ end of the adenoviral genome or the 3′ end of the adenoviral genome, or both the 5′ and 3′ ends of the adenoviral genome. The 5′ end of the adenoviral genome contains the 5′ cis-elements necessary for packaging and replication; i.e., the 5′ inverted terminal repeat (ITR) sequences (which functions as origins of replication) and the native 5′ packaging enhancer domains (that contain sequences necessary for packaging linear Ad genomes and enhancer elements for the E1 promoter). The 3′ end of the adenoviral genome includes the 3′ cis-elements (including the ITRs) necessary for packaging and encapsidation. Suitably, a recombinant adenovirus contains both 5′ and 3′ adenoviral cis-elements and the minigene is located between the 5′ and 3′ adenoviral sequences. Any adenoviral vector of the invention may also contain additional adenoviral sequences.

Suitably, these adenoviral vectors of the invention contain one or more adenoviral elements derived from an adenoviral genome of the invention. In one embodiment, the vectors contain adenoviral ITRs from Pan8, Pan6, Pan7, SV1, SV25 or SV39 and additional adenoviral sequences from the same adenoviral serotype. In another embodiment, the vectors contain adenoviral sequences that are derived from a different adenoviral serotype than that which provides the ITRs. As defined herein, a pseudotyped adenovirus refers to an adenovirus in which the capsid protein of the adenovirus is from a different serotype than the serotype which provides the ITRs. The selection of the serotype of the ITRs and the serotype of any other adenoviral sequences present in vector is not a limitation of the present invention. A variety of adenovirus strains are available from the American Type Culture Collection, Manassas, Va., or available by request from a variety of commercial and institutional sources. Further, the sequences of many such strains are available from a variety of databases including, e.g., PubMed and GenBank. Homologous adenovirus vectors prepared from other simian or from human adenoviruses are described in the published literature [see, for example, U.S. Pat. No. 5,240,846]. The DNA sequences of a number of adenovirus types are available from GenBank, including type Ad5 [GenBank Accession No. M73260]. The adenovirus sequences may be obtained from any known adenovirus serotype, such as serotypes 2, 3, 4, 7, 12 and 40, and further including any of the presently identified human types. Similarly adenoviruses known to infect non-human animals (e.g., simians) may also be employed in the vector constructs of this invention. See, e.g., U.S. Pat. No. 6,083,716.

The viral sequences, helper viruses, if needed, and recombinant viral particles, and other vector components and sequences employed in the construction of the vectors described herein are obtained as described above. The DNA sequences of the simian adenovirus sequences of the invention are employed to construct vectors and cell lines useful in the preparation of such vectors.

Modifications of the nucleic acid sequences forming the vectors of this invention, including sequence deletions, insertions, and other mutations may be generated using standard molecular biological techniques and are within the scope of this invention.

A. The “Minigene”

The methods employed for the selection of the transgene, the cloning and construction of the “minigene” and its insertion into the viral vector are within the skill in the art given the teachings provided herein.

1. The Transgene

The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a host cell.

The composition of the transgene sequence will depend upon the use to which the resulting vector will be put. For example, one type of transgene sequence includes a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is GFP or luciferase, the vector carrying the signal may be measured visually by color or light production in a luminometer.

However, desirably, the transgene is a non-marker sequence encoding a product which is useful in biology and medicine, such as proteins, peptides, RNA, enzymes, or catalytic RNAs. Desirable RNA molecules include tRNA, dsRNA, ribosomal RNA, catalytic RNAs, and antisense RNAs. One example of a useful RNA sequence is a sequence which extinguishes expression of a targeted nucleic acid sequence in the treated animal.

The transgene may be used for treatment, e.g., of genetic deficiencies, as a cancer therapeutic or vaccine, for induction of an immune response, and/or for prophylactic vaccine purposes. As used herein, induction of an immune response refers to the ability of a molecule (e.g., a gene product) to induce a T cell and/or a humoral immune response to the molecule. The invention further includes using multiple transgenes, e.g., to correct or ameliorate a condition caused by a multi-subunit protein. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., M. L. Donnelly, et al, J. Gen. Virol., 78(Pt 1):13-21 (January 1997); Furler, S., et al, Gene Ther., 8(11):864-873 (June 2001); Klump H., et al., Gene Ther., 8(10):811-817 (May 2001). This 2A peptide is significantly smaller than an IRES, making it well suited for use when space is a limiting factor. However, the selected transgene may encode any biologically active product or other product, e.g., a product desirable for study.

Suitable transgenes may be readily selected by one of skill in the art. The selection of the transgene is not considered to be a limitation of this invention.

2. Regulatory Elements

In addition to the major elements identified above for the minigene, the vector also includes conventional control elements necessary which are operably linked to the transgene in a manner that permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. For example, inducible promoters include the zinc-inducible sheep metallothionine (MT) promoter and the dexamethasone (Dex)-inducible mouse mammary tumor virus (MMTV) promoter. Other inducible systems include the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)]. Other systems include the FK506 dimer, VP16 or p65 using castradiol, diphenol murislerone, the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. The effectiveness of some inducible promoters increases over time. In such cases one can enhance the effectiveness of such systems by inserting multiple repressors in tandem, e.g., TetR linked to a TetR by an IRES. Alternatively, one can wait at least 3 days before screening for the desired function. One can enhance expression of desired proteins by known means to enhance the effectiveness of this system. For example, the Woodchuck Hepatitis Virus Posttranscriptional Regulatory Element (WPRE).

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of the transgene includes a transgene operably linked to a tissue-specific promoter. For instance, if expression in skeletal muscle is desired, a promoter active in muscle should be used. These include the promoters from genes encoding skeletal ∃-actin, myosin light chain 2A, dystrophin, muscle creatine kinase, as well as synthetic muscle promoters with activities higher than naturally occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

Optionally, vectors carrying transgenes encoding therapeutically useful or immunogenic products may also include selectable markers or reporter genes may include sequences encoding geneticin, hygromicin or purimycin resistance, among others. Such selectable reporters or marker genes (preferably located outside the viral genome to be packaged into a viral particle) can be used to signal the presence of the plasmids in bacterial cells, such as ampicillin resistance. Other components of the vector may include an origin of replication. Selection of these and other promoters and vector elements are conventional and many such sequences are available [see, e.g., Sambrook et al, and references cited therein].

These vectors are generated using the techniques and sequences provided herein, in conjunction with techniques known to those of skill in the art. Such techniques include conventional cloning techniques of cDNA such as those described in texts [Sambrook et al, Molecular Cloning: A Laboratory Manual, Cold Spring Harbor Press, Cold Spring Harbor, N.Y.], use of overlapping oligonucleotide sequences of the adenovirus genomes, polymerase chain reaction, and any suitable method which provides the desired nucleotide sequence.

### III. Production of the Recombinant Viral Particle

In one embodiment, the simian adenoviral plasmids (or other vectors) are used to produce recombinant adenoviral particles. At a minimum, a recombinant simian adenovirus (i.e., a viral particle) useful in the invention contains the simian adenovirus cis-elements necessary for replication and virion encapsidation, which cis-elements flank the heterologous gene. That is, the vector contains the cis-acting 5′ inverted terminal repeat (ITR) sequences of the adenoviruses which function as origins of replication), the native 5′ packaging/enhancer domains (that contain sequences necessary for packaging linear Ad genomes and enhancer elements for the E1 promoter), the heterologous molecule, and the 5′ ITR sequences. See, for example, the techniques described for preparation of a “minimal” human Ad vector in U.S. Pat. No. 6,203,975, which is incorporated by reference, can be readily adapted for the recombinant simian adenovirus. Optionally, the recombinant simian adenoviruses useful in this invention contain more than the minimal simian adenovirus sequences defined above

In one embodiment, the recombinant adenoviruses are functionally deleted in the E1a or E1b genes, and optionally bearing other mutations, e.g., temperature-sensitive mutations or deletions in other genes. In other embodiments, it is desirable to retain an intact E1a and/or E1b region in the recombinant adenoviruses. Such an intact E1 region may be located in its native location in the adenoviral genome or placed in the site of a deletion in the native adenoviral genome (e.g., in the E3 region).

In the construction of useful simian adenovirus vectors for delivery of a gene to the human (or other mammalian) cell, a range of adenovirus nucleic acid sequences can be employed in the vectors. For example, all or a portion of the adenovirus delayed early gene E3 may be eliminated from the simian adenovirus sequence which forms a part of the recombinant virus. The function of simian E3 is believed to be irrelevant to the function and production of the recombinant virus particle. Simian adenovirus vectors may also be constructed having a deletion of at least the ORF6 region of the E4 gene, and more desirably because of the redundancy in the function of this region, the entire E4 region. Still another vector of this invention contains a deletion in the delayed early gene E2a. Deletions may also be made in any of the late genes L1 through L5 of the simian adenovirus genome. Similarly, deletions in the intermediate genes IX and IVa₂ may be useful for some purposes. Other deletions may be made in the other structural or non-structural adenovirus genes. The above discussed deletions may be used individually, i.e., an adenovirus sequence for use in the present invention may contain deletions in only a single region. Alternatively, deletions of entire genes or portions thereof effective to destroy their biological activity may be used in any combination. For example, in one exemplary vector, the adenovirus sequence may have deletions of the E1 genes and the E4 gene, or of the E1, E2a and E3 genes, or of the E1 and E3 genes, or of E1, E2a and E4 genes, with or without deletion of E3, and so on. As discussed above, such deletions may be used in combination with other mutations, such as temperature-sensitive mutations, to achieve a desired result.

An adenoviral vector lacking any essential adenoviral sequences (e.g., E1a, E1b, E2a, E2b, E4ORF6, L1, L2, L3, L4 and L5) may be cultured in the presence of the missing adenoviral gene products which are required for viral infectivity and propagation of an adenoviral particle. These helper functions may be provided by culturing the adenoviral vector in the presence of one or more helper constructs (e.g., a plasmid or virus) or a packaging host cell. See, for example, the techniques described for preparation of a “minimal” human Ad vector in International Patent Application No. WO96/13597, published May 9, 1996, and incorporated herein by reference.

Regardless of whether the recombinant simian adenovirus contains only the minimal Ad sequences, or the entire Ad genome with only functional deletions in the E1 and/or E3 regions, in one embodiment, the recombinant virus contains a capsid derived from a simian adenovirus. Alternatively, in other embodiments, recombinant pseudotyped adenoviruses may be used in the methods of the invention. Such pseudotyped adenoviruses utilize simian adenovirus capsid proteins in which a nucleic acid molecule carrying heterologous simian adenovirus sequences, or non-simian adenovirus sequences have been packaged. These recombinant simian adenoviruses of the invention may be produced using methods that are known to those of skill in the art.

1. Helper Viruses

Thus, depending upon the simian adenovirus gene content of the viral vectors employed to carry the minigene, a helper adenovirus or non-replicating virus fragment may be necessary to provide sufficient simian adenovirus gene sequences necessary to produce an infective recombinant viral particle containing the minigene. Useful helper viruses contain selected adenovirus gene sequences not present in the adenovirus vector construct and/or not expressed by the packaging cell line in which the vector is transfected. In one embodiment, the helper virus is replication-defective and contains a variety of adenovirus genes in addition to the sequences described above. Such a helper virus is desirably used in combination with an E1-expressing cell line.

Helper viruses may also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus may optionally contain a second reporter minigene. A number of such reporter genes are known to the art. The presence of a reporter gene on the helper virus which is different from the transgene on the adenovirus vector allows both the Ad vector and the helper virus to be independently monitored. This second reporter is used to enable separation between the resulting recombinant virus and the helper virus upon purification.

2. Complementation Cell Lines

To generate recombinant simian adenoviruses (Ad) deleted in any of the genes described above, the function of the deleted gene region, if essential to the replication and infectivity of the virus, must be supplied to the recombinant virus by a helper virus or cell line, i.e., a complementation or packaging cell line. In many circumstances, a cell line expressing the human E1 can be used to transcomplement the chimp Ad vector. This is particularly advantageous because, due to the diversity between the chimp Ad sequences of the invention and the human AdE1 sequences found in currently available packaging cells, the use of the current human E1-containing cells prevents the generation of replication-competent adenoviruses during the replication and production process. However, in certain circumstances, it will be desirable to utilize a cell line which expresses the E1 gene products can be utilized for production of an E1-deleted simian adenovirus. Such cell lines have been described. See, e.g., U.S. Pat. No. 6,083,716.

If desired, one may utilize the sequences provided herein to generate a packaging cell or cell line that expresses, at a minimum, the adenovirus E1 gene from Pan8, Pan6, Pan7, SV1, SV25 or SV39 under the transcriptional control of a promoter for expression in a selected parent cell line. Inducible or constitutive promoters may be employed for this purpose. Examples of such promoters are described in detail elsewhere in this specification. A parent cell is selected for the generation of a novel cell line expressing any desired AdPan5, Pan6, Pan7, SV1, SV25 or SV39 gene. Without limitation, such a parent cell line may be HeLa [ATCC Accession No. CCL 2], A549 [ATCC Accession No. CCL 185], HEK 293, KB [CCL 17], Detroit [e.g., Detroit 510, CCL 72] and WI-38 [CCL 75] cells, among others. These cell lines are all available from the American Type Culture Collection, 10801 University Boulevard, Manassas, Va. 20110-2209. Other suitable parent cell lines may be obtained from other sources.

Such E1-expressing cell lines are useful in the generation of recombinant simian adenovirus E1 deleted vectors. Additionally, or alternatively, the invention provides cell lines that express one or more simian adenoviral gene products, e.g., E1a, E1b, E2a, and/or E4ORF6, can be constructed using essentially the same procedures for use in the generation of recombinant simian viral vectors. Such cell lines can be utilized to transcomplement adenovirus vectors deleted in the essential genes that encode those products, or to provide helper functions necessary for packaging of a helper-dependent virus (e.g., adeno-associated virus). The preparation of a host cell according to this invention involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus genomes, combined with polymerase chain reaction, synthetic methods, and any other suitable methods which provide the desired nucleotide sequence.

In still another alternative, the essential adenoviral gene products are provided in trans by the adenoviral vector and/or helper virus. In such an instance, a suitable host cell can be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, HEK 293 cells or PERC6 (both of which express functional adenoviral E1) [Fallaux, F J et al, (1998), Hum Gene Ther, 9:1909-1917], Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc.

3. Assembly of Viral Particle and Transfection of a Cell Line

Generally, when delivering the vector comprising the minigene by transfection, the vector is delivered in an amount from about 5 μg to about 100 μg DNA, and preferably about 10 to about 50 μg DNA to about 1×10⁴ cells to about 1×10¹³ cells, and preferably about 10⁵ cells. However, the relative amounts of vector DNA to host cells may be adjusted, taking into consideration such factors as the selected vector, the delivery method and the host cells selected.

The vector may be any vector known in the art or disclosed above, including naked DNA, a plasmid, phage, transposon, cosmids, episomes, viruses, etc. Introduction into the host cell of the vector may be achieved by any means known in the art or as disclosed above, including transfection, and infection. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously-added factors, for example.

Introduction of the molecules (as plasmids or viruses) into the host cell may also be accomplished using techniques known to the skilled artisan and as discussed throughout the specification. In a preferred embodiment, standard transfection techniques are used, e.g., CaPO₄ transfection or electroporation.

Assembly of the selected DNA sequences of the adenovirus (as well as the transgene and other vector elements into various intermediate plasmids, and the use of the plasmids and vectors to produce a recombinant viral particle are all achieved using conventional techniques. Such techniques include conventional cloning techniques of cDNA such as those described in texts [Sambrook et al, cited above], use of overlapping oligonucleotide sequences of the adenovirus genomes, polymerase chain reaction, and any suitable method which provides the desired nucleotide sequence. Standard transfection and co-transfection techniques are employed, e.g., CaPO₄ precipitation techniques. Other conventional methods employed include homologous recombination of the viral genomes, plaquing of viruses in agar overlay, methods of measuring signal generation, and the like.

For example, following the construction and assembly of the desired minigene-containing viral vector, the vector is transfected in vitro in the presence of a helper virus into the packaging cell line. Homologous recombination occurs between the helper and the vector sequences, which permits the adenovirus-transgene sequences in the vector to be replicated and packaged into virion capsids, resulting in the recombinant viral vector particles. The current method for producing such virus particles is transfection-based. However, the invention is not limited to such methods. The resulting recombinant simian adenoviruses are useful in transferring a selected transgene to a selected cell. In in vivo experiments with the recombinant virus grown in the packaging cell lines, the E1-deleted recombinant simian adenoviral vectors of the invention demonstrate utility in transferring a transgene to a non-simian, preferably a human, cell.

### IV. Use of the Recombinant Adenovirus Vectors

The recombinant simian adenovirus vectors of the invention are useful for gene transfer to a human or non-simian veterinary patient in vitro, ex vivo, and in vivo.

The recombinant adenovirus vectors described herein can be used as expression vectors for the production of the products encoded by the heterologous genes in vitro. For example, the recombinant adenoviruses containing a gene inserted into the location of an E1 deletion may be transfected into an E1-expressing cell line as described above. Alternatively, replication-competent adenoviruses may be used in another selected cell line. The transfected cells are then cultured in the conventional manner, allowing the recombinant adenovirus to express the gene product from the promoter. The gene product may then be recovered from the culture medium by known conventional methods of protein isolation and recovery from culture.

A Pan8, Pan6, Pan7, SV1, SV25 or SV39-derived recombinant simian adenoviral vector of the invention provides an efficient gene transfer vehicle that can deliver a selected transgene to a selected host cell in vivo or ex vivo even where the organism has neutralizing antibodies to one or more AAV serotypes. In one embodiment, the rAAV and the cells are mixed ex vivo; the infected cells are cultured using conventional methodologies; and the transduced cells are re-infused into the patient. These compositions are particularly well suited to gene delivery for therapeutic purposes and for immunization, including inducing protective immunity.

More commonly, the Pan 5, Pan6, Pan7, SV1, SV25, or SV39 recombinant adenoviral vectors of the invention will be utilized for delivery of therapeutic or immunogenic molecules, as described below. It will be readily understood for both applications, that the recombinant adenoviral vectors of the invention are particularly well suited for use in regimens involving repeat delivery of recombinant adenoviral vectors. Such regimens typically involve delivery of a series of viral vectors in which the viral capsids are alternated. The viral capsids may be changed for each subsequent administration, or after a pre-selected number of administrations of a particular serotype capsid (e.g., one, two, three, four or more). Thus, a regimen may involve delivery of a rAd with a first simian capsid, delivery with a rAd with a second simian capsid, and delivery with a third simian capsid. A variety of other regimens which use the Ad capsids of the invention alone, in combination with one another, or in combination with other Ad serotypes will be apparent to those of skill in the art. Optionally, such a regimen may involve administration of rAd with capsids of other non-human primate adenoviruses, human adenoviruses, or artificial serotypes such as are described herein. Each phase of the regimen may involve administration of a series of injections (or other delivery routes) with a single Ad serotype capsid followed by a series with another Ad serotype capsid. Alternatively, the recombinant Ad vectors of the invention may be utilized in regimens involving other non-adenoviral-mediated delivery systems, including other viral systems, non-viral delivery systems, protein, peptides, and other biologically active molecules.

The following sections will focus on exemplary molecules which may be delivered via the adenoviral vectors of the invention.

A. Ad-Mediated Delivery of Therapeutic Molecules

In one embodiment, the above-described recombinant vectors are administered to humans according to published methods for gene therapy. A simian viral vector bearing the selected transgene may be administered to a patient, preferably suspended in a biologically compatible solution or pharmaceutically acceptable delivery vehicle. A suitable vehicle includes sterile saline that may be formatted with a variety of buffering solutions (e.g., phosphate buffered saline). Other aqueous and non-aqueous isotonic sterile injection solutions and aqueous and non-aqueous sterile suspensions known to be pharmaceutically acceptable carriers and well known to those of skill in the art may be employed for this purpose. Other exemplary carriers include sterile saline, lactose, sucrose, calcium phosphate, gelatin, dextran, agar, pectin, peanut oil, sesame oil, and water. The selection of the carrier is not a limitation of the present invention.

The simian adenoviral vectors are administered in sufficient amounts to transduce the target cells and to provide sufficient levels of gene transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the retina and other intraocular delivery methods, direct delivery to the liver, inhalation, intranasal, intravenous, intramuscular, intratracheal, subcutaneous, intradermal, rectal, oral and other parenteral routes of administration. Routes of administration may be combined, if desired, or adjusted depending upon the transgene or the condition. The route of administration primarily will depend on the nature of the condition being treated.

Dosages of the viral vector will depend primarily on factors such as the condition being treated, the age, weight and health of the patient, and may thus vary among patients. For example, a therapeutically effective adult human or veterinary dosage of the viral vector is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles virus. Dosages will range depending upon the size of the animal and the route of administration. For example, a suitable human or veterinary dosage (for about an 80 kg animal) for intramuscular injection is in the range of about 1×10⁹ to about 5×10¹² particles per mL, for a single site. Optionally, multiple sites of administration may be delivered. In another example, a suitable human or veterinary dosage may be in the range of about 1×10¹¹ to about 1×10¹⁵ particles for an oral formulation. One of skill in the art may adjust these doses, depending the route of administration, and the therapeutic or vaccinal application for which the recombinant vector is employed. The levels of expression of the transgene, or for an immunogen, the level of circulating antibody, can be monitored to determine the frequency of dosage administration. Yet other methods for determining the timing of frequency of administration will be readily apparent to one of skill in the art.

An optional method step involves the co-administration to the patient, either concurrently with, or before or after administration of the viral vector, of a suitable amount of a short acting immune modulator. The selected immune modulator is defined herein as an agent capable of inhibiting the formation of neutralizing antibodies directed against the recombinant vector of this invention or capable of inhibiting cytolytic T lymphocyte (CTL) elimination of the vector. The immune modulator may interfere with the interactions between the T helper subsets (T_{H1} or T_{H2}) and B cells to inhibit neutralizing antibody formation. Alternatively, the immune modulator may inhibit the interaction between T_{H1} cells and CTLs to reduce the occurrence of CTL elimination of the vector. A variety of useful immune modulators and dosages for use of same are disclosed, for example, in Yang et al., J. Virol., 70(9) (September, 1996); International Patent Application No. WO96/12406, published May 2, 1996; and International Patent Application No. PCT/US96/03035, all incorporated herein by reference.

1. Therapeutic Transgenes

Useful therapeutic products encoded by the transgene include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor (TGF), platelet-derived growth factor (PDGF), insulin growth factors I and II (IGF-I and IGF-II), any one of the transforming growth factor superfamily, including TGF, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-25 (including, e.g., IL-2, IL-4, IL-12 and IL-18), monocyte chemoattractant protein, leukemia inhibitory factor, granulocyte-macrophage colony stimulating factor, Fas ligand, tumor necrosis factors and, interferons, and, stem cell factor, flk-2/flt3 ligand. Gene products produced by the immune system are also useful in the invention. These include, without limitation, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation, including the low density lipoprotein (LDL) receptor, high density lipoprotein (HDL) receptor, the very low density lipoprotein (VLDL) receptor, and the scavenger receptor. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SPI, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence.

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene are particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce self-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

The simian adenoviral vectors of the invention are particularly well suited for therapeutic regimens in which multiple adenoviral-mediated deliveries of transgenes is desired, e.g., in regimens involving redelivery of the same transgene or in combination regimens involving delivery of other transgenes. Such regimens may involve administration of a Pan5, Pan6, Pan7, SV1, SV25 or SV39 simian adenoviral vector, followed by re-administration with a vector from the same serotype adenovirus. Particularly desirable regimens involve administration of a Pan5, Pan6, Pan7, SV1, SV25 or SV39 simian adenoviral vector of the invention, in which the serotype of the viral vector delivered in the first administration differs from the serotype of the viral vector utilized in one or more of the subsequent administrations. For example, a therapeutic regimen involves administration of a Pan5, Pan6, Pan7, SV1, SV25 or SV39 vector and repeat administration with one or more adenoviral vectors of the same or different serotypes. In another example, a therapeutic regimen involves administration of an adenoviral vector followed by repeat administration with a Pan5, Pan6, Pan7, SV1, SV25 or SV39 vector of the invention which differs from the serotype of the first delivered adenoviral vector, and optionally further administration with another vector which is the same or, preferably, differs from the serotype of the vector in the prior administration steps. These regimens are not limited to delivery of adenoviral vectors constructed using the Pan5, Pan6, Pan7, SV1, SV25 or SV39 simian serotypes of the invention. Rather, these regimens can readily utilize vectors other adenoviral serotypes, including, without limitation, other simian adenoviral serotypes (e.g., Pan9 or C68, C1, etc), other non-human primate adenoviral serotypes, or human adenoviral serotypes, in combination with one or more of the Pan5, Pan6, Pan7, SV1, SV25 or SV39 vectors of the invention. Examples of such simian, other non-human primate and human adenoviral serotypes are discussed elsewhere in this document. Further, these therapeutic regimens may involve either simultaneous or sequential delivery of Pan 5, Pan6, Pan7, SV1, SV25, and/or SV39 adenoviral vectors of the invention in combination with non-adenoviral vectors, non-viral vectors, and/or a variety of other therapeutically useful compounds or molecules. The present invention is not limited to these therapeutic regimens, a variety of which will be readily apparent to one of skill in the art.

B. Ad-Mediated Delivery of Immunogenic Transgenes

The recombinant simian adenoviruses may also be employed as immunogenic compositions. As used herein, an immunogenic composition is a composition to which a humoral (e.g., antibody) or cellular (e.g., a cytotoxic T cell) response is mounted to a transgene product delivered by the immunogenic composition following delivery to a mammal, and preferably a primate. The present invention provides a recombinant simian Ad that can contain in any of its adenovirus sequence deletions a gene encoding a desired immunogen. The simian adenovirus is likely to be better suited for use as a live recombinant virus vaccine in different animal species compared to an adenovirus of human origin, but is not limited to such a use. The recombinant adenoviruses can be used as prophylactic or therapeutic vaccines against any pathogen for which the antigen(s) crucial for induction of an immune response and able to limit the spread of the pathogen has been identified and for which the cDNA is available.

Such vaccinal (or other immunogenic) compositions are formulated in a suitable delivery vehicle, as described above. Generally, doses for the immunogenic compositions are in the range defined above for therapeutic compositions. The levels of immunity of the selected gene can be monitored to determine the need, if any, for boosters. Following an assessment of antibody titers in the serum, optional booster immunizations may be desired.

Optionally, a vaccinal composition of the invention may be formulated to contain other components, including, e.g. adjuvants, stabilizers, pH adjusters, preservatives and the like. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin. Suitable exemplary adjuvants include, among others, immune-stimulating complexes (ISCOMS), LPS analogs including 3-O-deacylated monophosphoryl lipid A (Ribi Immunochem Research, Inc.; Hamilton, Mont.), mineral oil and water, aluminum hydroxide, Amphigen, Avirdine, L121/squalene, muramyl peptides, and saponins, such as Quil A, and any biologically active factor, such as cytokine, an interleukin, a chemokine, a ligands, and optimally combinations thereof. Certain of these biologically active factors can be expressed in vivo, e.g., via a plasmid or viral vector. For example, such an adjuvant can be administered with a priming DNA vaccine encoding an antigen to enhance the antigen-specific immune response compared with the immune response generated upon priming with a DNA vaccine encoding the antigen only.

The recombinant adenoviruses are administered in a “an immunogenic amount”, that is, an amount of recombinant adenovirus that is effective in a route of administration to transfect the desired cells and provide sufficient levels of expression of the selected gene to induce an immune response. Where protective immunity is provided, the recombinant adenoviruses are considered to be vaccine compositions useful in preventing infection and/or recurrent disease.

Alternatively, or in addition, the vectors of the invention may contain a transgene encoding a peptide, polypeptide or protein which induces an immune response to a selected immunogen. The recombinant adenoviruses of this invention are expected to be highly efficacious at inducing cytolytic T cells and antibodies to the inserted heterologous antigenic protein expressed by the vector. For example, immunogens may be selected from a variety of viral families. Example of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VPI, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family Filoviridae, which includes hemorrhagic fever viruses such as Marburg and Ebola virus, may be a suitable source of antigens. The paramyxovirus family includes parainfluenza Virus Type 1, parainfluenza Virus Type 3, bovine parainfluenza Virus Type 3, rubulavirus (mumps virus), parainfluenza Virus Type 2, parainfluenza virus Type 4, Newcastle disease virus (chickens), rinderpest, morbillivirus, which includes measles and canine distemper, and pneumovirus, which includes respiratory syncytial virus (e.g., the glyco-(G) protein and the fusion (F) protein, for which sequences are available from GenBank).

The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue).

The retrovirus family includes the sub-family oncorivirinal which encompasses such human and veterinary diseases as feline leukemia virus, HTLVI and HTLVII, lentivirinal (which includes human immunodeficiency virus (HIV), simian immunodeficiency virus (SIV), feline immunodeficiency virus (FIV), equine infectious anemia virus, and spumavirinal). Among the lentiviruses, many suitable antigens have been described and can readily be selected. Examples of suitable HIV and SIV antigens include, without limitation the gag, pol, Vif, Vpx, VPR, Env, Tat, Nef, and Rev proteins, as well as various fragments thereof. For example, suitable fragments of the Env protein may include any of its subunits such as the gp120, gp160, gp41, or smaller fragments thereof, e.g., of at least about 8 amino acids in length. Similarly, fragments of the tat protein may be selected. [See, U.S. Pat. No. 5,891,994 and U.S. Pat. No. 6,193,981.] See, also, the HIV and SW proteins described in D. H. Barouch et al, J. Virol., 75(5):2462-2467 (March 2001), and R. R. Amara, et al, Science, 292:69-74 (6 Apr. 2001). In another example, the HIV and/or SW immunogenic proteins or peptides may be used to form fusion proteins or other immunogenic molecules. See, e.g., the HIV-1 Tat and/or Nef fusion proteins and immunization regimens described in WO 01/54719, published Aug. 2, 2001, and WO 99/16884, published Apr. 8, 1999. The invention is not limited to the HW and/or SIV immunogenic proteins or peptides described herein. In addition, a variety of modifications to these proteins have been described or could readily be made by one of skill in the art. See, e.g., the modified gag protein that is described in U.S. Pat. No. 5,972,596. Further, any desired HIV and/or SW immunogens may be delivered alone or in combination. Such combinations may include expression from a single vector or from multiple vectors. Optionally, another combination may involve delivery of one or more expressed immunogens with delivery of one or more of the immunogens in protein form. Such combinations are discussed in more detail below.

The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (Human CMV), muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxyirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxyirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

The present invention may also encompass immunogens which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus (Haemophilus influenzae, Haemophilus somnus); moraxella; H. ducreyi (which causes chancroid); brucella; Franisella tularensis (which causes tularemia); yersinia (pasteurella); streptobacillus moniliformis and spirillum. Gram-positive bacilli include listeria monocytogenes; erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria.

Examples of specific bacterium species are, without limitation, Streptococcus pneumoniae, Streptococcus pyogenes, Streptococcus agalactiae, Streptococcus faecalis, Moraxella catarrhalis, Helicobacter pylori, Neisseria meningitidis, Neisseria gonorrhoeae, Chlamydia trachomatis, Chlamydia pneumoniae, Chlamydia psittaci, Bordetella pertussis, Salmonella typhi, Salmonella typhimurium, Salmonella choleraesuis, Escherichia coli, Shigella, Vibrio cholerae, Corynebacterium diphtheriae, Mycobacterium tuberculosis, Mycobacterium avium, Mycobacterium intracellulare complex, Proteus mirabilis, Proteus vulgaris, Staphylococcus aureus, Clostridium tetani, Leptospira interrogans, Borrelia burgdorferi, Pasteurella haemolytica, Pasteurella multocida, Actinobacillus pleuropneumoniae and Mycoplasma gallisepticum.

Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis (Cryptococcus), blastomycosis (Blastomyces), histoplasmosis (Histoplasma) and coccidioidomycosis (Coccidiodes); candidiasis (Candida), aspergillosis (Aspergillis), and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of mycoplasma and chlamydial infections include: mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis (e.g., caused by Leishmania major); trypanosomiasis; toxoplasmosis (e.g., caused by Toxoplasma gondii); Pneumocystis carinii; Trichans; Toxoplasma gondii; babesiosis; giardiasis (e.g., caused by Giardia); trichinosis (e.g., caused by Trichomonas); filariasis; schistosomiasis (e.g., caused by Schistosoma); nematodes; trematodes or flukes; and cestode (tapeworm) infections. Other parasitic infections may be caused by Ascaris, Trichuris, Cryptosporidium, and Pneumocystis carinii, among others.

Many of these organisms and/or toxins produced thereby have been identified by the Centers for Disease Control [(CDC), Department of Heath and Human Services, USA], as agents which have potential for use in biological attacks. For example, some of these biological agents, include, Bacillus anthracis (anthrax), Clostridium botulinum and its toxin (botulism), Yersinia pestis (plague), variola major (smallpox), Francisella tularensis (tularemia), and viral hemorrhagic fevers [filoviruses (e.g., Ebola, Marburg], and arenaviruses [e.g., Lassa, Machupo]), all of which are currently classified as Category A agents; Coxiella burnetti (Q fever); Brucella species (brucellosis), Burkholderia mallei (glanders), Burkholderia pseudomallei (meloidosis), Ricinus communis and its toxin (ricin toxin), Clostridium perfringens and its toxin (epsilon toxin), Staphylococcus species and their toxins (enterotoxin B), Chlamydia psittaci (psittacosis), water safety threats (e.g., Vibrio cholerae, Crytosporidium parvum), Typhus fever (Richettsia powazekii), and viral encephalitis (alphaviruses, e.g., Venezuelan equine encephalitis; eastern equine encephalitis; western equine encephalitis); all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

Administration of the vectors of the invention to deliver immunogens against the variable region of the T cells elicit an immune response including CTLs to eliminate those T cells. In RA, several specific variable regions of TCRs which are involved in the disease have been characterized. These TCRs include V-3, V-14, V-17 and Vα-17. Thus, delivery of a nucleic acid sequence that encodes at least one of these polypeptides will elicit an immune response that will target T cells involved in RA. In MS, several specific variable regions of TCRs which are involved in the disease have been characterized. These TCRs include V-7 and Vα-10. Thus, delivery of a nucleic acid sequence that encodes at least one of these polypeptides will elicit an immune response that will target T cells involved in MS. In scleroderma, several specific variable regions of TCRs which are involved in the disease have been characterized. These TCRs include V-6, V-8, V-14 and Vα-16, Vα-3C, Vα-7, Vα-14, Vα-15, Vα-16, Vα-28 and Vα-12. Thus, delivery of a recombinant simian adenovirus that encodes at least one of these polypeptides will elicit an immune response that will target T cells involved in scleroderma.

Further, desirable immunogens include those directed to eliciting a therapeutic or prophylactic anti-cancer effect in a vertebrate host, such as, without limitation, those utilizing a cancer antigen or tumor-associated antigen including, without limitation, prostate specific antigen, carcino-embryonic antigen, MUC-1, Her2, CA-125 and MAGE-3.

The examples provided below specifically illustrate the advantages of the methods and compositions of the invention utilizing a recombinant simian adenoviral vector from which an immunogenic peptide of rabies (glycoprotein G) or human immunodeficiency virus-1 (a modified gag protein) is expressed. Another desirable embodiment utilizes a simian adenovirus carrying an immunogenic peptide from human papilloma virus (e.g., E6, E7 and/or L1 (Seedorf, K. et al, Virol., 145:181-185 (1985))]. However, the invention is not limited to these sources of immunogens.

C. Ad-Mediated Delivery Methods

The therapeutic levels, or levels of immunity, of the selected gene can be monitored to determine the need, if any, for boosters. Following an assessment of CD8+ T cell response, or optionally, antibody titers, in the serum, optional booster immunizations may be desired. Optionally, the recombinant simian adenoviral vectors of the invention may be delivered in a single administration or in various combination regimens, e.g., in combination with a regimen or course of treatment involving other active ingredients or in a prime-boost regimen. A variety of such regimens has been described in the art and may be readily selected.

For example, prime-boost regimens may involve the administration of a DNA (e.g., plasmid) based vector to prime the immune system to second, booster, administration with a traditional antigen, such as a protein or a recombinant virus carrying the sequences encoding such an antigen. See, e.g., WO 00/11140, published Mar. 2, 2000, incorporated by reference. Alternatively, an immunization regimen may involve the administration of a recombinant simian adenoviral vector of the invention to boost the immune response to a vector (either viral or DNA-based) carrying an antigen, or a protein. In still another alternative, an immunization regimen involves administration of a protein followed by booster with a vector encoding the antigen.

In one embodiment, the invention provides a method of priming and boosting an immune response to a selected antigen by delivering a plasmid DNA vector carrying said antigen, followed by boosting with a recombinant simian adenoviral vector of the invention. In one embodiment, the prime-boost regimen involves the expression of multiproteins from the prime and/or the boost vehicle. See, e.g., R. R. Amara, Science, 292:69-74 (6 Apr. 2001) which describes a multiprotein regimen for expression of protein subunits useful for generating an immune response against HIV and SIV. For example, a DNA prime may deliver the Gag, Pol, Vif, VPX and Vpr and Env, Tat, and Rev from a single transcript. Alternatively, the SIV Gag, Pol and HIV-1 Env is delivered in a recombinant adenovirus construct of the invention. Still other regimens are described in WO 99/16884 and WO 01/54719.

However, the prime-boost regimens are not limited to immunization for HIV or to delivery of these antigens. For example, priming may involve delivering with a first chimp vector of the invention followed by boosting with a second chimp vector, or with a composition containing the antigen itself in protein form. In one example, the prime-boost regimen can provide a protective immune response to the virus, bacteria or other organism from which the antigen is derived. In another desired embodiment, the prime-boost regimen provides a therapeutic effect that can be measured using convention assays for detection of the presence of the condition for which therapy is being administered.

The priming composition may be administered at various sites in the body in a dose dependent manner, which depends on the antigen to which the desired immune response is being targeted. The invention is not limited to the amount or situs of injection(s) or to the pharmaceutical carrier. Rather, the regimen may involve a priming and/or boosting step, each of which may include a single dose or dosage that is administered hourly, daily, weekly or monthly, or yearly. As an example, the mammals may receive one or two doses containing between about 10 μg to about 50 μg of plasmid in carrier. A desirable amount of a DNA composition ranges between about 1 μg to about 10,000 μg of the DNA vector. Dosages may vary from about 1 μg to 1000 μg DNA per kg of subject body weight. The amount or site of delivery is desirably selected based upon the identity and condition of the mammal.

The dosage unit of the vector suitable for delivery of the antigen to the mammal is described herein. The vector is prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. The compositions of the invention may be administered to a mammal according to the routes described above, in a sustained release formulation using a biodegradable biocompatible polymer, or by on-site delivery using micelles, gels and liposomes. Optionally, the priming step of this invention also includes administering with the priming composition, a suitable amount of an adjuvant, such as are defined herein.

Preferably, a boosting composition is administered about 2 to about 27 weeks after administering the priming composition to the mammalian subject. The administration of the boosting composition is accomplished using an effective amount of a boosting composition containing or capable of delivering the same antigen as administered by the priming DNA vaccine. The boosting composition may be composed of a recombinant viral vector derived from the same viral source (e.g., adenoviral sequences of the invention) or from another source. Alternatively, the “boosting composition” can be a composition containing the same antigen as encoded in the priming DNA vaccine, but in the form of a protein or peptide, which composition induces an immune response in the host. In another embodiment, the boosting composition contains a DNA sequence encoding the antigen under the control of a regulatory sequence directing its expression in a mammalian cell, e.g., vectors such as well-known bacterial or viral vectors. The primary requirements of the boosting composition are that the antigen of the composition is the same antigen, or a cross-reactive antigen, as that encoded by the priming composition.

In another embodiment, the simian adenoviral vectors of the invention are also well suited for use in a variety of other immunization and therapeutic regimens. Such regimens may involve delivery of simian adenoviral vectors of the invention simultaneously or sequentially with Ad vectors of different serotype capsids, regimens in which adenoviral vectors of the invention are delivered simultaneously or sequentially with non-Ad vectors, regimens in which the adenoviral vectors of the invention are delivered simultaneously or sequentially with proteins, peptides, and/or other biologically useful therapeutic or immunogenic compounds. Such uses will be readily apparent to one of skill in the art.

Dosages of the viral vector will depend primarily on factors such as the condition being treated, the age, weight and health of the patient, and may thus vary among mammalian (including human) patients. Advantageously, the unexpected potency of the recombinant simian (e.g., chimpanzee) adenoviruses of the invention permits the use significantly lower amount of the recombinant chimpanzee adenovirus to provide an effective amount to induce the desired immunogenic effect (e.g., induction of a predetermined level of CD8+ T cells). For example, an effective dose of the recombinant simian adenovirus may be provided by 10⁴ pfu and 10⁶ pfu of the chimpanzee adenovirus. However, higher doses may be readily selected, e.g., depending upon the selected route of delivery. For example, the viral vector may be delivered in an amount which ranges from about 100 μL to about 100 ml, and more preferably, about 1 mL to about 10 mL, of carrier solution containing concentrations of ranging from about 1×10⁴ plaque forming units (pfu) to about 1×10¹³ pfu virus/ml, and about 1×10⁹ to about 1×10¹¹ pfu/ml virus, based upon an 80 kg adult weight. A preferred dosage is estimated to be about 50 ml saline solution at 2×10¹⁰ pfu/ml. A preferred dose is from about 1 to about 10 ml carrier (e.g., saline solution) at the above concentrations. The therapeutic levels, or levels of immunity, of the selected gene can be monitored to determine the need, if any, for boosters. Following an assessment of CD8+ T cell response, or optionally, antibody titers, in the serum, optional booster immunizations may be desired. Optionally, the recombinant simian adenoviruses may be delivered using a prime-boost regimen. A variety of such regimens has been described in the art and may be readily selected. One particularly desirable method is described in WO 00/11140, published Mar. 2, 2000, which is incorporated by reference.

In one desirable embodiment, the invention provides a method of preferentially inducing a CD8+ T cell response to a human immunodeficiency virus in a subject by delivering a recombinant simian adenovirus comprising a modified gag protein. The modified gag protein illustrated in the examples below has been optimized, e.g., as described in U.S. Pat. No. 5,972,596. The coding and protein sequences are reproduced herein in SEQ ID NO:45 and SEQ ID NO:46. See, also, G. Meyers et al., Eds. Human retroviruses and AIDS. A compilation and analysis of nucleic acid and amino acid sequences (Los Alamos National Laboratory, Los Alamos, N. Mex. 1991). However, any of a variety of methods to improve expression of the gag protein, or any other selected immunogen or antigen as described herein, are known to those of skill in the art and may be utilized, e.g., humanization of the HIV-1 gag codon sequences, removal of the HIV-1 gag splice site, insertion of additional leader sequences upstream of the HIV-1 gag codon sequences, insertion of a Kozak sequence upstream of the HIV-1 gag codon sequences. The selection of the optimization method is not a limitation of the present invention. Alternatively, the method of the invention may be used to deliver a recombinant simian adenovirus carrying an HIV envelope protein, or an HIV pol, to the subject. One desirable HIV envelope protein is HIV glycoprotein 120 for which sequences are available from GenBank. However, other suitable viral envelope proteins may be utilized. The sequence for HIV-1 pol is known, as are a variety of modified pol sequences. See, e.g., U.S. Pat. No. 5,972,596 and R. Scheider et al, J. Virol, 71(7):4892-4903 (July 1997). In another desirable embodiment, the invention provides a method of preferentially inducing a CD8+ T cell response to a tumor-associated protein specific for a selected malignancy by delivering a recombinant simian adenovirus comprising a tumor-associated protein to the subject. Such a protein includes cellular oncogenes such as mutated ras or p53.

Still another desirable embodiment involves delivering a recombinant simian adenovirus comprising a protein derived from human papilloma virus for prevention of infection therewith and for treatment and prophylaxis of associated conditions.

The following examples are provided to illustrate the invention and do not limit the scope thereof. One skilled in the art will appreciate that although specific reagents and conditions are outlined in the following examples, modifications can be made that are meant to be encompassed by the spirit and scope of the invention.

### Example 1

### Creation of an E1 Deleted Vector Based on Chimpanzee Adenovirus C68

A replication defective version of C68 was isolated for use in gene transfer. The classic strategy of creating a recombinant with E1 deleted, by homologous recombination in an E1 expressing cell line was pursued. The first step was creation of a plasmid containing m.u. 0 through 1.3 followed by addition of a minigene expressing enhanced green fluorescent protein (GFP) from a CMV promoter and C68 sequence spanning 9-16.7 m.u. This linearized plasmid was cotransfected into an E1 expressing cell line with Ssp I-digested C68 plasmid (SspI cuts at 3.6 m.u. leaving 4644 bp for homologous recombination). Experiments were initially conducted with 293 cells which harbor E1 from human Ad5 with the hope that this would suffice for transcomplementation. Indeed, plaques formed which represented the desired recombinant. The resulting vector was called C68-CMV-GFP.

The strategy for generating recombinants was modified to enable efficient and rapid isolation of recombinants. First, the alkaline phosphatase DNA in the initial shuttle vector was replaced with a prokaryotic GFP gene driven by the prokaryotic promoter from lacZ. This allowed efficient screening of bacterial transformations when attempting to incorporate a desired eukaryotic RNA pol II transcriptional unit into the shuttle vector. The resulting transformation can be screened for expression of GFP; white colonies are recombinants while green colonies are residual parental plasmid.

A green-white selection has been used to screen the products of cotransfection for the isolation of human Ad5 recombinants (A. R. Davis et al, Gene Thera., 5:1148-1152 (1998)); this was adapted to the C68 system. The initial shuttle vector was revised to include extended 3′ sequences from 9 to 26 MU. This vector was cotransfected with viral DNA from the original C68-CMV-GFP isolate that had been restricted with Xba I, which cuts at MU 16.5 allowing for 9.5 Kb of overlap for homologous recombination. The resulting plaques were screened under a phase contrast fluorescent microscope for non-fluorescing isolates that represent the desired recombinants. This greatly simplified screening in comparison to the standard methods based on structure or transgene expression.

A. Shuttle Plasmid

To construct a plasmid shuttle vector for creation of recombinant C68 virus, the plasmid pSP72 (Promega, Madison, Wis.) was modified by digestion with Bgl II followed by filling-in of the ends with Klenow enzyme (Boehringer Mannheim, Indianapolis, Ind.) and ligation with a synthetic 12 bp Pac I linker (New England Biolabs, Beverly, Mass.) to yield pSP72-Pac. A 456 bp Pac I/SnaB I fragment spanning map unit (m.u. or MU) 0-1.3 of the C68 genome was isolated from the pNEB-BamE plasmid containing BamHI E fragment of the C68 genome and cloned into Pac I and EcoR V treated pSP72-Pac to yield pSP-C68-MU 0-1.3. A minigene cassette consisting of the cytomegalovirus early promoter driving lacZ with a SV40 poly A signal was separated from pCMVβ (Clontech, Palo Alto, Calif.) as a 4.5 kb EcoRI/SalI fragment and ligated to pSP-C68-MU 0-1.3 restricted with the same set of enzymes, resulting in pSP-C68-MU 0-1.3-CMVLacZ.

For the initial step in the isolation of the 9-16.7 MU region of C68, both pGEM-3Z (Promega, Madison, Mich.) and pBS-C68-BamF were double-digested with BamHI and Sph I enzymes. Then the 293 bp fragment from pBS-C68-BamF was ligated with pGEM-3Z backbone to form pGEM-C68-MU 9-9.8. A 2.4 kb fragment including the C68 MU 9.8-16.7 was obtained from the pBS-C68 BamHB clone after XbaI digestion, filling in reaction and subsequent BamHI treatment and cloned into BamHI/SmaI double digested pGEM-C68-MU 9-9.8 to generate pGEM-C68-MU 9-16.7. The C68 9-16.7 m.u. region was isolated from pGEM-C68-MU 9-16.7 bp digestion with EcoRI, filling in of the ends with Klenow enzyme (Boehringer Mannheim, Indianapolis, Ind.), ligation of a synthetic 12 bp HindIII linker (NEB) and then digestion with HindIII. This 2.7 kb fragment spanning the C68 MU 9-16.7 was cloned into the HindIII site of pSP-C68-MU 0-1.3-CMVlacZ to form the final shuttle plasmid pC68-CMV-LacZ. In addition, an 820 bp alkaline phosphatase (AP) cDNA fragment was isolated from pAdCMVALP (K. J. Fisher, et al., J. Virol., 70:520-532 (1996)) and exchanged for lacZ at Not I sites of pC68-CMV-lacZ, resulting in pC68-CMV-AP.

B. Construction of Recombinant Virus

To create the E1-deleted recombinant C68-CMVEGFP vector, a pC68-CMV-EGFP shuttle plasmid was first constructed by replacing the lacZ transgene in pC68-CMV-lacZ with the enhanced green fluorescent protein (EGFP) gene. The replacement cloning process was carried out as the follows. An additional NotI restriction site was introduced into the 5′ end of the EGFP coding sequence in the pEGFP-1 (Clontech, Palo Alto, Calif.) by BamHI digestion, filling in reaction and ligation of a 8 bp synthetic NotI linker (NEB). After NotI restriction of both constructs, the EGFP sequence was isolated from the modified pEGFP-1 and used to replace the lacZ gene in the pC68-CMV-lacZ. The pC68-CMVEGFP construct (3 μg) was co-transfected with Ssp I-digested C68 genomic DNA (1 μg) into 293 cells for homologous recombination as previously described (G. Gao, et al, J. Virol, 70:8934-8943 (1996)). Green plaques visualized by fluorescent microscopy were isolated for 2 rounds of plaque purification, expansion and purification by CsCl gradient sedimentation (G. Gao, et al, cited above).

In an attempt to apply the convenient green/white selection process (A. R. Davis, et al., Gene Thera., 5:1148-1152 (1998)) to construction of recombinant C68 vectors, a 7.2 kb fragment spanning 9 to 36 MU was isolated from the pBSC68-BamB plasmid by treatment with AgeI and BsiwI restriction endonucleases and cloned into Asp718 and AgeI sites of pC68-CMV-AP shuttle plasmid, resulting in a new plasmid called pC68CMV-AP-MU36. A further modification was made to remove 26 to 36 m.u. from pC68CMV-AP-MU36 bp Eco47III and NruI digestions. The new shuttle plasmid called pC68CMV-AP-MU26 has a shorter region for homologous recombination (i.e., 16.7-26 MU) 3′ to the minigene. To make a recombinant C68 vector, alkaline phosphatase (AP) is replaced with the gene of interest. The resulting pC68CMV-Nugene-MU26 construct is co-transfected with Xba I (16.5 MU) restricted C68-CMVGFP viral DNA into 293 cells, followed by top agar overlay. The recombinant virus plaques (white) are generated through the homologous recombination in the region of 16.7-26 MU which is shared between pC68CMV-Nugene construct and C68 viral backbone; the recombinants which form white plaques are selected from green plaques of uncut C68-CMVGFP virus.

The green/white selection mechanism was also introduced to the process of cloning of the gene of interest into the pC68 shuttle plasmid. The AP gene in both pC68CMV-AP-MU36 and pC68CMV-AP-MU26 was replaced with a cassette of prokaryotic GFP gene driven by the lacZ promoter isolated from pGFPMU31 (Clontech, Palo Alto, Calif.). Thus, white colonies of bacterial transformants will contain the recombinant plasmid. This green/white selection process for bacterial colonies circumvented the need for making and characterizing large numbers of minipreped DNAs and so further enhanced the efficiency in creating recombinant C68 vectors.

### Example 2

### Expression of Antigen (Gag Secretion) in TK⁻ Cells Infected with Simian Adenovirus Vaccine Constructs

Adenoviral recombinants of the chimpanzee strain 68 (Adchimp68) and the human strain 5 (Adhu5) carrying a nucleotide sequence modified version of a truncated form of the gag gene of HIV-1 Glade B were constructed as described (in Example 1 and Z. Q. Xiang, et al, Virol. 219, 200 (1996)). Transcripts of structural proteins of HIV-1, including gag, contain genetic instability elements, which require the presence of rev protein for nuclear export and efficient expression in the cytoplasm (S. Schwartz et al., J Virol 66, 7176 (1992); S. Schwartz, et al, J Virol 66, 150-159 (1992); G. Nasioulas et al., J Virol 68, 2986 (1994)). Adenoviruses rely on nuclear transcription and thus require rev for expression of HIV-1 proteins. To circumvent Rev dependency, a codon-modified sequence of gag from which genetic instability elements had been removed by site directed mutagenesis (R. Schneider et al., J Virol 71, 4892 (1997); S. Schwartz et al., J Virol 66, 7176 (1992); S. Schwartz, et al., J Virol 66, 150 (1992)) was inserted into the adenoviral vector. The introduced gene encodes the truncated p37gag protein (p17 and p24 regions). The truncated gag protein does not form viral particles and is partially secreted into the supernatant of transfected human cells (R. Schneider et al., J Virol 71, 4892 (1997)). The mutated gag constructs have been used in vaccination experiments and result in the generation of cellular and humoral immune responses in mice and primates (J. T. Qiu et al. J Virol 73, 9145. (1999)).

The Adchimp68 and the Adhu5 recombinants were both generated and propagated on 293 cells transfected with the E1 of adenovirus of the human strain 5. The inventors have found that this heterologous E1 is suitable for complementing the E1-deleted Adchimp68 virus recombinants thus reducing the risk of recombination and reversion to replication-competent wild-type virus.

The presence of gag protein in the TK⁻ culture supernatants was analyzed by Western blotting using mouse monoclonal antibodies to gag. TK⁻ cells (1×10⁶) were infected for 48 hrs with Adhu5gag37 or Adchimp68gag37 virus (10 pfu per cell). Additional TK⁻ cells were infected with an Adhu5 or an Adchimp68 construct expressing the glycoprotein of rabies virus. Proteins in the culture supernatant were separated on a 12% denaturing polyacrylamide gel and transferred by electroblotting to a PVDF membrane. The blot was stained with the monoclonal antibody 183-H12-5C to HIV-1 p24 (B. Chelsebro, et al. J. Virol. 66: 6547 (1992).

The two adenoviral recombinant clones (Adhu5gag37, Adchimp68gag37) carrying this modified sequence of gag expressed the transgene product at comparable levels as shown by Western Blot analysis. A protein of the expected size (37 kDa) that bound to a monoclonal antibody to gag of HIV-1 was detected in the supernatants of TK⁻ cells infected with 10 plaque forming units (pfu) of either adenovirus gag recombinant. Control cells infected with Adhu5 or Adchimp68 recombinant expressing glycoprotein of rabies virus (Adhu5rab.gp and Adchimp68.gp) failed to produce this protein.

### Example 3

### Induction of CD8⁺ T Cell Responses to Gag in Mammals by Simian Adenovirus

The following experiment demonstrates that the splenocytes of mice injected intramuscularly (i.m.) with either the Adhu5gag37 or the Adchimp68gag37 recombinant responded to an immunodominant epitope (B. Doe and C. M. Walker, AIDS 10, 793 (1996)) of the gag protein by cytokine, i.e., interferon (IFN)-γ, release, as well as by target cell lysis.

A. Cytokine Release Assay

Groups of 3 Balb/c mice were immunized i.m. with 2×10⁵, 2×10⁶ or 2×10⁷ pfu of Adchimp68gag37 virus, 2×10⁶ pfu of Adhu5L1 virus (H. C. J. Ertl, et al., J. Virol, 63:2885 (1989)), 2×10⁶ pfu of Adhu5 gag37 virus or 2×10⁷ pfu of VVgag virus. Splenocytes were tested for CD8⁺ T cell response to gag 10 days later. To assay cytokine (IFN-γ) production, splenocytes (1×10⁶/sample) were cultured for 5 hrs at 37° C. with 3 μg/ml of the AMQMLKETI peptide (SEQ ID NO:40) which carries the immunodominant CD8⁺ T cell epitope for the H-2^{d} haplotype and 1 μg/ml Brefeldin A (GolgiPlug, PharMingen, San Diego, Calif.) in 96 well round-bottom microtiter plate wells in Dulbeccos modified Eagles medium (DMEM) supplemented with 2% fetal bovine serum (FBS) and 10⁻⁶M 2-mercaptoethanol. Cells were washed with PBS and incubated for 30 min at 4° C. with a FITC labeled antibody to murine CD8. Cells were washed and permeabilized in 1× Cytofix/Cytoperm (PharMingen) for 20 min at 4° C. Cells were washed 3 times with Perm/Wash (PharMingen) and incubated in the same buffer for 30 min at 4° C. with a PE labeled antibody to murine IFN-γ. After washing, cells were examined by two-color flow cytometry and data were analyzed by WinmDi software. The number in the right hand corner shows the percent of CD8⁺ cells over all CD8⁺ T cells that stained positive for INF-γ.

Seven to ten days after a single immunization, a sizable fraction of the entire splenic CD8⁺ T cell population produced IFN-γ in response to the gag peptide. Primary splenocytes assayed without further in vitro expansion lysed H-2 compatible target cells pre-treated with the gag peptide. Gag-specific CD8⁺ T cell activity was superior upon immunization with the Adchimp68 construct, which achieved CD8⁺ T cell frequencies to gag of ˜16-19% of the entire splenic CD8⁺ cell population. The response was dose-dependent as shown for the Adchimp68gag37 virus where a low dose of 2×10⁵ pfu of virus still elicited frequencies of nearly 10%. The Adhu5gag37 recombinant induced optimal frequencies of ˜9% at 2×10⁶ pfu. These frequencies were not significantly enhanced upon increasing the dose of this vaccine (data not shown). A vaccinia virus recombinant expressing full-length gag (VVgag, designated vDK1 in S. Chacarabarti et al. Mol. Cell. Biol. 5, 3403 (1985)) stimulated far lower frequencies of CD8⁺ T cells by intracellular cytokine staining.

B. Lysis of target cells.

Splenocytes from mice immunized 10 days previously with a single dose of the adenoviral recombinants as described in A or two doses of the VVgag recombinant the first given i.m. followed 2 weeks later by an intraperitoneal injection were tested in a 5 hr ⁵¹Cr-release assay at varied effector to target cell ratios on 1×10⁴ P815 cells that had been treated for 16-24 hrs at room temperature with either the peptide to gag (filled squares) or the control peptide 31D (X) delineated from the sequence of the rabies virus nucleoprotein (H. C. J. Ertl et al., J. Virol. 63: 2885 (1989)). Two immunizations with the VVgag vaccine were required to induce detectable T cell-mediated gag-specific primary cytolysis.

C. Kinetics of the CD8⁺ T cell response to gag

Groups of 4 Balb/c mice were immunized with 5×10⁶ pfu of Adhu5gag37 or Adchimp68gag37 virus. Splenocytes were harvested 6-12 days later and tested for IFN-γ production and target cell lysis as described above. The kinetics of the CD8⁺ T cell response to gag elicited by the two adenovirus recombinants differed. The response to gag presented by the Adhu5gag37 virus peaked 2-4 days earlier than the CD8⁺ T cell response to the Adchimp68gag37 recombinant.

### Example 4

### Effect of Prior Exposure to Human Adenovirus on Simian Adenovirus Vaccine

To study the impact of previous exposure to the common human strain 5 of adenovirus, mice were immunized with a single dose of an Adhu5recombinant expressing an irrelevant antigen (human papilloma virus L1). Two weeks later mice were vaccinated either with the Adhu5gag37 or the Adchimp68gag37 vaccine.

More particularly, mice were immunized i.m. with 10⁸ pfu of the Adhu5L1 vaccine. Two weeks later Adhu5-immune as well as naïve mice were injected with 2×10⁶ or 2×10⁷ pfu of Adhu5gag37 or Adchimp68gag37 recombinants (4-5 mice per group). Additional groups of Adhu5L1 immune or naïve mice were immunized with 2×10⁶ pfu of the Adhu5gag37 or the Adchimp68gag37 virus. Nine days later mice were injected intraperitoneally with 10⁶ pfu of a vaccinia virus recombinant expressing full-length gag. Mice were sacrificed five days after the vaccinia virus injection.

Mice pre-immune to Adhu5 virus failed to respond to gag after vaccination with the Adhu5gag37 vaccine. They showed frequencies of CD8⁺ gag-specific T cells similar to those seen in control mice and correspondingly, their splenocytes failed to lyse gag expressing target cells. In contrast, the CD8⁺ T cell response to gag was only slightly decreased in Adhu5-immune mice vaccinated with the Adchimp68gag37 construct. Frequencies of CD8⁺ T cells to gag were reduced by only ˜30% and the cytolytic activity of splenocytes was lowered by ˜50% comparing different effector to target cell ratios.

Thus, both adenoviral recombinants induce frequencies of CD8⁺ T cells to gag, surpassing those elicited by previously described vaccines such as naked DNA or poxvirus recombinants (S. Schwartz, et al, J Virol 66, 150-159 (1992)). Frequencies were also higher than those generally seen in chronically infected individuals (D. H. Barouch et al. Proc. Natl. Acad. Sci. USA. 97, 4192 (2000); T. U. Vogel et al. J. Immunol. 164, 4968 (2000); P. A. Goepfert et al. J. Virol. 74, 10249 (2000); C. R. Rinaldo Jr. et al. AIDS Res. & Hum. Retr. 14:1423 (1998)). These results emphasize the potency of adenoviral recombinant vaccines.

### Example 5

### Effect of Priming and Boosting of CD8⁺ T Cells to Antigen

Primary splenocycles from the cells of naïve or Adhu5-immune mice immunized with 2×10⁷ pfu of Adhu5gag37 or Adchimp68gag37 virus were compared with splenocytes from naïve or Adhu5 immune mice vaccinated with 2×10⁶ pfu of Adhu5gag37 or Adchimp68gag37 virus and then boosted with 10⁶ pfu of VVgag virus. Splenocytes were analyzed 5 days later for CD8 and intracellular IFN-γ. These assays were performed essentially as described above, with the exception that there was no further in vitro culture for lysis of P815 cells treated with the gag peptide or the control peptide 31D in a 5 hr ⁵¹Cr-release assay.

Priming or booster immunization with a heterologous vaccine construct, the VVgag recombinant, failed to restore the CD8 T cell response to gag presented by the Adhu5 recombinant vaccine. Although Adhu5 vaccinated animals boosted with Adhu5gagp37 and VVgag showed as much as 7.1% of splenic CD8⁺ T cells to produce IFN-γ-in response to the gag these CD8⁺ T cells totally lacked cytolytic activity against gag-presenting target cells. These results indicate that pre-exposure to the antigens of the vaccine carrier had not only a quantitative but also a qualitative influence on the CD8⁺ T cell response to the transgene product of the adenoviral recombinant. The CD8⁺ T cell response to gag in Adhu5 immune mice vaccinated with Adchimp68gag37 showed a booster effect upon VVgag immunization similar to that seen in naïve mice.

Frequencies of CD8⁺ T cells to gag as well as primary target cell lysis could be augmented further by priming (not shown) or boosting with a heterologous vaccine carrier, such as the VVgag recombinant. After i.m. priming with the adenoviral recombinants followed 9 days later by an i.p. booster immunization with the VVgag, CD8⁺ gag-specific T cells analyzed 5 days post-prime comprised ˜40% of the entire splenic CD8⁺ cell-population.

Pre-existing immunity to Adhu5 severely reduced the efficacy of the Adhu5gag37 vaccine but only slightly impaired the CD8⁺ T cell response to the Adchimp68gag37 virus. It was previously reported that mice immunized to Adhu5 virus developed a reduced B cell response to vaccination with an Adhu5 vaccine to rabies virus. Increasing the dose of the vaccine or using a DNA vaccine expressing the same antigen of rabies virus could readily circumvent the dampening effect of the pre-exposure to Adhu5 virus (Z. Q. Xiang, et al., J. Immunol. 162, 6716 (1999)).

In contrast, the CD8⁺ T cell response to gag presented by the Adhu5 recombinant vaccine was abolished in Adhu5 immune mice and could only partially be restored by additional immunizations with a heterologous vaccine to gag. This may indicate induction of CD8⁺ T cells to be more susceptible to interference by circulating virus neutralizing antibodies as compared to stimulation of B cells.

### Example 6

### Production of Recombinant Adenoviruses Containing Rabies Glycoprotein

Adenoviruses of the human serotypes 2, 4, 5, 7, 12 and the chimpanzee serotype 68 were propagated and titrated on human 293 cells. The recombinant adenoviruses based on the human serotype 5 expressing the glycoprotein of the ERA serotype of rabies virus or the L1 protein of the human papilloma virus (HPV)-16 have been described previously (Z. Q. Xiang, et al, Virology 219: 220-227 (1996); D. W. Kowalcyk, et al, (2001) Vaccine regimen for prevention of sexually transmitted infections with human papillomavirus type 16. Vaccine). An expression system based on adenoviruses of the chimpanzee serotype 68 was developed as described in Example 1.

Adenoviruses were propagated on E1 (derived from the human serotype 5)-transfected 293 cells (F. L. Graham, et. al., J. Gen. Virol. 36: 59-74 (1977)). Viruses were harvested by freeze thawing of the cells. For some experiments virus was purified by CsCl gradient purification. For other experiments, cleared supernatant of the infected cells necrotized through three rounds of freeze thawing was used. Viruses were titrated on 293 cells to determine plaque forming units (pfu).

The adenoviral recombinant of the chimpanzee 68 serotype expressing the rabies virus glycoprotein, termed Adchimp68rab.gp was generated in 293 cells transfected with E1 of adenovirus human serotype 5 as described in detail in this example. Viral clones were initially screened by indirect immunofluorescence with the monoclonal antibody 509-6 to a conformation-dependent epitope of the rabies virus glycoprotein. Upon selection of a stable adenoviral subclone, expression of full-length rabies virus glycoprotein by the Ad.chimp68rab.gp virus in infected TK⁻ cells was confirmed by immunoprecipitation, as described in the following example.

### Example 7

### Expression of the Transgene Product by the Adenoviral Recombinants

This example shows that the Adhu5 virus achieved markedly higher levels of rabies virus glycoprotein expression in TK⁻ cells as compared to the Adchimp68 construct. Transcript levels for this transgene paralleled protein expression indicating that the difference was unrelated to differences in post-translational modifications. TK⁻ cells are CAR positive and rates of transduction by the viral serotypes should thus be comparable.

For use in these experiments, mammalian cells, i.e., baby hamster kidney (BHK)-21 cells, E1-transfected 293 cells and TK⁻ cells, were propagated in Dulbecco's modified Eagle's medium (DMEM) supplemented with glutamine, Na-pyrovate, non-essential amino acids, HEPES buffer, antibiotic and 10% fetal bovine serum (FBS).

A. Immunoprecipitation

TK⁻ cells (10⁶ per sample) were infected with 5 pfu per cell of either the rabies virus glycoprotein expressing adenoviral recombinants or control constructs expressing an unrelated viral antigen. After 48 hrs cells were washed twice with sterile phosphate buffered saline (PBS) and then incubated for 90 min in serum-free medium prior to the addition of 20 μl of ³⁵5-labeled cystein and methionin (Promix, NEN, Boston, Mass.). After 4 hrs incubation, cells were washed with PBS and then treated for 20 min with 1 ml of protease inhibitors containing RIPA buffer. Cells and cell debris were removed from the wells, vortexed briefly and centrifuged for 2 min at 12.000 rpm. The supernatant was incubated for 90 min at 4° C. with 15 μl/ml of ascitic fluid containing the 509-6 monoclonal antibody to the rabies virus glycoprotein. Protein Sepharose G was added at 75 μl per sample and incubated at 4° C. under mild agitation for 30 min. The samples were pelleted by centrifugation and washed 4 times with RIPA buffer. The pellets were resuspended in 80 μl of loading buffer, boiled for 4 min. Samples (20 p. 1) were then separated over a 12% SDS-polyacrylamide (PAGE) gel in comparison to a molecular weight standard. Gels were dried onto filter papers which were exposed for 48 hrs to a Kodak Scientific Imaging Film (X-Omat Blue XB-1).

The Adchimp68rab.gp recombinant expressed a protein of the expected size that bound to the 509-6 antibody. The precipitate of TK⁻ cells infected with the Adhu5rab.gp virus showed a band of the identical size that was absent in lysates from cells infected with adenoviral recombinants expressing an unrelated transgene product. Expression of the rabies virus glycoprotein was more pronounced in cells infected with the Adhu5rab.gp construct. The difference in expression of the transgene product may reflect pre-translational events such as differences in viral uptake, rate of transcription or transcript stability. Alternatively, translational or post-translational differences such as distinct side chain modifications may result in quantitative differences in serologically detectable protein.

To further distinguish between these two possibilities, the total RNA was isolated from TK⁻ cells infected with either of the adenoviral recombinants. Reverse transcribed mRNA to the rabies virus glycoprotein and a housekeeping gene was amplified by real-time PCR performed as described in part B.

B. Real Time Reverse Transcription Polymerase Chain Reaction (PCR)

Confluent monolayers of TK⁻ cells were infected in duplicate samples with 10 pfu of either of the adenoviral recombinants. Cells were isolated 24 hrs later and RNA was extracted with the TR1 reagent according to the manufacturer's instructions (Mol. Res. Center, Cincinnati, Ohio). The RNA was treated with RNAse-free DNAse, purified by phenol extraction and adjusted to 50 ng of RNA per sample. The RNA was reverse transcribed and amplified with the Light Cycler-RNA amplification kit SYBR green (Roche, Mannheim, Germany; Z. He, et al, Virology 270: 146-1617 (2000)) using primers for the rabies virus glycoprotein (SEQ ID NO:41: 5′ AA GCA TTT CCG CCC AAC AC; SEQ ID NO:42: 3′ GGT TAG TGG AGC AGT AGG TAG A) and the housekeeping gene glutaraldehyde-3-phosphate dehydrogenase (GAPDH) (SEQ ID NO: 43: 5′ GGT GAA GGT CGG TGT GAA CGG ATT T; SEQ ID NO:44: 3′ AAT GCC AAA GTT GTC ATG GAT GAC C).

The data in Table 1 provides the results. The data show the mean values for duplicate measurements±SD.

TABLE 1

| | Relative Transcript Quantity | | |
|---|---|---|---|
| | | | Ratio |
| Source of RNA | GAPDH | rab.gp | (GAPDH/rab.gp) |
| TK⁻, Adhu5rab.gp | 3.2 ± .2 | 3494 ± 18 | 1082 |
| TK⁻, | 0.52 ± .01111 | 64 ± 6 | 64 |
| Adchimp68rab.gp | | | |

As shown by this data, the transgene transcripts adjusted to those of the housekeeping gene showed a quantitative difference comparable to that of serologically detectable protein.

In data not provided in this example, two other Adchimp68 recombinants expressing the green fluorescent protein and a codon-modified truncated gag protein of the human immunodeficiency virus-1 were compared to the Adhu5 recombinants expressing the same transgene products showed equivalent protein expression levels in TK⁻ cells. From this it has been concluded that the reduced expression of the rabies virus glycoprotein by the Adchimp68 virus reflects a difference neither in viral uptake nor in rate of transcription, which in both constructs is regulated by the same control elements.

### Example 8

### Immunization of Mice Using a Rabies Virus Antigen

The rabies virus-specific antibody response to the Ad.chimp68rab.gp virus was compared to that of the Adhu5rab.gp virus in inbred and outbred strains of mice. Mice were injected with serial dilutions of either of the recombinants given s.c. or i.n. Sera were harvested 14 days later and tested for antibodies to the rabies virus glycoprotein by an ELISA and a virus neutralization assay. Adenoviral recombinants expressing an unrelated transgene, i.e., the gag of HIV-1 (described in the Examples above) were used as controls. These recombinants failed to induce an antibody response to rabies virus detectable by either assay. A more detailed discussion of this study and the results follows.

Female 6-8 week old C3H/He and C57B1/6 mice were purchased from Jackson Laboratory, Bar Harbor Me. Outbred ICR mice were purchased from Charles River (Wilmington, Mass.).

Mice were injected with varied doses of the adenoviruses or the adenoviral recombinants given in 100 μl of saline subcutaneously (s.c.) or in 50 μl intranasally (i.n.). Mice were challenged with rabies virus of the CVS-11 strain given at 10 mean lethal doses (LD₅₀) intracerebrally (i.c.). Rabies virus of the Evelyn Rokitniki-Abelseth (ERA) and the Challenge Virus Standard (CVS)-11 strain were propagated on BHK-21 cells. ERA virus was purified over a sucrose gradient, inactivated by treatment with betapropionolactone and adjusted to a protein concentration of 0.1 mg/ml. CVS-11 virus was titrated on BHK-21 cells and by intracerebral injection of adult ICR mice (Z. Q. Xiang, Z. Q. & H. C. Ertl, J. Virol. Meth. 47: 103-16 (1994)). Upon challenge mice were checked every 24-48 hrs for at least 21 days. They were euthanized once they developed complete hindleg paralysis, which is indicative of terminal rabies virus encephalitis.

The serological assays included enzyme linked immunoadsorbant assay (ELISA), isotype profile of antibodies, and virus neutralization assays.

A. ELISA

Mice were bled a varied time intervals after immunization by retro-orbital puncture. Sera were prepared and tested for antibodies to rabies virus on plates coated with 0.1 μg/well of inactivated rabies virus. Sera were tested for antibodies to adenovirus on plates coated with 0.1 μg/well of purified E1-deleted adenovirus recombinants to GFP of the human serotype 5 or the chimpanzee serotype 68. ELISAs were performed basically as described before (Z. Q. Xiang, et al, Virology 219, 220-227 (1996)). Plates were coated over night. They were then blocked for 24 hrs with PBS containing 3% of bovine serum albumin (BSA). After washing, sera diluted in PBS-3% BSA were added for 60 min. After washing, a 1:100 dilution of alkaline phosphatase conjugated goat anti mouse Ig (Cappel) was added for 1 hr on ice. After washing, substrate was added for 20-30 min at room temperature. Optical density was read at 405 nm.

B. Isotypes of Antibodies

Isotypes of antibodies to rabies virus were determined by an ELISA on plates coated with inactivated ERA virus using the Calbiochem Hybridoma Subisotyping (LaJolla, Calif.) kit with some minor previously described modifications (Xiang, Virol, 1996, cited above).

The isotype profile of antibodies to also differed upon s.c. immunization but was comparable upon i.n. application of the two adenoviral vaccines. Both recombinants, upon delivery by either route of inoculation, elicited IgG2a antibodies to the antigen of rabies virus.

Both recombinants upon i.n. immunization and the Adhu5rab.gp vaccine upon s.c. administration induced a pronounced IgG1 response indicative of Th2 help, which was lacking in the response to the Ad.chimp68rab.gp construct given s.c.

C. Neutralizing Antibodies

Sera were tested for neutralizing antibodies to rabies virus of the CVS-11 strain, which is antigenically closely related to the ERA strain (Z. Q. Xiang, et al, Virology. 214: 398-404 (1996)). A WHO reference serum was used for comparison. Titers are expressed as International Units.

The Adchimp68rab.gp virus given s.c. induced a less potent B cell response to the transgene product as compared to the Adhu5rab.gp construct. The difference in magnitude of the antibody response, which was observed at all time points tested depended on the mouse strain and was less pronounced in outbred ICR than in inbred C3H/He mice. In contrast, upon in immunization both vaccines induced comparable titers of antibodies as determined by ELISA and by virus neutralization assay.

The pronounced Th1 response to the Adchimp68rab.gp recombinant upon s.c. immunization contrasting with the more balanced Th1/Th2 response upon injection of the Adhu5rab.gp argues for a difference in adjuvanticity. Upon application to the airways, the natural route of infection for Adhu5 virus and presumably for Adchimp68 viruses both recombinants induced antibody titers to the transgene product that were comparable in magnitude and in their isotype profile. This suggests that postulated differences in tropism and/or adjuvanticity are tissue dependent, i.e., lacking or less pronounced in the airways as compared to the subcutaneum.

### Example 9

### Preferential Induction of Cytotoxic T Cell Response with Recombinant Chimpanzee Adenovirus

Vaccine-induced protection to rabies virus correlates with virus-neutralizing antibodies (VNAs, F. L. Graham, et. al., J. Gen. Virol. 36, 59-74 (1977)). The studies with the rabies protein thus focused on stimulation of this arm of the immune system. Throughout all of the experiments, mice were immunized with the Adchimp68rab.gp virus and, in parallel, with the previously described Adrab.gp virus based on the human serotype 5. Within this application, this recombinant is referred to as Adhu5rab.gp virus.

Both adenoviral recombinants induced protection to challenge with rabies virus. C3H/He mice immunized with 5×10⁶ pfu of either of the adenoviral recombinants given s.c. remained disease-free when challenged 3 weeks later with 10 mean lethal doses (LD₅₀) of rabies virus of the CVS strain. This rabies virus strain is antigenically closely related to the ERA strain but is more virulent in rodents. At a lower vaccine dose of 5×10⁵ pfu, the Adhu5rab.gp virus still provided complete protection while a small percentage of Adchimp68rab.gp-immunized mice succumbed to the infection. Further reduction of the vaccine dose resulted in loss of efficacy of the Adchimp68rab.gp vaccine. Upon i.n. immunization, both vaccines provided complete protection if given at 5×10⁵ pfu. At a lower dose of 5×10⁴ pfu 50% of mice vaccinated with the Adhu5rab.gp vaccine developed progressive disease while those immunized with this dose of the Adchimp68rab.gp recombinant were protected. All of the mice immunized with adenoviral recombinants of either serotype expressing an unrelated antigen or with 5×10³ pfu of either of the adenoviral recombinants to the rabies virus glycoprotein developed a fatal rabies encephalitis.

### Example 10

### The Effect of Pre-Existing Immunity to Different Serotypes of Human Adenoviruses on the Antibody Response to Rabies Virus

To test if pre-exposure to any of the common serotypes of human adenoviruses (e.g., human serotype 2, 4, 5, 7 and 12) would inhibit the antibody response to the Adchimp68rab.gp vaccine, groups of C3H/He mice were immunized with 4×10⁸ pfu of replication-competent adenoviruses of the human serotypes 2, 4, 5, 7 or 12 or the chimpanzee serotype 68 (the latter serotype was E1-deleted). Two weeks later, mice were vaccinated s.c. with either Adhu5rab.gp or Adchimp68rab.gp virus. The Adhu5rab.gp recombinant was used at a dose of 2×10⁵ pfu per mouse, the Adchimp68rab.gp recombinant, which given s.c. only induces a marginal antibody response in C3H/He mice at such a low dose was injected at 2×10⁷ pfu per mouse. Sera were harvested 2 weeks later and tested for antibodies to the rabies virus glycoprotein by an ELISA. The rabies virus-specific response to Adhu5rab.gp was slightly superior in naïve mice to that elicited to the Adchimp68 virus. The response to Adhu5rab.gp virus was completely inhibited in Adhu5 pre-immune mice. Some reduction was also seen in mice pre-immune to adenovirus of the human serotypes 4, 2, 7 and 12. The response was not affected in mice that had been pre-exposed to the Adchimp68 virus. The response to the Adchimp68rab.gp virus was strongly inhibited in mice that were pre-immune to the homologous virus. Mice that had previously encountered adenovirus of the human serotype 2 showed a slight reduction of the antibody response to the rabies virus antigen presented by the Adchimp68 vaccine. Mice inoculated with any of the other serotypes of human adenoviruses developed antibody titers to rabies upon Adchimp68rab.gp virus that were either similar or increased in magnitude compared to those in mice that were naïve prior to vaccination. In particular, mice pre-immune to Adhu5 virus developed higher antibody titers upon vaccination with the Adchimp68rab.gp construct which might reflect the presence of cross-reactive T helper cells that promoted the B cell response to the transgene product.

To further determine if at equal vaccine doses the Adchimp68rab.gp vaccine induced superior antibody titers as compared to the Adhu5rab.gp virus in mice pre-immune to Adhu5 virus, a vaccine titration experiment was conducted. Groups of C3H/He mice were immunized s.c. with 4×10⁸ pfu of an E1-deleted adenoviral recombinant to the L1 antigen of HPV-16. Mice were vaccinated 2 weeks later with either Adhu5rab.gp or Adchimp68rab.gp virus given s.c. at varied doses. Mice were bled 2 weeks later and serum antibody titers to rabies virus were determined by an ELISA (not shown) and a virus neutralization assay. Neither assay showed a significant reduction for the antibody response to the Adchimp68rab.gp construct in Adhu5-immune mice. The severity of the reduction of antibody titers to rabies virus presented by the Adhu5 construct in mice pre-immune to the homologous virus depended on the vaccine dose. The antibody response to lower doses of vaccine was more affected than the response to higher vaccine doses. VNA titers were substantially more reduced than the ELISA titers. Titers of VNAs to the highest vaccine dose were halved in mice pre-immune to Adhu5 virus while at the two lower vaccine doses titers were reduced by more than 20 fold. At any of the doses tested, the Adchimp68rab.gp recombinant induced higher VNA titers to rabies in Adhu5 pre-immune mice compared to those achieved by an equal dose of the Adhu5rab.gp vaccine. The detrimental effect of pre-existing immunity to Adhu5 on the efficacy of the Adhu5 vaccine was demonstrated further in a protection experiment. Naïve mice immunized with 2×10⁵ pfu of Adhu5rab.gp or Adchimp68rab.gp virus were completely protected to challenge with CVS-11 virus. The majority (65%) of Adhu5 pre-immune mice immunized with this dose of the Adhu5rab.gp vaccine succumbed to a rabies virus infection while those vaccinated with the same dose of the Adchimp68rab.gp virus remained protected. Increasing the dose of the Adhu5rab.gp virus to 2×10⁶ pfu per mouse restored the efficacy of the vaccine.

The antibody response to the transgene product expressed by the Adchimp68 recombinant was not affected by pre-existing immunity to common human adenovirus serotypes, which inhibits the response to the corresponding recombinant of the human serotype 5. Upon pre-immunization with replication-competent viruses, the immune response to the Adhu5rab.gp vaccine was abolished in Adhu5 pre-immune mice and reduced in mice pre-immune to other human serotypes of adenovirus such as 2 and 4. The response to the Adchimp68 recombinant was as expected inhibited in mice pre-immune to the homologous virus. This is not of clinical concern as Adchimp68 virus does not circulate in the human population and common human serotypes do not share neutralizing epitopes with Adchimp68 virus.

Pre-exposure to replication-defective Adhu5 virus also reduced the antibody response to the rabies virus glycoprotein presented by the Adhu5 recombinants although the impact was not as severe as in mice previously infected with replication-competent virus. Sera from mice pre-immune to replication-defective Adhu5 virus developed reduced but readily detectable antibodies to rabies virus upon immunization with the Adhu5rab.gp vaccine. Increasing the dose of the Adhu5rab.gp construct could in part circumvent the impact of pre-existing immunity. Vaccine-induced protection against rabies virus requires VNAs, which were not induced as efficiently in pre-immune mice by the Adhu5 vaccine especially when used at lower doses. In Adhu5 pre-immune mice the VNA response to the Adchimp68rab.gp construct was superior at all doses tested to that of the Adhu5 vaccine thus more than compensating for the slightly lower potency of this vaccine upon s.c. immunization.

Adchimp68 recombinants thus provide an attractive alternative as a vaccine carrier for use in humans. As shown here they are efficacious even when applied at low doses of 2×10⁵ pfu through non invasive routes of administration such as the upper airways. Mucosal immunization by i.n. application has the added advantage of favoring induction of responses of the common mucosal immune system, which is distinct from, albeit interconnected with the central immune system targeted by injected vaccines.

### Example 11

### Chimpanzee C68 Virus Stock and Replication

Examples 11 through 15 which follow provide additional characterization of the chimpanzee C68. It will be appreciated by one of skill in the art that this information can be readily used in the construction of novel recombinant chimpanzee adenoviral constructs.

The C68 virus stock was obtained from ATCC (Rockville, Md.) and propagated in 293 cells (ATCC) cultured in DMEM (Sigma, St. Louis, Mo.) supplemented with 10% fetal calf serum (FCS; Sigma or Hyclone, Logan, Utah) and 1% Penicillin-Streptomycin (Sigma). Infection of 293 cells was carried out in DMEM supplemented with 2% FCS for the first 24 hours, after which FCS was added to bring the final concentration to 10%. Infected cells were harvested when 100% of the cells exhibited virus-induced cytopathic effect (CPE), collected, and concentrated by centrifugation. Cell pellets were resuspended in 10 mM Tris (pH8.0), and lysed by 3 cycles of freezing and thawing. Virus preparations were obtained following 2 ultra centrifuge steps on cesium chloride density gradients and stocks of virus were diluted to 1×10¹² particles/ml in 10 mM Tris/100 mM NaCl/50% glycerol and stored at −70° C.

### Example 12

### Cloning and Sequencing of Viral Genomic DNA

Genomic DNA was isolated from the purified virus preparation following standard methods and digested with a panel of 16 restriction enzymes following the manufacturer=s recommendations. Except as noted, all restriction and modifying enzymes were obtained from Boehringer Mannheim, Indianapolis, Ind. Genomic DNA was digested with BamHI, PstI, SalI, HindIII or XbaI and the fragments were subcloned into plasmids (K. L. Berkner and P. A. Sharp, Nucl. Acids Res., 11:6003-20 (1983)). After deproteination, synthetic 10 bp Pad linkers (New England Biolabs, Beverly, Mass.) were double digested with Pad and BamHI, or PstI.

The PstI, BamHI and HindIII clones generated from C68 are illustrated in FIG. 1, parts C, D and E, respectively. The fragments indicated by the shaded boxes were not cloned, but the sequence of the entire genome has been determined through sequencing overlapping clones and viral DNA directly (unshaded boxes). The cloned fragments are described in Table 2.

TABLE 2
C68 plasmid clones and insert sizes

| | Insert | | | | |
|---|---|---|---|---|---|
| | Size | | | 5′ End | 3′ End |
| | (base | Fragment | Fragment | Map | Map |
| Construct Name | pairs) | 5′ End | 3′ End | Unit | Unit |
| Pst-I Fragments | | | | | |
| C68-Pst-A | 6768 | 24784 | 31551 | 67.9% | 86.4% |
| pBS:C68-Pst-B | 6713 | 4838 | 11550 | 13.2% | 31.6% |
| pBS:C68-Pst-C | 5228 | 14811 | 20038 | 40.6% | 54.9% |
| pBS:C68-Pst-D | 2739 | 12072 | 14810 | 33.1% | 40.6% |
| pBS:C68-Pst-E | 2647 | 20039 | 22685 | 54.9% | 32.1% |
| pBS:C68-Pst-F | 1951 | 32046 | 33996 | 87.8% | 93.1% |
| PNEB:C68-Pst-G | 1874 | 1 | 1874 | 0.0% | 5.1% |
| pBS:C68-Pst-H | 1690 | 23094 | 24783 | 63.2% | 67.9% |
| pBS:C68-Pst-I | 1343 | 33997 | 35339 | 93.1% | 96.8% |
| PNEB:C68-Pst-J | 1180 | 35340 | 36519 | 96.8% | 100.0% |
| pBS:C68-Pst-K | 1111 | 2763 | 3873 | 7.6% | 10.6% |
| pBS:C68-Pst-L | 964 | 3874 | 4837 | 10.6% | 13.2% |
| pBS:C68-Pst-M | 888 | 1875 | 2762 | 5.1% | 7.6% |
| pBS:C68-Pst-N | 408 | 22686 | 23093 | 62.1% | 63.2% |
| C68-Pst-O | 380 | 31666 | 32045 | 86.7% | 87.7% |
| pBS:C68-Pst-P | 285 | 11551 | 11835 | 31.6% | 32.4% |
| C68-Pst-Q | 236 | 11836 | 12071 | 32.4% | 33.1% |
| pBS:C68-Pst-R | 114 | 31552 | 31665 | 86.4% | 86.7% |
| BamHI Fragments | | | | | |
| C68-Bam-A | 16684 | 19836 | 36519 | 54.3% | 100.0% |
| pBS:C68-Bam-B | 8858 | 3582 | 12439 | 9.8% | 34.1% |
| pBS:C68-Bam-C | 4410 | 12440 | 16849 | 34.1% | 46.1% |
| pBS:C68-Bam-D | 2986 | 16850 | 19835 | 46.1% | 54.3% |
| PNEB:C68-Bam-E | 2041 | 1 | 2041 | 0.0% | 5.6% |
| pBS:C68-Bam-F | 1540 | 2042 | 3581 | 5.6% | 9.8% |
| HindIII Fragments | | | | | |
| pBR:C68-Hind-B | 9150 | 23471 | 32620 | 64.3% | 89.3% |

pBS = pBluescript SK+ clone
pNEB = pNEB 193 clone
pBR = pBR322 clone
No prefix = fragment not cloned

Chimpanzee adenovirus, C68, was obtained from ATCC and propagated in human 293 cells. Viral genomic DNA was isolated from purified virions using established procedures (A. R. Davis, et al., Gene Thera., 5:1148-1152 (1998)) and digested with a panel of restriction enzymes; the data were consistent with previous studies (data not shown) (G. R. Kitchingman, Gene, 20:205-210 (1982); Q. L1 and G. Wadell, Arch Virol. 101:65-77 (1998); R. Wigand, et al., Intervirology. 30:1-9 (1989)). Restriction fragments spanning the entire genome of C68 were subcloned into plasmids. A schematic drawing of the C68 genome is shown in FIG. 1A, and the Pst-I, BamHI and HindIII fragments that were cloned into plasmid vectors are indicated by the unshaded boxes, in FIGS. 1B, 1C, and 1D, respectively. The cloned fragments, fragment sizes and genomic position are also listed in Table 2. Both plasmid clones and genomic DNA were used as templates for sequencing. The genome was sequenced by primer walking in both directions and each base was included in an average of approximately four reactions.

The C68 genome is 36521 bp in length [see, U.S. Pat. No. 6,083,716]. Preliminary comparison with GenBank sequences indicated varying degrees of similarity with other human and animal adenoviruses along the entire length of the viral genome. Regions with homology to all of the previously described adenoviral genetic units, early regions 1-4 and the major late genes, were found in the C68 genome (FIG. 1A). DNA homology between C68 and the human adenoviruses that have been completely sequenced, Ad2 (NC001405), Ad5 (NC001405), Ad12 (NC001460), Ad17 (NC002067) and Ad40 (NC01464), was used to order the clones. The open reading frames (ORF) were determined and the genes were identified based on homology to other human adenoviruses. All of the major adenoviral early and late genes are present in C68. The inverted terminal repeats (ITR=s) are 130 bp in length.

### Example 13

### Analysis of C68 Sequence

The complete nucleotide sequence of every member of the Mastadenovirus genus accessible from GenBank, including isolates from different species, were screened for identity to C68. The Ad4 minigenome was assembled from the following GenBank sequences: Left-hand ITR (J01964); E1A region (M14918); DNA pol and pTP (X74508, 74672); VA RNA-I, II (U10682); 52, 55K (U52535); pVII (U70921); hexon (X84646); endoprotease (M16692); DNA-binding protein (M12407); fiber (X76547); right-hand ITR (J01965). The Ad7 composite genome was created from the following sequence data: Mu 3-21 (X03000); VA RNA-I, II, pTP & 52, 55K (U52574); penton (AD001675); pVI, hexon and endoprotease (AF065065); DNA-binding protein (K02530); E3 and fiber region (AF104384); right-hand ITR (V00037).

The amino acid sequence alignment was generated with Clustal X, edited with Jalview (http://www.ebi.ac.uk/˜michele/jalview/), and analyzed with Boxshade (http://www.ch.embnet.org/software/BOX_form.html). Publicly available hexon protein sequences from all human adenovirus serotypes were initially aligned to identify the set showing the highest homology to C68.

The nucleotide sequence and predicted amino acid sequences of all significant open reading frames in the C68 genome were compared to known DNA and protein sequences. The nucleotide sequence of C68 is compared to sequences of Ad 2, 4, 5, 7, 12, 17 and 40. In agreement with previous restriction analysis (Kitchingman, cited above; L1 and Wadell, cited above) C68 is most similar to human Ad4 (subgroup E).

The E1A region of C68 extends from the TATA box at nt 480 to the poly A addition site at 1521. The consensus splice donor and acceptor sites are in the analogous position of the human Ad counterparts, and the 28.2K and 24.8K proteins are similar in size to the human Ad proteins. The ORF for the smallest E1A protein of C68 is predicted to encode 101 residues as opposed to approximately 60 amino acids for other adenoviruses. There is a TTA codon at residue 60 for C68 where other adenoviruses often have a TGA stop codon. The first 60 residues of C68 E1A 100R protein have 85% identity to the Ad4 homolog.

The C68 genome encodes genes for the four E1B proteins, 20.5K, 54.7K, 10.1K and 18.5K as well as pIX. All five C68 encoded proteins are similar in size to that of other Ad E1B and pIX proteins. The Ad4 homolog of the E1B 21K protein has only 142 amino acids, where C68 has 186 residues and other human adenoviruses have 163-178 residues. The C68 and Ad4 proteins share 95% identity over the first 134 aa, then the similarity ends and the Ad4 protein terminates at 142 amino acids.

The C68 genome encodes homologs of the E2A 55K DNA binding protein and the Iva2 maturation protein, as well as the E2B terminal protein and the DNA polymerase. All of the E2 region proteins are similar in size to their human Ad counterparts, and the E2B proteins are particularly well conserved. The C68 E2B 123.6K DNA polymerase is predicted to be 1124 residues, while Ad4 is predicted to have 1193 although the other human adenoviruses have smaller polymerases. Residues 1-71 of the Ad4 polymerase have no similarity to any other Ad polymerase, and it is possible that this protein actually initiates at an internal ATG codon. From amino acids 72-1193, Ad4 and C68 polymerases have 96% amino acid identity.

The E3 regions of human adenoviruses sequenced so far exhibit considerable sequence and coding capacity variability. Ad40 has five E3 region genes, Ad12 has six, C68 and Ad5 have seven, Ad38 has eight and Ad3 as well as Ad7 (subgroup B human adenoviruses) have nine putative E3 region genes. The Ad4 E3 region has not yet been sequenced. In comparison with the E3 region of Ad35, all 7 E3 gene homologs were identified in the C68 genome (C. F. Basler and M. S. Horwitz, Virology, 215: 165-177 (1996)).

The C68 E4 region has 6 ORFs and each is homologous to proteins in the human Ad5, 12 and 40 E4 region. The E4 nomenclature is confusing because the ORF2 homologs of C68, Ad12 and Ad40 are approximately 130 residues, while in Ad5 there are two ORFs encoding proteins of 64 and 67 residues with homology, respectively, to the amino and carboxy terminal ends of the larger ORF2 proteins. ORFS has been omitted in our nomenclature because the 5^{th} ORF in the E4 region is homologous to the widely studied ORF6 protein of human Ad5.

The major late promoter and the tri-partite leader sequences of the C68 genome were located. ORFs with the potential to encode the 15 major late proteins were located. All of the C68 late proteins are similar in size to their human Ad counterparts. The percent amino acid identity between chimpanzee and human Ad late proteins varies considerably. The C68 fiber protein is predicted to have 90% amino acid identity with the Ad4 protein, but much less similarity to the other human Ad fiber proteins. The CAR binding site in the fiber knob is present in C68.

### Example 14

### Virus Neutralizing Antibody Assays

Several studies were performed to determine if there is cross-reactivity between type specific antisera of C68 and human adenovirus. The neutralizing activity of sera was tested as follows. Panels of sera from normal human subjects (N=50), rhesus monkeys (N=52) and chimpanzees (N=20) were evaluated for neutralizing antibodies against Ad5 and C68 based vectors using 293 cells as an indicator cell line. Sera collected from individual humans, rhesus monkeys, or chimpanzees were inactivated at 56° C. for 30 minutes. A serial dilution of each sample (1:10, 1:20, 1:40, 1:80, 1:160, 1:320 in 100 μl of DMEM containing 10% FCS) was added to equal amounts of H5.010CMVEGFP (1000 PFU/well) or C68CMVEGFP virus and incubated at 4° C. for two hrs. One hundred and fifty microliters of the mixture were transferred onto 2×10 293 cells in 96 well flat bottom plates. Control wells were infected with equal amounts of virus (without addition of serum). Samples were incubated at 37° C. in 5% CO₂ for 48 hrs and examined under a fluorescent microscope. Sample dilutions that showed >50% reduction of green-fluorescent foci as compared to infected controls were scored positive for neutralizing antibodies.

As expected, approximately 35% of normal human subjects demonstrated neutralizing antibody against Ad5, a frequency much higher than observed in sera of rhesus monkeys and chimpanzee. Neutralizing antibody to C68 was observed in 80% of chimpanzee and only 2% of normal human subjects or rhesus monkeys. Titers of neutralizing antibodies in the non-target species were generally low.

To further evaluate cross-reactivity of C68 with human adenovirus vectors, mice were immunized with 2×10⁷ plaque forming units (pfu) of Ad 2, 4, 5, 7 and 12 as well as C68. Sera were harvested 2 weeks later and tested for antibodies that neutralized either Ad5 or C68 vectors. Neutralizing antibody to Ad5 vector was only detected in animals immunized with Ad5. Importantly, the only animals with neutralizing antibody to C68 vector were those immunized with C68 vector; none of the human serotypes tested, including Ad4, generated antibodies in mice that neutralized C68 in vitro.

Important to the utility of C68 vector in human trials is the absence of neutralizing antibody in the human population. In our study, a screen of 50 normal human subjects failed to detect any significant neutralizing antibodies (>1:10) using the same assay that showed neutralizing antibodies in >50% of chimpanzees. Furthermore, sera of mice immunized with multiple human Ad serotypes including Ad4, did not neutralize infection with C68.

In another study, groups of ten to twenty ICR mice were vaccinated with varied doses of the Adhu5rab.gp or the AdC68rab.gp vaccine given subcutaneously (s.c.), intranasally (i.n.) or orally (per os). Mice were bled 21 days later and viral neutralizing antibody (VNA) titers expressed as international units were determined Mice were challenged 4 weeks after vaccinated with 10 mean lethal doses of CVS-24 virus applied directly into the central nervous system.

| | VNA Titers (% survival upon challenge) | | | |
|---|---|---|---|---|
| Vaccine Dose | 5 × 10⁷ | 5 × 10⁶ | 5 × 10⁵ | 5 × 10⁴ |
| Adhu5rab.gp, s.c. | 972 (100) | 324 (100) | 108 (100) | 12 (100) |
| AdC68rab.gp, s.c. | 240 (100) | 36 (100) | 12 (80) | 8 (80) |
| Adhu5rab.gp, i.n. | nt | 162 (100) | 162 (100) | 18 (50) |
| AdC68rab.gp, i.n. | nt | 54 (100) | 162 (100) | 18 (50) |
| | 2 × 10⁷ | 2 × 10⁶ | 2 × 10⁵ | 2 × 10⁴ |
| Adhu5rab.gp, per os | 108 (100) | 54 (88) | 18 (80) | 4 (30) |
| AdC68rab.gp, | 108 (100) | 36 (78) | 12 (55) | 0.2 (0) |
| per os | | | | |

These data demonstrate that the AdC68 construct unexpectedly induces a better protective antibody response at low doses intranasally than human type 5.

### Example 15

### Structural Analysis of Hexon Proteins

The absence of neutralizing antibodies between C68 and human serotypes compelled us to more carefully evaluate structural differences in the regions of hexon presumed to harbor type specific epitopes. Previous studies have suggested that these epitopes are located within the 7 hypervariable regions of hexon determined by Crawford-Miksza and Schnurr (J. Virol, 70:1836-1844 (1996)). A comparison of the amino acid sequences of hexon proteins between C68 and several human adenoviruses is shown in FIG. 2. Indeed, C68 is substantially dissimilar in significant regions of these hypervariable sequences. More detailed modeling of the three dimensional structure of hexon of C68 was performed to map the unique sequences. Models of hexon structures from C68 and Ad4 were generated based on the x-ray crystal structures of hexons for Ad2 and Ad5.

The X-ray crystal structures of Ad5 hexon (Protein Data Bank identifier 1RUX) (J. J. Rux and R. M. Burnett, Mol. Ther. 1:18-30 (2000)), and that for Ad2 (F. K. Athappilly, et al, J. Mol. Biol., 242: 430-455 (1994)), have been further refined to yield the current hexon models (Rux and Burnett, to be published elsewhere). Models of the homologous C68 and Ad4 hexons were initially produced using the Swiss-PdbViewer protein-modeling environment (N. Guez and M. C. Peitsch, Electrophoresis, 18:2714-2723 (1997)). Its automated procedure was used to align the C68 and Ad4 hexon amino acid sequences to those of the Ad2 and Ad5 hexon crystal structures. The sequence alignments were used to guide the threading of the model sequences onto the known molecular structures. The side chain positions of residues not seen in the known structures were selected from a library of side chain protomers. These initial molecular models were then manually adjusted to improve the automated alignment by moving gaps to exposed variable regions and by optimizing the packing of side chains. The positions of external loop segments not observed in the Ad2 and Ad5 template structures were either selected from a library of known loop structures or fitted manually. The conformation of each model was further refined by energy minimization using the molecular mechanics program CHARMM (B. R. Brooks, et al, J. Comp. Chem., 4:187-217 (1983)). The structures of these C68 and Ad4 hexon models were then aligned and a new sequence alignment calculated. The differences between the two structurally aligned hexon sequences were used to color images of the homology models. Graphical images prepared within the Swiss-PdbViewer program were exported and rendered with the Persistence of Vision Ray Tracer program (POV-Ray 2000, Version 3.1 g).

While the overall C68 sequence is very similar to that of Ad4 hexon, the differences between the two sequences are primarily focused in the DE1 and FG1 loops, and these contain all seven hypervariable regions. It is the DE1, FG1, and FG2 loops, each from a different subunit, that intimately associate to form the tower domains at the top of the trimeric molecule. The hexon towers form much of the exterior surface of the virion and are the sites of antibody attachment. As the sides and base of the hexons pack together within the capsid, these regions are shielded from antibody binding and their sequences are conserved. In contrast, the sequences of C68 and Ad4 are quite different in the hexon towers. This immediately explains why antibodies raised to either of these viruses do not cross-react.

### Example 16

### Viral Propagation

The Pan5 [ATCC Accession No. VR-591], Pan6 [ATCC Accession No. VR-592], and Pan7 [ATCC Accession No. VR-593] viruses, originally isolated from lymph nodes from chimpanzees, were propagated in 293 cells [ATCC CRL1573]. Typically, these cells are cultured in Dulbecco's Modified Eagles Medium (DMEM; Sigma, St. Louis, Mo.) supplemented with 10% fetal calf serum (FCS) [Sigma or Hyclone, Logan, Utah] and 1% Penicillin-Streptomycin (Sigma). Infection of 293 cells is carried out in DMEM supplemented with 2% FCS for the first 24 hours, after which FCS is added to bring the final concentration to 10%. Infected cells are harvested when 100% of the cells exhibit virus-induced cytopathic effect (CPE), and are then collected, and concentrated by centrifugation. Cell pellets are resuspended in 10 mM Tris (pH 8.0), and lysed by 3 cycles of freezing and thawing. Virus preparations are obtained following two ultra centrifugation steps on cesium chloride density gradients and stocks of virus are diluted to 1 to 5×10¹² particles/ml in 10 mM Tris/100 mM NaCl/50% glycerol and stored at −70° C.

The ability of 293 cells to propagate these adenoviruses exceeded expectations which were based on knowledge of other non-human adenovirus serotypes.

| Virus | Yield (virus particles produced in 8 × 10⁸ cells) |
|---|---|
| Pan5 | 8.8 × 10¹³ |
| Pan6 | 1.6 × 10¹⁴ |
| Pan7 | 8.8 × 10¹³ |

### Example 17

### Characterization of Viral Genomic DNA

Genomic DNA was isolated from the purified virus preparations of Example 16 and digested with HindIII or BamHI restriction enzymes following the manufacturers' recommendations. The results (not shown) revealed that that the Pan8, Pan6, Pan7 genomes of the invention and the published Pan 9 (C68) genome show different restriction patterns, and thus, are distinct from each other.

The nucleotide sequences of Pan8, Pan6 and Pan7 were determined The nucleotide sequence of the top strand of Pan8 DNA is reported in SEQ ID NO: 1. The nucleotide sequence of the top strand of Pan6 DNA is reported in SEQ ID NO: 5. The nucleotide sequence of the top strand of Pan7 DNA is reported in SEQ ID NO: 9.

Regulatory and coding regions in the viral DNA sequences were identified by homology to known adenoviral sequences using the “Clustal W” program described above at conventional settings. See the tables above providing the adenoviral sequences. Open reading frames were translated and the predicted amino acid sequences examined for homology to previously described adenoviral protein sequences, Ad4, Ad5, Ad7, Ad12, and Ad40.

Analysis of the sequence revealed a genome organization that is similar to that present in human adenoviruses, with the greatest similarity to human Ad4. However, substantial differences in the hexon hypervariable regions were noted between the chimpanzee adenoviruses and other known adenoviruses, including AdHu4. These differences fit well with the serological cross-reactivity data that has been obtained (see below).

An alignment of a portion of the hexon sequences is shown in FIG. 1. The portion shown is from the region of the hexon that corresponds to the outwardly disposed extended loops DE1 and FG1 where the most variability between serotypes is observed. An intervening portion that contributes to the base of the hexon (corresponding to residues 308-368 of the published AdC68 sequence; U.S. Pat. No. 6,083,716), and is highly conserved between serotypes, is also present. The following table summarizes the pair-wise comparisons of the amino acids in the hexon proteins.

| Comparison | | Hexon amino-acid |
|---|---|---|
| #1 | #2 | Similarity (%) |
| AdC5 | AdC7 | 99.0 |
| AdC5 | AdC68 | 98.3 |
| AdC5 | AdC6 | 88.0 |
| AdC5 | AdC1 | 84.9 |
| AdC6 | AdC7 | 87.7 |
| AdC6 | AdC68 | 87.3 |
| AdC6 | AdC1 | 84.9 |
| AdC7 | AdC68 | 97.5 |
| AdC7 | AdC1 | 84.8 |
| AdC68 | AdC1 | 84.9 |

Analysis of the fiber knob domain (which is responsible for receptor binding) of the chimpanzee adenoviruses shows an overall similarity in structure (FIG. 2).

The degree of sequence similarity between the E1 proteins of huAd5 and C68 (see Tables below) is similar to that between huAd5 and Pan-5, Pan-6, and Pan-7.

| Comparison | | E1a (13S) amino- |
|---|---|---|
| #1 | #2 | acid identity (%) |
| AdHu5 | AdC5 | 36.6 |
| AdHu5 | AdC6 | 28.5 |
| AdHu5 | AdC7 | 34.9 |
| AdHu5 | AdC68 | 35.6 |
| AdHu5 | AdC1 | 35.6 |
| AdC5 | AdC6 | 68.3 |
| AdC5 | AdC7 | 96.9 |
| AdC5 | AdC68 | 80.4 |
| AdC5 | AdC1 | 51.3 |
| AdC6 | AdC7 | 69.3 |
| AdC6 | AdC68 | 59.4 |
| AdC6 | AdC1 | 37.7 |
| AdC7 | AdC68 | 81.5 |
| AdC7 | AdC1 | 51.0 |
| AdC68 | AdC1 | 54.9 |

Sequence Identity with
human Ad5

| | E1b Small T | E1b Large T |
|---|---|---|
| | Protein | Protein |
| C68 | 47.3% | 55.8% |
| Pan-5 | 43.2% | 54.5% |
| Pan-6 | 45.3% | 54.5% |
| Pan-7 | 46.4% | 53.8% |

Replication-defective versions of AdC5, AdC6 and AdC7 were created by molecular cloning methods described in the following examples in which minigene cassettes were inserted into the place of the E1a and E1b genes. The molecular clones of the recombinant viruses were rescued and grown up in 293 cells for large-scale purification using the published CsCl sedimentation method [K. Fisher et al., J. Virol., 70:520 (1996)]. Vector yields were based on 50 plate (150 mm) preps in which approximately 1×10⁹ 293 cells were infected with the corresponding viruses. Yields were determined by measuring viral particle concentrations spectrophotometrically. After having constructed E1-deleted vectors, it was determined that HEK 293 cells (which express human adenovirus serotype 5 E1 functions) trans-complement the E1 deletions of the novel viral vectors and allow for the production of high titer stocks. Examples of virus yields for a few of these recombinant viruses are shown in the table below.

The transgenes for these vectors, β-galactosidase (LacZ), green fluorescent protein (GFP), alpha-1-anti-trypsin (A1AT), ebola glycoprotein (ebo), a soluble ebola glycoprotein variant lacking the transmembrane and cytoplasmic domains (sEbo), and three deletion mutants of the ebola glycoprotein (EboΔ2, EboΔ3, and EboΔ4), were expressed by the cytomegalovirus promoter (CMV). In the following table, ND indicates that the study has not yet been done.

Viral backbone/Vector yield
(Viral particles × 10¹³)

| Transgene | AdHu5 | AdC7 | AdC68 | AdC6 |
|---|---|---|---|---|
| CMVLacZ | 1.5 | 1.4 | 3.3 | 6.1 |
| CMVGFP | 2.5 | 3.6 | 8 | 10 |
| CMVA1AT | 3.7 | 6 | 10 | ND |
| CMVEbo | 1.1 | 4.3 | ND | ND |
| CMVsEbo | 4.9 | 5.4 | ND | ND |
| CMVEboΔ2 | 1 | 9.3 | ND | ND |
| CMVEboΔ3 | 0.8 | 9.5 | ND | ND |
| CMVEboΔ4 | 1.4 | 6.2 | ND | ND |

The ability of human adenovirus E1 to trans-complement the E1-deleted chimpanzee viruses of the invention is highly advantageous, as it permits the production of E1-deleted chimpanzee adenoviral vectors of the invention, while reducing or eliminating the risk of homologous recombination due to the differences in sequences between human Ad and the chimpanzee adenoviruses described herein.

### Example 18

### Serological Studies of Pan 5, 6, and 7 Viruses

Because of the differences in the hexon hypervariable region, it was anticipated that the C5, C6, and C7 viruses would be serologically distinct from human adenoviruses, including AdHu4.

1. Cross-Reactivity of Wild-type Viruses

For screening of wild-type viruses in order to make a determination of antibody cross-reactivity, the replication competent viruses were used and inhibition of cytopathic effects (CPE) was measured. Briefly, preparations of adenoviruses (Adhu5, Pan-5, Pan-6, Pan-7 and AdC68) stored at 5×10¹² particles/ml were diluted 1/600 for the assays. This concentration of virus was selected since it results in 100% CPE within 48 hours in the absence of neutralization. Prior to adding the virus to 293 cells (4×10⁴ cells/well in a 96 well dish), 1:20 dilutions of sera were added. The assay is read as the presence or absence of CPE; full neutralization would read as no cytopathic effect. The results are summarized in the Table below. The fact that 9/36 human sera neutralized Adhu5 induced CPE is consistent with previous estimates of neutralizing antibodies in the human population. The numbers indicate the total individuals who showed neutralization (numerator) versus the total number screened (denominator). ND=not determined

Neutralization by 1/20 diln of serum

| | Human | Rhesus | Chimpanzee |
|---|---|---|---|
| | (N = 36) | (N = 52) | (N = 20) |
| Adhu5 | 9/36 | ND | ND |
| AdC68 | 1/36 | 0/52 | 12/20 |
| Pan 5 | 0/36 | 0/52 | 10/20 |
| Pan 6 | 0/36 | 0/52 | 9/20 |
| Pan 7 | 0/36 | 0/52 | 12/20 |

Of all human sera screened, 35/36 were negative for neutralization to AdC68 while 36/36 were negative for neutralization to Pan-5, Pan-6 and Pan-7. Of 52 rhesus monkeys screened, none showed neutralization to any chimpanzee adenovirus; rhesus monkey is the preferred pre-clinical model for evaluating HIV vaccines. Between 9 to 12 out of 20 chimpanzees had substantial neutralization to one or another of the chimpanzee adenoviruses consistent with the fact these are indeed endemic chimpanzee-specific pathogens. Interestingly, there are chimpanzees with neutralizing antibodies only to Pan-5, Pan-6 or AdC68 supporting the hypothesis that several of these chimpanzee adenoviral vectors will not cross neutralize each other and are distinct serotypes.

The same assay was carried out for 20 chimpanzee serum samples. Fifty percent (50%) of the samples reacted serologically, in different degrees to Pan8; 40% to Pan6; 55% to Pan7 and 60% to C68. Among the positive serum samples, one of them had strong neutralizing activity to all four chimp viruses.

2. Cross-neutralization with Recombinant Viruses

High-titer polyclonal antibodies were obtained to each of the simian adenoviruses in order to more precisely gauge the degree of cross-neutralization among the different serotypes. This was done by intramuscular immunization of rabbits using a recombinant virus containing GFP based on previously the described C68 chimpanzee adenovirus as an adjuvant. The serum was then used to assay for neutralizing activity against each of the three chimpanzee adenoviruses of the invention, AdC5, AdC6 and AdC7. A rabbit was injected with 5×10¹² viral particle per kg of C68CMVGFP vector intramuscularly and boosted 5 weeks later using the same dose. A bleed collected at the 9 week time point revealed extremely potent neutralizing activity against C68 as well as Pan-5 and Pan-7 but not against Pan-6 (see Table below), indicating that the administration of a C68 (or Pan-5 and Pan-7) based vaccine could be effectively followed by a boost using a vector based on Pan-6. However, it has been found that this level of inter-relatedness does not necessarily prevent with re-administration in a setting where antiviral antibody titers were not as high as was achieved in this rabbit. In the following table, + indicates 33% CPE; ++ indicates 66% CPE; +++ indicates 100% CPE.

| Infection on 293 cells with virus | | | | | |
|---|---|---|---|---|---|
| | | | | C68 | Serum |
| Pan5 | Pan6 | Pan7 | Pan9(C68) | GFP | Dilution |
| − | +++ | − | − | − | 1/20 |
| − | +++ | − | − | − | 1/40 |
| − | +++ | − | − | − | 1/80 |
| − | +++ | − | − | − | 1/160 |
| − | +++ | − | − | − | 1/320 |
| − | +++ | − | − | − | 1/640 |
| − | +++ | − | − | − | 1/1,280 |
| − | +++ | − | − | − | 1/2,560 |
| − | +++ | − | − | − | 1/5,120 |
| + | +++ | − | − | − | 1/10,240 |
| + | +++ | ++ | − | − | 1/20,480 |
| ++ | +++ | +++ | − | − | 1/40,960 |
| ++ | +++ | +++ | + | + | 1/81,920 |
| +++ | +++ | +++ | ++ | ++ | 1/163,840 |
| +++ | +++ | +++ | +++ | +++ | 1/327,680 |
| +++ | +++ | +++ | +++ | +++ | 1/665,360 |
| +++ | +++ | +++ | +++ | +++ | 1/1,310,720 |
| +++ | +++ | +++ | +++ | +++ | 1/2,621,440 |

3. Quantitative Assay for Detection of Neutralizing Antibody

The result was validated by a more quantitative-based assay for detecting neutralizing antibody, which is based on transduction of a GFP vector. Briefly, groups of C57BL/6 mice were immunized intramuscularly or intravenously with 5.0×10¹⁰ particles/ml Pan8, Pan6, Pan7 or C68. Sera from day 28 bleeds were tested for cross-neutralizing activity against C68CMVEGFP at dilutions of 1/20 and 1/80. In summary, when a pharmaceutical preparation of human immunoglobulin was tested for serological reactions to Pan 5, 6, and 7, and C68, some low levels of neutralizing activities against Pan 7 and C68 were detected. No neutralizing activity against Pan8 or Pan6 was detected. Serum samples from 36 human subjects were run for the same assay. Serum samples were tested at a 1/20 dilution. The results indicated that only one individual has clear neutralizing activity to C68. No neutralizing activity to Pan8, Pan6 or Pan7 was detected.

4. In Vitro Cross-Neutralization

Cross-neutralization of the simian adenoviruses by high-titer rabbit polyclonal antibodies raised against each of the adenoviruses Pan-5, Pan-6, Pan-7, and C68 was tested.

Rabbits were immunized with intra-muscular injections of 10¹³ particles of each of the chimpanzee adenoviruses and boosted 40 days later with the same dose with incomplete Freund's adjuvant. Sera were analyzed or the presence of neutralizing antibodies by incubating serial two-fold dilutions with 10⁹ genome copies of each of the appropriate chimpanzee adenovirus vector expressing GFP and testing for the attenuation of GFP expression when applied to 293 cells. The serum dilution which produced a 50% reduction of GFP expression was scored as the neutralizing antibody titer against that particular virus.

The results are shown in the Table. The data are consistent with the expectation from sequence analysis of the hexon amino-acid sequences, which indicated that Ad Pan-6 was likely to be the most serologically distinct compared to the other chimpanzee adenoviruses.

| Serum from | | | | |
|---|---|---|---|---|
| rabbit | | | | |
| immunized | Infection of 293 cells with 10⁹ genome copies of | | | |
| with: | Ad Pan-5 | Ad Pan-6 | Ad Pan-7 | Ad C68 |
| Ad Pan-5 | 1/5120 | <1/20 | 1/2560 | 1/2560 |
| Ad Pan-6 | No | 1/20,480 | <1/20 | <1/20 |
| | neutralization | | | |
| Ad Pan-7 | 1/2560 | 1/160 | 1/163,840 | 1/2560 |
| Ad C68 | No | <1/20 | <1/20 | 1/5120 |
| | neutralization | | | |

In order to determine whether antibodies cross-reacting with the simian adenoviruses were likely to be of low prevalence in humans, simian adenoviruses SV1, SV39, and SV25 were tested for their ability to withstand neutralization when incubated with commercially available pooled human immunoglobulins (Ig). The same assay was also performed with Adhu5 and the chimpanzee adenoviruses Pan-5, Pan-6, Pan-7, and C68. In a further study, sera from mice has been immunized with one of the chimpanzee adenoviruses C5, C6, C7, and C68 and their ability to cross-neutralize the simian adenoviruses SV-15, SV-23, SA-17, and Baboon Adenovirus has been tested. No cross-reactivity was observed in any case.

### Example 19

### Generation of Recombinant E1-Deleted Pan5 Vector

A modified pX plasmid was prepared by destroying the FspI site in the bla gene region of pX (Clontech) by site-directed mutagenesis. The resulting modified plasmid, termed pX′, is a circular plasmid of 3000 bp which contains an fl ori and an ampicillin resistance gene (AmpR-cds).

A. Production of Pan-5 Adenovirus Plasmid

A polylinker for sequential cloning of the Pan5 DNA fragments into pX′ is created. The polylinker is substituted for the existing pX′ polylinker following digestion with MluI and EcoRI. The blunt-FseI fragment of the Pan 5 is inserted into the SmaI and FseI sites of the polylinker. This fragment contains the 5′ end of the adenoviral genome (bp 1 to 3606, SEQ ID NO:1). The SnaBI-FspI fragment of Pan 5 (bp 455 to 3484, SEQ ID NO:1) is replaced with a short sequence flanked by I-Ceu and PI-Sce sites from pShuttle (Clontech), to eliminate the E1 region of the adenoviral genome. The EcoRI-blunt fragment of Pan5 (bp 28658 to 36462, SEQ ID NO:1) is inserted into the EcoRI and EcoRV sites of the polylinker (to provide the 3′ end of the adenoviral genome); the FseI-MluI fragment (bp 3606 to 15135, SEQ ID NO:1) is inserted into the polylinker; and the MluI-EcoRI fragment is inserted into the polylinker (bp 15135 to 28658, SEQ ID NO:1). Optionally, a desired transgene is inserted into I-CeuI and PI-SceI sites of the newly created pX′Pan5)E1 vector.

B. Alternative Method of Generating pX′Pan5)E1.

The initial plasmid pX is derived from pAdX adenovirus plasmid available from Clontech, as described above. Thereafter, a PacI-XhoI region of pX′ was deleted and the blunt-ended Pan5 polylinker was inserted into the FspI site to generate pX′PLNK (2994 bp). The 5′ end-FseI region of Pan 5 (bp 1-3607, SEQ ID NO:1) was inserted into SmaI and FseI sites of pX′LNK to generate the pX′Pan5-5′ plasmid (6591 bp). The SnaBi-NdeI region of pX′Pan5-5′ was excised and replaced with the Ceu/Sce cassette, which had been PCR amplified from pRCS to create pX′Pan5-5′)E1 (4374 bp). Briefly, a sequence containing I-CeuI and PI-SceI rare cutter sites was PCR amplified from pRCS (3113 bp). The 3′ PCR primer was introduced an NdeI site into the PCR product.

To extend the Pan8 DNA in pX′Pan8-5′)E1 (4374 bp), the FseI-MluI region of Pan 5 (bp 3607-15135, SEQ ID NO:1) is added, to create pX′Pan5-5′Mlu (15900 bp). The remaining MluI-3′ end of the Pan8 sequence (bp 15135-36462, SEQ ID NO:1) is added to the vector between the MluI and EcoRV sites of the vector polylinker to form pX′Pan5)E1 which contains the full-length Pan8 sequence containing a deletion in the E1 region.

C. Generation of Recombinant Viruses

To generate the recombinant adenoviruses from pX′Pan5)E1, the plasmid is co-transfected with a helper expressing E1, or from an E1-expressing packaging cell line, such as 293 cell line or a cell line prepared as described herein. The expression of E1 in the packaging cell permits the replication and packaging of the Pan5)E1 into a virion capsid. In another embodiment, the packaging cell transfected with pX′Pan5)E1 is transfected with an adenovirus vector as described above bearing the transgene of interest. Homologous recombination occurs between the helper and the plasmid, which permits the adenovirus-transgene sequences in the vector to be replicated and packaged into virion capsids, resulting in the recombinant adenovirus.

Transfection is followed by an agar overlay for 2 weeks, after which the viruses are plagued, expanded and screened for expression of the transgene. Several additional rounds of plaque purification are followed by another expansion of the cultures. Finally the cells are harvested, a virus extract prepared and the recombinant chimpanzee adenovirus containing the desired transgene is purified by buoyant density ultracentrifugation in a CsCl gradient or by alternative means known to those of skill in the art.

### Example 20

### Generation of Recombinant E1-Deleted Pan6 Vector

A. Strategy for Construction of Pan-6 Adenoviral Plasmid

1. Cloning of Terminal Fragments

Pan 6 virus is deproteinated by pronase and proteanase K treatment and phenol extraction. Synthetic 12 bp Pme I linkers are ligated onto the viral DNA as described by Berkner and Sharp, Nucleic Acids Research, 11: 6003 (1983). The viral DNA is then digested with Xba Ito isolate a 5′ terminal fragment (6043 bp). The Ad6 XbaI 5′ fragment is then ligated into pX link at Sma I and Xba I sites to form pX-AdPan6-0-16.5. The viral DNA with Pme I linkers is also digested with Pac Ito isolate the 6475 bp 3′ terminal fragment and cloned into pX link at Pac I and Sma I sites, resulting in pXAdPan6-82-100.

2. Deletion of E1 from the 5′ clone

To delete E1 (m.u.1.2-9), the BsiWi-Xba I fragment in pX-AdPan6-0-16.5 is replaced with a PCR fragment spanning m.u.9-16.7 fragment treated with BsiWi and Xba I, leading to pX-Ad-Pan6 m.u.0-1, 9-16.5.

3. Fusion of 5′ and 3′ Clones and to Create an Anchor Site to Accept the Middle Hind III Fragment

First, the 5′ clone, pX-Ad-Pan6 m.u.0-1, 9-16.5, is further expanded by inserting the 2^{nd} Xba I fragment (4350 bp, m.u.16.5-28) from Pan 6 genome into the Xba I site in the pX-Ad-Pan6 m.u.0-1, 9-16.5. This construct is named pXAd-Pan6-mu 0-1, 9-28.

Second, the 3′ clone is also expanded by inserting the 15026 bp Mlu I/Pac I fragment covering m.u.41-82 from Pan 6 genome into the Mlu I/Pac I sites of pXAdPan6-82-100, generating pXAdPan6-m.u.41-100.

Then, a 8167 bp Hind III/Eco 47111 Pan 6 fragment is isolated from pXAd-Pan6-mu 0-1, 9-28 and subcloned into pXAdPan6-m.u.41-100 at Hind III and Xba I blunt sites. This 5′ and 3′ fusion clone is called pXAdPan6mu0-1, 9-19.5, 64-100.

4. Drop of the Middle Fragment of the Genome into the Fusion Clone

A 16335 bp Hind III fragment (m.u.19.5-64) from Pan 6 is inserted into Hind III site of pXAdPan6mu0-1, 9-19.5, 64-100 to form pXAdPan6-O-1, 9-100.

5. Introduction of a PKGFP Selective Marker into the Final Construct for Direct Cloning the Gene of Interest and Green/White Selection of Recombinant Transformants.

A minigene cassette that expresses GFP under a lac promoter and is flanked with recognition sites of rare intron encoding restriction enzymes, PI-Sce I and I-Ceu I, was isolated from pShuttle-pkGFP (bare) by Sap I and Dra III digestions followed by filling-in reaction. The pShuttle-pkGFP (bare) plasmid is 4126 bp in length, and contains a ColE1-Ori, a kanamycin resistance gene, plac, a LacZ promoter-GFPmut3-1 cds (Clontech), and a GFPmut3-1 cds (Clontech). This cassette is subcloned into Srf I cut and blunted pXAdPan6-O-1, 9-100. This final construct is called pX-Pan6-pkGFP mu.0-1, 9-100, which is useful for generating recombinant E1-deleted Pan 6 molecular clones carrying genes of interest by direct ligation and green/white selection in combination with the generic pShuttlepkGFP vectors.

B. Alternative Strategy for Generation of Pan-6 Plasmid

1. Cloning of 5′ Terminal Fragment

The Pan 6 virus is deproteinated by pronase and proteanase K treatment and phenol extraction as described above and synthetic 12 bp Pme I linkers are ligated onto the viral DNA as described. The AdPan6 5′ XbaI fragment is isolated and ligated into pX to form pX-AdPan6-0-16.5 (9022 bp) as described in Part A above.

2. Deletion of E1 from the 5′ Clone

To delete E1 (m.u. 1.2-9), pX-AdPan6-0-16.5 is digested with SnaBI and NdeI to remove the regions encoding the E1a and E1b proteins (3442-6310 bp). This vector is subsequently digested with BsiWI in preparation for blunting with the minigene cassette carrying a selective marker.

3. Introduction of a Selective Marker

A minigene cassette that expressed GFP under a lac promoter and which is flanked with recognition sites of rare intron encoding restriction enzymes, PI-XceI and I-CeuI, was isolated from pShuttle-pkGFP as described above. The DraIII-SapI fragment is then ligated with the digested pX-AdPan6-0-16.5 to form pX-AdPan6 MU 0-16.5)E1 (7749 bp).

4. Extension of Pan-6 Adenoviral Sequences

pX-AdPan6 MU 0-16.5)E1 was subjected to XbaI digestion to permit insertion of an XbaI-RsrII linker. An XbaI/RsrII digestion fragment from the AdPan6 genome was isolated (mu 28-100, 26240 bp) and ligated into the Xba/RsrII-digested pX-AdPan6 MU 0-16.5)E1 to provide pX-AdPan6 MU 0-1, 9-16.5, 28-100. A second XbaI fragment from the Pan6 genome (mu 16.5-28, 4350 bp) is then ligated into this plasmid to form pX-AdPan6 MU 0-1, 9-100 (38551 bp).

C. Generation of Recombinant Adenoviruses

To generate the recombinant adenoviruses from a E1-deleted Pan6 plasmid prepared as described in Parts A or b, the plasmid is co-transfected with a helper expressing E1, or from an E1-expressing packaging cell line, such as 293 cell line or a cell line prepared as described herein. The expression of E1 in the packaging cell permits the replication and packaging of the Pan6-pkGFP mu.0-1, 9-100 into a virion capsid. Alternatively, the packaging cell transfected with pX-Pan6-pkGFP mu.0-1, 9-100 is transfected with an adenovirus vector as described above bearing another transgene of interest.

### Example 21

### Generation of Recombinant E1-Deleted Pan7 Vector

A. Generation of Pan7 Plasmids

A synthetic linker containing the restriction sites PacI-SmaI-FseI-MluI-EcoRV-PacI was cloned into pBR322 that was cut with EcoRI and NdeI. The left end (bp 1 to 3618) of Ad Pan7 was cloned into the linker between the SmaI and FseI sites. The adenovirus E1 was then excised from the cloned left end by cutting with SnaBI and NdeI and inserting an I-CeuI-GFP-PI-SceI cassette from pShuttle (Clontech) in its place. The resulting plasmid was cut with FseI and MluI and Ad Pan7 fragment FseI (bp 3618) to MluI (bp 155114 was inserted to extend the left end. The construct (pPan7pGFP) was completed by inserting the 21421 bp Ad Pan7 right end fragment from the MluI site (bp 15114) into the above plasmid between MluI and EcoRV to generate a complete molecular clone of E1 deleted adenovirus Pan7 suitable for the generation of recombinant adenoviruses. Optionally, a desired transgene is inserted into the I-CeuI and PI-SceI sites of the newly created pPan7 vector plasmid.

B. Construction of E1-Deleted Pan7 Viral Vectors

To generate the recombinant adenoviruses from pPan7)E1, the plasmid is co-transfected with a helper expressing E1, or from an E1-expressing packaging cell line, such as 293 cell line or a cell line prepared as described herein. The expression of E1 in the packaging cell permits the replication and packaging of the Pan7)E1 into a virion capsid. In another embodiment, the packaging cell transfected with pX′Pan7)E1 is transfected with an adenovirus vector as described above bearing the transgene of interest. Homologous recombination occurs between the helper and the plasmid, which permits the adenovirus-transgene sequences in the vector to be replicated and packaged into virion capsids, resulting in the recombinant adenovirus. Transfection and purification is as described above.

### Example 22

### Generation of Plasmid Vectors Expressing the E1 Genes

Plasmid vectors are constructed which encode the Pan8 E1 region gene, and these plasmids are used to generate stable cell lines expressing viral E1 proteins.

The E1 region of Pan8 is cloned into pX′, essentially as described in Example 19 above, prior to replacement of this region with the fragment from pShuttle (Clontech). The expression plasmid contains the Pan8 adenoviral genome sequence spanning at least by 1 to 3959 in the Pan8 genomic sequence. Thus, the expression plasmid contains the sequence encoding E1a and E1b of chimpanzee Ad Pan8 under the control of a heterologous promoter. Similar expression plasmids can be generated using the Ad Pan6 and AdPan 7 E1 regions, identified in the tables above.

### Example 23

### Generation of Cell Lines Expressing Chimpanzee Adenovirus E1 Proteins

Cell lines expressing viral E1 proteins are generated by transfecting HeLa (ATCC Acc. No. CCL2) with the plasmid of Example 21. These cell lines are useful for the production of E1-deleted recombinant chimpanzee adenoviruses by co-transfection of genomic viral DNA and the expression plasmids described above. Transfection of these cell lines, as well as isolation and purification of recombinant chimpanzee adenoviruses therefrom are performed by methods conventional for other adenoviruses, i.e., human adenoviruses [see, e.g., Horwitz, cited above and other standard texts].

A. Cell lines expressing Pan5 E1 Proteins

HeLa cells in 10 cm dishes are transfected with 10 μg of pX-Pan51-E1 DNA using a Cellphect™ kit (Pharmacia, Uppsala, Sweden) and following the manufacturer's protocol. 22 hours post-transfection, the cells are subjected to a three minute glycerol shock (15% glycerol in Hepes Buffered Saline, pH 7.5) washed once in DMEM (HeLa) or F12K (A549; Life Technologies, Inc., Grand Island, N.Y.) media supplemented with 10% FCS, 1% Pen-Strep, then incubated for six hours at 37° C. in the above described media. The transfected cells are then split into duplicate 15 cm plates at ratios of 1:20, 1:40, 1:80, 1:160, and 1:320. Following incubation at 37° C. overnight, the media is supplemented with G418 (Life Technologies, Inc.) at a concentration of 1 μg/ml. The media is replaced every 5 days and clones are isolated 20 days post-transfection.

HeLa E1 cell clones are isolated and assayed for their ability to augment adeno-associated virus (AAV) infection and expression of recombinant LacZ protein as described below.

B. AAV Augmentation Assay for Screening E1 Expressing Cell Lines

AAV requires adenovirus-encoded proteins in order to complete its life cycle. The adenoviral E1 proteins as well as the E4 region-encoded ORF6 protein are necessary for the augmentation of AAV infection. An assay for E1 expression based on AAV augmentation is used. Briefly, the method for identifying adenoviral E1-expressing cells comprises the steps of infecting in separate cultures a putative adenovirus E1-expressing cell and a cell containing no adenovirus sequence, with both an adeno-associated virus (AAV) expressing a marker gene and an AAV expressing the ORF6 of the E4 gene of human adenovirus, for a suitable time. The marker gene activity in the resulting cells is measured and those cells with significantly greater measurable marker activity than the control cells are selected as confirmed E1-expressing cells. In the following experiment, the marker gene is a lacZ gene and the marker activity is the appearance of blue stain.

For example, the cell lines described above, as well as untransfected control cells (HeLa) are infected with 100 genomes per cell of an AAV vector bearing a marker gene, e.g., AV.LacZ [K. Fisher et al., J. Virol., 70:520 (1996)] and an AAV vector expressing the ORF6 region of human 5 (AV.orf6). The DNA sequence of the plasmid generates a novel recombinant adeno-associated virus (rAAV) containing the LacZ transgene and the Ad E4ORF 6, which is an open reading frame whose expression product facilitates single-stranded (ss) to double-stranded (ds) conversion of rAAV genomic DNA. These vectors are incubated in medium containing 2% FCS and 1% Pen-Strep at 37° C. for 4 hours, at which point an equal volume of medium containing 10% FCS is added. It should be understood by one of skill in the art that any marker gene (or reporter gene) may be employed in the first AAV vector of this assay, e.g., alkaline phosphatase, luciferase, and others. An antibody-enzyme assay can also be used to quantitate levels of antigen, where the marker expresses an antigen. The assay is not limited by the identity of the marker gene. Twenty to twenty-four hours post-infection, the cells are stained for LacZ activity using standard methods. After 4 hours the cells are observed microscopically and cell lines with significantly more blue cells than the A549 or HeLa cell controls are scored as positive.

### Example 24

### Delivery of Transgene to Host Cell

The resulting recombinant chimpanzee adenovirus described in Example 19, or 21 above is then employed to deliver the transgene to a mammalian, preferably human, cell. For example, following purification of the recombinant virus, human embryonic kidney 293 cells are infected at an MOI of 50 particles per cell. GFP expression was documented 24 hours post-infection.

A. Gene Transfer in Mouse Models via Pan-6, Pan-7, and Pan-9 Vectors

Gene transfer efficiencies and toxicological profile of recombinant chimpanzee adenoviruses were compared in mouse liver directed gene transfer, mouse lung directed gene transfer, and mouse muscle directed gene transfer.

E1-deleted adenoviral vectors containing LacZ under the control of the CMV promoter were constructed using the techniques herein for human Ad5, chimpanzee Pan 6, chimpanzee Pan 7 and chimpanzee Pan 9 (C68). The vectors were delivered to immune-deficient NCR nude mice (80 for each study) as follows. For the liver study, 100 μl (1×10¹¹ particles) were injected into the tail vein. For the lung study, 50 μl (5×10¹⁰ particles) were delivered intratracheally. For the muscle study, 25 μl (5×10¹⁰ particles) were injected into tibialis anterior. The mice were sacrificed on days 3, 7, 14 and 28 post-vector injection (5 animals per group at each time point). At each necropsy, the liver/lung/muscle tissue was harvested and prepared for cryoblocks and paraffin embedding. The cryoblocks were sectioned for X-gal staining and the paraffin sections are H&E stained for histopathic analysis. At each time point, terminal bleeding was performed. Serum samples were subjected to liver function tests.

It was observed in this experiment the chimpanzee adenoviruses Pan-6, Pan-7, and Pan-9 were less efficient than huAd5 in gene transfer to the liver and to the lung. However, this may be desirable in certain circumstances, to reduce liver toxicity observed for huAd5. The gene transfer efficiency in muscle varied less between serotypes.

B. Mouse Study to Feasibility of Re-Administration of Adenovirus Vectors by Serotype Switching Between Adhu5, Pan-6, Pan-7, and Pan-9 Vectors

Mice were administered (C57/B16; 4/group) LacZ vectors based on huAd5, Pan-6, Pan-7, and Pan-9 (H5.040CMVLacZ, Pan6.000CMVLacZ, Pan7.000CMVLacZ, Pan9.000CMVLacZ; 10¹¹ particles/injection) by tail vein. Thirty days later the mice were re-administered adenovirus vectors expressing α1-antitrypsin (H5.040CMVhA1AT, Pan6.000CMVhA1AT, 1×10¹¹ particles, Pan7.000CMVhA1AT, Pan9.000CMVhA1AT, 10¹¹ particles/injection). Successful transduction by the re-administered vector is monitored by measuring serum α1-antitrypsin on days 3 and 7, following re-administration.

The ability of adenovirus vectors based on huAd5, Pan-6, Pan-7, and Pan-9 respectively to transduce the livers of mice in the presence of neutralizing antibodies to the other serotypes was determined. The results are tabulated here.

| 1^{st} injection | 2^{nd} injection | Cross-neutralization |
|---|---|---|
| Adhu5 | Adhu5 | Yes (+ve control) |
| | Pan-6 | No |
| | Pan-7 | No |
| | Pan-9 (C68) | No |
| Pan-6 | Adhu5 | No |
| | Pan-6 | Yes (+ve control) |
| | Pan-7 | Yes |
| | Pan-9 (C68) | No |
| Pan-7 | Adhu5 | No |
| | Pan-6 | Yes |
| | Pan-7 | Yes (+ve control) |
| | Pan-9 (C68) | Yes |
| Pan-9 (C68) | Adhu5 | No |
| | Pan-6 | No |
| | Pan-7 | Yes |
| | Pan-9 (C68) | Yes (+ve control) |

C. Ability of Vectors to Transduce Murine Liver in the Presence of Neutralizing Antibodies to Other Serotypes.

Thus, immunization with huAd5 does not prevent re-administration with either of the chimpanzee adenovirus vectors Pan-6, Pan-7, or Pan-9 (C68). This experiment also appears to indicate that Pan-7 is between Pan-6 and Pan-9 in the spectrum of antigenic relatedness and cross-reacts with both; however Pan-6 and Pan-9 do not neutralize each other. This is a surprising result based on homology comparisons, which indicates that Pan-6 is quite distinct from Pan-7 and Pan-9. Evaluation of antisera generated against Pan-9 indicated no cross-neutralization against Pan-6 but some neutralization against Pan-7, arguing that Pan-6 is distinct from the others.

### Example 25

### Generation of Recombinant E1-Deleted SV-25 Vector

A plasmid was constructed containing the complete SV-25 genome except for an engineered E1 deletion. At the site of the E1 deletion recognition sites for the restriction enzymes I-CeuI and PI-SceI which would allow insertion of transgene from a shuttle plasmid where the transgene expression cassette is flanked by these two enzyme recognition sites were inserted.

A synthetic linker containing the restriction sites SwaI-SnaBI-SpeI-AfIII-EcoRV-SwaI was cloned into pBR322 that was cut with EcoRI and NdeI. This was done by annealing together two synthetic oligomers SV25T (5′-AAT TTA AAT ACG TAG CGC ACT AGT CGC GCT AAG CGC GGA TAT CAT TTA AA-3′, SEQ ID NO: 38) and SV25B (5′-TAT TTA AAT GAT ATC CGC GCT TAA GCG CGA CTA GTG CGC TAC GTA TTT A-3′, SEQ ID NO:39) and inserting it into pBR322 digested with EcoRI and NdeI. The left end (bp 1 to 1057, SEQ ID NO:29) of Ad SV25 was cloned into the above linker between the SnaBI and SpeI sites. The right end (bp28059 to 31042, SEQ ID NO: 29) of Ad SV25 was cloned into the linker between the AfIII and EcoRV sites. The adenovirus E1 was then excised between the EcoRI site (bp 547) to XhoI (bp 2031) from the cloned left end as follows. A PCR generated I-CeuI-PI-SceI cassette from pShuttle (Clontech) was inserted between the EcoRI and SpeI sites. The 10154 bp XhoI fragment of Ad SV-25 (bp2031 to 12185, SEQ ID NO:29) was then inserted into the SpeI site. The resulting plasmid was digested with HindIII and the construct (pSV25) was completed by inserting the 18344 bp Ad SV-25 HindIII fragment (bp11984 to 30328, SEQ ID NO:29) to generate a complete molecular clone of E1 deleted adenovirus SV25 suitable for the generation of recombinant adenoviruses. Optionally, a desired transgene is inserted into the I-CeuI and PI-SceI sites of the newly created pSV25 vector plasmid.

To generate an AdSV25 carrying a marker gene, a GFP (green fluorescent protein) expression cassette previously cloned in the plasmid pShuttle (Clontech) was excised with the restriction enzymes I-CeuI and PI-SceI and ligated into pSV25 (or another of the Ad chimp plasmids described herein) digested with the same enzymes. The resulting plasmid (pSV25GFP) was digested with SwaI to separate the bacterial plasmid backbone and transfected into the E1 complementing cell line HEK 293. About 10 days later, a cytopathic effect was observed indicating the presence of replicative virus. The successful generation of an Ad SV25 based adenoviral vector expressing GFP was confirmed by applying the supernatant from the transfected culture on to fresh cell cultures. The presence of secondarily infected cells was determined by observation of green fluorescence in a population of the cells.

### Example 26

### Construction of E3 Deleted Pan-5, Pan-6, Pan-7 and C68 Vectors

In order to enhance the cloning capacity of the adenoviral vectors, the E3 region can be deleted because this region encodes genes that are not required for the propagation of the virus in culture. Towards this end, E3-deleted versions of Pan-5, Pan-6, Pan-7, and C68 have been made (a 3.5 kb Nru-AvrII fragment containing E31-9 is deleted).

A. E3 Deleted Pan5 Based Vector

E1-deleted pPan5-pkGFP plasmid was treated with Avr II endonuclease to isolate a 5.8 kb fragment containing the E3 region and re-circulate pPan5-pkGFP with Avr II deletion to form construct pPan5-pkGFP-E3-Avr II. Subsequently, the 5.8 kb Avr II fragment was subcloned into pSL-Pan5-E3-Avr II for a further deletion of E3 region by Nru I digestion. This led to a plasmid pSL-Pan5-E3-deletion. The final construct pPan5-E3-pkGFP was produced by removing a 4.3 kb Avr II/Spe I fragment from pSL-Pan5-E3-deletion plasmid and inserting into pPan5-pkGFP-E3-Avr II at Avr II site. In this final construct, a 3.1 kb deletion in E3 region was accomplished.

B. E3 deletion in Pan6 Based Vector

E1-deleted pPan6-pkGFP molecular clone was digested with Sbf I and Not Ito isolate 19.3 kb fragment and ligated back at Sbf I site. The resulting construct pPan6-Sbf I-E3 was treated with Eco 47 III and Swa I, generating pPan6-E3. Finally, 21 kb Sbf I fragment from Sbf I digestion of pPan6-pkGFP was subcloned into pPan6-E3 to create pPan6-E3-pkGFP with a 4 kb deletion in E3.

C. E3 Deleted Pan 7 and Pan9 Vectors

The same strategy was used to achieve E3 deletions in both vectors. First, a 5.8 kb Avr II fragment spanning the E3 region was subcloned pSL-1180, followed by deletion of E3 bp Nru I digestion. The resulting plasmids were treated with Spe I and Avr II to obtain 4.4 kb fragments and clone into pPan7-pkGFP and pPan9-pkGFP at Avr II sites to replace the original E3 containing Avr II fragments, respectively. The final pPan7-E3-pkGFP and pPan9-E3-pkGFP constructs have 3.5 kb E3-deletions.

### Example 27

### Construction of E3- and E4-Deleted Pan-7 Vector

Although the deletion of the E1 region of adenoviruses (first generation adenovirus vectors) renders them replication-incompetent, expression of the adenoviral vector backbone genes is not fully abolished. Deletion of the E4 region considerably attenuates this residual gene expression and may confer a safety advantage. An E4-deleted Pan-7 vector containing a 2.5 kb deletion (a PvuII-AgeI fragment containing E4ORF1-ORF7 is deleted) has been constructed. High titer stocks of this virus were generated using a HEK 293-based cell line, which in addition to E1, expresses an essential E4 gene (orf 6).

1. E4 deletion in the molecular clone of Pan7

A 19 kb Xba I fragment was deleted from pPan7-pkGFP to create pPan7-Xba I from which a 2.5 kb E4 fragment was deleted by Age I and Pvu II partial digestion, resulting in pPan7-Xba I-E4. pPan7-E4-pkGFP plasmid was generated from pPan7-Xba I-E4 in two sequential cloning steps, adding 19 kb Xba I and 15 kb I-Ceu I/Mlu

I fragments, both of which came from pPan7-pkGFP construct.

2. Introduction of E3 and E4 Deletions in Pan9 Vector

An 11 kb plasmid, pPan9-EcoRI, containing E4 region was created by retrieving 11 kb EcoRI fragment from pPan9 pkGFP after EcoRI digestion and self-ligation. E4 region was deleted from this construct by Age I digestion/filled in and Pvu II partial digestion and slef-ligation to generate pPan9-EcoR I-E4. A 23 kb EcoR I fragment was isolated from pPan9-pkGFP and inserted into pPan9-EcoR I-E4 at EcoR I site, followed by adding 5.8 kb Avr II fragment from pPan9-pkGFP, to form the final product pPan9-E3-E4-pkGF. Compared to the genome size of wild type Pan9, this E1-E3-E4-deleted vector could have a transgene capacity up to 8 kb.

3. Introduction of Minigene Cassettes with Genes of Interest Including Reporter Genes, Glyco- and Nuclear Proteins of Ebo into Molecular Clones of Pan Vectors

A highly efficient direct cloning and green/white selection procedure was employed for creating molecular clones of recombinant viruses. Briefly, genes of interest were cloned into pShuttlepkGFP by screening white colonies for recombinants. Subsequently, the minigene cassettes were transferred into chimpanzee adenovirus backbone plasmids, pPanX-pkGFP with various deletions, easily by swapping with pkGFP cassette at I-Ceu I and PI-Sce I sites and screening a few white colonies for correct recombinants.

4. Rescue of Molecular Clones of Pan Vectors with Multiple Deletions in Early Regions and Virus Propagation

For rescue of E1-E3-deleted molecular clones of chimpanzee adenovirus vectors, the clones were linearized with appropriate restriction enzymes and transfected into regular 293 cells. Once a full cytopathic effect (CPE) observed in the transfected cells, crude lysate was harvested and expanded in 293 cells to large-scale infections. The viruses were purified by CsCl sedimentation method.

For E1-E4 and E1-E3-E4-deleted Pan vectors, 10⁻³ cells, a 293-based E1-E4-complementing cell line, were used for rescue and propagation of vectors. E4 ORF6 gene expression in 10⁻³ cells was induced by addition of 150 μM ZnSO₄ to the culture medium.

### Example 28

### Vaccination with Adenovirus Vectors Expressing Wild Type and Variant EboZ GP

AdHu5 or AdC7 vectors expressing Ebola envelope chimeras were produced for in vivo immunization experiments in C57BL/6 mice. Recombinant viruses with different viral backbones were created by molecular cloning method in which the minigene cassettes were inserted into the place of E1-deletions. The molecular clones of all recombinant viruses were rescued and grown up in 293 cells for large-scale purification using CsCl sedimentation method. Five EboZ variants encoded by AdHu5 or AdPan7 (C7) were selected and produced to evaluate their relative immunogenicity following an intramuscular Ad injection. The wt Ebo, a soluble Ebo variant, EboΔ1, EboΔ2, EboΔ3, EboΔ4, EboΔ5S, EboΔ6S, EboΔ7S and EboΔ8S were evaluated in the initial vaccine studies. For the data summarized in the following table, the number of viral particles (per ml or total) produced and amplified from infected 293 cells was established by spectrophotometry reading.

TABLE
Production of Adhu5 or AdC7 Adenoviral vector encoding EboZ variant.

| | |
|---|---|
| HuAd5 | AdC7 |

| | | Total | | Total |
|---|---|---|---|---|
| | Titer | yield | Titer | yield |
| | (VP × | (VP × | (VP × | (VP × |
| Gene | 10¹²/ml) | 10¹²) | 10¹²/ml) | 10¹²) |
| Ebo wt | 2.6 | 12 | 4.3 | 43 |
| EboS | 4.9 | 49 | 4.6 | 55 |
| EboΔ2 | 2.1 | 9 | 5.8 | 93 |
| EboΔ3 | 1.7 | 8 | 5.3 | 95 |
| EboΔ4 | 3 | 12 | 4.1 | 62 |

Vector was administered intramuscularly (10¹¹ genome copies/cell) in C57BL/6 mice and the presence of virus neutralizing antibody (VNA0 was evaluated 28 days later as a first measure of an immune response generated against the Ebola envelope glycoprotein. VNA is defined here as serum antibody able to inhibit transduction of HeLa cells mediated by HIV-based vector pseudotyped with the wild-type Ebola envelope.

VNA to the EboZ pseudotypes was detected with AdPan7 (C7) yielding higher titers than AdHu5. The EboZΔ3 elicited the highest VNA in terms of the transgene targets. For the data summarized in the following table, neutralizing antibody titers to HIV-EboZ-GFP pseudotypes (reciprocal dilution) are provided (N=5 animals/group).

| | VNA Titers | | |
|---|---|---|---|
| | EboZ wildtype | EboZs | EboZΔ3 |
| AdHu5 | 12 | 16 | 12 |
| AdC7 | 44 | 12 | 140 |

### Example 29

### Pan7-Mediated Expression of Ebola Proteins Mouse studies to evaluate Pan-7 vectors expressing Ebola envelope proteins and the Ebola nuclear antigen have been initiated. These are directed towards evaluation of neutralizing antibodies in C57B1/6 mice injected intramuscularly (IM) with Adhu5 or Pan-7 expressing each of 4 Ebola env constructs.

A. Evaluation of CTL from C57B1/6 Mice Injected IM with Adhu5 or Pan-7 Expressing the Ebola Env Constructs.

1. Challenge Experiment in Mice with Ebola Virus.

Neutralizing antibody (NAB) responses to the Ebola envelope were analyzed by looking at immunized mouse sera mediated neutralization of a lentiviral (HIV) vector pseudotyped with the several constructs (eEbo, NTD2, NTD3, NTD4) of the Ebola envelope glycoprotein. C57BL/6 or BALB/c mice received a single intramuscular injection of 5×10¹⁰ particles per mouse of C7 (Ad Pan-7) encoding Ebola envelope variant. Neutralizing antibody was evaluated 30 days post-vaccination. Briefly, Ebola Zaire pseudotyped HIV vector encoding for β-galactosidase (EboZ-HIV-LacZ) was incubated for 2 hr at 37° C. with different dilution of heat inactivated mouse serum. Following the incubation with serum, EboZ-HIV-LacZ was then used to infect HeLa cells for 16 hr at 37° C. Infectivity was revealed by X-gal staining of transduced HeLa cells positive for β-galactosidase. Neutralizing titer represent the serum reciprocal dilution where a 50% decrease in the number of β-galactosidase positive blue cells was observed.

Sera were collected 30 days post-immunization, which consisted in a single intramuscular (I.M.) administration of 5×10¹⁰ particles/animal. Neutralizing antibody to Ebola pseudotyped HIV could be detected from all groups with antibody titers ranging from 20 for Ad-EboZ (Adhu5 expressing EboZ), Ad-NTD3 (Adhu5 expressing NTD3) and C7-sEbo (Ad Pan-7 expressing soluble EboZ) to over 130 for C7-NTD3 (Ad Pan-7 expressing soluble NTD3) and C7-NTD4 (Ad Pan-7 expressing soluble NTD3). The same immunization strategy in BALB/c mice resulted in lower neutralizing antibody titers for Ad- and C7-NTD2, and NTD4.

B. Cellular Immune Response

The cellular immune response to the Ebola envelope in C57BL/6 mice was evaluated 8 days after a single I.M. administration of 5×10¹⁰ particles of C7-LacZ or C7-Ebola envelope variant per animal. Mice were vaccinated I.M. with 5×10¹⁰ particles of C7 encoding LacZ or Ebola envelope variant. Splenic lymphocytes from immunized mice were collected 8 days post vaccination and stimulated in vitro with feeder cells (splenic lymphocytes from untreated mice infected with human Adenovirus serotype 5 encoding for the wild-type Ebola envelope and irradiated). Standard 5-hr CTL assays were performed using ⁵¹Cr-labeled syngenic C57 cells transfected with an expressor of EboZ.

A positive MHC-restricted cytotoxic T lymphocyte (CTL) response was observed from all AdPan-7 encoding for Ebola envelope variants with a higher response from NTD2, NTD3 or NTD4 immunized mice. Indeed, effector cells from C7 encoding Ebola envelope variant immunized mice recognized EboZ transfected target cells and gave recall CTL responses up to 30% specific lysis. Less than 5% lysis was seen with effector cells from naïve or LacZ immunized control mice confirming that lysis was specific for Ebola envelope antigens.

C. Protection Studies

The most direct means of evaluating C7 (Ad Pan-7) encoding for the EboZ variants as a successful vaccine in mice was to assess protection against weight loss and death following lethal challenge with mouse adapted Ebola Zaire virus. BALB/c mice were immunized with a single dose of 5×10¹⁰ particles per animal as performed previously and vaccinated animals were challenged with 200 LD₅₀ of mouse adapted Ebola Zaire 21 days later. All control mice (vehicle and C7-LacZ) died between day 5 to day 9 post-challenge. In contrast, all vaccinated mice but one, (from the C7-sEbo group), survived the challenge with Ebola Zaire.

Weight loss was observed from mice vaccinated with C7-sEbo from day 4 to day 7. Signs of illness such as pilo-erection and from light to severe lethargy were also noted from mice vaccinated with C7-sEbo, NTD2 and NTD3 between day 4 to day 7. Mice immunized with C7-EboZ and C7-NTD4 did not show sign of illness. Overall, a single dose of C7-EboZ and C7-NTD4 completely protected immunized mice from illness and death possibly due to a significant T cell mediated immunity.

All publications cited in this specification, and the sequence listing, are incorporated herein by reference. While the invention has been described with reference to particular embodiments, it will be appreciated that modifications can be made without departing from the spirit of the invention. Such modifications are intended to fall within the scope of the appended claims.

What is claimed is:

1. A replication defective simian adenoviral Pan7 particle comprising a minigene containing adenoviral sequences comprising simian adenoviral cis-elements and a heterologous gene operably linked to expression control sequences, said minigene packaged in an adenovirus Pan7 capsid, wherein the simian adenoviral particle is replication defective due to the absence of the ability to express adenoviral E1a and E1b.

2. The simian adenoviral particle according to claim 1 wherein the delayed early gene E3 is eliminated.

3. The simian adenoviral particle according to claim 1 having a functional deletion in the E4 gene.

4. The simian adenoviral particle according to claim 1 which contains a deletion in the delayed early gene E2a.

5. The simian adenoviral particle according to claim 1 having a deletion in any of the late genes L1 to L5 of the simian adenoviral genome.

6. The simian adenoviral particle according to claim 1 wherein the heterologous gene is directed to the prevention and treatment of disease caused by a virus selected from the group consisting of Human immunodeficiency virus, Simian immunodeficiency virus, Respiratory syncytial virus, Parainfluenza virus types 1-3, Influenza virus, Herpes simplex virus, Human cytomegalovirus, hepatitis viruses, Human papillomavirus, poliovirus, rotavirus, caliciviruses, Measles virus, Mumps virus, Rubella virus, adenovirus, rabies virus, canine distemper virus, rinderpest virus, coronavirus, parvovirus, infectious rhinotracheitis viruses, feline leukemia virus, feline infectious peritonitis virus, avian infectious bursal disease virus, Newcastle disease virus, Marek's disease virus, porcine respiratory and reproductive syndrome virus, equine arteritis virus and Encephalitis viruses.

7. The simian adenoviral particle according to claim 1 wherein the heterologous gene is directed to the prevention and treatment of disease caused by a bacterium selected from the group consisting of Haemophilus influenzae, Haemophilus somnus, Moraxella catarrhalis, Streptococcus pneumoniae, Streptococcus pyogenes, Streptococcus agalactiae, Streptococcus faecalis, Helicobacter pylori, Neisseria meningitidis, Neisseria gonorrhoeae, Chlamydia trachomatis, Chlamydia pneumoniae, Chlamydia psittaci, Bordetella pertussis, Salmonella typhi, Salmonella typhimurium, Salmonella choleraesuis, Escherichia coli, Shigella, Vibrio cholerae, Corynebacterium diphtheriae, Mycobacterium tuberculosis, Mycobacterium avium, Mycobacterium intracellulare complex, Proteus mirabilis, Proteus vulgaris, Staphylococcus aureus, Clostridium tetani, Leptospira interrogans, Borrelia burgdorferi, Pasteurella haemolytica, Pasteurella multocida, Actinobacillus pleuropneumoniae and Mycoplasma gallisepticum.

8. The simian adenoviral particle according to claim 1 wherein the heterologous gene is directed to the prevention and treatment of disease caused by a fungus selected from the group consisting of Aspergillis, Blastomyces, Candida, Coccidiodes, Cryptococcus and Histoplasma.

9. The simian adenoviral particle according to claim 1 wherein the heterologous gene is directed to the prevention and treatment of disease caused by a parasite selected from the group consisting of Leishmania major, Ascaris, Trichuris, Giardia, Schistosoma, Cryptosporidium, Trichomonas, Toxoplasma gondii and Pneumocystis carinii.

10. The simian adenoviral particle according to claim 1 wherein the heterologous gene is directed to eliciting an anti-cancer effect utilizing a cancer antigen or tumor-associated antigen selected from the group consisting of prostate specific antigen, carcino-embryonic antigen, MUC-1, Her2, CA-125 and MAGE-3.

11. A method of producing a simian adenoviral particle according to claim 1.

12. A method of delivering a therapeutic or immunogenic molecule comprising administering a simian adenoviral particle according to claim 1, wherein the heterologous gene is or encodes the therapeutic or immunogenic molecule.

13. A composition comprising a delivery vehicle and a replication defective simian adenoviral particle comprising a minigene containing adenoviral sequences comprising simian adenoviral cis-elements and a heterologous gene operably linked to expression control sequences, said minigene packaged in an adenovirus Pan7 capsid, wherein the simian adenoviral particle is replication defective due to the absence of the ability to express adenoviral E1a and E1b.

14. The composition according to claim 13, wherein the composition further comprises one or more adjuvants, stabilizers, or pH adjusters.

15. The composition according to claim 13, wherein the composition comprises an antigenic protein in combination with the adenoviral particle.
